SEQUENCE LISTING (127 sequences)

SEQ ID NO: 1 - protein, 10 aa, synthetic construct
GVYDGREHTV

SEQ ID NO: 2 - protein, 9 aa, synthetic construct
KLVELEHTL

SEQ ID NO: 3 - protein, 9 aa, synthetic construct
SPSSASLAL

SEQ ID NO: 4 - protein, 10 aa, synthetic construct
KPRPDVTNEL

SEQ ID NO: 5 - protein, 10 aa, synthetic construct
KEFAFLEHSL

SEQ ID NO: 6 - protein, 9 aa, synthetic construct
HELGFKVVL

SEQ ID NO: 7 - protein, 8 aa, synthetic construct
LKPDHIQR

SEQ ID NO: 8 - protein, 9 aa, synthetic construct
SLFGARPGR

SEQ ID NO: 9 - protein, 9 aa, synthetic construct
KTEVHGRLK

SEQ ID NO: 10 - protein, 9 aa, synthetic construct
NLRPPTQEL

SEQ ID NO: 11 - protein, 10 aa, synthetic construct
RVSLPKLGYK

SEQ ID NO: 12 - protein, 8 aa, synthetic construct
LRPPTQEL

SEQ ID NO: 13 - protein, 9 aa, synthetic construct
RPASPRPAP

SEQ ID NO: 14 - protein, 9 aa, synthetic construct
IATKIALQM

SEQ ID NO: 15 - protein, 9 aa, synthetic construct
SLYQTIRLK

SEQ ID NO: 16 - protein, 9 aa, synthetic construct
KTPLHTLLK

SEQ ID NO: 17 - protein, 10 aa, synthetic construct
RLHSFTLRQK

SEQ ID NO: 18 - protein, 10 aa, synthetic construct
RVFTSSLKTK

SEQ ID NO: 19 - protein, 9 aa, synthetic construct
RVLAKGLAK

SEQ ID NO: 20 - protein, 10 aa, synthetic construct
GPRPSPTRSV

SEQ ID NO: 21 - protein, 9 aa, synthetic construct
GQHLHLETF

SEQ ID NO: 22 - protein, 9 aa, synthetic construct
SPRSPSPSL

SEQ ID NO: 23 - protein, 10 aa, synthetic construct
VPQEAVRAPL

SEQ ID NO: 24 - protein, 8 aa, synthetic construct
IYPFINSH

SEQ ID NO: 25 - protein, 9 aa, synthetic construct
SPSSASLTL

SEQ ID NO: 26 - protein, 8 aa, synthetic construct
LNKVKTSL

SEQ ID NO: 27 - protein, 8 aa, synthetic construct
NLKTHLRL

SEQ ID NO: 28 - protein, 9 aa, synthetic construct
FRGVFVHRY

SEQ ID NO: 29 - protein, 9 aa, synthetic construct
TSGPVTEKY

SEQ ID NO: 30 - protein, 9 aa, synthetic construct
VVAAHLAGA

SEQ ID NO: 31 - protein, 9 aa, synthetic construct
KSYSKVLVR

SEQ ID NO: 32 - protein, 9 aa, synthetic construct
MTYKIHFKK

SEQ ID NO: 33 - protein, 9 aa, synthetic construct
VPASPALSR

SEQ ID NO: 34 - protein, 9 aa, synthetic construct
SPSARPPSL

SEQ ID NO: 35 - protein, 9 aa, synthetic construct
SPTLNVSAL

SEQ ID NO: 36 - DNA, 686 bp, synthetic construct
  source 1..686: mol_type = other DNA
cctttcactcattagatgcatgtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccggttaagcttggtacc

SEQ ID NO: 37 - DNA, 12 bp, synthetic construct
  source 1..12: mol_type = other DNA
cccgccgccacc

SEQ ID NO: 38 - skipped

SEQ ID NO: 39 - DNA, 30 bp, synthetic construct
  source 1..30: mol_type = other DNA
ggagtttacgatggcagagagcacaccgtc

SEQ ID NO: 40-42 - skipped (3 entries)

SEQ ID NO: 43 - DNA, 303 bp, synthetic construct
  source 1..303: mol_type = other DNA
gcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 44 - DNA, 597 bp, synthetic construct
  source 1..597: mol_type = other DNA
ttaattaacatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagtcgaagcttggatccggtacctctagaattctcgagcggccgctagcgacatcgatcacaagtttgtacaaaaaagcaggctccaccatggtaactataacggtcctaaggtagcgaagaaccaattcagtcgag

SEQ ID NO: 45 - DNA, 30671 bp, synthetic construct
  source 1..30671: mol_type = other DNA
cttctgaggcggaaagaaccagctggggctcgaggggggatcgatccgtcgagatatctagggataacagggtaattagacccagctttcttgtacaaagtggtgatcgattcgacagatcactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcgaatcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttaattaa

SEQ ID NO: 46 - DNA, 32305 bp, synthetic construct
  source 1..32305: mol_type = other DNA
ttaattaacatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagtcgaagcttggatccggtacctctagaattctcgagcggccgctagcgacatcgatcacaagtttgtacaaaaaagcaggctccaccatggtaactataacggtcctaaggtagcgaagaaccaattcagtcgagcctttcactcattagatgcatgtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccggttaagcttggtacccccgccgccaccatgggagtttacgatggcagagagcacaccgtctgagcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgaggggggatcgatccgtcgagatatctagggataacagggtaattagacccagctttcttgtacaaagtggtgatcgattcgacagatcactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcgaatcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttaattaa

SEQ ID NO: 47 - DNA, 270 bp, synthetic construct
  source 1..270: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagctagttattaatagtaatcaattacggggtcattagttcatagcccatatatggagttccg

SEQ ID NO: 48 - DNA, 217 bp, synthetic construct
  source 1..217: mol_type = other DNA
ctcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 49 - DNA, 1706 bp, synthetic construct
  source 1..1706: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagctagttattaatagtaatcaattacggggtcattagttcatagcccatatatggagttccgacaagtttgtacaaaaaagcaggctccaccatggtaactataacggtcctaaggtagcgaagaaccaattcagtcgagcctttcactcattagatgcatgtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccggttaagcttggtacccccgccgccaccatgggagtttacgatggcagagagcacaccgtctgagcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgaggggggatcgatccgtcgagatatctagggataacagggtaattagacccagctttcttgtacaaagtggtctcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 50 - DNA, 130 bp, synthetic construct
  source 1..130: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 51 - DNA, 130 bp, synthetic construct
  source 1..130: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 52 - DNA, 30 bp, synthetic construct
  source 1..30: mol_type = other DNA
ggcgtttacgacggcagagaacacaccgtg

SEQ ID NO: 53 - DNA, 27 bp, synthetic construct
  source 1..27: mol_type = other DNA
aagctggtggaactggaacacaccctg

SEQ ID NO: 54 - DNA, 27 bp, synthetic construct
  source 1..27: mol_type = other DNA
tctcctagctctgcctctctggccctg

SEQ ID NO: 55 - DNA, 30 bp, synthetic construct
  source 1..30: mol_type = other DNA
aagcccagacctgacgtgaccaacgagctg

SEQ ID NO: 56 - DNA, 30 bp, synthetic construct
  source 1..30: mol_type = other DNA
aaggaattcgccttcctggaacacagcctg

SEQ ID NO: 57 - DNA, 27 bp, synthetic construct
  source 1..27: mol_type = other DNA
cacgagctgggcttcaaggtggtgctg

SEQ ID NO: 58 - DNA, 24 bp, synthetic construct
  source 1..24: mol_type = other DNA
ctgaagcccgaccacatccagaga

SEQ ID NO: 59 - DNA, 27 bp, synthetic construct
  source 1..27: mol_type = other DNA
agcctgtttggcgccagacctggcaga

SEQ ID NO: 60 - DNA, 27 bp, synthetic construct
  source 1..27: mol_type = other DNA
aagacagaggtgcacggccggctgaag

SEQ ID NO: 61 - DNA, 27 bp, synthetic construct
  source 1..27: mol_type = other DNA
aacctgcggcctcctacacaagagctg

SEQ ID NO: 62 - DNA, 30 bp, synthetic construct
  source 1..30: mol_type = other DNA
agagtgtccctgcctaagctgggctacaag

SEQ ID NO: 63 - DNA, 24 bp, synthetic construct
  source 1..24: mol_type = other DNA
ctcagaccccccactcaggaacta

SEQ ID NO: 64 - DNA, 27 bp, synthetic construct
  source 1..27: mol_type = other DNA
aggcctgcctctcctagacctgctcct

SEQ ID NO: 65 - DNA, 27 bp, synthetic construct
  source 1..27: mol_type = other DNA
atcgctacgaagatcgccctacaaatg

SEQ ID NO: 66 - DNA, 27 bp, synthetic construct
  source 1..27: mol_type = other DNA
agcctgtaccagaccatccggctgaag

SEQ ID NO: 67 - DNA, 27 bp, synthetic construct
  source 1..27: mol_type = other DNA
aaaacccctctccatacacttctcaag

SEQ ID NO: 68 - DNA, 30 bp, synthetic construct
  source 1..30: mol_type = other DNA
cggctccattccttcacgttgcgccagaaa

SEQ ID NO: 69 - DNA, 30 bp, synthetic construct
  source 1..30: mol_type = other DNA
agagtttttacttcgagtctcaagaccaaa

SEQ ID NO: 70 - DNA, 27 bp, synthetic construct
  source 1..27: mol_type = other DNA
cgagtgctagcaaagggcttagcaaag

SEQ ID NO: 71 - DNA, 30 bp, synthetic construct
  source 1..30: mol_type = other DNA
ggcccgcgcccgtccccgacacgctcggtg

SEQ ID NO: 72 - DNA, 27 bp, synthetic construct
  source 1..27: mol_type = other DNA
ggccaacatcttcacttggagacattt

SEQ ID NO: 73 - DNA, 27 bp, synthetic construct
  source 1..27: mol_type = other DNA
agccctagaagcccatctcctagcctg

SEQ ID NO: 74 - DNA, 30 bp, synthetic construct
  source 1..30: mol_type = other DNA
gtgcctcaagaagccgttagagcccctctg

SEQ ID NO: 75 - DNA, 24 bp, synthetic construct
  source 1..24: mol_type = other DNA
atatatccgttcataaattcgcat

SEQ ID NO: 76 - DNA, 27 bp, synthetic construct
  source 1..27: mol_type = other DNA
agtcctagtagtgcctcgctaactctg

SEQ ID NO: 77 - DNA, 24 bp, synthetic construct
  source 1..24: mol_type = other DNA
cttaacaaggtgaaaacaagcttg

SEQ ID NO: 78 - DNA, 24 bp, synthetic construct
  source 1..24: mol_type = other DNA
aaccttaaaactcacttacgactt

SEQ ID NO: 79 - DNA, 27 bp, synthetic construct
  source 1..27: mol_type = other DNA
ttccgaggggtgttcgtacatagatat

SEQ ID NO: 80 - DNA, 27 bp, synthetic construct
  source 1..27: mol_type = other DNA
acgtccggcccggtgacggagaaatat

SEQ ID NO: 81 - DNA, 27 bp, synthetic construct
  source 1..27: mol_type = other DNA
gtagtggccgctcacttggcaggggct

SEQ ID NO: 82 - DNA, 27 bp, synthetic construct
  source 1..27: mol_type = other DNA
aaaagctactcaaaagtgttagtcaga

SEQ ID NO: 83 - DNA, 27 bp, synthetic construct
  source 1..27: mol_type = other DNA
atgacatataaaattcacttcaagaag

SEQ ID NO: 84 - DNA, 27 bp, synthetic construct
  source 1..27: mol_type = other DNA
gtgcctgctagccctgctctgagcaga

SEQ ID NO: 85 - DNA, 27 bp, synthetic construct
  source 1..27: mol_type = other DNA
agcccatcagctaggcctccatcccta

SEQ ID NO: 86 - DNA, 27 bp, synthetic construct
  source 1..27: mol_type = other DNA
agccctacccttaatgtttctgcgctg

SEQ ID NO: 87 - DNA, 30 bp, synthetic construct; identical to SEQ ID NO: 39
  source 1..30: mol_type = other DNA

SEQ ID NO: 88 - DNA, 27 bp, synthetic construct
  source 1..27: mol_type = other DNA
aagctggtggaactggaacacaccctc

SEQ ID NO: 89 - DNA, 27 bp, synthetic construct
  source 1..27: mol_type = other DNA
tctccaagttctgcttctctggccctc

SEQ ID NO: 90 - DNA, 30 bp, synthetic construct
  source 1..30: mol_type = other DNA
aagcctagacctgatgtgaccaacgaactc

SEQ ID NO: 91 - DNA, 30 bp, synthetic construct
  source 1..30: mol_type = other DNA
aaggaatttgcctttctggaacacagcctc

SEQ ID NO: 92 - DNA, 27 bp, synthetic construct
  source 1..27: mol_type = other DNA
cacgagctgggttttaaagtggtgctc

SEQ ID NO: 93 - DNA, 24 bp, synthetic construct
  source 1..24: mol_type = other DNA
ctgaagcctgatcacattcagaga

SEQ ID NO: 94 - DNA, 27 bp, synthetic construct
  source 1..27: mol_type = other DNA
tctctgtttggagctagacctggcaga

SEQ ID NO: 95 - DNA, 27 bp, synthetic construct
  source 1..27: mol_type = other DNA
aagacagaagttcacggccggctgaaa

SEQ ID NO: 96 - DNA, 27 bp, synthetic construct
  source 1..27: mol_type = other DNA
aatctgaggcctccaacacaagaactc

SEQ ID NO: 97 - DNA, 30 bp, synthetic construct
  source 1..30: mol_type = other DNA
agagtgtctctgcctaagctgggctacaaa

SEQ ID NO: 98 - DNA, 24 bp, synthetic construct
  source 1..24: mol_type = other DNA
ctgaggcctcctacacaagagctg

SEQ ID NO: 99 - DNA, 27 bp, synthetic construct
  source 1..27: mol_type = other DNA
aggcctgcttctccaagacctgctcct

SEQ ID NO: 100 - DNA, 27 bp, synthetic construct
  source 1..27: mol_type = other DNA
atcgccacaaagatcgccctgcagatg

SEQ ID NO: 101 - DNA, 27 bp, synthetic construct
  source 1..27: mol_type = other DNA
agcctgtaccagaccatcagactgaaa

SEQ ID NO: 102 - DNA, 27 bp, synthetic construct
  source 1..27: mol_type = other DNA
aagacccctctgcacaccctgctgaag

SEQ ID NO: 103 - DNA, 30 bp, synthetic construct
  source 1..30: mol_type = other DNA
agactgcacagcttcaccctgcggcagaag

SEQ ID NO: 104 - DNA, 30 bp, synthetic construct
  source 1..30: mol_type = other DNA
agagtgttcaccagcagcctcaagaccaag

SEQ ID NO: 105 - DNA, 27 bp, synthetic construct
  source 1..27: mol_type = other DNA
agagtgctggccaaaggactggccaag

SEQ ID NO: 106 - DNA, 30 bp, synthetic construct
  source 1..30: mol_type = other DNA
ggccctagaccttctccaaccagaagcgtg

SEQ ID NO: 107 - DNA, 27 bp, synthetic construct
  source 1..27: mol_type = other DNA
ggacagcatctgcacctggaaaccttc

SEQ ID NO: 108 - DNA, 27 bp, synthetic construct
  source 1..27: mol_type = other DNA
tctccaagatcaccttctccaagcctc

SEQ ID NO: 109 - DNA, 30 bp, synthetic construct
  source 1..30: mol_type = other DNA
gttcctcaagaagctgttagagccccactc

SEQ ID NO: 110 - DNA, 24 bp, synthetic construct
  source 1..24: mol_type = other DNA
atctaccccttcatcaacagccac

SEQ ID NO: 111 - DNA, 27 bp, synthetic construct
  source 1..27: mol_type = other DNA
tctcctagcagcgcctctctgacactg

SEQ ID NO: 112 - DNA, 24 bp, synthetic construct
  source 1..24: mol_type = other DNA
ctgaacaaagtcaagaccagcctg

SEQ ID NO: 113 - DNA, 24 bp, synthetic construct
  source 1..24: mol_type = other DNA
aacctgaaaacccacctgagactg

SEQ ID NO: 114 - DNA, 27 bp, synthetic construct
  source 1..27: mol_type = other DNA
ttcagaggcgtgttcgtgcaccggtac

SEQ ID NO: 115 - DNA, 27 bp, synthetic construct
  source 1..27: mol_type = other DNA
acatctggccctgtgaccgagaagtac

SEQ ID NO: 116 - DNA, 27 bp, synthetic construct
  source 1..27: mol_type = other DNA
gtggttgctgctcatcttgctggcgct

SEQ ID NO: 117 - DNA, 27 bp, synthetic construct
  source 1..27: mol_type = other DNA
aagtcctactctaaggtgctcgtccgc

SEQ ID NO: 118 - DNA, 27 bp, synthetic construct
  source 1..27: mol_type = other DNA
atgacctacaagatccacttcaagaaa

SEQ ID NO: 119 - DNA, 27 bp, synthetic construct
  source 1..27: mol_type = other DNA
gttccagcttctccagctctgagcaga

SEQ ID NO: 120 - DNA, 27 bp, synthetic construct
  source 1..27: mol_type = other DNA
tctccatctgctagacctccaagcctc

SEQ ID NO: 121 - DNA, 27 bp, synthetic construct
  source 1..27: mol_type = other DNA
tctccaacactgaatgtgtctgccctc

SEQ ID NO: 122 - DNA, 1183 bp, synthetic construct
  source 1..1183: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 123 - protein, 9 aa, synthetic construct
NLKTEILRL

SEQ ID NO: 124 - protein, 8 aa, synthetic construct
PSSASLAL

SEQ ID NO: 125 - RNA, 5000 nt, synthetic construct
  source 1..5000: mol_type = other RNA
  misc_difference 1..5000: note = This sequence may encompass 100-5000 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 126 - RNA, 13 nt, synthetic construct
  source 1..13: mol_type = other RNA
gccgccrccatgg

SEQ ID NO: 127 - protein, 10 aa, synthetic construct
MTYKIFIFKK

(19) United States
(12) Patent Application Publication
(10) Pub. No.: US 2026/0284226 A1
(43) Pub. Date: Sep. 24, 2026

(54) METHODS AND COMPOSITIONS FOR CANCER TREATMENT USING RECOMBINANT POLYPEPTIDES

(71) Applicant: Infinitopes Limited, Oxford (GB)

(72) Inventors: Lian Ni LEE, Oxford (GB); Jonathan KWOK, Oxford (GB); Orion TONG, Oxford (GB); Senthil CHINNAKANNAN, Oxford (GB)

(21) Appl. No.: 19/350,488

(22) Filed: Oct. 6, 2025

Related U.S. Application Data
(63) Continuation of application No. PCT/US2024/023410, filed on Apr. 5, 2024.
(60) Provisional application No. 63/457,918, filed on Apr. 7, 2023.

(51) Int. Cl. A61K 48/00; A61K 35/17; A61K 38/08; A61K 39/00; A61K 40/11; A61K 40/42; C12N 15/86

(52) U.S. Cl. CPC A61K 48/005; A61K 35/17; A61K 38/08; A61K 39/001111; A61K 40/11; A61K 40/421; C12N 15/86; A61K 2239/38; A61K 2239/46; C12N 2710/10343; C12N 2800/40

(57) ABSTRACT

Disclosed hereopin are methods for the treatment of cancer in a human subject comprising administration of a recombinant polypeptide comprising a cancer-specific CD8+ T cell epitope. Peptides recognized by major histocompatibility complex (MHC) molecules can activate a T cell immune response to target cancer cells in a subject. The present disclosure furthermore provides cancer-specific CD8+ T cell epitopes restricted to MHC molecules expressed by specific HLA-Alleles in a subject. The present disclosure furthermore provides a composition encoding the recombinant polypeptide which is capable of eliciting an inflating memory CD8+ T cell response.

### CROSS-REFERENCE TO RELATED APPLICATIONS

This application is a continuation of International Patent Application No. PCT/US24/23410, filed Apr. 5, 2024, which claims priority to U.S. Provisional Patent Application No. 63/457,918, filed on Apr. 7, 2023, the content of which is entirely incorporated herein by reference.

### SEQUENCE LISTING

The instant application contains a Sequence Listing which has been submitted electronically in XML file format and is hereby incorporated by reference in its entirety. Said XML copy, created on Apr. 22, 2026, is named 63274-702_301_SL.xml and is 183,354 bytes in size.

### BACKGROUND

Esophageal cancer is one of the most lethal cancers in the world, currently ranking sixth among cancers in mortality. Esophageal cancer, including esophageal squamous cell carcinoma and esophageal adenocarcinoma, involves the abnormal proliferation of esophageal epithelial cells.

Radiotherapy, surgical resection, and chemotherapy are the primary clinical treatments for esophageal cancer. Outcomes are suboptimal with only 20% of patients surviving at least 5 years following diagnosis. The survival rate of patients treated solely with chemotherapy is still low, and there is a significant need for novel, targeted therapies that demonstrate long-lasting effects.

### SUMMARY OF THE DISCLOSURE

Disclosed herein, in some embodiments, are methods and compositions to treat cancer in a subject, e.g., human subject using a composition comprising an epitope, e.g., T cell epitope, e.g., cancer specific CD8+ T cell epitope.

In an aspects, the present disclosure provides a method of treating cancer in a subject in need thereof comprising administering to the subject a composition comprising: (i) a recombinant polypeptide comprising a cancer-specific CD8+ T cell epitope; (ii) a polynucleotide encoding a recombinant polypeptide comprising a cancer-specific CD8+ T cell epitope; (iii) a vector comprising (i) or (ii); (iv) antigen presenting cells (APCs) comprising (i), (ii), or (iii); or (v) T cells stimulated with APCs comprising (i) or (ii); wherein: the subject expresses an MHC encoded by an HLA-A*03:01 allele and the cancer-specific CD8+ T cell epitope is LKPDHIQR (SEQ ID NO: 7), KTEVHGRLK (SEQ ID NO: 9), RVSLPKLGYK (SEQ ID NO: 11), or SLFGARPGR (SEQ ID NO: 8); the subject expresses an MHC encoded by an HLA-B*07:02 allele and the cancer-specific CD8+ T cell epitope is NLRPPTQEL (SEQ ID NO: 10); the subject expresses an MHC encoded by an HLA-A*31:01 allele and the cancer-specific CD8+ T cell epitope is SLFGARPGR (SEQ ID NO: 8); and/or the subject expresses an MHC encoded by an HLA-B*07:02 allele and the cancer-specific CD8+ T cell epitope is LRPPTQEL (SEQ ID NO: 12).

In an aspect, the present disclosure provides a method of treating cancer in a subject in need thereof comprising administering to the subject a composition comprising: (i) a recombinant polypeptide comprising a cancer-specific CD8+ T cell epitope; (ii) a polynucleotide encoding a recombinant polypeptide comprising a cancer-specific CD8+ T cell epitope; (iii) a vector comprising (i) or (ii); (iv) antigen presenting cells (APCs) comprising (i), (ii), or (iii); or (v) T cells stimulated with APCs comprising (i) or (ii); wherein: the subject expresses an MHC encoded by an HLA-A*02:01 allele and the cancer-specific CD8+ T cell epitope is GVYDGREHTV (SEQ ID NO: 1) or KLVELEHTL (SEQ ID NO: 2); the subject expresses an MHC encoded by an HLA-B*07:02 allele and the cancer-specific CD8+ T cell epitope is SPSSASLAL (SEQ ID NO: 3) or KPRPDVTNEL (SEQ ID NO: 4); the subject expresses an MHC encoded by an HLA-B*35:03 allele and the cancer-specific CD8+ T cell epitope is SPSSASLAL (SEQ ID NO: 3) or KPRPDVTNEL (SEQ ID NO: 4); the subject expresses an MHC encoded by an HLA-B*40:01 allele and the cancer-specific CD8+ T cell epitope is KEFAFLEHSL (SEQ ID NO: 5) or HELGFKVVL (SEQ ID NO: 6); the subject expresses an MHC encoded by an HLA-B*40:02 allele and the cancer-specific CD8+ T cell epitope is KEFAFLEHSL (SEQ ID NO: 5) or HELGFKVVL (SEQ ID NO: 6); the subject expresses an MHC encoded by an HLA-B*44:02 allele and the cancer-specific CD8+ T cell epitope is KEFAFLEHSL (SEQ ID NO: 5) or HELGFKVVL (SEQ ID NO: 6); and/or the subject expresses an MHC encoded by an HLA-B*18:01 allele and the cancer-specific CD8+ T cell epitope is HELGFKVVL (SEQ ID NO: 6).

In an aspect, the present disclosure provides a method of treating cancer in a subject in need thereof comprising administering to the subject a composition comprising: (i) a recombinant polypeptide comprising a cancer-specific CD8+ T cell epitope; (ii) a polynucleotide encoding a recombinant polypeptide comprising a cancer-specific CD8+ T cell epitope; (iii) a vector comprising (i) or (ii); (iv) antigen presenting cells (APCs) comprising (i), (ii), or (iii); or (v) T cells stimulated with APCs comprising (i) or (ii); wherein: the subject expresses an MHC encoded by an HLA-A*03:01 allele and the cancer-specific CD8+ T cell epitope is SLYQTIRLK (SEQ ID NO: 15), KTPLHTLLK (SEQ ID NO: 16), RLHSFTLRQK (SEQ ID NO: 17) or RVFTSSLKTK (SEQ ID NO: 18); the subject expresses an MHC encoded by an HLA-B*07:02 allele and the cancer-specific CD8+ T cell epitope is RPASPRPAP (SEQ ID NO: 13); and/or the subject expresses an MHC encoded by an HLA-C*03:04 allele and the cancer-specific CD8+ T cell epitope is IATKIALQM (SEQ ID NO: 14).

In an aspect, the present disclosure provides a method of treating cancer in a subject in need thereof comprising administering to the subject a composition comprising: (i) a recombinant polypeptide comprising a cancer-specific CD8+ T cell epitope; (ii) a polynucleotide encoding a recombinant polypeptide comprising a cancer-specific CD8+ T cell epitope; (iii) a vector comprising (i) or (ii); (iv) antigen presenting cells (APCs) comprising (i), (ii), or (iii); or (v) T cells stimulated with APCs comprising (i) or (ii); wherein: the subject expresses an MHC encoded by an HLA-A*02:01 allele and the cancer-specific CD8+ T cell epitope is GVYDGREHTV (SEQ ID NO: 1) or KLVELEHTL (SEQ ID NO: 2); the subject expresses an MHC encoded by an HLA-A*03:01 allele and the cancer-specific CD8+ T cell epitope is LKPDHIQR (SEQ ID NO: 7), RVSLPKLGYK (SEQ ID NO: 11), SLFGARPGR (SEQ ID NO: 8), or KTEVHGRLK (SEQ ID NO: 9); the subject expresses an MHC encoded by an HLA-A*31:01 allele and the cancer-specific CD8+ T cell epitope is SLFGARPGR (SEQ ID NO: 8); the subject expresses an MHC encoded by an HLA-B*07:02 allele and the cancer-specific CD8+ T cell epitope is SPSSASLAL (SEQ ID NO: 3), RPASPRPAP (SEQ ID NO: 13), NLRPPTQEL (SEQ ID NO: 10), or KPRPDVTNEL (SEQ ID NO: 4); the subject expresses an MHC encoded by an HLA-B*35:03 allele and the cancer-specific CD8+ T cell epitope is SPSSASLAL (SEQ ID NO: 3), RPASPRPAP (SEQ ID NO: 13), NLRPPTQEL (SEQ ID NO: 10), or KPRPDVTNEL (SEQ ID NO: 4); the subject expresses an MHC encoded by an HLA-B*40:01 allele and the cancer-specific CD8+ T cell epitope is KEFAFLEHSL (SEQ ID NO: 5) or HELGFKVVL (SEQ ID NO: 6); the subject expresses an MHC encoded by an HLA-B*40:02 allele and the cancer-specific CD8+ T cell epitope is KEFAFLEHSL (SEQ ID NO: 5) or HELGFKVVL (SEQ ID NO: 6); the subject expresses an MHC encoded by an HLA-B*44:02 allele and the cancer-specific CD8+ T cell epitope is HELGFKVVL (SEQ ID NO: 6) or KEFAFLEHSL (SEQ ID NO: 5); the subject expresses an MHC encoded by an HLA-B*18:01 allele and the cancer-specific CD8+ T cell epitope is HELGFKVVL (SEQ ID NO: 6); and/or the subject expresses an MHC encoded by an HLA-C*07:02 allele and the cancer-specific CD8+ T cell epitope is LRPPTQEL (SEQ ID NO: 12), NLRPPTQEL (SEQ ID NO: 10) or KPRPDVTNEL (SEQ ID NO: 4).

In an aspect, the present disclosure provides a method of treating cancer in a subject in need thereof comprising administering to the subject a composition comprising: (i) a recombinant polypeptide comprising a cancer-specific CD8+ T cell epitope; (ii) a polynucleotide encoding a recombinant polypeptide comprising a cancer-specific CD8+ T cell epitope; (iii) a vector comprising (i) or (ii); (iv) antigen presenting cells (APCs) comprising (i), (ii), or (iii); or (v) T cells stimulated with APCs comprising (i) or (ii); wherein: the subject expresses an MHC encoded by an HLA-A*15:01 allele and the cancer-specific CD8+ T cell epitope is GQHLHLETF (SEQ ID NO: 21); the subject expresses an MHC encoded by an HLA-C*03:04 allele and the cancer-specific CD8+ T cell epitope is IATKIALQM (SEQ ID NO: 14); the subject expresses an MHC encoded by an HLA-C*07:02 allele and the cancer-specific CD8+ T cell epitope is LRPPTQEL (SEQ ID NO: 12), NLRPPTQEL (SEQ ID NO: 10) or KPRPDVTNEL (SEQ ID NO: 4); the subject expresses an MHC encoded by an HLA-A*03:01 allele and the cancer-specific CD8+ T cell epitope is LKPDHIQR (SEQ ID NO: 7), RVSLPKLGYK (SEQ ID NO: 11), SLFGARPGR (SEQ ID NO: 8), KTEVHGRLK (SEQ ID NO: 9), RLHSFTLRQK (SEQ ID NO: 17), RVFTSSLKTK (SEQ ID NO: 18), RVLAKGLAK (SEQ ID NO: 19), KTPLHTLLK (SEQ ID NO: 16), KSYSKVLVR (SEQ ID NO: 31), MTYKIHFKK (SEQ ID NO: 32) or SLYQTIRLK (SEQ ID NO: 15); the subject expresses an MHC encoded by an HLA-B*07:02 allele and the cancer-specific CD8+ T cell epitope is SPSSASLAL (SEQ ID NO: 3), KPRPDVTNEL (SEQ ID NO: 4), NLRPPTQEL (SEQ ID NO: 10), RPASPRPAP (SEQ ID NO: 13), GPRPSPTRSV (SEQ ID NO: 20), SPRSPSPSL (SEQ ID NO: 22), VPQEAVRAPL (SEQ ID NO: 23), VPASPALSR (SEQ ID NO: 33), SPSARPPSL (SEQ ID NO: 34), SPTLNVSAL (SEQ ID NO: 35), or SPSSASLTL (SEQ ID NO: 25); the subject expresses an MHC encoded by an HLA-B*08:01 allele and the cancer-specific CD8+ T cell epitope is LNKVKTSL (SEQ ID NO: 26) or NLKTEILRL (SEQ ID NO: 123); the subject expresses an MHC encoded by an HLA-C*07:01 allele and the cancer-specific CD8+ T cell epitope is FRGVFVHRY (SEQ ID NO: 28); the subject expresses an MHC encoded by an HLA-B*35:01 allele and the cancer-specific CD8+ T cell epitope is TSGPVTEKY (SEQ ID NO: 29); the subject expresses an MHC encoded by an HLA-A*02:05 allele and the cancer-specific CD8+ T cell epitope is VVAAHLAGA (SEQ ID NO: 30); the subject expresses an MHC encoded by an HLA-A*02:01 allele and the cancer-specific CD8+ T cell epitope is GVYDGREHTV (SEQ ID NO: 1) or KLVELEHTL (SEQ ID NO: 2); the subject expresses an MHC encoded by an HLA-B*40:01 allele and the cancer-specific CD8+ T cell epitope is KEFAFLEHSL (SEQ ID NO: 5) or HELGFKVVL (SEQ ID NO: 6); the subject expresses an MHC encoded by an HLA-B*44:02 allele and the cancer-specific CD8+ T cell epitope is KEFAFLEHSL (SEQ ID NO: 5) or HELGFKVVL (SEQ ID NO: 6); the subject expresses an MHC encoded by an HLA-B*18:01 allele and the cancer-specific CD8+ T cell epitope is HELGFKVVL (SEQ ID NO: 6); the subject expresses an MHC encoded by an HLA-A*31:01 allele and the cancer-specific CD8+ T cell epitope is SLFGARPGR (SEQ ID NO: 8).

In an aspect, the present disclosure provides method of treating cancer in a subject in need thereof, the method comprising administering to the subject a composition comprising (i) a recombinant polypeptide comprising a cancer-specific CD8+ T cell epitope, or (ii) a polynucleotide encoding a recombinant polypeptide comprising a cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii).

In some embodiments, the subject expresses an MHC encoded by an HLA-A*03:01 allele and the cancer-specific CD8+ T cell epitope is LKPDHIQR (SEQ ID NO: 7), KTEVHGRLK (SEQ ID NO: 9), RVSLPKLGYK (SEQ ID NO: 11), or SLFGARPGR (SEQ ID NO: 8).

In some embodiments, the subject expresses an MHC encoded by an HLA-B*07:02 allele and the cancer-specific CD8+ T cell epitope is NLRPPTQEL (SEQ ID NO: 10).

In some embodiments, the subject expresses an MHC encoded by an HLA-A*31:01 allele and the cancer-specific CD8+ T cell epitope is SLFGARPGR (SEQ ID NO: 8).

In some embodiments, the subject expresses an MHC encoded by an HLA-B*07:02 allele and the cancer-specific CD8+ T cell epitope is LRPPTQEL (SEQ ID NO: 12).

In some embodiments, the subject expresses an MHC encoded by an HLA-A*02:01 allele and the cancer-specific CD8+ T cell epitope is GVYDGREHTV (SEQ ID NO: 1) or KLVELEHTL (SEQ ID NO: 2).

In some embodiments, the subject expresses an MHC encoded by an HLA-B*07:02 allele and the cancer-specific CD8+ T cell epitope is SPSSASLAL (SEQ ID NO: 3) or KPRPDVTNEL (SEQ ID NO: 4).

In some embodiments, the subject expresses an MHC encoded by an HLA-B*35:03 allele and the cancer-specific CD8+ T cell epitope is PSSASLAL (SEQ ID NO: 124), RPASPRPAP (SEQ ID NO: 13), NLRPPTQEL (SEQ ID NO: 10), or KPRPDVTNEL (SEQ ID NO: 4).

In some embodiments, the subject expresses an MHC encoded by an HLA-B*35:03 allele and the cancer-specific CD8+ T cell epitope is SPSSASLAL (SEQ ID NO: 3) or KPRPDVTNEL (SEQ ID NO: 4).

In some embodiments, the subject expresses an MHC encoded by an HLA-B*40:01 allele and the cancer-specific CD8+ T cell epitope is KEFAFLEHSL (SEQ ID NO: 5) or HELGFKVVL (SEQ ID NO: 6).

In some embodiments, the subject expresses an MHC encoded by an HLA-B*40:02 allele and the cancer-specific CD8+ T cell epitope is KEFAFLEHSL (SEQ ID NO: 5) or HELGFKVVL (SEQ ID NO: 6). In some embodiments, the subject expresses an MHC encoded by an HLA-B*44:02 allele and the cancer-specific CD8+ T cell epitope is KEFAFLEHSL (SEQ ID NO: 5) or HELGFKVVL (SEQ ID NO: 6).

In some embodiments, the subject expresses an MHC encoded by an HLA-B*18:01 allele and the cancer-specific CD8+ T cell epitope is HELGFKVVL (SEQ ID NO: 6).

In some embodiments, the subject expresses an MHC encoded by an HLA-A*03:01 allele and the cancer-specific CD8+ T cell epitope is SLYQTIRLK (SEQ ID NO: 15), KTPLHTLLK (SEQ ID NO: 16), RLHSFTLRQK (SEQ ID NO: 17) or RVFTSSLKTK (SEQ ID NO: 18).

In some embodiments, the subject expresses an MHC encoded by an HLA-B*07:02 allele and the cancer-specific CD8+ T cell epitope is RPASPRPAP (SEQ ID NO: 13).

In some embodiments, the subject expresses an MHC encoded by an HLA-C*03:04 allele and the cancer-specific CD8+ T cell epitope is IATKIALQM (SEQ ID NO: 14).

In some embodiments, the subject expresses an MHC encoded by an HLA-A*03:01 allele and the cancer-specific CD8+ T cell epitope is LKPDHIQR (SEQ ID NO: 7), RVSLPKLGYK (SEQ ID NO: 11), SLFGARPGR (SEQ ID NO: 8), KSYSKVLVR (SEQ ID NO: 31), MTYKIHFKK (SEQ ID NO: 32), or KTEVHGRLK (SEQ ID NO: 9).

In some embodiments, the subject expresses an MHC encoded by an HLA-A*31:01 allele and the cancer-specific CD8+ T cell epitope is SLFGARPGR (SEQ ID NO: 8).

In some embodiments, the subject expresses an MHC encoded by an HLA-B*07:02 allele and the cancer-specific CD8+ T cell epitope is SPSSASLAL (SEQ ID NO: 3), RPASPRPAP (SEQ ID NO: 13), NLRPPTQEL (SEQ ID NO: 10), VPASPALSR (SEQ ID NO: 33), SPSARPPSL (SEQ ID NO: 34), SPTLNVSAL (SEQ ID NO: 35), or KPRPDVTNEL (SEQ ID NO: 4).

In some embodiments, the subject expresses an MHC encoded by an HLA-C*07:02 allele and the cancer-specific CD8+ T cell epitope is LRPPTQEL (SEQ ID NO: 12).

In some embodiments, the subject expresses an MHC encoded by an HLA-C*07:02 allele and the cancer-specific CD8+ T cell epitope is KPRPDVTNEL (SEQ ID NO: 4) or NLRPPTQEL (SEQ ID NO: 10).

In some embodiments, the subject expresses an MHC encoded by an HLA-C*03:03 allele and the cancer-specific CD8+ T cell epitope is NLRPPTQEL (SEQ ID NO: 10).

In some embodiments, the subject expresses an MHC encoded by an HLA-B*15:01 allele and the cancer-specific CD8+ T cell epitope is GQHLHLETF (SEQ ID NO: 21).

In some embodiments, the subject expresses an MHC encoded by an HLA-C*03:04 allele and the cancer-specific CD8+ T cell epitope is IATKIALQM (SEQ ID NO: 14).

In some embodiments, the subject expresses an MHC encoded by an HLA-C*07:02 allele and the cancer-specific CD8+ T cell epitope is LRPPTQEL (SEQ ID NO: 12).

In some embodiments, the subject expresses an MHC encoded by an HLA-A*03:01 allele and the cancer-specific CD8+ T cell epitope is LKPDHIQR (SEQ ID NO: 7), RVSLPKLGYK (SEQ ID NO: 11), SLFGARPGR (SEQ ID NO: 8), KTEVHGRLK (SEQ ID NO: 9), RLHSFTLRQK (SEQ ID NO: 17), RVFTSSLKTK (SEQ ID NO: 18), RVLAKGLAK (SEQ ID NO: 19), KTPLHTLLK (SEQ ID NO: 16), KSYSKVLVR (SEQ ID NO: 31), MTYKIHFKK (SEQ ID NO: 32), or SLYQTIRLK (SEQ ID NO: 15).

In some embodiments, the subject expresses an MHC encoded by an HLA-B*07:02 allele and the cancer-specific CD8+ T cell epitope is SPSSASLAL (SEQ ID NO: 3), KPRPDVTNEL (SEQ ID NO: 4), NLRPPTQEL (SEQ ID NO: 10), RPASPRPAP (SEQ ID NO: 13), GPRPSPTRSV (SEQ ID NO: 20), SPRSPSPSL (SEQ ID NO: 22), VPQEAVRAPL (SEQ ID NO: 23), VPASPALSR (SEQ ID NO: 33), SPSARPPSL (SEQ ID NO: 34), SPTLNVSAL (SEQ ID NO: 35), or SPSSASLTL (SEQ ID NO: 25).

In some embodiments, the subject expresses an MHC encoded by an HLA-B*08:01 allele and the cancer-specific CD8+ T cell epitope is LNKVKTSL (SEQ ID NO: 26) or NLKTHLRL (SEQ ID NO: 27).

In some embodiments, the subject expresses an MHC encoded by an HLA-C*07:01 allele and the cancer-specific CD8+ T cell epitope is FRGVFVHRY (SEQ ID NO: 28).

In some embodiments, the subject expresses an MHC encoded by an HLA-B*35:01 allele and the cancer-specific CD8+ T cell epitope is TSGPVTEKY (SEQ ID NO: 29).

In some embodiments, the subject expresses an MHC encoded by an HLA-A*02:05 allele and the cancer-specific CD8+ T cell epitope is VVAAHLAGA (SEQ ID NO: 30).

In some embodiments, the cancer can be a cancer selected from selected from a colorectal cancer, prostate cancer, esophageal cancer, liver cancer, renal cancer, lung cancer, bladder cancer, breast cancer, pancreatic cancer, brain cancer, hepatocellular cancer, lymphoma, leukaemia, gastric cancer, cervical cancer, ovarian cancer, thyroid cancer, melanoma, carcinoma, head and neck cancer, skin cancer, nasopharyngeal cancer, Epstein Barr driven cancers, Human Papilloma virus driven cancers and soft tissue sarcoma.

In some embodiments, the polynucleotide can be a vector.

In an aspect, the present disclosure provides a composition comprising a recombinant polypeptide or a polynucleotide encoding the recombinant polypeptide, wherein the recombinant polypeptide comprises a cancer-specific CD8+ T cell epitope which can include e.g. GVYDGREHTV (SEQ ID NO: 1), KLVELEHTL (SEQ ID NO: 2), LKPDHIQR (SEQ ID NO: 7), RVSLPKLGYK (SEQ ID NO: 11), SLFGARPGR (SEQ ID NO: 8), KTEVHGRLK (SEQ ID NO: 9), SLFGARPGR (SEQ ID NO: 8), SPSSASLAL (SEQ ID NO: 3), RPASPRPAP (SEQ ID NO: 13), NLRPPTQEL (SEQ ID NO: 10), KPRPDVTNEL (SEQ ID NO: 4), KEFAFLEHSL (SEQ ID NO: 5), HELGFKVVL (SEQ ID NO: 6), LRPPTQEL (SEQ ID NO: 12), IATKIALQM (SEQ ID NO: 14), SLYQTIRLK (SEQ ID NO: 15), KTPLHTLLK (SEQ ID NO: 16), RLHSFTLRQK (SEQ ID NO: 17), RVFTSSLKTK (SEQ ID NO: 18), RVLAKGLAK (SEQ ID NO: 19), GPRPSPTRSV (SEQ ID NO: 20), GQHLHLETF (SEQ ID NO: 21), SPRSPSPSL (SEQ ID NO: 22), VPQEAVRAPL (SEQ ID NO: 23), SPSSASLTL (SEQ ID NO: 25), LNKVKTSL (SEQ ID NO: 26), NLKTHLRL (SEQ ID NO: 27), FRGVFVHRY (SEQ ID NO: 28), TSGPVTEKY (SEQ ID NO: 29), KSYSKVLVR (SEQ ID NO: 31), MTYKIHFKK (SEQ ID NO: 32), VPASPALSR (SEQ ID NO: 33), SPSARPPSL (SEQ ID NO: 34), SPTLNVSAL (SEQ ID NO: 35), and/or VVAAHLAGA (SEQ ID NO: 30) or any combination thereof.

In an aspect, the present disclosure provides a composition comprising a recombinant polypeptide or a polynucleotide encoding the recombinant polypeptide, wherein the recombinant polypeptide or the recombinant polypeptide encoded by the polynucleotide comprises a cancer-specific CD8+ T cell epitope which can include GVYDGREHTV (SEQ ID NO: 1), KLVELEHTL (SEQ ID NO: 2), LKPDHIQR (SEQ ID NO: 7), RVSLPKLGYK (SEQ ID NO: 11), SLFGARPGR (SEQ ID NO: 8), KTEVHGRLK (SEQ ID NO: 9), SLFGARPGR (SEQ ID NO: 8), SPSSASLAL (SEQ ID NO: 3), RPASPRPAP (SEQ ID NO: 13), NLRPPTQEL (SEQ ID NO: 10), KPRPDVTNEL (SEQ ID NO: 4), KEFAFLEHSL (SEQ ID NO: 5), HELGFKVVL (SEQ ID NO: 6), and/or LRPPTQEL (SEQ ID NO: 12), or any combination thereof.

In some embodiments, the composition comprises the recombinant polypeptide or the recombinant polypeptide encoded by the polynucleotide comprises a single cancer-specific CD8+ T cell epitope which can include e.g., GVYDGREHTV (SEQ ID NO: 1), KLVELEHTL (SEQ ID NO: 2), SPSSASLAL (SEQ ID NO: 3), KPRPDVTNEL (SEQ ID NO: 4), KEFAFLEHSL (SEQ ID NO: 5) and/or HELGFKVVL (SEQ ID NO: 6), or any combination thereof.

In some embodiments, the composition comprises the recombinant polypeptide or the recombinant polypeptide encoded by the polynucleotide comprises a single cancer-specific CD8+ T cell epitope which can include e.g., LKPDHIQR (SEQ ID NO: 7), SLFGARPGR (SEQ ID NO: 8), KTEVHGRLK (SEQ ID NO: 9), NLRPPTQEL (SEQ ID NO: 10), RVSLPKLGYK (SEQ ID NO: 11), and/or LRPPTQEL (SEQ ID NO: 12), or any combination thereof.

In some embodiments, the composition comprises the recombinant polypeptide or the recombinant polypeptide encoded by the polynucleotide comprises a single cancer-specific CD8+ T cell epitope which can include e.g., RPASPRPAP (SEQ ID NO: 13), IATKIALQM (SEQ ID NO: 14), SLYQTIRLK (SEQ ID NO: 15), KTPLHTLLK (SEQ ID NO: 16), RLHSFTLRQK (SEQ ID NO: 17), and/or RVFTSSLKTK (SEQ ID NO: 18), or any combination thereof.

In some embodiments, the recombinant polypeptide or the recombinant polypeptide encoded by the polynucleotide comprises a single cancer-specific CD8+ T cell epitope which can include e.g. IATKIALQM (SEQ ID NO: 14), SLYQTIRLK (SEQ ID NO: 15), KTPLHTLLK (SEQ ID NO: 16), RLHSFTLRQK (SEQ ID NO: 17), RVFTSSLKTK (SEQ ID NO: 18), RVLAKGLAK (SEQ ID NO: 19), GPRPSPTRSV (SEQ ID NO: 20), GQHLHLETF (SEQ ID NO: 21), SPRSPSPSL (SEQ ID NO: 22), VPQEAVRAPL (SEQ ID NO: 23), SPSSASLTL (SEQ ID NO: 25), LNKVKTSL (SEQ ID NO: 26), NLKTHLRL (SEQ ID NO: 27), FRGVFVHRY (SEQ ID NO: 28), TSGPVTEKY (SEQ ID NO: 29), and/or VVAAHLAGA (SEQ ID NO: 30) or any combination thereof.

In an aspect, the present disclosure provides a composition comprising a vector encoding a recombinant polypeptide or a polynucleotide encoding the recombinant polypeptide, wherein the recombinant polypeptide comprises c a cancer-specific CD8+ T cell epitope which can include e.g. GVYDGREHTV (SEQ ID NO: 1), KLVELEHTL (SEQ ID NO: 2), LKPDHIQR (SEQ ID NO: 7), RVSLPKLGYK (SEQ ID NO: 11), SLFGARPGR (SEQ ID NO: 8), KTEVHGRLK (SEQ ID NO: 9), SLFGARPGR (SEQ ID NO: 8), SPSSASLAL (SEQ ID NO: 3), RPASPRPAP (SEQ ID NO: 13), NLRPPTQEL (SEQ ID NO: 10), KPRPDVTNEL (SEQ ID NO: 4), KEFAFLEHSL (SEQ ID NO: 5), HELGFKVVL (SEQ ID NO: 6), LRPPTQEL (SEQ ID NO: 12), IATKIALQM (SEQ ID NO: 14), SLYQTIRLK (SEQ ID NO: 15), KTPLHTLLK (SEQ ID NO: 16), RLHSFTLRQK (SEQ ID NO: 17), RVFTSSLKTK (SEQ ID NO: 18), RVLAKGLAK (SEQ ID NO: 19), GPRPSPTRSV (SEQ ID NO: 20), GQHLHLETF (SEQ ID NO: 21), SPRSPSPSL (SEQ ID NO: 22), VPQEAVRAPL (SEQ ID NO: 23), SPSSASLTL (SEQ ID NO: 25), LNKVKTSL (SEQ ID NO: 26), NLKTHLRL (SEQ ID NO: 27), FRGVFVHRY (SEQ ID NO: 28), TSGPVTEKY (SEQ ID NO: 29), KSYSKVLVR (SEQ ID NO: 31), MTYKIHFKK (SEQ ID NO: 32), VPASPALSR (SEQ ID NO: 33), SPSARPPSL (SEQ ID NO: 34), SPTLNVSAL (SEQ ID NO: 35), and/or VVAAHLAGA (SEQ ID NO: 30) or any combination thereof.

In some embodiments, the vector encodes a single cancer-specific CD8+ T cell epitope which can include e.g. GVYDGREHTV (SEQ ID NO: 1), KLVELEHTL (SEQ ID NO: 2), LKPDHIQR (SEQ ID NO: 7), RVSLPKLGYK (SEQ ID NO: 11), SLFGARPGR (SEQ ID NO: 8), KTEVHGRLK (SEQ ID NO: 9), SLFGARPGR (SEQ ID NO: 8), SPSSASLAL (SEQ ID NO: 3), RPASPRPAP (SEQ ID NO: 13), NLRPPTQEL (SEQ ID NO: 10), KPRPDVTNEL (SEQ ID NO: 4), KEFAFLEHSL (SEQ ID NO: 5), HELGFKVVL (SEQ ID NO: 6), and/or LRPPTQEL (SEQ ID NO: 12), or any combination thereof.

In some embodiments, the vector encodes a single cancer-specific CD8+ T cell epitope which can include e.g., GVYDGREHTV (SEQ ID NO: 1), KLVELEHTL (SEQ ID NO: 2), SPSSASLAL (SEQ ID NO: 3), KPRPDVTNEL (SEQ ID NO: 4), KEFAFLEHSL (SEQ ID NO: 5) and/or HELGFKVVL (SEQ ID NO: 6), or any combination thereof.

In some embodiments, the vector encodes a single cancer-specific CD8+ T cell epitope which can include e.g., LKPDHIQR (SEQ ID NO: 7), SLFGARPGR (SEQ ID NO: 8), KTEVHGRLK (SEQ ID NO: 9), NLRPPTQEL (SEQ ID NO: 10), RVSLPKLGYK (SEQ ID NO: 11), and/or LRPPTQEL (SEQ ID NO: 12), or any combination thereof.

In some embodiments, the vector encodes a single cancer-specific CD8+ T cell epitope which can include e.g., RPASPRPAP (SEQ ID NO: 13), IATKIALQM (SEQ ID NO: 14), SLYQTIRLK (SEQ ID NO: 15), KTPLHTLLK (SEQ ID NO: 16), RLHSFTLRQK (SEQ ID NO: 17), and/or RVFTSSLKTK (SEQ ID NO: 18) or any combination thereof.

In some embodiments, the vector encodes a single cancer-specific CD8+ T cell epitope which can include e.g., IATKIALQM (SEQ ID NO: 14), SLYQTIRLK (SEQ ID NO: 15), KTPLHTLLK (SEQ ID NO: 16), RLHSFTLRQK (SEQ ID NO: 17), RVFTSSLKTK (SEQ ID NO: 18), RVLAKGLAK (SEQ ID NO: 19), GPRPSPTRSV (SEQ ID NO: 20), GQHLHLETF (SEQ ID NO: 21), SPRSPSPSL (SEQ ID NO: 22), VPQEAVRAPL (SEQ ID NO: 23), SPSSASLTL (SEQ ID NO: 25), LNKVKTSL (SEQ ID NO: 26), NLKTHLRL (SEQ ID NO: 27), FRGVFVHRY (SEQ ID NO: 28), TSGPVTEKY (SEQ ID NO: 29), and/or VVAAHLAGA (SEQ ID NO: 30) or any combination thereof.

In some embodiments, the cancer-specific CD8+ T cell epitope can be an esophageal cancer-specific CD8+ T cell epitope.

In some embodiments, the vector can be a vector selected from a plasmid, a cosmid, a bacterial vector, a viral vector, an artificial chromosome, a liposome, a lipid nanoparticle, or an exosome.

In some embodiments, the viral vector can be a viral vector, e.g., an adenovirus vector, e.g., an Ad5 vector, e.g., a replication deficient Adhu5 vector. In some embodiments, the vector is an adenoviral-associated (AAV) vector.

In some embodiments, the recombinant polypeptide is from 8 to 100 amino acids in length. In some embodiments, the recombinant polypeptide can be from 8 to 100 amino acids in length, e.g., 8 to 12 amino acids in length, e.g., 9 to 10 amino acids in length, e.g., 9 amino acids in length or 10 amino acids in length.

In some embodiments, the cancer-specific CD8+ T cell epitope can bind to a MHC class I molecule, e.g., a with a binding affinity of 500 nM or less.

In some embodiments, the cancer-specific CD8+ T cell epitope can bind to a MHC class I molecule, e.g., a with a predicted binding rank score of 2% or less.

In some embodiments, the recombinant polypeptide can comprise a single cancer-specific CD8+ T cell epitope.

In some embodiments, the vector can encode a single cancer-specific CD8+ T cell epitope.

In some embodiments, the recombinant polypeptide can comprise at least two different cancer-specific CD8+ T cell epitopes.

In some embodiments, the recombinant polypeptide can comprise at least two different recombinant polypeptides. In some cases, each polypeptide can comprise a cancer-specific CD8+ T cell epitope.

In some embodiments, the composition can further comprise an adjuvant.

In some embodiments, the method can further comprise administering a second therapeutic agent, e.g., an immune checkpoint inhibitor.

In some embodiments, the polynucleotide encoding the recombinant polypeptide can be DNA.

In some embodiments, the polynucleotide encoding the recombinant polypeptide can be RNA, e.g., mRNA. In some embodiments, the polynucleotide encoding the recombinant polypeptide is from 18 to 45 nucleotides in length.

In some embodiments, the composition can be for use in treating a cancer, e.g., a cancer selected from a colorectal cancer, prostate cancer, esophageal cancer, liver cancer, renal cancer, lung cancer, bladder cancer, breast cancer, pancreatic cancer, brain cancer, hepatocellular cancer, lymphoma, leukaemia, gastric cancer, cervical cancer, ovarian cancer, thyroid cancer, melanoma, carcinoma, head and neck cancer, skin cancer, nasopharyngeal cancer, Epstein Barr driven cancers, Human Papilloma virus driven cancers and soft tissue sarcoma, e.g., an esophageal cancer.

In some embodiments, the composition can be for use in treating a virally driven cancer. The virally driven cancer may be a cancer caused by HPV (human papilloma virus), HTLV (human T-lymphotropic virus), or EBV (Epstein Barr virus).

In some embodiments, the composition can induce epitope-specific T cell response and/or an inflating memory CD8+ T cell response when administered to a subject.

In some embodiments, the composition can be capable of inducing production of CD8+ T cells characterized by markers which can include e.g., CX3CR1+, KLRG-1+, CD44+, and/or CD62L-, or any combination thereof. In some embodiments, the composition can be capable of inducing production of CD8+ T cells characterized by markers which can include CX3CR1+, KLRG-1+, CD44+, CD62L-, CD27-(low), and/or CD127-(low), or any combination thereof.

In some embodiments, the composition can be capable of inducing production of CD8+/CX3CR1+/KLRG-1+/CD44+ T cells. In some embodiments, the composition can be capable of inducing production of CD8+/CX3CR1+/KLRG-1+/CD62L-T cells. In some embodiments, the composition can be capable of inducing production of CD8+/CX3CR1+/KLRG-1+/CD44+/CD62L-T cells. In some embodiments, the composition can be capable of inducing production of CD8+/CX3CR1+/KLRG-1+/CD44+/CD62L-/CD27-/CD127-T cells, CD8+/CX3CR1+/KLRG-1+/CD44+/CD62L-/CD27 (low)/CD127-T cells, CD8+/CX3CR1+/KLRG-1+/CD44+/CD62L-/CD27-/CD127 (low) T cells, or CD8+/CX3CR1+/KLRG-1+/CD44+/CD62L-/CD27 (low)/CD127 (low) T cells.

In some embodiments, the composition can be capable of inducing production of CD8+/CX3CR1+/KLRG-1+ T cells are further characterized by the phenotype CCR7- and/or CD45RA+/−.

In some embodiments, the composition can be capable of inducing production of CD8+/CX3CR1+/KLRG-1+ T cells that have sustained expression of Tbx21 and/or E2f2.

In some embodiments, the composition can be capable of inducing production of CD8+/CX3CR1+/KLRG-1+ T cells that have low expression of Eomes.

In some embodiments, the composition can be capable of inducing production of CD8+/CX3CR1+/KLRG-1+ T cells that can form about 0.01 percent to 20 percent of total circulating CD8+ T cells in the subject.

In some embodiments, the composition can be capable of inducing production of CD8+/CX3CR1+/KLRG-1+ T cells that maintain a memory effector phenotype for at least 30 days.

In some embodiments, the composition can be capable of inducing production of CD8+/CX3CR1+/KLRG-1+ T cells that have low expression of PD-1, Tim-3, and/or Lag-3.

In some embodiments, the composition can be capable of inducing production of an inflating memory CD8+ T cell response that can be capable of controlling tumor growth in the subject greater than 50 days after the composition is administered.

In some embodiments, the composition can be capable of inducing production of a nucleotide sequence encoding the cancer specific CD8+ T cell epitope that encodes a polypeptide comprising the cancer specific CD8+ T cell epitope, in some cases where the polypeptide comprising the cancer specific CD8+ T cell epitope is not processed by antigen presenting cells of the subject when administered; and in some cases when a pharmaceutical composition comprising a corresponding viral vector comprising a nucleotide sequence encoding the cancer specific CD8+ T cell epitope that encodes a polypeptide comprising the cancer specific CD8+ T cell epitope that is processed by antigen presenting cells of the subject when administered, the inflating memory CD8+ T cell response is not induced in the subject.

In some embodiments, the cancer-specific CD8+ T cell epitope can be a viral antigen, a tumor associated antigen that is overexpressed in a cancer cell, or an antigen that is mutated in a cancer cell.

In some embodiments, the polypeptide comprising the cancer specific CD8+ T cell epitope can be not processed by antigen presenting cells.

In some embodiments, the viral vector can include a CMV promoter and a TATA box.

In some embodiments, the viral vector can lack a sequence encoding the E1 and E3 proteins.

In some embodiments, the composition can include at least 2, 3, 4, 5, or 6 vectors, wherein each of the at least 2, 3, 4, 5, or 6 vectors is a viral vector, and wherein each of the at least 2, 3, 4, 5, or 6 viral vectors can encode a different single cancer specific CD8+ T cell epitope.

In some embodiments, the composition can include at least 1, 2, 3, 4, 5, or 6 vectors, wherein each of the at least 1, 2, 3, 4, 5, or 6 vectors is a viral vector, and wherein each of the at least 1, 2, 3, 4, 5, or 6 vectors can encode two different single cancer specific CD8+ T cell epitopes.

In an aspect, the present disclosure provides a pharmaceutical composition which can include the composition described herein, and a pharmaceutically acceptable carrier, diluent, excipient, or adjuvant.

In some embodiments, the present disclosure provides a method of treating or preventing a cancer which can include administering a therapeutically effective amount of the pharmaceutical composition described herein to a subject in need thereof.

In some embodiments, the present disclosure provides a method of inducing an inflating memory CD8+ T cell response which can include administering a therapeutically effective amount of the pharmaceutical composition described herein to a subject in need thereof, wherein the inflating memory CD8+ T cell response can include production of CD8+/CX3CR1+/KLRG-1+ T cells.

In some embodiments, the vector or composition can be administered intravenously or intramuscularly.

In some embodiments, the composition can be administered as a single dose.

In some embodiments, the composition can be administered as multiple doses, e.g., in two doses.

In some embodiments, the first dose is a prime dose.

In some embodiments, the first dose elicits a measurable immune response in a subject as compared to the immune response in the subject in the absence of administration of the first dose.

In some embodiments, the second dose is a booster dose.

In some embodiments, the second dose elicits a measurable immune response in a subject as compared to the immune response in the subject in the absence of administration of the second dose.

In some embodiments, the second dose is administered 24 weeks after the first dose.

In some embodiments, the second dose elicits an immune response that is at least 2 fold greater than the immune response elicited by the first dose.

In some embodiments, the composition can be administered prophylactically to the subject.

In some embodiments, the composition can be administered in combination with a second therapeutic agent, e.g., selected from an immune checkpoint inhibitor, a chemotherapeutic agent, a small molecule inhibitor, or radiotherapy.

In some embodiments, the immune check point inhibitor can be an inhibitor of an immune checkpoint protein which can include e.g., CTLA-4, PD-1, PD-L1, PD-L2, TIM-3, LAG-3, B7-H3, B7-H4, B7-H6, A2aR, BTLA, GALS and/or IDO, or any combination thereof.

In some embodiments, the method of treating or preventing a cancer, further comprises administering a chemotherapy course. In some embodiments, the method of treating or preventing a cancer, further comprises administering a first chemotherapy course and a second chemotherapy course. In some embodiments, the booster dose is administered 2 weeks prior to the second chemotherapy course.

In some embodiments, the method of treating or preventing a cancer, further comprises a surgery, e.g., surgical resection. In some embodiments, the booster dose is administered 1 week after post-surgical recovery.

In an aspect, the present disclosure provides a method of producing the composition described herein comprising (i) synthesising a nucleotide sequence encoding the single cancer specific CD8+ T cell epitope, as a sense and antisense primer, (ii) cloning the nucleotide sequence encoding the single cancer specific CD8+ T cell epitope synthesized in (i) into a first plasmid, (iii) cloning a sequence comprising the nucleotide sequence encoding the single cancer specific CD8+ T cell epitope from the first plasmid of (ii) into a second vector comprising adenoviral DNA.

In an aspect, the present disclosure provides a method of treating or preventing a cancer that can include administering a composition described herein to a subject in need thereof, wherein at least two viral vectors are present in the pharmaceutical composition at an amount that is not therapeutically effective individually, thereby treating the cancer in the subject.

In some embodiments, the viral vector can comprise a sequence with at least 90% sequence identity to SEQ ID NO: 44 and a sequence with at least 90% sequence identity to SEQ ID NO: 45.

In some embodiments, the viral vector can further comprise a sequence with at least 90% sequence identity to SEQ ID NO: 47 and a sequence with at least 90% sequence identity to SEQ ID NO: 48.

In one aspect, the present disclosure provides a method of treating cancer in a subject in need thereof comprising administering to the subject a composition comprising: (i) a recombinant polypeptide comprising a cancer-specific CD8+ T cell epitope, wherein the recombinant polypeptide binds to a TCR; (ii) a polynucleotide encoding a recombinant polypeptide comprising a cancer-specific CD8+ T cell epitope wherein the recombinant polypeptide encoded by the polynucleotide binds to a TCR; (iii) a cell comprising a recombinant polypeptide or a polynucleotide of (i) or (ii); (iv) a recombinant polypeptide comprising an antigen binding domain wherein the antigen binding domain binds to a cancer-specific CD8+ T cell epitope; (v) a polynucleotide encoding a recombinant polypeptide comprising an antigen binding domain wherein the antigen binding domain binds to a cancer-specific CD8+ T cell epitope; or (vi) a cell comprising a recombinant polypeptide or a polynucleotide of (iii) or (iv); thereby treating the

In some embodiments, the viral vector comprises one or more of a promoter, a translation initiating sequence, a start codon, a T cell epitope codon, a stop codon, and a polyadenylation sequence, wherein (i) the promoter is selected from a CMV promoter, an RSV promoter, and an EF 1 a promoter, optionally wherein the promoter comprises a sequence as set forth in SEQ ID NO: 36; (ii) the translation initiation sequence comprises a sequence as set forth in SEQ ID NO: 37; and (iii) the polyadenylation sequence comprises a sequence as set forth in SEQ ID NO 43.

In some embodiments, the vector or viral vector comprises a deletion or a functional deletion in the E1 gene at an E1 gene locus. In some embodiments, the vector or viral vector comprises a transgene insertion at the E1 gene locus.

In some embodiments, the method comprises administering to the subject (i) a first adenoviral vector comprising a sequence encoding a single cancer-specific CD8+ T cell epitope having the sequence GVYDGREHTV (SEQ ID NO: 1), and (ii) a second adenoviral vector comprising a sequence encoding a single cancer-specific CD8+ T cell epitope having the sequence KLVELEHTL (SEQ ID NO: 2); wherein the subject expresses an MHC encoded by an HLA-A*02:01 allele.

In some embodiments, the method comprises administering to the subject (i) a first adenoviral vector comprising a sequence encoding a single cancer-specific CD8+ T cell epitope having the sequence SPSSASLAL (SEQ ID NO: 3), and (ii) a second adenoviral vector comprising a sequence encoding a single cancer-specific CD8+ T cell epitope having the sequence KPRPDVTNEL (SEQ ID NO: 4); wherein the subject expresses an MHC encoded by an HLA-B*07:02 allele.

In some embodiments, the method comprises administering to the subject (i) a first adenoviral vector comprising a sequence encoding a single cancer-specific CD8+ T cell epitope having the sequence SPSSASLAL (SEQ ID NO: 3), and (ii) a second adenoviral vector comprising a sequence encoding a single cancer-specific CD8+ T cell epitope having the sequence KPRPDVTNEL (SEQ ID NO: 4); wherein the subject expresses an MHC encoded by an HLA-B*35:03 allele.

In some embodiments, the method comprises administering to the subject (i) a first adenoviral vector comprising a sequence encoding a single cancer-specific CD8+ T cell epitope having the sequence KEFAFLEHSL (SEQ ID NO: 5), and (ii) a second adenoviral vector comprising a sequence encoding a single cancer-specific CD8+ T cell epitope having the sequence HELGFKVVL (SEQ ID NO: 6); wherein the subject expresses an MHC encoded by an HLA-B*40:01 allele.

In some embodiments, the method comprises administering to the subject (i) a first adenoviral vector comprising a sequence encoding a single cancer-specific CD8+ T cell epitope having the sequence KEFAFLEHSL (SEQ ID NO: 5), and (ii) a second adenoviral vector comprising a sequence encoding a single cancer-specific CD8+ T cell epitope having the sequence HELGFKVVL (SEQ ID NO: 6); wherein the subject expresses an MHC encoded by an HLA-B*40:02 allele.

In some embodiments, the method comprises administering to the subject (i) a first adenoviral vector comprising a sequence encoding a single cancer-specific CD8+ T cell epitope having the sequence KEFAFLEHSL (SEQ ID NO: 5), and (ii) a second adenoviral vector comprising a sequence encoding a single cancer-specific CD8+ T cell epitope having the sequence HELGFKVVL (SEQ ID NO: 6); wherein the subject expresses an MHC encoded by an HLA-B*44:02 allele.

In some embodiments, the method comprises administering to the subject (i) a first adenoviral vector comprising a sequence encoding a single cancer-specific CD8+ T cell epitope having the sequence KEFAFLEHSL (SEQ ID NO: 5), and (ii) a second adenoviral vector comprising a sequence encoding a single cancer-specific CD8+ T cell epitope having the sequence HELGFKVVL (SEQ ID NO: 6); wherein the subject expresses an MHC encoded by an HLA-B*18:01 allele.

In some embodiments, the composition comprises (i) a first adenoviral vector comprising a sequence encoding a single cancer-specific CD8+ T cell epitope having the sequence GVYDGREHTV (SEQ ID NO: 1), and (ii) a second adenoviral vector comprising a sequence encoding a single cancer-specific CD8+ T cell epitope having the sequence KLVELEHTL (SEQ ID NO: 2).

In some embodiments, the composition is for use in treating esophageal cancer in a subject.

In some embodiments, the composition is for use in treating esophageal cancer in a subject, wherein the subject expresses an MHC encoded by an HLA-A*02:01 allele.

In some embodiments, the composition comprises (i) a first adenoviral vector comprising a sequence encoding a single cancer-specific CD8+ T cell epitope having the sequence SPSSASLAL (SEQ ID NO: 3), and (ii) a second adenoviral vector comprising a sequence encoding a single cancer-specific CD8+ T cell epitope having the sequence KPRPDVTNEL (SEQ ID NO: 4).

In some embodiments, the composition is for use in treating esophageal cancer in a subject.

In some embodiments, the composition is for use in treating esophageal cancer in a subject, wherein the subject expresses an MHC encoded by an HLA-B*07:02 allele.

In some embodiments, the composition is for use in treating esophageal cancer in a subject, wherein the subject expresses an MHC encoded by an HLA-B*35:03 allele.

In some embodiments, the composition comprises (i) a first adenoviral vector comprising a sequence encoding a single cancer-specific CD8+ T cell epitope having the sequence KEFAFLEHSL (SEQ ID NO: 5), and (ii) a second adenoviral vector comprising a sequence encoding a single cancer-specific CD8+ T cell epitope having the sequence HELGFKVVL (SEQ ID NO: 6).

In some embodiments, the composition is for use in treating esophageal cancer in a subject.

In some embodiments, the composition is for use in treating esophageal cancer in a subject, wherein the subject expresses an MHC encoded by an HLA-B*40:01 allele.

In some embodiments, the composition is for use in treating esophageal cancer in a subject, wherein the subject expresses an MHC encoded by an HLA-B*40:02 allele.

In some embodiments, the composition is for use in treating esophageal cancer in a subject, wherein the subject expresses an MHC encoded by an HLA-B*44:02 allele.

In some embodiments, the composition is for use in treating esophageal cancer in a subject, wherein the subject expresses an MHC encoded by an HLA-B*18:01 allele.

In some embodiments, the first adenoviral vector is and Ad5 adenoviral vector and/or the second adenoviral vector is and Ad5 adenoviral vector.

In some embodiments, the first adenoviral vector comprises a deletion or a functional deletion in the E1 gene at an E1 gene locus and/or the second adenoviral vector comprises a deletion or a functional deletion in the E1 gene at an E1 gene locus.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The features of the disclosure are set forth with particularity in the appended claims. A better understanding of the features and the disclosure will be obtained by reference to the following detailed description that sets forth illustrative embodiments, in which the principles of the invention are utilized, and the accompanying drawings (also “Figure” and “FIG.” herein), of which:

FIG. 1 shows a schematic diagram of the proposed clinical trial protocol.

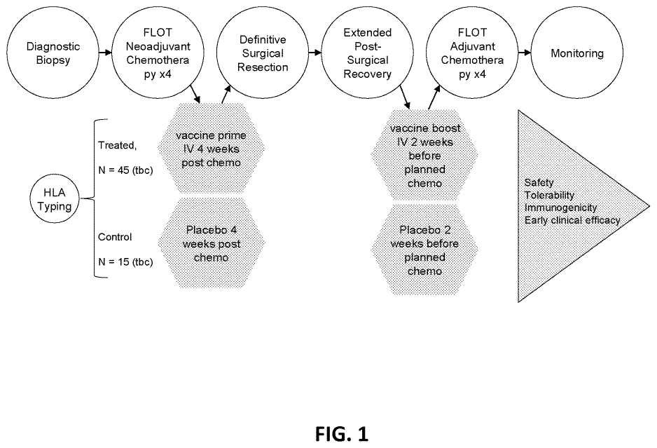

FIG. 2 shows a schematic diagram of a proposed clinical trial study design.

FIG. 3 shows events at timepoints of a proposed clinical trial design.

### DETAILED DESCRIPTION OF THE DISCLOSURE

Immunotherapeutic approaches have been broadly used to treat cancer by modulating T cells to recognize cancerous cells, causing leukocytes to attack the cancer. Epitope-based cancer vaccines are one strategy that has been used to activate a T cell response to specific tumor associated antigens (TAAs). TAAs are highly expressed on tumor cells and expressed to a lesser extent on normal tissue. Initially, peptide-based single epitope vaccines were used, however these provided poor clinical responses as they did not adequately active the innate immune system.

The present disclosure provides recombinant polypeptides comprising at least one T cell epitope, e.g., a CD8+ T cell epitope, e.g., a cancer-specific CD8+ T cell epitope, polynucleotides encoding the T cell epitopes, e.g., a CD8+ T cell epitopes, vectors, cells, compositions, pharmaceutical compositions, formulation, and methods of using such.

The practice of the present disclosure will employ, unless otherwise indicated, conventional techniques of immunology, molecular biology, chemistry, biochemistry and recombinant DNA technology, which are within the skill of the art. Such techniques are explained fully in the literature, see, e.g., Green and Sambrook et al., Molecular Cloning: A Laboratory Manual, 4th ed., Cold Spring Harbor Laboratory Press, Cold Spring Harbor, N.Y. (2012).

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art. The terminology used herein is for the purpose of describing particular embodiments only and is not intended to be limiting.

As used in the specification and claims, the singular forms “a”, “an” and “the” include plural references unless the context clearly dictates otherwise. For example, the term “a sample” includes a plurality of samples, including mixtures thereof.

Furthermore, to the extent that the terms “including”, “includes”, “having”, “has”, “with”, or variants thereof as used herein mean “comprising”. Unless otherwise specified, the words “comprising”, “comprise”, “comprises”, “having”, “have”, “has”, “including”, “includes”, “include”, “containing”, “contains” and “contain” are inclusive or open-ended and do not exclude additional, unrecited elements or method steps.

The term “and/or” as used in a phrase such as “X and/or Y” herein is intended to include both X and Y; X or Y; X (alone); and Y (alone).

Reference to “some embodiments”, “an embodiment”, “one embodiment”, or “other embodiments” means that a particular feature or characteristic described in connection with the embodiments is included in at least one or more embodiments, but not necessarily all embodiments, of the present disclosure.

The term “about” or “approximately” as disclosed herein refers to in relation to a numerical value means a range of values that fall within 15% greater than or 15% less than the value.

Ranges: throughout this disclosure, various aspects of the present disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

As used herein, the terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of can include determining the amount of something present in addition to determining whether it is present or absent depending on the context.

As used herein, the term “cancer” as used herein refers to diseases with abnormal cell growth, as used herein the term refers to both a primary tumor and metastasis of the primary tumor. Cancer cells can spread locally or through the bloodstream and lymphatic system to other parts of the body. Examples of various cancers include but are not limited to colorectal cancer, prostate cancer, esophageal cancer, liver cancer, renal cancer, lung cancer, breast cancer, breast cancer, pancreatic cancer, brain cancer, hepatocellular cancer, lymphoma, leukemia, gastric cancer, cervical cancer, ovarian cancer, thyroid cancer, melanoma, carcinoma, head and neck cancer, skin cancer, nasopharyngeal cancer, Epstein Barr driven cancers, Human Papilloma virus driven cancers and soft tissue sarcoma. The cancer described herein can be a stage I cancer, a stage II cancer, a stage III cancer, or a stage IV cancer. As used herein, the term “tumor” refers to the physiological condition in mammals characterized by deregulated cell growth.

As used herein the term “individual,” “patient,” or “subject” refers to individuals diagnosed with, suspected of being afflicted with, or at-risk of developing at least one disease for which the described compositions and methods are useful for treating. In certain embodiments, the individual is a human.

In some embodiments, the individual is a mammal. In some embodiments, the mammal is a non-human primate (e.g., rhesus or other types of macaques), mouse, rat, rabbit, dog, cat, horse, cow, sheep, pig, goat, llama, alpaca, or yak. In certain embodiments, the individual is a human.

The term “linker” as used herein refers to a molecule linking two other molecules or moieties. The linker can be an amino acid sequence in the case of a linker joining two fusion proteins. The linker can also be a nucleotide sequence in the case of joining two nucleotide sequences together. In some cases, a linker may have various lengths, depending on the application of a linker or the sequences or molecules being linked by a linker.

As used herein, the terms “treatment” or “treating” are used in reference to a pharmaceutical or other intervention regimen used for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made. Skilled artisans will recognize that given a population of potential individuals for treatment not all will respond or respond equally to the treatment. Such individuals are considered treated if administered the compositions including the pharmaceutical compositions described herein.

As used herein, the term “effective amount”, “therapeutically effective amount” or “pharmaceutically effective amount” refers to an amount that is sufficient to effect treatment, as defined below, when administered to a subject (e.g., a mammal, such as a human) in need of such treatment. The therapeutically or pharmaceutically effective amount will vary depending upon the subject and disease condition being treated, the weight and age of the subject, the severity of the disease condition, the manner of administration and the like, which can readily be determined by one of ordinary skill in the art. For example, a “therapeutically effective amount” or a “pharmaceutically effective amount” of AAV2-derived capsid as described herein is an amount sufficient to generate an immune response in a subject (e.g., a human). In some embodiments, the immune response is sufficient to raise AAV capsid neutralizing antibodies against the relevant capsid(s) in the subject.

The term “sequence identity” as used herein refers to the subunit sequence identity between two polymeric molecules, for example, between two polynucleotide or polypeptide sequences. An analysis of sequence identity begins by aligning two sequences. Identical sequences (100% sequence identity) have the same nucleotide or amino acid at each position of the alignment. “Percent sequence identity” is determined by comparing the number of positions that are identical to the total number of subunits in the sequence alignment. Percent sequence identity can be determined over a fraction of the sequences or over the whole of the sequences. Percent sequence identity can be determined using a sequence comparison algorithm. Test and reference sequences are input into a computer, subsequence coordinates are designated, if necessary, and sequence algorithm program parameters are designated. The sequence comparison algorithm then aligns the sequences and calculates the percent sequence identity based on the designated program parameters.

Sequence identity is typically measured using sequence analysis software. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., Current Protocols in Molecular Biology). Such software matches identical or similar sequences by assigning degrees of homology to various substitutions, deletions, and/or other modifications. In an exemplary approach to determining the degree of identity, a BLAST program may be used, with a probability score between e-3 and e-100 indicating a closely related sequence. One example of algorithm that is suitable for determining percent sequence identity and sequence similarity is the BLAST algorithm, which is described in Altschul et al., J. Mol. Biol. 215:403 (1990). Software for performing BLAST analyses is publicly available through the National Center for Biotechnology Information (publicly accessible through the National Institutes of Health NCBI internet server). Typically, default program parameters can be used to perform the sequence comparison, although customized parameters can also be used. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)). In some embodiments, the sequence identities are determined by Needleman-Wunsch alignment of two sequences with Gap Costs set to Existence: 11 Extension: 1 where percent identity is calculated by dividing the number of identities by the length of the alignment. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

As used herein, the term “antigen presenting cell” or “APC” refers to an immune system cell such as an accessory cell (e.g., a B-cell, a dendritic cell, and the like) that displays a foreign antigen complexed with major histocompatibility complexes (MHC's) on its surface. T cells may recognize these complexes using their T cell receptors (TCRs). APCs process antigens and present them to T cells.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, RNA, and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain one or more introns.

The term “T cell” and its grammatical equivalents as used herein can refer to a T cell from any origin. For example, a T cell can be a primary T cell, e.g., an autologous T cell, an allogenic T cell, a cell line, etc. The T cell can also be human or non-human in origin.

The term “CD8” as used herein refers to the Cluster of Differentiation 8 protein, a transmembrane glycoprotein that serves as a co-receptor for the T-cell receptor (TCR).

As used herein, the term “effector T cell” includes T helper (e.g., CD4+) cells and cytotoxic (e.g., CD8+) T cells. CD4+ effector T cells contribute to the development of several immunologic processes, including maturation of B cells into plasma cells and memory B cells, and activation of cytotoxic T cells and macrophages. CD8+ effector T cells destroy virus-infected cells and tumor cells.

As used herein, the term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The term “promoter” refers to a DNA sequence recognized by the transcription machinery of the cell, or introduced synthetic machinery, required to initiate the specific transcription of a polynucleotide sequence.

The term “promoter/regulatory sequence” refers to a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

As used herein, the term “vector” refers to a nucleic acid sequence capable of transporting into a cell another nucleic acid to which the vector sequence has been linked. Numerous vectors are known in the art including, but not limited to, linear polynucleotides, polynucleotides associated with ionic or amphiphilic compounds, plasmids, and viruses. Examples of viral vectors include, but are not limited to, adenoviral vectors, adeno-associated virus vectors (AAVs), retroviral vectors, lentiviral vectors, and the like. A vector of the present disclosure can be adenoviral and comprises the nucleotide sequence encoding a single cancer specific CD8+ T cell epitope containing a gene construct in a form suitable for expression by a cell (e.g., linked to a transcriptional control element).

As used herein, the term “epitope” refers to a part of an antigen that is recognized by the immune system which may be a short protein sequence. A “cancer specific CD8+ T cell epitope” refers to an epitope that may be presented by an antigen presenting cell bound to an MHC molecule which are then recognized by the T-cell receptor (TCR). CD8+ T cells express the CD8 coreceptor, which binds to MHC I, and recognize peptides presented by MHC I molecules.

As used herein, the term “HLA binding affinity” or “MHC binding affinity” means affinity of binding between a specific antigen and a specific MHC allele.

As used herein, the term “HLAbinding rank score” or “MHC binding rank score” means the predicted rank score. The predicted rank score is a percentage rank position of a query sequence (antigen) in relation to a distribution of prediction scores for the MHC in question, estimated from a set of random natural peptides. A rank score below the value of 2% indicates binding between a specific antigen and a specific MHC allele. Provided herein are methods to treat cancer in a human subject, e.g., by administering recombinant polypeptides (or polynucleotides encoding the recombinant polypeptide) comprising a T cell epitope that is capable of binding to one or more MHC molecules, presented by the one or more MHC molecules, being immunogenic and/or capable of activating T cells to become cytotoxic and compositions thereof. In some embodiments, an epitope described herein (e.g., a single cancer-specific CD8+ T cell epitope) may bind to a MHC (e.g., a MHC class I) molecule with a rank score (e.g., a binding rank score) of at least about 0.5%, at least about 1%, at least about 1.5%, at least about 2%, at least about 2.5%, at least about 3%, at least about 3.5%, at least about 4%, at least about 5%, or greater than about 5%. In some embodiments, an epitope described herein (e.g., a single cancer-specific CD8+ T cell epitope) may bind to a MHC (e.g., a MHC class I) molecule with a rank score (e.g., a binding rank score) of at most about 5%, at most about 4%, at most about 3.5%, at most about 3%, at most about 2.5%, at most about 2%, at most about 1.5%, at most about 1%, at most about 0.5%, or less than about 0.5%.

### Recombinant Polypeptides

In one aspect, the present disclosure provides a recombinant polypeptide comprising a single epitope, e.g., a single T cell epitope, e.g., a single cancer-specific CD8+ and/or a CD4+ epitope, e.g., a single cancer-specific CD8+ epitope. In some embodiments, the single epitope, e.g., the single T cell epitope, e.g., the single cancer-specific CD8+ and/or a CD4+ epitope, e.g., the single cancer-specific CD8+ epitope is an immunogenic epitope, e.g., it elicits an immune response. In some embodiments, the single epitope, e.g., the single T cell epitope, e.g., the single cancer-specific CD8+ and/or a CD4+ epitope, e.g., the single cancer-specific CD8+ epitope binds to a major histocompatibility complex (MHC complex), e.g., an MHC associated with a cell, e.g., a cancer cell. In some embodiments, the single epitope, e.g., the single T cell epitope, e.g., the single cancer-specific CD8+ and/or a CD4+ epitope, e.g., the single cancer-specific CD8+ epitope is capable of binding and presenting on an MHC molecule, an MHC molecule associated with a cell, e.g., a cancer cell.

MHC molecules are highly polymorphic proteins that regulate T cell responses (Schwartz, B. D., The human major histocompatibility complex HLA in basic & clinical immunology Stites et al, eds., Lange Medical Publication: Los Altos, pp. 52-64, 4th ed.). The MHC system is responsible for presenting epitopes to responding T cells. The species-specific MHC homologues that display cytotoxic T-cell lymphocyte (CD8+) epitopes or helper T-cell lymphocyte (CD4+) epitopes in humans are termed human leukocyte antigen (“HLA”). HLA class I and class II molecules can be divided into several families or “supertypes” based upon their ability to bind similar repertoires of peptides. HLA class I (HLA-A,B, and C) and peptide complexes are recognized by CD8+ T cells which may then target the presenting cell for destruction. HLA class II (HLA-DP,DQ, and DR) and peptide complexes are recognized by CD4+ T cell which may then initiate an immune response towards the presenting cell. In order to trigger an immune response, the tumor-specific peptide must be bound to an MHC molecule. This binding is dependent on the allele of the MHC molecule and specific polymorphisms of the amino acid sequence of the epitope. In some cases, the recombinant polypeptide can comprise a cancer-specific CD8+ T cell epitope that can bind to a MHC class I molecule. In some cases, the recombinant polypeptide can comprise a cancer-specific CD4+ T cell epitope that can bind to a MHC class II molecule.

In some embodiments, the epitope e.g., the single T cell epitope, e.g., the single cancer-specific CD8+ epitope binds to an MHC encoded by an HLA, e.g., an HLA-A allele, e.g., an HLA-B allele, e.g., an HLA-C allele. In some embodiments the HLA-A allele is an HLA-A*11 allele (e.g., an HLA-A*11:01 allele), an HLA-A*02 allele (e.g., an HLA-A*02:01 allele, e.g., an HLA-A*02:05 allele), an HLA-A*03 allele (e.g., an HLA-A*03:01 allele,), or an HLA-A*31 allele (e.g., an HLA-A*31:01 allele). In some embodiments the HLA-B allele is an HLA-B*07 allele (e.g., an HLA-B*07:02 allele), an HLA-B*08 allele (e.g., an HLA-B*08:01 allele), an HLA-B*15 allele (e.g., an HLA-B*15:01 allele), an HLA-B*18 allele (e.g., an HLA-B*18:01 allele), an HLA-B*35 allele (e.g., an HLA-B*35:01 allele, an HLA-B*35:03 allele), an HLA-B*40 allele (e.g., an HLA-B*40:01 allele, e.g., an HLA-B*40:02 allele), or an HLA-B*44 allele (e.g., an HLA-B*44:01 allele, e.g., an HLA-B*44:02 allele). In some embodiments the HLA-C allele is an HLA-C*03 allele, (e.g. an HLA-C*03:04 allele), or an HLA-C*07 allele, (e.g., an HLA-C*07:01 allele, e.g., an HLA-C*07:02 allele). In some embodiments, the single T cell epitope, e.g., the single cancer-specific CD8+ and/or CD4+ epitope, e.g., the single cancer-specific CD8+ epitope can bind to multiple MHCs encoded by different HLAs, e.g., a single T cell epitope. In some embodiments, a single T cell epitope, e.g., a single cancer-specific CD8+ epitope disclosed herein can bind to an MHC encoded by an HLA selected from HLA-B*40:01, HLA-B*44:01, HLA-B*44:02, or HLA-B*18:01. In some embodiments, the epitope, e.g., a single T cell epitope, e.g., a single cancer-specific CD8+ epitope disclosed herein binds to an MHC encoded by an HLAalleles it is predicted to bind to.

Predicted binding affinities and ranks for MHC molecules can help prioritize peptides for experimental testing. Multiple methods are known in the art to predict the binding of peptides to MHC class I and MHC class II complexes and can include, but are not limited to, NetMHC, NetMHCpan, NetMHCllpan, stabilized matrix method (SMM), and/or average relative binding matrix (ARB). In some instances, the predicted binding rank score can be between 4% and less than 0.1% including 0%, between 3.5% and 02%, between 3% and 0.5%, between 2.5% and 1%, between 2.25% and 1.5%, between 2% and 1%, between 2% and 0.5%, between 0.5% and 0.4%, between 0.4% and 0.3%, between 0.3% and 0.2%, between 0.2% and 0.1%, or less than 0.1% including 0%.

In some instances, the binding affinity can be about 500 nM or less than 500 nM. For example, the binding affinity can be about between 500 nM and 400 nM, about between 400 nM and 300 nM, about between 300 nM and 200 nM, about between 200 nM and 100 nM, or about less than 100 nM including 0 nM. In some embodiments, an epitope described herein (e.g., a single cancer-specific CD8+ T cell epitope) may bind to a MHC (e.g., a MHC class I) molecule with a binding affinity of at most about 750 nM, at most about 500 nM, at most about 400 nM, at most about 300 nM, at most about 200 nM, at most about 100 nM, at most about 50 nM, or less than about 50 nM.

In some embodiments, binding of the single epitope, e.g., the single T cell epitope, e.g., the single cancer-specific CD8+ and/or CD4+ epitope, e.g., the single cancer-specific CD8+ epitope to the MHC complex initiates a subsequent immune response, e.g., an inflating memory T cell response, e.g., an inflating memory CD8+ and/or CD4+ T cell response, e.g., an inflating memory CD8+ T cell response. In some embodiments, the inflating memory T cell response, e.g., the inflating memory CD8+ and/or CD4+ T cell response, e.g., the inflating memory CD8+ T cell response targets cancer cells. In some embodiments, the recombinant polypeptide comprising the single epitope, e.g., the single T cell epitope, e.g., the single cancer-specific CD8+ and/or CD4+ epitope, e.g., the single cancer-specific CD8+ epitope binds an HLA type encoding an MHC of a patient's cancer cells. In some embodiments, the recombinant polypeptide comprising the single epitope, e.g., the single T cell epitope, e.g., the single cancer-specific CD8+ and/or epitope, e.g., the single cancer-specific CD8+ epitope does not bind to an HLA type encoding an MHC of a patient's non-cancer cells. In some embodiments, the recombinant polypeptide comprising the single epitope, e.g., the single T cell epitope, e.g., the single cancer-specific CD8+ and/or CD4+ epitope, e.g., the single cancer-specific CD8+ epitope does not cause T cell exhaustion while binding to the HLA type encoding the MHC of the patient's cancer cells.

There are multiple methods known in the art to identify epitopes which bind the MHC and therefore produce an immune response, e.g., peptide-MHC binding prediction models of which there are multiple programs publicly available.

In some embodiments, the recombinant polypeptide comprises a single epitope, e.g., a single T cell epitope, e.g., the single cancer-specific CD4+ and/or CD8+ epitope, e.g., a single cancer-specific CD8+ epitope comprising an amino acid sequence selected from Table 1. In some embodiments, the recombinant polypeptide comprises a single epitope, e.g., a single T cell epitope, e.g., the single cancer-specific CD4+ and/or CD8+ epitope, e.g., a single cancer-specific CD8+ epitope comprising an amino acid sequence as set forth in any of the SEQ ID NOs: 1-35. In some embodiments, the recombinant polypeptide comprises a single epitope, e.g., a single T cell epitope, e.g., the single cancer-specific CD4+ and/or CD8+ epitope, e.g., a single cancer-specific CD8+ epitope comprising an amino acid sequence with 90-99% identity to an amino acid sequence as set forth in any of the SEQ ID NOs: 1-35. In some embodiments, the recombinant polypeptide comprises a single epitope, e.g., a single T cell epitope, e.g., the single cancer-specific CD4+ and/or CD8+ epitope, e.g., a single cancer-specific CD8+ epitope comprising an amino acid sequence with 1, 2, or at most 3 amino acid mutations compared to an amino acid sequence as set forth in any of the SEQ ID NOs: 1-35.

In some embodiments, the single epitope, e.g., the single T cell epitope, e.g., the single cancer-specific CD4+ and/or CD8+ epitope, e.g., the single cancer-specific CD8+ epitope comprises an amino acid sequence as set forth in any of the SEQ ID NOs: 1-13. In some embodiments, the recombinant polypeptide comprises a single epitope, e.g., a single T cell epitope, e.g., the single cancer-specific CD4+ and/or CD8+ epitope, e.g., a single cancer-specific CD8+ epitope comprising an amino acid sequence with 90-99% identity to an amino acid sequence as set forth in any of the SEQ ID NOs: 1-13. In some embodiments, the recombinant polypeptide comprises a single epitope, e.g., a single T cell epitope, e.g., the single cancer-specific CD4+ and/or CD8+ epitope, e.g., a single cancer-specific CD8+ epitope comprising an amino acid sequence with 1, 2, or at most 3 amino acid mutations compared to an amino acid sequence as set forth in any of the SEQ ID NOs: 1-13.

In some embodiments, the single epitope, e.g., the single T cell epitope, e.g., the single cancer-specific CD4+ and/or CD8+ epitope, e.g., the single cancer-specific CD8+ epitope comprises an amino acid sequence as set forth in any of the SEQ ID NOs: 1-6. In some embodiments, the recombinant polypeptide comprises a single epitope, e.g., a single T cell epitope, e.g., the single cancer-specific CD4+ and/or CD8+ epitope, e.g., a single cancer-specific CD8+ epitope comprising an amino acid sequence with 90-99% identity to an amino acid sequence as set forth in any of the SEQ ID NOs: 1-6. In some embodiments, the recombinant polypeptide comprises a single epitope, e.g., a single T cell epitope, e.g., the single cancer-specific CD4+ and/or CD8+ epitope, e.g., a single cancer-specific CD8+ epitope comprising an amino acid sequence with 1, 2, or at most 3 amino acid mutations compared to an amino acid sequence as set forth in any of the SEQ ID NOs: 1-6.

In some embodiments, the single epitope, e.g., the single T cell epitope, e.g., the single cancer-specific CD4+ and/or CD8+ epitope, e.g., the single cancer-specific CD8+ epitope comprises an amino acid sequence as set forth in any of the SEQ ID NOs: 7-12. In some embodiments, the recombinant polypeptide comprises a single epitope, e.g., a single T cell epitope, e.g., the single cancer-specific CD4+ and/or CD8+ epitope, e.g., a single cancer-specific CD8+ epitope comprising an amino acid sequence with 90-99% identity to an amino acid sequence as set forth in any of the SEQ ID NOs: 7-12. In some embodiments, the recombinant polypeptide comprises a single epitope, e.g., a single T cell epitope, e.g., the single cancer-specific CD4+ and/or CD8+ epitope, e.g., a single cancer-specific CD8+ epitope comprising an amino acid sequence with 1, 2, or at most 3 amino acid mutations compared to an amino acid sequence as set forth in any of the SEQ ID NOs: 7-12.

In some embodiments, the single epitope, e.g., the single T cell epitope, e.g., the single cancer-specific CD8+ epitope comprises an amino acid sequence as set forth in any of the SEQ ID NOs: 13-18. In some embodiments, the recombinant polypeptide comprises a single epitope, e.g., a single T cell epitope, e.g., a single cancer-specific CD8+ epitope comprising an amino acid sequence with 90-99% identity to an amino acid sequence as set forth in any of the SEQ ID NOs: 13-18. In some embodiments, the recombinant polypeptide comprises a single epitope, e.g., a single T cell epitope, e.g., a single cancer-specific CD8+ epitope comprising an amino acid sequence with 1, 2, or at most 3 amino acid mutations compared to an amino acid sequence as set forth in any of the SEQ ID NOs: 13-18. In some embodiments, the single epitope, e.g., the single T cell epitope, e.g., the single cancer-specific CD8+ epitope comprises an amino acid sequence selected from any of the SEQ ID NOs: 19-35, an amino acid sequence with 90-99% identity to an amino acid sequence as set forth in any of the SEQ ID NOs: 19-35, or an amino acid sequence with 1, 2, or at most 3 amino acid mutations compared to an amino acid sequence as set forth in any of the SEQ ID NOs: 19-35.

TABLE 1
Exemplary Epitope Sequences

| SEQ | SEQ | | HLA |
|---|---|---|---|
| Identifier | ID NO. | Sequence | Restriction |
| A1 | 1 | GVYDGREHTV | HLA-A*02:01 |
| A2 | 2 | KL VELEHTL | HLA-A*02:01 |
| A3 | 3 | SPSSASLAL | HLA-B*07:02 |
| | | | HLA-B*35:03 |
| A4 | 4 | KPRPDVTNEL | HLA-B*07:02 |
| | | | HLA-B*35:03 |
| | | | HLA-C*07:02 |
| A5 | 5 | KEFAFLEHSL | HLA-B*40:01 |
| | | | HLA-B*40:02 |
| | | | HLA-B*44:02 |
| A6 | 6 | HELGFKVVL | HLA-B*40:01 |
| | | | HLA-B*40:02 |
| | | | HLA-B*44:02 |
| | | | HLA-B*18:01 |
| A7 | 7 | LKPDHIQR | HLA-A*03:01 |
| | | | HLA-A*11:01 |
| A8 | 8 | SLFGARPGR | HLA-A*03:01 |
| | | | HLA-A*31:01 |
| | | | HLA-A*11:01 |
| A9 | 9 | KTEVHGRLK | HLA-A*03:01 |
| | | | HLA-A*11:01 |
| A10 | 10 | NLRPPTQEL | HLA-B*07:02 |
| | | | HLA-B*35:03 |
| | | | HLA-C*07:02 |
| | | | HLA-C*03:03 |
| A11 | 11 | RVSLPKLGYK | HLA-A*03:01 |
| A12 | 12 | LRPPTQEL | HLA-C*07:02 |
| A13 | 13 | RPASPRPAP | HLA-B*07:02 |
| | | | HLA-B*35:03 |
| A14 | 14 | IATKIALQM | HLA-C*03:04 |
| A15 | 15 | SLYQTIRLK | HLA-A*03:01 |
| | | | HLA-A*11:01 |
| A16 | 16 | KTPLHTLLK | HLA-A*03:01 |
| | | | HLA-A*11:01 |
| A17 | 17 | RLHSFTLRQK | HLA-A*03:01 |
| | | | HLA-A*11:01 |
| A18 | 18 | RVFTSSLKTK | HLA-A*03:01 |
| | | | HLA-A*11:01 |
| A19 | 19 | RVLAKGLAK | HLA-A*03:01 |
| | | | HLA-A*11:01 |
| A20 | 20 | GPRPSPTRSV | HLA-B*07:02 |
| | | | HLA-B*35:03 |
| A21 | 21 | GQHLHLETF | HLA-B*15:01 |
| A22 | 22 | SPRSPSPSL | HLA-B*07:02 |
| | | | HLA-B*35:03 |
| A23 | 23 | VPQEAVRAPL | HLA-B*07:02 |
| | | | HLA-B*35:03 |
| A24 | 24 | IYPFINSH | HLA-C*07:02 |
| A25 | 25 | SPSSASLTL | HLA-B*07:02 |
| | | | HLA-B*35:03 |
| A26 | 26 | LNKVKTSL | HLA-B*08:01 |
| A27 | 27 | NLKTHLRL | HLA-B*08:01 |
| A28 | 28 | FRGVFVHRY | HLA-C*07:01 |
| A29 | 29 | TSGPVTEKY | HLA-B*35:01 |
| A30 | 30 | VVAAHLAGA | HLA-A*02:05 |
| A31 | 31 | KSYSKVLVR | HLA-A*03:01 |
| | | | HLA-A*11:01 |
| A32 | 32 | MTYKIHFKK | HLA-A*03:01 |
| | | | HLA-A*11:01 |
| A33 | 33 | VPASPALSR | HLA-B*07:02 |
| | | | HLA-B*35:03 |
| A34 | 34 | SPSARPPSL | HLA-B*07:02 |
| | | | HLA-B*35:03 |
| A35 | 35 | SPTLNVSAL | HLA-B*07:02 |
| | | | HLA-B*35:03 |

In some embodiments, the single epitope, e.g., the single T cell epitope, e.g., the single cancer-specific CD4+ and/or CD8+ epitope, e.g., the single cancer-specific CD8+ epitope comprises an amino acid sequence as set forth in any of the SEQ ID NOs: 1-35. In some embodiments, the single epitope, e.g., the single T cell epitope, e.g., the single cancer-specific CD4+ and/or CD8+ epitope, e.g., the single cancer-specific CD8+ epitope comprises an amino acid sequence with 90-99% identity to an amino acid sequence as set forth in any of the SEQ ID NOs: 1-35. In some embodiments, the single epitope, e.g., the single T cell epitope, e.g., the single cancer-specific CD4+ and/or CD8+ epitope, e.g., the single cancer-specific CD8+ epitope comprises an amino acid sequence 1, 2, or at most 3 amino acid mutations compared to an amino acid sequence as set forth in any of the SEQ ID NOs: 1-35.

In some embodiments, the single cancer-specific T cell epitope (e.g., the single cancer specific CD8+ T cell epitope) may comprise an amino acid sequence having at least about 90%, at least about 91%, at least about 92%, at least about 93%, at least about 94%, at least about 95%, at least about 96%, at least about 97%, at least about 98%, at least about 98.5%, at least about 99%, at least about 99.5%, at least about 99.9%, or greater than about 99.9% sequence identity to an amino acid sequence as set forth in any one of SEQ ID NOs: 1-35. In some embodiments, the single cancer-specific T cell epitope (e.g., the single cancer specific CD8+ T cell epitope) may comprise an amino acid sequence having at most about 99.9%, at most about 99.5%, at most about 99%, at most about 98.5%, at most about 98%, at most about 97%, at most about 96%, at most about 95%, at most about 94%, at most about 93%, at most about 92%, at most about 91%, at most about 90%, or less than about 90% sequence identity to an amino acid sequence as set forth in any one of SEQ ID NOs: 1-35.

In some embodiments, the single cancer-specific T cell epitope (e.g., the single cancer specific CD8+ T cell epitope) may comprise an amino acid sequence having from about 90% to about 99.9%. In some embodiments, the single cancer-specific T cell epitope (e.g., the single cancer specific CD8+ T cell epitope) may comprise an amino acid sequence having from about 90% to about 91%, about 90% to about 92%, about 90% to about 93%, about 90% to about 94%, about 90% to about 95%, about 90% to about 96%, about 90% to about 97%, about 90% to about 98%, about 90% to about 99%, about 90% to about 99.5%, about 90% to about 99.9%, about 91% to about 92%, about 91% to about 93%, about 91% to about 94%, about 91% to about 95%, about 91% to about 96%, about 91% to about 97%, about 91% to about 98%, about 91% to about 99%, about 91% to about 99.5%, about 91% to about 99.9%, about 92% to about 93%, about 92% to about 94%, about 92% to about 95%, about 92% to about 96%, about 92% to about 97%, about 92% to about 98%, about 92% to about 99%, about 92% to about 99.5%, about 92% to about 99.9%, about 93% to about 94%, about 93% to about 95%, about 93% to about 96%, about 93% to about 97%, about 93% to about 98%, about 93% to about 99%, about 93% to about 99.5%, about 93% to about 99.9%, about 94% to about 95%, about 94% to about 96%, about 94% to about 97%, about 94% to about 98%, about 94% to about 99%, about 94% to about 99.5%, about 94% to about 99.9%, about 95% to about 96%, about 95% to about 97%, about 95% to about 98%, about 95% to about 99%, about 95% to about 99.5%, about 95% to about 99.9%, about 96% to about 97%, about 96% to about 98%, about 96% to about 99%, about 96% to about 99.5%, about 96% to about 99.9%, about 97% to about 98%, about 97% to about 99%, about 97% to about 99.5%, about 97% to about 99.9%, about 98% to about 99%, about 98% to about 99.5%, about 98% to about 99.9%, about 99% to about 99.5%, about 99% to about 99.9%, or about 99.5% to about 99.9%.

In some embodiments, the single epitope, e.g., the single T cell epitope, e.g., the single cancer-specific CD4+ and/or CD8+ epitope, e.g., the single cancer-specific CD8+ epitope comprises an amino acid sequence as set forth in any of the SEQ ID NOs: 1-13. In some embodiments, the single epitope, e.g., the single T cell epitope, e.g., the single cancer-specific CD4+ and/or CD8+ epitope, e.g., the single cancer-specific CD8+ epitope comprises an amino acid sequence with 90-99% identity to an amino acid sequence as set forth in any of the SEQ ID NOs: 1-13. In some embodiments, the single epitope, e.g., the single T cell epitope, e.g., the single cancer-specific CD4+ and/or CD8+ epitope, e.g., the single cancer-specific CD8+ epitope comprises an amino acid sequence 1, 2, or at most 3 amino acid mutations compared to an amino acid sequence as set forth in any of the SEQ ID NOs: 1-13.

In some embodiments, the single epitope, e.g., the single T cell epitope, e.g., the single cancer-specific CD4+ and/or CD8+ epitope, e.g., the single cancer-specific CD8+ epitope comprises an amino acid sequence as set forth in any of the SEQ ID NOs: 1-6. In some embodiments, the single epitope, e.g., the single T cell epitope, e.g., the single cancer-specific CD4+ and/or CD8+ epitope, e.g., the single cancer-specific CD8+ epitope comprises an amino acid sequence with 90-99% identity to an amino acid sequence as set forth in any of the SEQ ID NOs: 1-6. In some embodiments, the single epitope, e.g., the single T cell epitope, e.g., the single cancer-specific CD4+ and/or CD8+ epitope, e.g., the single cancer-specific CD8+ epitope comprises an amino acid sequence 1, 2, or at most 3 amino acid mutations compared to an amino acid sequence as set forth in any of the SEQ ID NOs: 1-6.

In some embodiments, the single epitope, e.g., the single T cell epitope, e.g., the single cancer-specific CD4+ and/or CD8+ epitope, e.g., the single cancer-specific CD8+ epitope comprises an amino acid sequence as set forth in any of the SEQ ID NOs: 7-12. In some embodiments, the single epitope, e.g., the single T cell epitope, e.g., the single cancer-specific CD4+ and/or CD8+ epitope, e.g., the single cancer-specific CD8+ epitope comprises an amino acid sequence with 90-99% identity to an amino acid sequence as set forth in any of the SEQ ID NOs: 7-12. In some embodiments, the single epitope, e.g., the single T cell epitope, e.g., the single cancer-specific CD4+ and/or CD8+ epitope, e.g., the single cancer-specific CD8+ epitope comprises an amino acid sequence 1, 2, or at most 3 amino acid mutations compared to an amino acid sequence as set forth in any of the SEQ ID NOs: 7-12.

In some embodiments, the single epitope, e.g., the single T cell epitope, e.g., the single cancer-specific CD4+ and/or CD8+ epitope, e.g., the single cancer-specific CD8+ epitope comprises an amino acid sequence as set forth in any of the SEQ ID NOs: 13-18. In some embodiments, the single epitope, e.g., the single T cell epitope, e.g., the single cancer-specific CD4+ and/or CD8+ epitope, e.g., the single cancer-specific CD8+ epitope comprises an amino acid sequence with 90-99% identity to an amino acid sequence as set forth in any of the SEQ ID NOs: 13-18. In some embodiments, the single epitope, e.g., the single T cell epitope, e.g., the single cancer-specific CD4+ and/or CD8+ epitope, e.g., the single cancer-specific CD8+ epitope comprises an amino acid sequence 1, 2, or at most 3 amino acid mutations compared to an amino acid sequence as set forth in any of the SEQ ID NOs: 13-18.

In some embodiments overexpressed antigens can have lower expression levels in normal tissues and high expression levels in tumors.

In some embodiments, the single epitope, e.g., the single T cell epitope, e.g., the single cancer-specific CD8+ and/or CD4+ epitope, e.g., the single cancer-specific CD8+ epitope comprises an amino acid sequence as set forth in SEQ ID NO: 1. In some embodiments the epitope, e.g., the T cell epitope, e.g., the single cancer-specific CD8+ and/or CD4+ epitope, e.g., the cancer-specific CD8+ epitope comprises an amino acid sequence with 90-99% identity to the amino acid sequence of the epitope as set forth in SEQ ID NO: 1. In some embodiments, the epitope, e.g., the T cell epitope, e.g., the single cancer-specific CD8+ and/or CD4+ epitope, e.g., the cancer-specific CD8+ epitope comprises an amino acid sequence as set forth in SEQ ID NO: 2. In some embodiments the epitope, e.g., the T cell epitope, e.g., the single cancer-specific CD8+ and/or CD4+ epitope, e.g., the cancer-specific CD8+ epitope comprises an amino acid sequence with 90-99% identity to the amino acid sequence of the epitope as set forth in SEQ ID NO: 2. In some embodiments, the epitope, e.g., the T cell epitope, e.g., the single cancer-specific CD8+ and/or CD4+ epitope, e.g., the cancer-specific CD8+ epitope comprises an amino acid sequence as set forth in SEQ ID NO: 3. In some embodiments, the epitope, e.g., the T cell epitope, e.g., the single cancer-specific CD8+ and/or CD4+ epitope, e.g., the cancer-specific CD8+ epitope comprises an amino acid sequence with 90-99% identity to the amino acid sequence of the epitope as set forth in SEQ ID NO: 3. In some embodiments, the epitope, e.g., the T cell epitope, e.g., the single cancer-specific CD8+ and/or CD4+ epitope, e.g., the cancer-specific CD8+ epitope comprises an amino acid sequence as set forth in SEQ ID NO: 4. In some embodiments, the epitope, e.g., the T cell epitope, e.g., the single cancer-specific CD8+ and/or CD4+ epitope, e.g., the cancer-specific CD8+ epitope comprises an amino acid sequence with 90-99% identity to the amino acid sequence of the epitope as set forth in SEQ ID NO: 4. In some embodiments, the epitope, e.g., the T cell epitope, e.g., the single cancer-specific CD8+ and/or CD4+ epitope, e.g., the cancer-specific CD8+ epitope comprises an amino acid sequence as set forth in SEQ ID NO: 5. In some embodiments, the epitope, e.g., the T cell epitope, e.g., the single cancer-specific CD8+ and/or CD4+ epitope, e.g., the cancer-specific CD8+ epitope comprises an amino acid sequence with 90-99% identity to the amino acid sequence of the epitope as set forth in SEQ ID NO: 5. In some embodiments, the epitope, e.g., the T cell epitope, e.g., the single cancer-specific CD8+ and/or CD4+ epitope, e.g., the cancer-specific CD8+ epitope comprises an amino acid sequence as set forth in SEQ ID NO: 6. In some embodiments, the epitope, e.g., the T cell epitope, e.g., the single cancer-specific CD8+ and/or CD4+ epitope, e.g., the cancer-specific CD8+ epitope comprises an amino acid sequence with 90-99% identity to the amino acid sequence of the epitope as set forth in SEQ ID NO: 6.

In some embodiments, the single epitope, e.g., the single T cell epitope, e.g., the single cancer-specific CD8+ and/or CD4+ epitope, e.g., the single cancer-specific CD8+ epitope comprises an amino acid sequence as set forth in SEQ ID NO: 7. In some embodiments the epitope, e.g., the T cell epitope, e.g., the single cancer-specific CD8+ and/or CD4+ epitope, e.g., the cancer-specific CD8+ epitope comprises an amino acid sequence with 90-99% identity to the amino acid sequence of the epitope as set forth in SEQ ID NO: 7. In some embodiments, the epitope, e.g., the T cell epitope, e.g., the single cancer-specific CD8+ and/or CD4+ epitope, e.g., the cancer-specific CD8+ epitope comprises an amino acid sequence as set forth in SEQ ID NO: 8. In some embodiments the epitope, e.g., the T cell epitope, e.g., the single cancer-specific CD8+ and/or CD4+ epitope, e.g., the cancer-specific CD8+ epitope comprises an amino acid sequence with 90-99% identity to the amino acid sequence of the epitope as set forth in SEQ ID NO: 8. In some embodiments, the epitope, e.g., the T cell epitope, e.g., the single cancer-specific CD8+ and/or CD4+ epitope, e.g., the cancer-specific CD8+ epitope comprises an amino acid sequence as set forth in SEQ ID NO: 9. In some embodiments, the epitope, e.g., the T cell epitope, e.g., the single cancer-specific CD8+ and/or CD4+ epitope, e.g., the cancer-specific CD8+ epitope comprises an amino acid sequence with 90-99% identity to the amino acid sequence of the epitope as set forth in SEQ ID NO: 9. In some embodiments, the epitope, e.g., the T cell epitope, e.g., the single cancer-specific CD8+ and/or CD4+ epitope, e.g., the cancer-specific CD8+ epitope comprises an amino acid sequence as set forth in SEQ ID NO: 10. In some embodiments, the epitope, e.g., the T cell epitope, e.g., the single cancer-specific CD8+ and/or CD4+ epitope, e.g., the cancer-specific CD8+ epitope comprises an amino acid sequence with 90-99% identity to the amino acid sequence of the epitope as set forth in SEQ ID NO: 10. In some embodiments, the epitope, e.g., the T cell epitope, e.g., the single cancer-specific CD8+ and/or CD4+ epitope, e.g., the cancer-specific CD8+ epitope comprises an amino acid sequence as set forth in SEQ ID NO: 11. In some embodiments, the epitope, e.g., the T cell epitope, e.g., the single cancer-specific CD8+ and/or CD4+ epitope, e.g., the cancer-specific CD8+ epitope comprises an amino acid sequence with 90-99% identity to the amino acid sequence of the epitope as set forth in SEQ ID NO: 11. In some embodiments, the epitope, e.g., the T cell epitope, e.g., the single cancer-specific CD8+ and/or CD4+ epitope, e.g., the cancer-specific CD8+ epitope comprises an amino acid sequence as set forth in SEQ ID NO: 12. In some embodiments, the epitope, e.g., the T cell epitope, e.g., the single cancer-specific CD8+ and/or CD4+ epitope, e.g., the cancer-specific CD8+ epitope comprises an amino acid sequence with 90-99% identity to the amino acid sequence of the epitope as set forth in SEQ ID NO: 12.

In some embodiments, the single epitope, e.g., the single T cell epitope, e.g., the single cancer-specific CD8+ and/or CD4+ epitope, e.g., the single cancer-specific CD8+ epitope comprises an amino acid sequence as set forth in SEQ ID NO: 13. In some embodiments the epitope, e.g., the T cell epitope, e.g., the single cancer-specific CD8+ and/or CD4+ epitope, e.g., the cancer-specific CD8+ epitope comprises an amino acid sequence with 90-99% identity to the amino acid sequence of the epitope as set forth in SEQ ID NO: 13. In some embodiments, the epitope, e.g., the T cell epitope, e.g., the single cancer-specific CD8+ and/or CD4+ epitope, e.g., the cancer-specific CD8+ epitope comprises an amino acid sequence as set forth in SEQ ID NO: 14. In some embodiments the epitope, e.g., the T cell epitope, e.g., the single cancer-specific CD8+ and/or CD4+ epitope, e.g., the cancer-specific CD8+ epitope comprises an amino acid sequence with 90-99% identity to the amino acid sequence of the epitope as set forth in SEQ ID NO: 14. In some embodiments, the epitope, e.g., the T cell epitope, e.g., the single cancer-specific CD8+ and/or CD4+ epitope, e.g., the cancer-specific CD8+ epitope comprises an amino acid sequence as set forth in SEQ ID NO: 15. In some embodiments, the epitope, e.g., the T cell epitope, e.g., the single cancer-specific CD8+ and/or CD4+ epitope, e.g., the cancer-specific CD8+ epitope comprises an amino acid sequence with 90-99% identity to the amino acid sequence of the epitope as set forth in SEQ ID NO: 15. In some embodiments, the epitope, e.g., the T cell epitope, e.g., the single cancer-specific CD8+ and/or CD4+ epitope, e.g., the cancer-specific CD8+ epitope comprises an amino acid sequence as set forth in SEQ ID NO: 16. In some embodiments, the epitope, e.g., the T cell epitope, e.g., the single cancer-specific CD8+ and/or CD4+ epitope, e.g., the cancer-specific CD8+ epitope comprises an amino acid sequence with 90-99% identity to the amino acid sequence of the epitope as set forth in SEQ ID NO: 16. In some embodiments, the epitope, e.g., the T cell epitope, e.g., the single cancer-specific CD8+ and/or CD4+ epitope, e.g., the cancer-specific CD8+ epitope comprises an amino acid sequence as set forth in SEQ ID NO: 17. In some embodiments, the epitope, e.g., the T cell epitope, e.g., the single cancer-specific CD8+ and/or CD4+ epitope, e.g., the cancer-specific CD8+ epitope comprises an amino acid sequence with 90-99% identity to the amino acid sequence of the epitope as set forth in SEQ ID NO: 17. In some embodiments, the epitope, e.g., the T cell epitope, e.g., the single cancer-specific CD8+ and/or CD4+ epitope, e.g., the cancer-specific CD8+ epitope comprises an amino acid sequence as set forth in SEQ ID NO: 18. In some embodiments, the epitope, e.g., the T cell epitope, e.g., the single cancer-specific CD8+ and/or CD4+ epitope, e.g., the cancer-specific CD8+ epitope comprises an amino acid sequence with 90-99% identity to the amino acid sequence of the epitope as set forth in SEQ ID NO: 18.

In some embodiments, the recombinant polypeptide is from 8 to 100 amino acids in length. In some embodiments, the recombinant polypeptide is from 5 to 15 amino acids (e.g., 5, 6, 7, 8, 9, 10, 11, 12, 13, 14, or 15) in length. In some embodiments, the recombinant polypeptide is from 8 to 12 amino acids in length. In some embodiments, the recombinant polypeptide is from 9 to 10 amino acids in length. In some embodiments, the recombinant polypeptide is 8 amino acids in length. In some embodiments, the recombinant polypeptide is 9 amino acids in length. In some embodiments, the recombinant polypeptide is 10 amino acids in length. In some embodiments, the recombinant polypeptide is 11 amino acids in length. In some embodiments, the recombinant polypeptide is 12 amino acids in length.

In some embodiments, the recombinant polypeptide comprises more than one, e.g., 2, 3, 4, 5, 6, 7, 8, 9, 10, 15, 20, or 30 epitopes, e.g., T cell epitopes, e.g., cancer-specific CD8+ and/or CD4+ epitopes, e.g., cancer-specific CD8+ epitopes. In some embodiments, the recombinant polypeptide comprises at least two epitopes, e.g., at least two T cell epitopes, e.g., at least two cancer-specific CD8+ and/or CD4+ epitopes, e.g., at least two cancer-specific CD8+ epitopes. In some embodiments, the epitopes are identical. In some embodiments, the epitopes are different. In a non-limiting example, in some embodiments, a recombinant polypeptide disclosed herein may comprise three epitopes e.g., T cell epitopes, e.g., cancer-specific CD8+ and/or CD4+ epitopes, e.g., cancer-specific CD8+ epitopes of which two are identical and one different. In some embodiments, the first epitope e.g., the first T cell epitope, e.g., the first cancer-specific CD8+ and/or CD4+ epitope, e.g., the first cancer-specific CD8+ epitope comprises an amino acid sequence set forth in any of SEQ ID NOs: 1-35. In some embodiments, the second epitope e.g., the second T cell epitope, e.g., the second cancer-specific CD8+ and/or CD4+ epitope, e.g., the second cancer-specific CD8+ epitope comprises an amino acid sequence set forth in any of SEQ ID NOs: 1-35. In some embodiments, the third epitope e.g., the third T cell epitope, e.g., the third cancer-specific CD8+ and/or CD4+ epitope, e.g., the third cancer-specific CD8+ epitope comprises an amino acid sequence set forth in any of SEQ ID NOs: 1-35. In some embodiments, the fourth epitope e.g., the fourth T cell epitope, e.g., the fourth cancer-specific CD8+ and/or CD4+ epitope, e.g., the fourth cancer-specific CD8+ epitope comprises an amino acid sequence set forth in any of SEQ ID NOs: 1-35. In some embodiments, the fifth epitope e.g., the fifth T cell epitope, e.g., the fifth cancer-specific CD8+ and/or CD4+ epitope, e.g., the fifth cancer-specific CD8+ epitope comprises an amino acid sequence set forth in any of SEQ ID NOs: 1-35. In some embodiments, the sixth epitope e.g., the sixth T cell epitope, e.g., the sixth cancer-specific CD8+ and/or CD4+ epitope, e.g., the sixth cancer-specific CD8+ epitope comprises an amino acid sequence set forth in any of SEQ ID NOs: 1-35. In some embodiments, the seventh epitope e.g., the seventh T cell epitope, e.g., the seventh cancer-specific CD8+ and/or CD4+ epitope, e.g., the seventh cancer-specific CD8+ epitope comprises an amino acid sequence set forth in any of SEQ ID NOs: 1-35. In some embodiments, the eighth epitope e.g., the eighth T cell epitope, e.g., the eighth cancer-specific CD8+ and/or CD4+ epitope, e.g., the eighth cancer-specific CD8+ epitope comprises an amino acid sequence set forth in any of SEQ ID NOs: 1-35. In some embodiments, the ninth epitope e.g., the ninth T cell epitope, e.g., the ninth cancer-specific CD8+ and/or CD4+ epitope, e.g., the ninth cancer-specific CD8+ epitope comprises an amino acid sequence set forth in any of SEQ ID NOs: 1-35. In some embodiments, the tenth epitope e.g., the tenth T cell epitope, e.g., the tenth cancer-specific CD8+ and/or CD4+ epitope, e.g., the tenth cancer-specific CD8+ epitope comprises an amino acid sequence set forth in any of SEQ ID NOs: 1-35.

In some embodiments, the epitope, e.g., the T cell epitope, e.g., the cancer-specific CD8+ and/or CD4+ epitope, e.g., the cancer-specific CD8+ epitope is derived from a tumor associated antigen (TAA). A TAA is an antigenic product produced by a cancer and it provides a biomarker for targeted identification of a tumor. TAAs can be broadly categorized into aberrantly expressed self-antigens, mutated self-antigens and tumor specific antigens. As such, the TAA may be upregulated or over-expressed in the cancer cell. The TAA may be mutated within the cancer cell. The TAA may be specific for the cancer cell and only expressed within the cancer cell, this may also be referred to as a tumor specific antigen. The TAA may be overexpressed antigens that have lower expression levels in normal tissues and high expression levels in tumors.

In some embodiments the TAA is associated with aggressive tumor behavior and/or show cancer-restricted expression. In some embodiments, the TAA enhances a CD8+ T cell immune response. In some embodiments, the tumor-associated antigen (TAA) is a tumor specific antigen. In some embodiments, the single epitope, e.g., the single T cell epitope, e.g., the single cancer-specific CD8+ epitope is a cancer testis antigen, e.g., a cancer testis antigen that is NY-ESO-1 or a member of the MAGE-A family. In some embodiments, the single epitope, e.g., the single T cell epitope, e.g., the single cancer-specific CD8+ epitope is an endogenous retroviral protein.

In some embodiments the TAA is associated with aggressive tumor behavior and/or show cancer-restricted expression. In some embodiments, the TAA enhances a CD4+ T cell immune response. In some embodiments, the tumor-associated antigen (TAA) is a tumor specific antigen. In some embodiments, the single epitope, e.g., the single T cell epitope, e.g., the single cancer-specific CD4+ epitope is a cancer testis antigen, e.g., a cancer testis antigen that is NY-ESO-1 or a member of the MAGE-A family. In some embodiments, the single epitope, e.g., the single T cell epitope, e.g., the single cancer-specific CD4+ epitope is an endogenous retroviral protein.

In some embodiments, the single epitope, e.g., the single T cell epitope, e.g., the single cancer-specific CD8+ and/or CD4+ epitope, e.g., the single cancer-specific CD8+ epitope is mutated in a cancer cell. In some embodiments, the single epitope, e.g., the single T cell epitope, e.g., the single cancer-specific CD8+ and/or CD4+ epitope, e.g., the single cancer-specific CD8+ epitope is overexpressed in a cancer cell. In some embodiments, the single epitope, e.g., the single T cell epitope, e.g., the single cancer-specific CD8+ and/or CD4+ epitope, e.g., the single cancer-specific CD8+ epitope is or comprises a non-coding tumor specific epitope. As used herein the term “non-coding tumor specific epitope” refers to a peptide found on a cancer cell, wherein the peptide is derived from a nucleotide sequence that is epigenetically suppressed in healthy cells. These peptide sequences are aberrantly expressed within tumor cells.

In some embodiments, the single epitope, e.g., the single T cell epitope, e.g., the single cancer-specific CD8+ and/or CD4+ epitope, e.g., the single cancer-specific CD8+ epitope is not a cryptic epitope. As used herein a “cryptic epitope” refers to an epitope which is not immunogenic in immunocompetent individuals. In some embodiments, the single epitope, e.g., the single T cell epitope, e.g., the single cancer-specific CD8+ and/or CD4+ epitope, e.g., the single cancer-specific CD8+ epitope is a cryptic epitope.

In some embodiments, the single epitope, e.g., the single T cell epitope, e.g., the single cancer-specific CD8+ and/or CD4+ epitope, e.g., the single cancer-specific CD8+ epitope can be a viral epitope optionally associated with a virally driven cancer. The virally driven cancer may be a cancer caused by HPV (human papilloma virus), HTLV (human T-lymphotropic virus), or EBV (Epstein Barr virus).

In some embodiments, the single epitope, e.g., the single T cell epitope, e.g., the single cancer-specific CD8+ epitope is or is derived from a tumor associated antigen selected from the group comprising: TRP-1, CEA, TAG-72, 9D7, Ep-CAM, EphA3, telomerase, mesothelin, SAP-1 Melan-A/MART-1, tyrosinase, CLPP, cyclin-Al, cyclin-B1 MAGE-Al, MAGE-C1, MAGE-C2, SSX2, XAGE1b/GAGED2a, CD45, glypican-3, IGF2B3, kallikrein-4, KIF20A, lengsin, meloe, MUC5AC, survivin, PRAME, SSX-2, NY-ESO-1/LAGE1, gp70, MC1R, TRP-1/-2, 0-catenin, BRCA1/2, or CDK4.

In some embodiments, the single epitope, e.g., the single T cell epitope, e.g., the single cancer-specific CD4+ epitope is or is derived from a tumor associated antigen selected from the group comprising: EBNA2, TARP, EBV LMP1, STEAP, WT1, HTLV-1 env, HER2/neu, MAGE-A3, BRAF-V600E, NY-ESO-1, or gp100.

In some embodiments, the single epitope, e.g., the single T cell epitope, e.g., the single cancer-specific CD8+ and/or CD4+ epitope, e.g., the single cancer-specific CD8+ epitope is an epitope associated with colorectal cancer, prostate cancer, esophageal cancer, liver cancer, renal cancer, lung cancer, bladder cancer, breast cancer, pancreatic cancer, brain cancer, hepatocellular cancer, lymphoma, leukaemia, gastric cancer, cervical cancer, ovarian cancer, thyroid cancer, melanoma, carcinoma, head and neck cancer, skin cancer, nasopharyngeal cancer, Epstein Barr driven cancers, Human Papilloma virus driven cancers or soft tissue sarcoma. In some embodiments, the cancer is a primary cancer. In some embodiments, the cancer is a relapsed and/or a refractory cancer.

In some embodiments, the single epitope, e.g., the single T cell epitope, e.g., the single cancer-specific CD8+ and/or CD4+ epitope, e.g., the single cancer-specific CD8+ epitope is an esophageal cancer-specific CD8+ T cell epitope. In some embodiments, the single epitope, e.g., the single T cell epitope, e.g., the single cancer-specific CD8+ and/or CD4+ epitope, e.g., the single cancer-specific CD8+ epitope is a colorectal cancer-specific CD8+ T cell epitope. In some embodiments, the single epitope, e.g., the single T cell epitope, e.g., the single cancer-specific CD8+ and/or CD4+ epitope, e.g., the single cancer-specific CD8+ epitope is a prostate cancer-specific CD8+ T cell epitope. In some embodiments, the single epitope, e.g., the single T cell epitope, e.g., the single cancer-specific CD8+ and/or CD4+ epitope, e.g., the single cancer-specific CD8+ epitope is a liver cancer-specific CD8+ T cell epitope. In some embodiments, the single epitope, e.g., the single T cell epitope, e.g., the single cancer-specific CD8+ and/or CD4+ epitope, e.g., the single cancer-specific CD8+ epitope is a renal cancer-specific CD8+ T cell epitope. In some embodiments, the single epitope, e.g., the single T cell epitope, e.g., the single cancer-specific CD8+ and/or CD4+ epitope, e.g., the single cancer-specific CD8+ epitope is a breast cancer-specific CD8+ T cell epitope. In some embodiments, the single epitope, e.g., the single T cell epitope, e.g., the single cancer-specific CD8+ and/or CD4+ epitope, e.g., the single cancer-specific CD8+ epitope is a bladder cancer-specific CD8+ T cell epitope. In some embodiments, the single epitope, e.g., the single T cell epitope, e.g., the single cancer-specific CD8+ and/or CD4+ epitope, e.g., the single cancer-specific CD8+ epitope is a pancreatic cancer-specific CD8+ T cell epitope. In some embodiments, the single epitope, e.g., the single T cell epitope, e.g., the single cancer-specific CD8+ and/or CD4+ epitope, e.g., the single cancer-specific CD8+ epitope is a lung cancer-specific CD8+ T cell epitope. In some embodiments, the single epitope, e.g., the single T cell epitope, e.g., the single cancer-specific CD8+ and/or CD4+ epitope, e.g., the single cancer-specific CD8+ epitope is a brain cancer-specific CD8+ T cell epitope. In some embodiments, the single epitope, e.g., the single T cell epitope, e.g., the single cancer-specific CD8+ and/or CD4+ epitope, e.g., the single cancer-specific CD8+ epitope is a hepatocellular cancer-specific CD8+ T cell epitope. In some embodiments the single epitope, e.g., the single T cell epitope, e.g., the single cancer-specific CD8+ and/or CD4+ epitope, e.g., the single cancer-specific CD8+ epitope is a gastric cancer-specific CD8+ T cell epitope. In some embodiments, the single epitope, e.g., the single T cell epitope, e.g., the single cancer-specific CD8+ and/or CD4+ epitope, e.g., the single cancer-specific CD8+ epitope is a cervical cancer-specific CD8+ T cell epitope. In some embodiments, the single epitope, e.g., the single T cell epitope, e.g., the single cancer-specific CD8+ and/or CD4+ epitope, e.g., the single cancer-specific CD8+ epitope is an ovarian cancer-specific CD8+ T cell epitope. In some embodiments, the single epitope, e.g., the single T cell epitope, e.g., the single cancer-specific CD8+ and/or CD4+ epitope, e.g., the single cancer-specific CD8+ epitope is a thyroid cancer-specific CD8+ T cell epitope. In some embodiments, the single epitope, e.g., the single T cell epitope, e.g., the single cancer-specific CD8+ and/or CD4+ epitope, e.g., the single cancer-specific CD8+ epitope is a CD8+ T cell epitope specific for melanoma. In some embodiments, the single epitope, e.g., the single T cell epitope, e.g., the single cancer-specific CD8+ and/or CD4+ epitope, e.g., the single cancer-specific CD8+ epitope is a CD8+ T cell epitope specific for lymphoma. In some embodiments, the single epitope, e.g., the single T cell epitope, e.g., the single cancer-specific CD8+ and/or CD4+ epitope, e.g., the single cancer-specific CD8+ epitope is a CD8+ T cell epitope specific for leukemia. In some embodiments, the single epitope, e.g., the single T cell epitope, e.g., the single cancer-specific CD8+ and/or CD4+ epitope, e.g., the single cancer-specific CD8+ epitope specific for carcinoma. In some embodiments, the single epitope, e.g., the single T cell epitope, e.g., the single cancer-specific CD8+ and/or CD4+ epitope, e.g., the single cancer-specific CD8+ epitope is a CD8+ T cell epitope specific for head and neck cancer. In some embodiments, the single epitope, e.g., the single T cell epitope, e.g., the single cancer-specific CD8+ and/or CD4+ epitope, e.g., the single cancer-specific CD8+ epitope is a CD8+ T cell epitope specific for skin cancer. In some embodiments, the single epitope, e.g., the single T cell epitope, e.g., the single cancer-specific CD8+ and/or CD4+ epitope, e.g., the single cancer-specific CD8+ epitope is a CD8+ T cell epitope specific for soft tissue sarcoma. In some embodiments, the single epitope, e.g., the single T cell epitope, e.g., the single cancer-specific CD8+ and/or CD4+ epitope, e.g., the single cancer-specific CD8+ epitope is a CD8+ T cell epitope specific for nasopharyngeal cancer.

In some embodiments, the single epitope, e.g., the single T cell epitope, e.g., the single cancer-specific CD8+ and/or CD4+ epitope, e.g., the single cancer-specific CD8+ epitope may be a private epitope. As used herein the term “private epitope” refers to an epitope which is found exclusively on a single antigen in a cancer of a single person. In some embodiments, the single epitope, e.g., the single T cell epitope, e.g., the single cancer-specific CD8+ and/or CD4+ epitope, e.g., the single cancer-specific CD8+ epitope may be a public epitope. As used herein the term “public epitope” refers to an epitope that is found on a cancer of two or more people.

In some embodiments, the recombinant polypeptide comprising the single epitope, e.g., the single T cell epitope, e.g., the single cancer-specific CD8+ and/or CD4+ epitope, e.g., the single cancer-specific CD8+ epitope is capable of inducing an epitope-specific T cell response. In some embodiments, the recombinant polypeptide comprising the single epitope, e.g., the single T cell epitope, e.g., the single cancer-specific CD8+ and/or CD4+ epitope, e.g., the single cancer-specific CD8+ epitope is capable of inducing an inflating memory CD8+ T cell response. In some embodiments, the recombinant polypeptide comprising the single epitope, e.g., the single T cell epitope, e.g., the single cancer-specific CD8+ and/or CD4+ epitope, e.g., the single cancer-specific CD8+ epitope, is not processed by a proteasome or immunoproteasome in antigen presenting cells (APCs).

In some embodiments, the single epitope, e.g., the single T cell epitope, e.g., the single cancer-specific CD8+ and/or CD4+ epitope, e.g., the single cancer-specific CD8+ epitope may be a neoepitope. As used herein the term “neoepitope” refers to epitopes which have arisen through mutations within the tumor cells, in particular somatic or passenger mutations may lead to the production of a neoepitope. In some embodiments, single epitope, e.g., the single T cell epitope, e.g., the single cancer-specific CD8+ and/or CD4+ epitope, e.g., the single cancer-specific CD8+ epitope is not a neoepitope. In some embodiments, the recombinant polypeptide can be a glycoconjugate protein. In some embodiments, the recombinant polypeptide can be a ubiquitinated protein. In some embodiments, the recombinant polypeptide can be a phosphorylated protein. In some embodiments, the recombinant polypeptide can be an acetylated protein. In some embodiments, the recombinant polypeptide can be a methylated protein. In some embodiments, the recombinant polypeptide comprises a signal peptide. “Signal peptide” as used herein refers to an amino acid sequence that can be linked at the amino terminus of recombinant polypeptide disclosed herein. Signal peptides typically direct localization of a protein.

### Polynucleotides

In one aspect, the disclosure provides a polynucleotide encoding a recombinant polypeptide comprising a single epitope, e.g., a single T cell epitope, e.g., a single cancer-specific CD8+ and/or CD4+ epitope, e.g., a single cancer-specific CD8+ epitope described herein. In some embodiments, a polynucleotide encodes a recombinant polypeptide comprising a single epitope, e.g., a single T cell epitope, e.g., a single cancer-specific CD8+ and/or CD4+ epitope, e.g., a single cancer-specific CD8+ epitope comprising an amino acid sequence as set forth in any of SEQ ID NOs: 1-35. In some embodiments, a polynucleotide encodes a recombinant polypeptide comprising a single epitope, e.g., a single T cell epitope, e.g., a single cancer-specific CD8+ and/or CD4+ epitope, e.g., a single cancer-specific CD8+ epitope comprising an amino acid sequence as set forth in any of SEQ ID NOs: 1-13. In some embodiments, a polynucleotide encodes a recombinant polypeptide comprising a single epitope, e.g., a single T cell epitope, e.g., a single cancer-specific CD8+ and/or CD4+ epitope, e.g., a single cancer-specific CD8+ epitope comprising an amino acid sequence as set forth in any of SEQ ID NOs: 1-6. In some embodiments, a polynucleotide encodes a recombinant polypeptide comprising a single epitope, e.g., a single T cell epitope, e.g., a single cancer-specific CD8+ and/or CD4+ epitope, e.g., a single cancer-specific CD8+ epitope comprising an amino acid sequence as set forth in any of SEQ ID NOs: 7-12. In some embodiments, a polynucleotide encodes a recombinant polypeptide comprising a single epitope, e.g., a single T cell epitope, e.g., a single cancer-specific CD8+ and/or CD4+ epitope, e.g., a single cancer-specific CD8+ epitope comprising an amino acid sequence as set forth in any of SEQ ID NOs: 13-18.

Polynucleotides includes, for example, genomic DNA, cDNA, RNA, e.g., mRNA, and DNA-RNA hybrid molecules. Polynucleotides can be naturally occurring, recombinant, or synthetic. In addition, polynucleotides can be single-stranded, double-stranded or triple-stranded. In certain embodiments, polynucleotides can be modified. In the case of a double-stranded polymer, “nucleic acid” can refer to either or both strands of the molecule. In some embodiments, the polynucleotide is provided as DNA. In some embodiments, the polynucleotide is provided as RNA, e.g., an mRNA. In some embodiments, a polynucleotide disclosed herein comprises a nucleic acid sequence as set forth in any of SEQ ID NOs: 52-86. In some embodiments, a polynucleotide disclosed herein comprises a nucleic acid sequence as shown in Table 2.

TABLE 2
Nucleotide sequences for selected
peptide candidates

| SEQ | SEQ ID | |
|---|---|---|
| Identifier | NO. | Sequence |
| A52 | 52 | GGCGTTTACGACGGCAGAGAACACACCGTG |
| A53 | 53 | AAGCTGGTGGAACTGGAACACACCCTG |
| A54 | 54 | TCTCCTAGCTCTGCCTCTCTGGCCCTG |
| A55 | 55 | AAGCCCAGACCTGACGTGACCAACGAGCTG |
| A56 | 56 | AAGGAATTCGCCTTCCTGGAACACAGCCTG |
| A57 | 57 | CACGAGCTGGGCTTCAAGGTGGTGCTG |
| A58 | 58 | CTGAAGCCCGACCACATCCAGAGA |
| A59 | 59 | AGCCTGTTTGGCGCCAGACCTGGCAGA |
| A60 | 60 | AAGACAGAGGTGCACGGCCGGCTGAAG |
| A61 | 61 | AACCTGCGGCCTCCTACACAAGAGCTG |
| A62 | 62 | AGAGTGTCCCTGCCTAAGCTGGGCTACAAG |
| A63 | 63 | CTCAGACCCCCCACTCAGGAACTA |
| A64 | 64 | AGGCCTGCCTCTCCTAGACCTGCTCCT |
| A65 | 65 | ATCGCTACGAAGATCGCCCTACAAATG |
| A66 | 66 | AGCCTGTACCAGACCATCCGGCTGAAG |
| A67 | 67 | AAAACCCCTCTCCATACACTTCTCAAG |
| A68 | 68 | CGGCTCCATTCCTTCACGTTGCGCCAGAAA |
| A69 | 69 | AGAGTTTTTACTTCGAGTCTCAAGACCAAA |
| A70 | 70 | CGAGTGCTAGCAAAGGGCTTAGCAAAG |
| A71 | 71 | GGCCCGCGCCCGTCCCCGACACGCTCGGTG |
| A72 | 72 | GGCCAACATCTTCACTTGGAGACATTT |
| A73 | 73 | AGCCCTAGAAGCCCATCTCCTAGCCTG |
| A74 | 74 | GTGCCTCAAGAAGCCGTTAGAGCCCCTCTG |
| A75 | 75 | ATATATCCGTTCATAAATTCGCAT |
| A76 | 76 | AGTCCTAGTAGTGCCTCGCTAACTCTG |
| A77 | 77 | CTTAACAAGGTGAAAACAAGCTTG |
| A78 | 78 | AACCTTAAAACTCACTTACGACTT |
| A79 | 79 | TTCCGAGGGGTGTTCGTACATAGATAT |
| A80 | 80 | ACGTCCGGCCCGGTGACGGAGAAATAT |
| A81 | 81 | GTAGTGGCCGCTCACTTGGCAGGGGCT |
| A82 | 82 | AAAAGCTACTCAAAAGTGTTAGTCAGA |
| A83 | 83 | ATGACATATAAAATTCACTTCAAGAAG |
| A84 | 84 | GTGCCTGCTAGCCCTGCTCTGAGCAGA |
| A85 | 85 | AGCCCATCAGCTAGGCCTCCATCCCTA |
| A86 | 86 | AGCCCTACCCTTAATGTTTCTGCGCTG |

In some embodiments, the polynucleotides disclosed herein can be codon optimized. usage bias has been reported for numerous organisms, from viruses to eukaryotes. Since the genetic code is degenerate (i.e., each amino acid can be coded by on average three different codons), the DNA sequence can be modified by synonymous nucleotide substitutions without altering the amino acid sequence of the encoded protein. Such synonymous codon optimization has been performed for the purpose of optimizing expression in a desired host, as described in the scientific literature and in patent documents. See U.S. Pat. Nos. 5,786,464 and 6,114,14. In some embodiments, the nucleic acid described herein can be modified to improve cloning efficiency. In some embodiments, the nucleic acids described herein are subjected to codon optimization to increase the efficiency of gene expression, e.g., SEQ ID NOs 87-121. are subjected to codon optimization. In some embodiments, the polypeptide disclosed herein are encoded by polynucleotides whose sequence has been codon optimized for expression in a mammalian cell, e.g., expressional in a human cell. Exemplary codon optimized sequences are disclosed in Table 3. In some embodiments, polypeptides encoded by a codon optimized nucleotide have increased expression in a mammalian cell, e.g., a human cell compared to a polypeptide that is encoded by a non-codon optimized polynucleotide.

TABLE 3
Codon optimized nucleotide sequences for
selected peptide candidates

| SEQ | SEQ | |
|---|---|---|
| Identi- | ID | |
| fier | NO. | Sequence |
| A87 | 87 | GGAGTTTACGATGGCAGAGAGCACACCGTC |
| A88 | 88 | AAGCTGGTGGAACTGGAACACACCCTC |
| A89 | 89 | TCTCCAAGTTCTGCTTCTCTGGCCCTC |
| A90 | 90 | AAGCCTAGACCTGATGTGACCAACGAACTC |
| A91 | 91 | AAGGAATTTGCCTTTCTGGAACACAGCCTC |
| A92 | 92 | CACGAGCTGGGTTTTAAAGTGGTGCTC |
| A93 | 93 | CTGAAGCCTGATCACATTCAGAGA |
| A94 | 94 | TCTCTGTTTGGAGCTAGACCTGGCAGA |
| A95 | 95 | AAGACAGAAGTTCACGGCCGGCTGAAA |
| A96 | 96 | AATCTGAGGCCTCCAACACAAGAACTC |
| A97 | 97 | AGAGTGTCTCTGCCTAAGCTGGGCTACAAA |
| A98 | 98 | CTGAGGCCTCCTACACAAGAGCTG |
| A99 | 99 | AGGCCTGCTTCTCCAAGACCTGCTCCT |
| A100 | 100 | ATCGCCACAAAGATCGCCCTGCAGATG |
| A101 | 101 | AGCCTGTACCAGACCATCAGACTGAAA |
| A102 | 102 | AAGACCCCTCTGCACACCCTGCTGAAG |
| A103 | 103 | AGACTGCACAGCTTCACCCTGCGGCAGAAG |
| A104 | 104 | AGAGTGTTCACCAGCAGCCTCAAGACCAAG |
| A105 | 105 | AGAGTGCTGGCCAAAGGACTGGCCAAG |
| A106 | 106 | GGCCCTAGACCTTCTCCAACCAGAAGCGTG |
| A107 | 107 | GGACAGCATCTGCACCTGGAAACCTTC |
| A108 | 108 | TCTCCAAGATCACCTTCTCCAAGCCTC |
| A109 | 109 | GTTCCTCAAGAAGCTGTTAGAGCCCCACTC |
| A110 | 110 | ATCTACCCCTTCATCAACAGCCAC |
| All! | 111 | TCTCCTAGCAGCGCCTCTCTGACACTG |
| A112 | 112 | CTGAACAAAGTCAAGACCAGCCTG |
| A113 | 113 | AACCTGAAAACCCACCTGAGACTG |
| A114 | 114 | TTCAGAGGCGTGTTCGTGCACCGGTAC |
| A115 | 115 | ACATCTGGCCCTGTGACCGAGAAGTAC |
| A116 | 116 | GTGGTTGCTGCTCATCTTGCTGGCGCT |
| A117 | 117 | AAGTCCTACTCTAAGGTGCTCGTCCGC |
| A118 | 118 | ATGACCTACAAGATCCACTTCAAGAAA |
| A119 | 119 | GTTCCAGCTTCTCCAGCTCTGAGCAGA |
| A120 | 120 | TCTCCATCTGCTAGACCTCCAAGCCTC |
| A121 | 121 | TCTCCAACACTGAATGTGTCTGCCCTC |

In some embodiments, a polypeptide comprising an amino acid sequence as set forth SEQ ID NO: 1, can be encoded by a polynucleotide having a sequence as set forth in SEQ ID NO: 52 or 87. In some embodiments, a polypeptide comprising an amino acid sequence as set forth SEQ ID NO: 2, can be encoded by a polynucleotide having a sequence as set forth in SEQ ID NO: 53 or 88. In some embodiments, a polypeptide comprising an amino acid sequence as set forth SEQ ID NO: 3, can be encoded by a polynucleotide having a sequence as set forth in SEQ ID NO: 54 or 89. In some embodiments, a polypeptide comprising an amino acid sequence as set forth SEQ ID NO: 4, can be encoded by a polynucleotide having a sequence as set forth in SEQ ID NO: 55 or 90. In some embodiments, a polypeptide comprising an amino acid sequence as set forth SEQ ID NO: 5, can be encoded by a polynucleotide having a sequence as set forth in SEQ ID NO: 56 or 91. In some embodiments, a polypeptide comprising an amino acid sequence as set forth SEQ ID NO: 6, can be encoded by a polynucleotide having a sequence as set forth in SEQ ID NO: 57 or 92. In some embodiments, a polypeptide comprising an amino acid sequence as set forth SEQ ID NO: 7, can be encoded by a polynucleotide having a sequence as set forth in SEQ ID NO: 58 or 93. In some embodiments, a polypeptide comprising an amino acid sequence as set forth SEQ ID NO: 8, can be encoded by a polynucleotide having a sequence as set forth in SEQ ID NO: 59 or 94. In some embodiments, a polypeptide comprising an amino acid sequence as set forth SEQ ID NO: 9, can be encoded by a polynucleotide having a sequence as set forth in SEQ ID NO: 60 or 95. In some embodiments, a polypeptide comprising an amino acid sequence as set forth SEQ ID NO: 10, can be encoded by a polynucleotide having a sequence as set forth in SEQ ID NO: 61 or 96. In some embodiments, a polypeptide comprising an amino acid sequence as set forth SEQ ID NO: 11, can be encoded by a polynucleotide having a sequence as set forth in SEQ ID NO: 62 or 97. In some embodiments, a polypeptide comprising an amino acid sequence as set forth SEQ ID NO: 12, can be encoded by a polynucleotide having a sequence as set forth in SEQ ID NO: 63 or 98. In some embodiments, a polypeptide comprising an amino acid sequence as set forth SEQ ID NO: 13, can be encoded by a polynucleotide having a sequence as set forth in SEQ ID NO: 64 or 99. In some embodiments, a polypeptide comprising an amino acid sequence as set forth SEQ ID NO: 14, can be encoded by a polynucleotide having a sequence as set forth in SEQ ID NO: 65 or 100. In some embodiments, a polypeptide comprising an amino acid sequence as set forth SEQ ID NO: 15, can be encoded by a polynucleotide having a sequence as set forth in SEQ ID NO: 66 or 101. In some embodiments, a polypeptide comprising an amino acid sequence as set forth SEQ ID NO: 16, can be encoded by a polynucleotide having a sequence as set forth in SEQ ID NO: 67 or 102. In some embodiments, a polypeptide comprising an amino acid sequence as set forth SEQ ID NO: 17, can be encoded by a polynucleotide having a sequence as set forth in SEQ ID NO: 68 or 103. In some embodiments, a polypeptide comprising an amino acid sequence as set forth SEQ ID NO: 18, can be encoded by a polynucleotide having a sequence as set forth in SEQ ID NO: 69 or 104. In some embodiments, a polypeptide comprising an amino acid sequence as set forth SEQ ID NO: 19, can be encoded by a polynucleotide having a sequence as set forth in SEQ ID NO: 70 or 105. In some embodiments, a polypeptide comprising an amino acid sequence as set forth SEQ ID NO: 20, can be encoded by a polynucleotide having a sequence as set forth in SEQ ID NO: 71 or 106. In some embodiments, a polypeptide comprising an amino acid sequence as set forth SEQ ID NO: 21, can be encoded by a polynucleotide having a sequence as set forth in SEQ ID NO: 72 or 107. In some embodiments, a polypeptide comprising an amino acid sequence as set forth SEQ ID NO: 22, can be encoded by a polynucleotide having a sequence as set forth in SEQ ID NO: 73 or 108. In some embodiments, a polypeptide comprising an amino acid sequence as set forth SEQ ID NO: 23, can be encoded by a polynucleotide having a sequence as set forth in SEQ ID NO: 74 or 109. In some embodiments, a polypeptide comprising an amino acid sequence as set forth SEQ ID NO: 24, can be encoded by a polynucleotide having a sequence as set forth in SEQ ID NO: 75 or 110. In some embodiments, a polypeptide comprising an amino acid sequence as set forth SEQ ID NO: 25, can be encoded by a polynucleotide having a sequence as set forth in SEQ ID NO: 76 or 111. In some embodiments, a polypeptide comprising an amino acid sequence as set forth SEQ ID NO: 26, can be encoded by a polynucleotide having a sequence as set forth in SEQ ID NO: 77 or 112. In some embodiments, a polypeptide comprising an amino acid sequence as set forth SEQ ID NO: 27, can be encoded by a polynucleotide having a sequence as set forth in SEQ ID NO: 78 or 113. In some embodiments, a polypeptide comprising an amino acid sequence as set forth SEQ ID NO: 28, can be encoded by a polynucleotide having a sequence as set forth in SEQ ID NO: 79 or 114. In some embodiments, a polypeptide comprising an amino acid sequence as set forth SEQ ID NO: 29, can be encoded by a polynucleotide having a sequence as set forth in SEQ ID NO: 80 or 115. In some embodiments, a polypeptide comprising an amino acid sequence as set forth SEQ ID NO: 30, can be encoded by a polynucleotide having a sequence as set forth in SEQ ID NO: 81 or 116. In some embodiments, a polypeptide comprising an amino acid sequence as set forth SEQ ID NO: 31, can be encoded by a polynucleotide having a sequence as set forth in SEQ ID NO: 82 or 117. In some embodiments, a polypeptide comprising an amino acid sequence as set forth SEQ ID NO: 32, can be encoded by a polynucleotide having a sequence as set forth in SEQ ID NO: 83 or 118. In some embodiments, a polypeptide comprising an amino acid sequence as set forth SEQ ID NO: 33, can be encoded by a polynucleotide having a sequence as set forth in SEQ ID NO: 84 or 119. In some embodiments, a polypeptide comprising an amino acid sequence as set forth SEQ ID NO: 34, can be encoded by a polynucleotide having a sequence as set forth in SEQ ID NO: 85 or 120. In some embodiments, a polypeptide comprising an amino acid sequence as set forth SEQ ID NO: 35, can be encoded by a polynucleotide having a sequence as set forth in SEQ ID NO: 86 or 121.

In some embodiments, a polynucleotide disclosed herein can encode multiple epitopes, e.g., 2, 3, 4, 5, 6, 7, 8, 9, or 10 epitopes. In some embodiments, the polynucleotide encodes multiple epitopes as discrete peptide epitopes. In some embodiments, the polynucleotide encodes multiple epitopes as polyepitopic peptides. In some embodiments, the epitopes are identical. In some embodiments, the epitopes are different. In some embodiments, a polynucleotide may comprise a sequence encoding at least two or more epitopes described herein, of which both epitopes are the same epitopes. In some embodiments, a polynucleotide may comprise a sequence encoding at least two or more epitopes described herein, of which both epitopes are different epitopes. In a non-limiting example, in some embodiments, a polynucleotide disclosed herein can encode three epitopes e.g., T cell epitopes, e.g., cancer-specific CD8+ and/or CD4+ epitopes, e.g., cancer-specific CD8+ epitopes of which two are the same epitopes and one is a different epitope. In some embodiments, the polynucleotide comprises a nucleotide sequence that encodes spacer amino acid residues between the first and the second epitope.

As will be appreciated by those of skill in the art, in some aspects a nucleic acid comprises regulatory sequences from a plasmid. The nucleic acid sequence can include, for example, one or more of a promoter sequence a selection marker sequence, or a locus-targeting sequence. An example of a promoter that is capable of expressing a transgene in a mammalian T cell is a EFla promoter (SEQ ID NO: 122). The native EFla promoter drives expression of the alpha subunit of the elongation factor-1 complex, which is responsible for the enzymatic delivery of aminoacyl tRNAs to the ribosome. In some embodiments, the polynucleotide comprises an EFla promoter. Another example of a promoter is the immediate early cytomegalovirus (CMV) promoter sequence. This promoter sequence is a strong constitutive promoter sequence capable of driving high levels of expression of any polynucleotide sequence operatively linked thereto. In some embodiments, the polynucleotide comprises a CMV promoter. In some embodiments, the CMV promoter comprises a sequence with 80-100% sequence identity as that set forth in SEQ ID NO: 36. In some embodiments, the polynucleotide of the present disclosure may comprise a promoter selected from the group consisting of, but not limited to, a simian virus 40 (SV40) early promoter, a mouse mammary tumor virus (MMTV), a human immunodeficiency virus (HIV) long terminal repeat (LTR) promoter, a MoMuLV promoter, an avian leukemia virus promoter, an Epstein-Barr virus immediate early promoter, a Rous sarcoma virus (RSV) promoter, an actin promoter, a myosin promoter, an elongation factor-la promoter, a hemoglobin promoter, and a creatine kinase promoter. In some instances, the polynucleotide comprises a sequence encoding a poly (A) tail. In some instances, the polynucleotide comprises a 3′ UTR sequence. In some instances, the polynucleotide comprises a 5′ UTR sequence. In some embodiments, a polynucleotide disclosed herein comprises multiple smaller and discrete nucleotide sequences that are typically heterologous and exhibit different and measurable function(s), a promoter sequence, a translation initiating sequence, a start codon, a polyadenylation sequence, a stop codon, a nucleotide sequence encoding an epitope, e.g., a T cell epitope, e.g., a cancer-specific CD8+ and/or CD4+ epitope, e.g., a cancer-specific CD8+ epitope, e.g., an epitope comprising an amino acid sequence as set forth in any of SEQ ID NOs 1-35, and/or a linker encoding sequence. In some embodiments, the polynucleotide is codon optimized, e.g., codon optimized for optimal expression in a mammalian cell, e.g., a human cell.

In some embodiments, the polynucleotide, e.g., the RNA, e.g., the mRNA encoding the epitope, e.g., the T cell epitope, e.g., the cancer-specific CD8+ and/or CD4+ epitope, e.g., the cancer-specific CD8+ epitope is introduced into cells. In some embodiments, in vitro transcribed RNA can be introduced to a cell as a form of transient transfection. In some embodiments, the polynucleotide can include some or all of the 5′ and/or 3′ untranslated regions (UTRs). The polynucleotide can include exons and introns. In some embodiments, the DNA to be used for PCR is a human nucleic acid sequence. In some embodiments, the DNA to be used for PCR is a human nucleic acid sequence including the 5′ and 3′ UTRs. The DNA can alternatively be an artificial DNA sequence that is not normally expressed in a naturally occurring organism. An exemplary artificial DNA sequence is one that contains portions of genes that are ligated together to form an open reading frame that encodes a fusion protein. The portions of DNA that are ligated together can be from a single organism or from more than one organism.

Any DNA polymerase useful for PCR can be used in the methods disclosed herein. The reagents and polymerase are commercially available from a number of sources. Chemical structures with the ability to promote stability and/or translation efficiency may also be used. The RNA preferably has 5′ and 3′ UTRs. In some embodiments, the 5′ UTR is between one and 3,000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths that can be used to achieve optimal translation efficiency following transfection of the transcribed RNA.

The 5′ and 3′ UTRs can be the naturally occurring, endogenous 5′ and 3′ UTRs for the polynucleotide of interest. Alternatively, UTR sequences that are not endogenous to the polynucleotide of interest can be added by incorporating the UTR sequences into the forward and reverse primers or by any other modifications of the template. The use of UTR sequences that are not endogenous to the polynucleotide of interest can be useful for modifying the stability and/or translation efficiency of the RNA. For example, it is known that AU-rich elements in 3′UTR sequences can decrease the stability of mRNA. Therefore, 3′ UTRs can be selected or designed to increase the stability of the transcribed RNA based on properties of UTRs that are well known in the art.

In some embodiments, the 5′ UTR can contain the Kozak sequence of the endogenous nucleic acid. Alternatively, when a 5′ UTR that is not endogenous to the nucleic acid of interest is being added by PCR as described above, a consensus Kozak sequence can be redesigned by adding the 5′ UTR sequence. Kozak sequences can increase the efficiency of translation of some RNA transcripts. In some embodiments, the 5′ UTR can be 5′UTR of an RNA virus whose RNA genome is stable in cells. In some embodiments, various nucleotide analogues can be used in the 3′ or 5′ UTR to impede exonuclease degradation of the mRNA.

In some embodiments, the polynucleotide further comprises a promoter of transcription e.g., a T7 polymerase promoter. Other useful promoters include, but are not limited to, T3 and SP6 RNA polymerase promoters. Consensus nucleotide sequences for T7, T3 and SP6 promoters are known in the art.

In some embodiments, the polynucleotide, e.g., the mRNA has both a cap on the 5′ end and a 3′ poly(A) tail which determine ribosome binding, initiation of translation and stability mRNA in the cell. In some embodiments, the polynucleotide comprises a Poly (A) tail, e.g., a Poly (A) tails providing stability to the mRNA and/or reduce mRNA degradation. In some embodiment, the poly(A) sitail is between 100 and 5000 adenosines (SEQ ID NO: 125). In some embodiments, the polynucleotide may comprise immunostimulatory sequences (e.g., ISSs or CpGs).

In some embodiments, the polynucleotide, can also contain an internal ribosome entry site (IRES) sequence. The IRES sequence may be any viral, chromosomal or artificially designed sequence which initiates cap-independent ribosome binding to mRNA and facilitates the initiation of translation. Any solutes suitable for cell electroporation, which can contain factors facilitating cellular permeability and viability such as sugars, peptides, lipids, proteins, antioxidants, and surfactants can be included.

In some embodiments, the polynucleotide of this disclosure can encode any recombinant polypeptide described herein. For example, the polynucleotide can encode a recombinant polypeptide comprising a cancer-specific T cell epitope having a sequence identity of at least about 90-100% to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, or SEQ ID NO: 35. In some embodiments, the polynucleotide can encode a recombinant polypeptide having at least about 1, 2, 3, 4, 5, 6, 7, 8, 9, 10, 11, 12, 13, 14, or more amino acid residues of SEQ ID NO: 1, SEQ ID NO: 2, SEQ ID NO: 3, SEQ ID NO: 4, SEQ ID NO: 5, SEQ ID NO: 6,SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, or SEQ ID NO: 35. In some embodiments, the polynucleotide can encode a recombinant polypeptide having at least about 1, 2, 3, 4, 5, 6, 7, 8, 9, 10, 11, 12, 13, 14, or more amino acid substitutions of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, or SEQ ID NO: 35.

In some embodiments, the polynucleotide encodes a recombinant polypeptide comprising a cancer-specific CD8+ T cell epitope that can bind to an HLA molecule e.g., an HLA-A allele, e.g., an HLA-B allele, e.g., an HLA-C allele. In some embodiments the HLA-A allele is an HLA-A*15 allele, (e.g., an HLA-A*15:01 allele), an HLA-A*02 allele (e.g., an HLA-A*02:01 allele, e.g., an HLA-A*02:05 allele), an HLA-A*03 allele (e.g., an HLA-A*03:01 allele, an HLA-A*11 allele (e.g., an HLA-A*11:01 allele), or an HLA-A*31 allele (e.g., an HLA-A*31:01 allele). In some embodiments the HLA-B allele is an HLA-B*07 allele (e.g., an HLA-B*07:02 allele), an HLA-B*08 allele (e.g., an HLA-B*08:01 allele), an HLA-B*15 allele (e.g., an HLA-B*15:01 allele), an HLA-B*18 allele (e.g., an HLA-B*18:01 allele), an HLA-B*35 allele (e.g., an HLA-B*35:01 allele, an HLA-B*35:03 allele), an HLA-B*40 allele (e.g., an HLA-B*40:01 allele, an HLA-B*40:02 allele), or an HLA-B*44 allele (e.g., an HLA-B*44:01 allele, e.g., an HLA-B*44:02 allele). In some embodiments the HLA-C allele is an HLA-C*03 allele, (e.g. an HLA-C*03:04 allele), or an HLA-C*07 allele, (e.g., an HLA-C*07:01 allele, e.g., an HLA-C*07:02 allele).

The nucleotide sequence encoding an epitope e.g., a single T cell epitope, e.g., a single cancer-specific CD8+ and/or CD4+ epitope, e.g., a single cancer-specific CD8+ epitope may comprise from approximately 12 to approximately 45 base pairs, e.g., approximately 15 to approximately 45 base pairs, approximately 18 to approximately 45 base pairs, approximately 21 to approximately 45 base pairs, approximately 24 to approximately 45 base pairs.

In some embodiments, a polynucleotide disclosed herein can be a spherical nucleic acid (SNA). In some embodiments, an SNA disclosed herein comprises a nanoparticle conjugated with a polynucleotide sequence disclosed herein. In some embodiments, a SNA disclosed herein comprises an unilamellar liposome core. In some embodiments, a SNA disclosed herein comprises one or more CpG oligonucleotides. In some embodiments, an SNA disclosed herein comprises a polynucleotide, e.g., a polynucleotide comprising any of SEQ ID NOs: 52-121 encapsulated within the liposome core. In some embodiments, a polynucleotide, e.g., a polynucleotide comprising any of SEQ ID NOs: 52-121 is located on the surface of an SNA disclosed herein. In some embodiments, a polynucleotide, e.g., a polynucleotide comprising any of SEQ ID NOs: 52121 is chemically conjugated to the liposomal surface of an SNA disclosed herein. In some embodiments, a polynucleotide, e.g., a polynucleotide comprising any of SEQ ID NOs: 52121 is hybridized to one or more CpG oligonucleotides and adsorbed to the liposome surface. In some embodiments, an SNA disclosed herein can be a liposomal SNA.

### Vectors

In one aspect, the disclosure provides a vector, e.g., a vector comprising a recombinant polypeptide described herein, a vector comprising a polynucleotide encoding a recombinant polypeptide described herein. In some embodiments, the vector is a viral vector. In some embodiments, the vector is a non-viral vector.

In some embodiments, a viral vector can be an adenovirus vector, an adeno-associated virus (AAV) vector (e.g., AAV type 5 and type 2), an alphavirus vectors (e.g., Venezuelan equine encephalitis virus (VEE), a sindbis virus (SIN), semliki forest virus (SFV), and VEE-SIN chimeras), a flaviviral vector, a herpes virus vector (e.g. vectors derived from cytomegaloviruses, like rhesus cytomegalovirus (RhCMV), an arena virus vector (e.g. lymphocytic choriomeningitis virus (LCMV) vectors), a measles virus vector, a BCG vector, a vaccinia vector, a papillomaviral vector, and a poxvirus vector.

In some embodiments, the vector comprises an adenoviral vector. In some embodiments, the adenoviral vector can be or derived from a human adenovirus vector, a simian adenovirus, etc., or comprises a species B adenovirus vector (e.g., Ad3, 11p, and 35), a species C adenovirus vector (e.g., Adl,Ad 2, Ad5 and Ad6), a species E adenovirus vector (e.g., Ad4), a species F adenovirus vector (e.g., Ad41) or a chimeric Ad5 vector comprising the Adll or Ad35 fibers (Ad5/11 and Ad5/35). an adenovirus 6 vector, a PanAd3 vector, or an adenovirus C3 vector. In some embodiments, the adenoviral vector is a human serotype 5 (AdHu5). In some embodiments the vector may be an animal derived adenoviral vector for example canine, simian in particular rhesus monkey and chimpanzee. In some embodiments, the adenoviral vector may be a rare serotype vector derived from a non-human primate. Vectors derived from chimpanzee may be suitable for the vector for the present disclosure, examples include but are not limited to ChAd63, ChAd3, ChAdY25.

In some embodiments, the vector is an adenoviral vector, e.g., an adenoviral vector comprising a polynucleotide sequence encoding a single epitope, e.g., a single T cell epitope, e.g., a single cancer-specific CD8+ epitope, optionally wherein the vector is capable of inducing an inflating memory CD8+ T cell response. In some embodiments, the adenoviral vector comprises a polynucleotide sequence encoding a single epitope, e.g., a single T cell epitope, e.g., a single cancer-specific CD8+ epitope, and the vector does not comprise any additional cancer specific CD8+ T cell epitopes. In some embodiments, the adenoviral vector comprises a polynucleotide sequence encoding multiple epitopes, e.g., multiple T cell epitopes, e.g., multiple cancer-specific CD8+ epitopes.

In some embodiments, the vector is or comprises an AAV vector. In some embodiments, the AAV vector may be an AAV1, AAV2, AAVS or any combination thereof. In some embodiments, the AAV vector is selected for delivery to a particular cell, tissue, or organ, e.g., AAV serotypes 1, 2, 5 or a hybrid capsid AAV1, AAV2, AAVS or any combination thereof for targeting brain or neuronal cells; AAV4 for targeting cardiac tissue, and AAV8 for delivery to the liver.

In some embodiments, the vector may be modified to reduce the immunogenicity and improve biosafety of the vector. In some embodiments, the vector may be replication-deficient. In some embodiments, the vector may lack the E1 and E3 proteins.

In some embodiments, the vector may be a recombinant (e.g., engineered) adenoviral vector, wherein the backbone of the recombinant adenoviral vector is an adenovirus serotype 5 (Ad5) nucleic acid backbone. The recombinant adenoviral vector may comprise a deletion or a functional deletion of an E1 gene. The recombinant adenoviral vector may comprise a deletion or a functional deletion of an E3 gene. The recombinant adenoviral vector may comprise a deletion or a functional deletion of the E1 and E3 genes. The recombinant adenoviral vector may comprise a transgene inserted at the locus of the deleted or functionally deleted E1 gene. The recombinant adenoviral vector may comprise a transgene inserted at the locus of the deleted or functionally deleted E3 gene. The transgene insertion may comprise a single cancer-specific CD8+ T cell epitope described herein. The transgene insertion may comprise a single cancer-specific CD4+ T cell epitope described herein. The transgene insertion may comprise more than one cancer-specific CD4+ T cell epitope. The transgene insertion may comprise a cancer-specific CD8+ T cell epitope and a cancer-specific CD4+ T cell epitope. A nucleic acid sequence encoding the single cancer-specific CD8+ T cell epitope may be inserted in the E1 region.

In some embodiments, the vector further comprises a promoter. In some embodiments, the vector is an in vitro transcribed vector. In some embodiments, the vector may comprise a strong promoter, examples include but are not limited to a CMV promoter, an RSV promoter, an EFla promoter, a simian virus 40 (SV40) early promoter, a mouse mammary tumor virus (MMTV), a human immunodeficiency virus (HIV) long terminal repeat (LTR) promoter, a MoMuLV promoter, an avian leukemia virus promoter, an Epstein-Barr virus immediate early promoter, a Rous sarcoma virus (RSV) promoter, as well as human gene promoters such as, but not limited to, the actin promoter, the myosin promoter, the elongation factor-la promoter, the hemoglobin promoter, and the creatine kinase promoter. In some embodiments, the vector comprises a CMV promoter. An exemplary sequence for a CMV promotor is provided in SEQ ID NO: 31.

In some embodiments, the vector may comprise an inducible promoter. Examples of inducible promoters include, but are not limited to a metallothionine promoter, a glucocorticoid promoter, a progesterone promoter, and a tetracycline-regulated promoter.

In some embodiments, a vector disclosed herein can comprise additional promoter elements, e.g., enhancers and/or silencers.

In some embodiments, the vector may comprise a TATA box. In some embodiments, the vector comprises a translation initiation sequence, for example a Kozak sequence. A Kozak sequence has the consensus sequence (gcc) gccRccAUGG (SEQ ID NO: 126), a suitable Kozak sequence is provided in SEQ ID NO: 37. In some embodiments, the vector comprises a termination sequence and/or a polyadenylation sequence. A suitable polyadenylation sequence is provided in SEQ ID NO: 43. The AAV vector may comprise inverted terminal repeat (ITR) sequences. A suitable 3′ ITR sequence is provided in SEQ ID NO: 51. A suitable 5′ ITR sequence is provided in SEQ ID NO: 50.

In some embodiments, the viral vector comprises the vector back bone, a promoter region and a nucleotide sequence encoding a single epitope, e.g., a single T cell epitope, e.g., a single cancer-specific CD8+ and/or CD4+ epitope, e.g., a single cancer-specific CD8+ epitope.

In some embodiments, the ITR sequences flank the encoded the epitope, e.g., the T cell epitope, e.g., the cancer-specific CD8+ and/or CD4+ epitope, e.g., the cancer-specific CD8+ epitope. In some embodiments, there may be an ITR sequence present 5′ to the cancer specific epitope. In some embodiments, there may be an ITR sequence 3′ to the cancer specific epitope. In some embodiments, there may be an ITR sequence present 5′ to the cancer specific epitope and/or an ITR sequence 3′ to the cancer specific epitope. In some embodiments, the 5′ ITR sequence may comprise SEQ ID NO: 50. In some embodiments, the 3′ ITR sequence may comprise SEQ ID NO: 51. In some embodiments, the vector may comprise sequences 5′ to the cancer specific epitope for example SEQ ID NOs: 44 or 47. In some embodiments, the vector may comprise sequences 3′ to the cancer specific epitope for example SEQ ID NOs: 45 or 48. In some embodiments, in order to produce the viral vector comprising the cancer specific CD8+ T cell epitope, helper plasmids may be used. In some embodiments, a helper plasmid or plasmids may be used to provide genes required for viral vector replication or packaging. In some embodiments, helper plasmid encodes E2A, E4 and VA adenoviral proteins and or encodes the rep and cap genes of AAV. In some embodiments, the vector may comprise a sequence as set forth in SEQ ID NOs: 46 or 49.

In some embodiments, the vector may be modified to reduce the immunogenicity and improve biosafety of the vector. In some embodiments, the vector may be replication-deficient. In some embodiments, the vector may lack the E1 and E3 proteins. The vector may comprise sequences 5′ to the cancer specific epitope for example SEQ ID NOs: 44 or 47. The vector may comprise sequences 3′ to the cancer specific epitope for example SEQ ID NOs: 45 or 48.

In some embodiments, a vector may also be derived from retroviruses such as the lentivirus. In some embodiments, a vector disclosed herein, e.g., a lentiviral vector can transduce non-proliferating cells, such as hepatocytes. In some embodiments, a vector disclosed herein has low immunogenicity.

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses.

The term “lentiviral vector” refers to a vector derived from at least a portion of a lentivirus genome, including especially a self-inactivating lentiviral vector as provided in Milone et al., Mol. Ther. 17 (8): 1453-1464 (2009). Other examples of lentivirus vectors that may be used in the clinic, include but are not limited to, e.g., the LENTIVECTOR TM gene delivery technology from Oxford BioMedica, the LENTIMAXTM vector system from Lentigen, and the like. Nonclinical types of lentiviral vectors are also available and would be known to one skilled in the art.

The recombinant polypeptide described herein may be used in multicistronic vectors or vectors expressing several polypeptides in the same transcriptional unit. Such vectors may use internal ribosomal entry sites (IRES). Since IRES are not functional in all hosts and do not allow for the stoichiometric expression of multiple protein, self-cleaving peptides may be used instead. For example, several viral peptides are cleaved during translation and allow for the expression of multiple proteins form a single transcriptional unit. Such peptides include 2A-peptides, or 2A-like sequences, from members of the Picornaviridae virus family. See for example Szymczak et al., 2004, Nature Biotechnology; 22:589-594. In some embodiments, the recombinant nucleic acid described herein encodes the recombinant polypeptide in frame with the agent, with the two sequences separated by a self-cleaving peptide, such as a 2A sequence, or a T2A sequence.

In some embodiments, a vector disclosed herein can also contain a selectable marker gene and/or a reporter gene or both. In some embodiments, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479:79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In some embodiments, the vector is a non-viral vector, e.g., a plasmid, a cosmid, a virus-like particle, a bacterial vector, an artificial chromosome, a liposome, a lipid nanoparticle, or an exosome. In some embodiments, the vector is a bacterial vector, e.g., an attenuated Salmonella typhimurium, Salmonella typhi, Shigella, Bacillus, Lactobacillus, Bacille Calmette-Guerin (BCG), E. coli, Vibrio cholerae, Campylobacter, Listeria. In some embodiments, the vector is an attenuated and/or a non-pathogenic vector.

In the case where a non-viral vector is utilized, an exemplary non-viral vector is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DIVIPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5:505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

In some embodiments, the polynucleotide of the present disclosure can be cloned into a number of types of vectors. For example, the polynucleotide can be cloned into a vector including, but not limited to a plasmid, a phagemid, a phage derivative, a virus, and a cosmid. In some embodiments, the polynucleotide can be cloned into a vector including, but not limited to an expression vector, a replication vector, a probe generation vector, and a sequencing vector. In some embodiments, a vector disclosed herein comprises a recombinant polypeptide disclosed herein, e.g., a recombinant polypeptide comprising a single epitope, e.g., a single T cell epitope, e.g., a single cancer-specific CD8+ and/or CD4+ epitope, e.g., a single cancer-specific CD8+ epitope, e.g., a single cancer-specific CD8+ epitope comprising an amino acid sequence as set forth in any of SEQ ID NOs: 1-35. In some embodiments, a vector disclosed herein comprises a polynucleotide disclosed herein, e.g., a polynucleotide encoding a recombinant polypeptide disclosed herein, e.g., a recombinant polypeptide comprising a single epitope, e.g., a single T cell epitope, e.g., a single cancer-specific CD8+ and/or CD4+ epitope, e.g., a single cancer-specific CD8+ epitope, comprising an amino acid sequence as set forth in any of SEQ ID NOs: 1-35.

In some embodiments, the vector comprises a single epitope, e.g., a single T cell epitope, e.g., a single cancer-specific CD8+ and/or CD4+ epitope, e.g., a single cancer-specific CD8+ epitope, e.g., a single cancer-specific CD8+ epitope comprising an amino acid sequence as set forth in any of SEQ ID NOs: 1-35.

In some embodiments, the vector can encode a single cancer-specific CD8+ T cell epitope as set forth in SEQ ID NO: 1. In some embodiments, the vector can encode a single cancer-specific CD8+ T cell epitope as set forth in SEQ ID NO: 2. In some embodiments, the vector can encode a single cancer-specific CD8+ T cell epitope as set forth in SEQ ID NO: 3. In some embodiments, the vector can encode a single cancer-specific CD8+ T cell epitope as set forth in SEQ ID NO: 4. In some embodiments, the vector can encode a single cancer-specific CD8+ T cell epitope as set forth in SEQ ID NO: 5. In some embodiments, the vector can encode a single cancer-specific CD8+ T cell epitope as set forth in SEQ ID NO: 6. In some embodiments, the vector can encode a single cancer-specific CD8+ T cell epitope as set forth in SEQ ID NO: 7. In some embodiments, the vector can encode a single cancer-specific CD8+ T cell epitope as set forth in SEQ ID NO: 8. In some embodiments, the vector can encode a single cancer-specific CD8+ T cell epitope as set forth in SEQ ID NO: 9. In some embodiments, the vector can encode a single cancer-specific CD8+ T cell epitope as set forth in SEQ ID NO: 10. In some embodiments, the vector can encode a single cancer-specific CD8+ T cell epitope as set forth in SEQ ID NO: 11. In some embodiments, the vector can encode a single cancer-specific CD8+ T cell epitope as set forth in SEQ ID NO: 12. In some embodiments, the vector can encode a single cancer-specific CD8+ T cell epitope as set forth in SEQ ID NO: 13. In some embodiments, the vector can encode a single cancer-specific CD8+ T cell epitope as set forth in SEQ ID NO: 14. In some embodiments, the vector can encode a single cancer-specific CD8+ T cell epitope as set forth in SEQ ID NO: 15. In some embodiments, the vector can encode a single cancer-specific CD8+ T cell epitope as set forth in SEQ ID NO: 16. In some embodiments, the vector can encode a single cancer-specific CD8+ T cell epitope as set forth in SEQ ID NO: 17. In some embodiments, the vector can encode a single cancer-specific CD8+ T cell epitope as set forth in SEQ ID NO: 18. In some embodiments, the vector can encode a single cancer-specific CD8+ T cell epitope as set forth in SEQ ID NO: 19. In some embodiments, the vector can encode a single cancer-specific CD8+ T cell epitope as set forth in SEQ ID NO: 20. In some embodiments, the vector can encode a single cancer-specific CD8+ T cell epitope as set forth in SEQ ID NO: 21. In some embodiments, the vector can encode a single cancer-specific CD8+ T cell epitope as set forth in SEQ ID NO: 22. In some embodiments, the vector can encode a single cancer-specific CD8+ T cell epitope as set forth in SEQ ID NO: 23. In some embodiments, the vector can encode a single cancer-specific CD8+ T cell epitope as set forth in SEQ ID NO: 24. In some embodiments, the vector can encode a single cancer-specific CD8+ T cell epitope as set forth in SEQ ID NO: 25. In some embodiments, the vector can encode a single cancer-specific CD8+ T cell epitope as set forth in SEQ ID NO: 26. In some embodiments, the vector can encode a single cancer-specific CD8+ T cell epitope as set forth in SEQ ID NO: 27. In some embodiments, the vector can encode a single cancer-specific CD8+ T cell epitope as set forth in SEQ ID NO: 28. In some embodiments, the vector can encode a single cancer-specific CD8+ T cell epitope as set forth in SEQ ID NO: 29. In some embodiments, the vector can encode a single cancer-specific CD8+ T cell epitope as set forth in SEQ ID NO: 30. In some embodiments, the vector can encode a single cancer-specific CD8+ T cell epitope as set forth in SEQ ID NO: 31. In some embodiments, the vector can encode a single cancer-specific CD8+ T cell epitope as set forth in SEQ ID NO: 32. In some embodiments, the vector can encode a single cancer-specific CD8+ T cell epitope as set forth in SEQ ID NO: 33. In some embodiments, the vector can encode a single cancer-specific CD8+ T cell epitope as set forth in SEQ ID NO: 34. In some embodiments, the vector can encode a single cancer-specific CD8+ T cell epitope as set forth in SEQ ID NO: 35.

In some embodiments, the vector encodes a single epitope, e.g., a single T cell epitope, e.g., a single cancer-specific CD8+ and/or CD4+ epitope, e.g., a single cancer-specific CD8+ epitope comprising approximately 5 to approximately 15 amino acids, approximately 6 to approximately 15 amino acids, approximately 7 to approximately 15 amino acids, or approximately 8 to approximately 15 amino acids.

In some embodiments, the vector encodes a single epitope, e.g., a single T cell epitope, e.g., a single T cell epitope, e.g., a single cancer-specific CD8+ and/or CD4+ epitope, e.g., a single cancer-specific CD8+ epitope selected from GVYDGREHTV (SEQ ID NO: 1), KLVELEHTL (SEQ ID NO: 2), SPSSASLAL (SEQ ID NO: 3), KPRPDVTNEL (SEQ ID NO: 4), KEFAFLEHSL (SEQ ID NO: 5), and HELGFKVVL (SEQ ID NO: 6). In some embodiments, the vector encodes two or more (e.g., 2, 3, 4, 5, 6, or more) cancer-specific CD8+ and/or CD4+ T cell epitopes selected from GVYDGREHTV (SEQ ID NO: 1), KLVELEHTL (SEQ ID NO: 2), SPSSASLAL (SEQ ID NO: 3), KPRPDVTNEL (SEQ ID NO: 4), KEFAFLEHSL (SEQ ID NO: 5), and HELGFKVVL (SEQ ID NO: 6), or any combination thereof.

In some embodiments, the vector encodes a single epitope, e.g., a single T cell epitope, e.g., a single T cell epitope, e.g., a single cancer-specific CD8+ and/or CD4+ epitope, e.g., a single cancer-specific CD8+ epitope selected from GVYDGREHTV (SEQ ID NO: 1), KLVELEHTL (SEQ ID NO: 2), SPSSASLAL (SEQ ID NO: 3), KPRPDVTNEL (SEQ ID NO: 4), KEFAFLEHSL (SEQ ID NO: 5), HELGFKVVL (SEQ ID NO: 6), LKPDHIQR (SEQ ID NO: 7), SLFGARPGR (SEQ ID NO: 8), KTEVHGRLK (SEQ ID NO: 9), NLRPPTQEL (SEQ ID NO: 10), RVSLPKLGYK (SEQ ID NO: 11), LRPPTQEL (SEQ ID NO: 12), and RPASPRPAP (SEQ ID NO: 13). In some embodiments, the vector encodes two or more (e.g., 2, 3, 4, 5, 6, or more) cancer-specific CD8+ and/or CD4+ T cell epitopes selected from GVYDGREHTV (SEQ ID NO: 1), KLVELEHTL (SEQ ID NO: 2), SPSSASLAL (SEQ ID NO: 3), KPRPDVTNEL (SEQ ID NO: 4), KEFAFLEHSL (SEQ ID NO: 5), HELGFKVVL (SEQ ID NO: 6), LKPDHIQR (SEQ ID NO: 7), SLFGARPGR (SEQ ID NO: 8), KTEVHGRLK (SEQ ID NO: 9), NLRPPTQEL (SEQ ID NO: 10), RVSLPKLGYK (SEQ ID NO: 11), LRPPTQEL (SEQ ID NO: 12), and RPASPRPAP (SEQ ID NO: 13), or any combination thereof.

In some embodiments, the vector encodes a single epitope, e.g., a single T cell epitope, e.g., a single T cell epitope, e.g., a single cancer-specific CD8+ and/or CD4+ epitope, e.g., a single cancer-specific CD8+ epitope selected from GVYDGREHTV (SEQ ID NO: 1), KLVELEHTL (SEQ ID NO: 2), SPSSASLAL (SEQ ID NO: 3), KPRPDVTNEL (SEQ ID NO: 4), KEFAFLEHSL (SEQ ID NO: 5), HELGFKVVL (SEQ ID NO: 6), LKPDHIQR (SEQ ID NO: 7), SLFGARPGR (SEQ ID NO: 8), KTEVHGRLK (SEQ ID NO: 9), NLRPPTQEL (SEQ ID NO: 10), RVSLPKLGYK (SEQ ID NO: 11), LRPPTQEL (SEQ ID NO: 12), RPASPRPAP (SEQ ID NO: 13), IATKIALQM (SEQ ID NO: 14), SLYQTIRLK (SEQ ID NO: 15), KTPLHTLLK (SEQ ID NO: 16), RLHSFTLRQK (SEQ ID NO: 17), RVFTSSLKTK (SEQ ID NO: 18), RVLAKGLAK (SEQ ID NO: 19), GPRPSPTRSV (SEQ ID NO: 20), GQHLHLETF (SEQ ID NO: 21), SPRSPSPSL (SEQ ID NO: 22), VPQEAVRAPL (SEQ ID NO: 23), IYPFINSH (SEQ ID NO: 24), SPSSASLTL (SEQ ID NO: 25), LNKVKTSL (SEQ ID NO: 26), NLKTHLRL (SEQ ID NO: 27), FRGVFVHRY (SEQ ID NO: 28), TSGPVTEKY (SEQ ID NO: 29), and VVAAHLAGA (SEQ ID NO: 30). In some embodiments, the vector encodes two or more (e.g., 2, 3, 4, 5, 6, or more) cancer-specific CD8+ and/or CD4+ T cell epitopes selected from GVYDGREHTV (SEQ ID NO: 1), KLVELEHTL (SEQ ID NO: 2), SPSSASLAL (SEQ ID NO: 3), KPRPDVTNEL (SEQ ID NO: 4), KEFAFLEHSL (SEQ ID NO: 5), HELGFKVVL (SEQ ID NO: 6), LKPDHIQR (SEQ ID NO: 7), SLFGARPGR (SEQ ID NO: 8), KTEVHGRLK (SEQ ID NO: 9), NLRPPTQEL (SEQ ID NO: 10), RVSLPKLGYK (SEQ ID NO: 11), LRPPTQEL (SEQ ID NO: 12), RPASPRPAP (SEQ ID NO: 13), IATKIALQM (SEQ ID NO: 14), SLYQTIRLK (SEQ ID NO: 15), KTPLHTLLK (SEQ ID NO: 16), RLHSFTLRQK (SEQ ID NO: 17), RVFTSSLKTK (SEQ ID NO: 18), RVLAKGLAK (SEQ ID NO: 19), GPRPSPTRSV (SEQ ID NO: 20), GQHLHLETF (SEQ ID NO: 21), SPRSPSPSL (SEQ ID NO: 22), VPQEAVRAPL (SEQ ID NO: 23), IYPFINSH (SEQ ID NO: 24), SPSSASLTL (SEQ ID NO: 25), LNKVKTSL (SEQ ID NO: 26), NLKTHLRL (SEQ ID NO: 27), FRGVFVHRY (SEQ ID NO: 28), TSGPVTEKY (SEQ ID NO: 29), and VVAAHLAGA (SEQ ID NO: 30) or any combinations thereof. In some embodiments, the vector encodes two or more (e.g., 2, 3, 4, 5, 6, or more) cancer-specific CD8+ and/or CD4+ T cell epitopes selected from SEQ ID NOs 1-6 or any combinations thereof. In some embodiments, the vector encodes two or more (e.g., 2, 3, 4, 5, 6, or more) cancer-specific CD8+ and/or CD4+ T cell epitopes selected from SEQ ID NOs 7-12 or any combinations thereof. In some embodiments, the vector encodes two or more (e.g., 2, 3, 4, 5, 6, or more) cancer-specific CD8+ and/or CD4+ T cell epitopes selected from SEQ ID NOs 13-18 or any combinations thereof. In some embodiments, the vector comprises: (i) a recombinant a recombinant polypeptide comprising a cancer-specific CD8+ T cell epitope); wherein the cancer-specific CD8+ T cell epitope is GVYDGREHTV (SEQ ID NO: 1). In some embodiments, the vector comprises: (i) a recombinant polypeptide consisting of a cancer-specific CD8+ T cell epitope, or (ii) a polynucleotide encoding a recombinant polypeptide consisting of a cancer-specific CD8+ T cell epitope); wherein the cancer-specific CD8+ T cell epitope is GVYDGREHTV (SEQ ID NO: 1).

In some embodiments, the vector comprises: (i) a recombinant polypeptide comprising a cancer-specific CD8+ T cell epitope, or (ii) a polynucleotide encoding a recombinant polypeptide comprising a cancer-specific CD8+ T cell epitope); wherein the cancer-specific CD8+ T cell epitope is KLVELEHTL (SEQ ID NO: 2). In some embodiments, the vector comprises: (i) a recombinant polypeptide consisting of a cancer-specific CD8+ T cell epitope, or (ii) a polynucleotide encoding a recombinant polypeptide consisting of a cancer-specific CD8+ T cell epitope); wherein the cancer-specific CD8+ T cell epitope is KLVELEHTL (SEQ ID NO: 2).

In some embodiments, the vector comprises: (i) a recombinant polypeptide comprising a cancer-specific CD8+ T cell epitope, or (ii) a polynucleotide encoding a recombinant polypeptide comprising a cancer-specific CD8+ T cell epitope); wherein the cancer-specific CD8+ T cell epitope is SPSSASLAL (SEQ ID NO: 3). In some embodiments, the vector comprises: (i) a recombinant polypeptide consisting of a cancer-specific CD8+ T cell epitope, or (ii) a polynucleotide encoding a recombinant polypeptide consisting of a cancer-specific CD8+ T cell epitope); wherein the cancer-specific CD8+ T cell epitope is SPSSASLAL (SEQ ID NO: 3).

In some embodiments, the vector comprises: (i) a recombinant polypeptide comprising a cancer-specific CD8+ T cell epitope, or (ii) a polynucleotide encoding a recombinant polypeptide comprising a cancer-specific CD8+ T cell epitope); wherein the cancer-specific CD8+ T cell epitope is KPRPDVTNEL (SEQ ID NO: 4). In some embodiments, the vector comprises: (i) a recombinant polypeptide consisting of a cancer-specific CD8+ T cell epitope, or (ii) a polynucleotide encoding a recombinant polypeptide consisting of a cancer-specific CD8+ T cell epitope); wherein the cancer-specific CD8+ T cell epitope is KPRPDVTNEL (SEQ ID NO: 4).

In some embodiments, the vector comprises: (i) a recombinant polypeptide comprising a cancer-specific CD8+ T cell epitope, or (ii) a polynucleotide encoding a recombinant polypeptide comprising a cancer-specific CD8+ T cell epitope); wherein the cancer-specific CD8+ T cell epitope is KEFAFLEHSL (SEQ ID NO: 5). In some embodiments, the vector comprises: (i) a recombinant polypeptide consisting of a cancer-specific CD8+ T cell epitope, or (ii) a polynucleotide encoding a recombinant polypeptide consisting of a cancer-specific CD8+ T cell epitope); wherein the cancer-specific CD8+ T cell epitope is KEFAFLEHSL (SEQ ID NO: 5).

In some embodiments, the vector comprises: (i) a recombinant polypeptide comprising a cancer-specific CD8+ T cell epitope, or (ii) a polynucleotide encoding a recombinant polypeptide comprising a cancer-specific CD8+ T cell epitope); wherein the cancer-specific CD8+ T cell epitope is HELGFKVVL (SEQ ID NO: 6). In some embodiments, the vector comprises: (i) a recombinant polypeptide consisting of a cancer-specific CD8+ T cell epitope, or (ii) a polynucleotide encoding a recombinant polypeptide consisting of a cancer-specific CD8+ T cell epitope); wherein the cancer-specific CD8+ T cell epitope is HELGFKVVL (SEQ ID NO: 6).

In some embodiments, the vector comprises: (i) a recombinant polypeptide comprising at least two cancer-specific CD8+ T cell epitopes, or (ii) a polynucleotide encoding a recombinant polypeptide comprising at least two cancer-specific CD8+ T cell epitopes, wherein the at least two cancer-specific CD8+ T cell epitopes are GVYDGREHTV (SEQ ID NO: 1) and KLVELEHTL (SEQ ID NO: 2). In some embodiments, the vector comprises: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes wherein the at least two cancer-specific CD8+ T cell epitopes are GVYDGREHTV (SEQ ID NO: 1) and KLVELEHTL (SEQ ID NO: 2).

In some embodiments, the vector comprises: (i) a recombinant polypeptide comprising at least two cancer-specific CD8+ T cell epitopes, or (ii) a polynucleotide encoding a recombinant polypeptide comprising at least two cancer-specific CD8+ T cell epitopes, wherein the at least two cancer-specific CD8+ T cell epitopes are GVYDGREHTV (SEQ ID NO: 1) and SPSSASLAL (SEQ ID NO: 3). In some embodiments, the vector comprises: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes wherein the at least two cancer-specific CD8+ T cell epitopes are GVYDGREHTV (SEQ ID NO: 1) and SPSSASLAL (SEQ ID NO: 3).

In some embodiments, the vector comprises: (i) a recombinant polypeptide comprising at least two cancer-specific CD8+ T cell epitopes, or (ii) a polynucleotide encoding a recombinant polypeptide comprising at least two cancer-specific CD8+ T cell epitopes, wherein the at least two cancer-specific CD8+ T cell epitopes are GVYDGREHTV (SEQ ID NO: 1) and KPRPDVTNEL (SEQ ID NO: 4). In some embodiments, the vector comprises: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes wherein the at least two cancer-specific CD8+ T cell epitopes are GVYDGREHTV (SEQ ID NO: 1) and KPRPDVTNEL (SEQ ID NO: 4).

In some embodiments, the vector comprises: (i) a recombinant polypeptide comprising at least two cancer-specific CD8+ T cell epitopes, or (ii) a polynucleotide encoding a recombinant polypeptide comprising at least two cancer-specific CD8+ T cell epitopes, wherein the at least two cancer-specific CD8+ T cell epitopes are GVYDGREHTV (SEQ ID NO: 1) and KEFAFLEHSL (SEQ ID NO: 5). In some embodiments, the vector comprises: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes wherein the at least two cancer-specific CD8+ T cell epitopes are GVYDGREHTV (SEQ ID NO: 1) and KEFAFLEHSL (SEQ ID NO: 5).

In some embodiments, the vector comprises: (i) a recombinant polypeptide comprising at least two cancer-specific CD8+ T cell epitopes, or (ii) a polynucleotide encoding a recombinant polypeptide comprising at least two cancer-specific CD8+ T cell epitopes, wherein the at least two cancer-specific CD8+ T cell epitopes are GVYDGREHTV (SEQ ID NO: 1) and HELGFKVVL (SEQ ID NO: 6). In some embodiments, the vector comprises: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes wherein the at least two cancer-specific CD8+ T cell epitopes are GVYDGREHTV (SEQ ID NO: 1) and HELGFKVVL (SEQ ID NO: 6).

In some embodiments, the vector comprises: (i) a recombinant polypeptide comprising at least two cancer-specific CD8+ T cell epitopes, or (ii) a polynucleotide encoding a recombinant polypeptide comprising at least two cancer-specific CD8+ T cell epitopes, wherein the at least two cancer-specific CD8+ T cell epitopes are KLVELEHTL (SEQ ID NO: 2) and SPSSASLAL (SEQ ID NO: 3). In some embodiments, the vector comprises: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes wherein the at least two cancer-specific CD8+ T cell epitopes are KLVELEHTL (SEQ ID NO: 2) and SPSSASLAL (SEQ ID NO: 3).

In some embodiments, the vector comprises: (i) a recombinant polypeptide comprising at least two cancer-specific CD8+ T cell epitopes, or (ii) a polynucleotide encoding a recombinant polypeptide comprising at least two cancer-specific CD8+ T cell epitopes, wherein the at least two cancer-specific CD8+ T cell epitopes are KLVELEHTL (SEQ ID NO: 2) and KPRPDVTNEL (SEQ ID NO: 4). In some embodiments, the vector comprises: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes wherein the at least two cancer-specific CD8+ T cell epitopes are KLVELEHTL (SEQ ID NO: 2) and KPRPDVTNEL (SEQ ID NO: 4).

In some embodiments, the vector comprises: (i) a recombinant polypeptide comprising at least two cancer-specific CD8+ T cell epitopes, or (ii) a polynucleotide encoding a recombinant polypeptide comprising at least two cancer-specific CD8+ T cell epitopes, wherein the at least two cancer-specific CD8+ T cell epitopes are KLVELEHTL (SEQ ID NO: 2) and KEFAFLEHSL (SEQ ID NO: 5). In some embodiments, the vector comprises: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes wherein the at least two cancer-specific CD8+ T cell epitopes are KLVELEHTL (SEQ ID NO: 2) and KEFAFLEHSL (SEQ ID NO: 5).

In some embodiments, the vector comprises: (i) a recombinant polypeptide comprising at least two cancer-specific CD8+ T cell epitopes, or (ii) a polynucleotide encoding a recombinant polypeptide comprising at least two cancer-specific CD8+ T cell epitopes, wherein the at least two cancer-specific CD8+ T cell epitopes are KLVELEHTL (SEQ ID NO: 2) and HELGFKVVL (SEQ ID NO: 6). In some embodiments, the vector comprises: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes wherein the at least two cancer-specific CD8+ T cell epitopes are KLVELEHTL (SEQ ID NO: 2) and HELGFKVVL (SEQ ID NO: 6).

In some embodiments, the vector comprises: (i) a recombinant polypeptide comprising at least two cancer-specific CD8+ T cell epitopes, or (ii) a polynucleotide encoding a recombinant polypeptide comprising at least two cancer-specific CD8+ T cell epitopes, wherein the at least two cancer-specific CD8+ T cell epitopes are SPSSASLAL (SEQ ID NO: 3) and KPRPDVTNEL (SEQ ID NO: 4). In some embodiments, the vector comprises: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes wherein the at least two cancer-specific CD8+ T cell epitopes are SPSSASLAL (SEQ ID NO: 3) and KPRPDVTNEL (SEQ ID NO: 4).

In some embodiments, the vector comprises: (i) a recombinant polypeptide comprising at least two cancer-specific CD8+ T cell epitopes, or (ii) a polynucleotide encoding a recombinant polypeptide comprising at least two cancer-specific CD8+ T cell epitopes, wherein the at least two cancer-specific CD8+ T cell epitopes are SPSSASLAL (SEQ ID NO: 3) and KEF AFLEHSL (SEQ ID NO: 5). In some embodiments, the vector comprises: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes wherein the at least two cancer-specific CD8+ T cell epitopes are SPSSASLAL (SEQ ID NO: 3) and KEFAFLEHSL (SEQ ID NO: 5).

In some embodiments, the vector comprises: (i) a recombinant polypeptide comprising at least two cancer-specific CD8+ T cell epitopes, or (ii) a polynucleotide encoding a recombinant polypeptide comprising at least two cancer-specific CD8+ T cell epitopes, wherein the at least two cancer-specific CD8+ T cell epitopes are SPSSASLAL (SEQ ID NO: 3) and HELGFKVVL (SEQ ID NO: 6). In some embodiments, the vector comprises: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes wherein the at least two cancer-specific CD8+ T cell epitopes are SPSSASLAL (SEQ ID NO: 3) and HELGFKVVL (SEQ ID NO: 6).

In some embodiments, the vector comprises: (i) a recombinant polypeptide comprising at least two cancer-specific CD8+ T cell epitopes, or (ii) a polynucleotide encoding a recombinant polypeptide comprising at least two cancer-specific CD8+ T cell epitopes, wherein the at least two cancer-specific CD8+ T cell epitopes are KPRPDVTNEL (SEQ ID NO: 4) and KEFAFLEHSL (SEQ ID NO: 5). In some embodiments, the vector comprises: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes wherein the at least two cancer-specific CD8+ T cell epitopes are KPRPDVTNEL (SEQ ID NO: 4) and KEF AFLEHSL (SEQ ID NO: 5).

In some embodiments, the vector comprises: (i) a recombinant polypeptide comprising at least two cancer-specific CD8+ T cell epitopes, or (ii) a polynucleotide encoding a recombinant polypeptide comprising at least two cancer-specific CD8+ T cell epitopes, wherein the at least two cancer-specific CD8+ T cell epitopes are KPRPDVTNEL (SEQ ID NO: 4) and HELGFKVVL (SEQ ID NO: 6). In some embodiments, the vector comprises: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes wherein the at least two cancer-specific CD8+ T cell epitopes are KPRPDVTNEL (SEQ ID NO: 4) and HELGFKVVL (SEQ ID NO: 6).

In some embodiments, the vector comprises: (i) a recombinant polypeptide comprising at least two cancer-specific CD8+ T cell epitopes, or (ii) a polynucleotide encoding a recombinant polypeptide comprising at least two cancer-specific CD8+ T cell epitopes, wherein the at least two cancer-specific CD8+ T cell epitopes are KEFAFLEHSL (SEQ ID NO: 5) and HELGFKVVL (SEQ ID NO: 6). In some embodiments, the vector comprises: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes wherein the at least two cancer-specific CD8+ T cell epitopes are KEFAFLEHSL (SEQ ID NO: 5) and HELGFKVVL (SEQ ID NO: 6).

In some embodiments, the vector comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, wherein the at least three cancer-specific CD8+ T cell epitopes are GVYDGREHTV (SEQ ID NO: 1) and KLVELEHTL (SEQ ID NO: 2) and SPSSASLAL (SEQ ID NO: 3). In some embodiments, the vector comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes wherein the at least two cancer-specific CD8+ T cell epitopes are GVYDGREHTV (SEQ ID NO: 1) and KLVELEHTL (SEQ ID NO: 2) and SPSSASLAL (SEQ ID NO: 3).

In some embodiments, the vector comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, wherein the at least three cancer-specific CD8+ T cell epitopes are GVYDGREHTV (SEQ ID NO: 1) and KLVELEHTL (SEQ ID NO: 2) and KEFAFLEHSL (SEQ ID NO: 5). In some embodiments, the vector comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes wherein the at least two cancer-specific CD8+ T cell epitopes are GVYDGREHTV (SEQ ID NO: 1) and KLVELEHTL (SEQ ID NO: 2) and KEFAFLEHSL (SEQ ID NO: 5).

In some embodiments, the vector comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, wherein the at least three cancer-specific CD8+ T cell epitopes are GVYDGREHTV (SEQ ID NO: 1) and KLVELEHTL (SEQ ID NO: 2) and KEFAFLEHSL (SEQ ID NO: 5). In some embodiments, the vector comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes wherein the at least two cancer-specific CD8+ T cell epitopes are GVYDGREHTV (SEQ ID NO: 1) and KLVELEHTL (SEQ ID NO: 2) and KEFAFLEHSL (SEQ ID NO: 5).

In some embodiments, the vector comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, wherein the at least three cancer-specific CD8+ T cell epitopes are GVYDGREHTV (SEQ ID NO: 1) and KLVELEHTL (SEQ ID NO: 2) and HELGFKVVL (SEQ ID NO: 6). In some embodiments, the vector comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes wherein the at least two cancer-specific CD8+ T cell epitopes are GVYDGREHTV (SEQ ID NO: 1) and KLVELEHTL (SEQ ID NO: 2) and HELGFKVVL (SEQ ID NO: 6).

In some embodiments, the vector comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, wherein the at least three cancer-specific CD8+ T cell epitopes are GVYDGREHTV (SEQ ID NO: 1) and SPSSASLAL (SEQ ID NO: 3) and KPRPDVTNEL (SEQ ID NO: 4). In some embodiments, the vector comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes wherein the at least two cancer-specific CD8+ T cell epitopes are GVYDGREHTV (SEQ ID NO: 1) and SPSSASLAL (SEQ ID NO: 3) and KPRPDVTNEL (SEQ ID NO: 4).

In some embodiments, the vector comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, wherein the at least three cancer-specific CD8+ T cell epitopes are GVYDGREHTV (SEQ ID NO: 1) and SPSSASLAL (SEQ ID NO: 3) and KEFAFLEHSL (SEQ ID NO: 5). In some embodiments, the vector comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes wherein the at least two cancer-specific CD8+ T cell epitopes are GVYDGREHTV (SEQ ID NO: 1) and SPSSASLAL (SEQ ID NO: 3) and KEFAFLEHSL (SEQ ID NO: 5).

In some embodiments, the vector comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, wherein the at least three cancer-specific CD8+ T cell epitopes are GVYDGREHTV (SEQ ID NO: 1) and SPSSASLAL (SEQ ID NO: 3) and HELGFKVVL (SEQ ID NO: 6). In some embodiments, the vector comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes wherein the at least two cancer-specific CD8+ T cell epitopes are GVYDGREHTV (SEQ ID NO: 1) and SPSSASLAL (SEQ ID NO: 3) and HELGFKVVL (SEQ ID NO: 6).

In some embodiments, the vector comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, wherein the at least three cancer-specific CD8+ T cell epitopes are GVYDGREHTV (SEQ ID NO: 1) and KPRPDVTNEL (SEQ ID NO: 4) and KEFAFLEHSL (SEQ ID NO: 5). In some embodiments, the vector comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes wherein the at least two cancer-specific CD8+ T cell epitopes are GVYDGREHTV (SEQ ID NO: 1) and KPRPDVTNEL (SEQ ID NO: 4) and KEFAFLEHSL (SEQ ID NO: 5).

In some embodiments, the vector comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, wherein the at least three cancer-specific CD8+ T cell epitopes are GVYDGREHTV (SEQ ID NO: 1) and KPRPDVTNEL (SEQ ID NO: 4) and HELGFKVVL (SEQ ID NO: 6). In some embodiments, the vector comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes wherein the at least two cancer-specific CD8+ T cell epitopes are GVYDGREHTV (SEQ ID NO: 1) and KPRPDVTNEL (SEQ ID NO: 4) and HELGFKVVL (SEQ ID NO: 6).

In some embodiments, the vector comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, wherein the at least three cancer-specific CD8+ T cell epitopes are GVYDGREHTV (SEQ ID NO: 1) and KPRPDVTNEL (SEQ ID NO: 4) and HELGFKVVL (SEQ ID NO: 6). In some embodiments, the vector comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes wherein the at least two cancer-specific CD8+ T cell epitopes are GVYDGREHTV (SEQ ID NO: 1) and KPRPDVTNEL (SEQ ID NO: 4) and HELGFKVVL (SEQ ID NO: 6).

In some embodiments, the vector comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, wherein the at least three cancer-specific CD8+ T cell epitopes are KLVELEHTL (SEQ ID NO: 2) and SPSSASLAL (SEQ ID NO: 3) and KPRPDVTNEL (SEQ ID NO: 4). In some embodiments, the vector comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes wherein the at least two cancer-specific CD8+ T cell epitopes are KLVELEHTL (SEQ ID NO: 2) and SPSSASLAL (SEQ ID NO: 3) and KPRPDVTNEL (SEQ ID NO: 4).

In some embodiments, the vector comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, wherein the at least three cancer-specific CD8+ T cell epitopes are KLVELEHTL (SEQ ID NO: 2) and SPSSASLAL (SEQ ID NO: 3) and KEFAFLEHSL (SEQ ID NO: 5). In some embodiments, the vector comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes wherein the at least two cancer-specific CD8+ T cell epitopes are KLVELEHTL (SEQ ID NO: 2) and SPSSASLAL (SEQ ID NO: 3) and KEFAFLEHSL (SEQ ID NO: 5).

In some embodiments, the vector comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, wherein the at least three cancer-specific CD8+ T cell epitopes are KLVELEHTL (SEQ ID NO: 2) and SPSSASLAL (SEQ ID NO: 3) and HELGFKVVL (SEQ ID NO: 6). In some embodiments, the vector comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes wherein the at least two cancer-specific CD8+ T cell epitopes are KLVELEHTL (SEQ ID NO: 2) and SPSSASLAL (SEQ ID NO: 3) and HELGFKVVL (SEQ ID NO: 6).

In some embodiments, the vector comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, wherein the at least three cancer-specific CD8+ T cell epitopes are KLVELEHTL (SEQ ID NO: 2) and KPRPDVTNEL (SEQ ID NO: 4) and KEFAFLEHSL (SEQ ID NO: 5). In some embodiments, the vector comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes wherein the at least two cancer-specific CD8+ T cell epitopes are KLVELEHTL (SEQ ID NO: 2) and KPRPDVTNEL (SEQ ID NO: 4) and KEFAFLEHSL (SEQ ID NO: 5).

In some embodiments, the vector comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, wherein the at least three cancer-specific CD8+ T cell epitopes are KLVELEHTL (SEQ ID NO: 2) and KPRPDVTNEL (SEQ ID NO: 4) and HELGFKVVL (SEQ ID NO: 6). In some embodiments, the vector comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes wherein the at least two cancer-specific CD8+ T cell epitopes are KLVELEHTL (SEQ ID NO: 2) and KPRPDVTNEL (SEQ ID NO: 4) and HELGFKVVL (SEQ ID NO: 6).

In some embodiments, the vector comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, wherein the at least three cancer-specific CD8+ T cell epitopes are KLVELEHTL (SEQ ID NO: 2) and KEFAFLEHSL (SEQ ID NO: 5) and HELGFKVVL (SEQ ID NO: 6). In some embodiments, the vector comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes wherein the at least two cancer-specific CD8+ T cell epitopes are KLVELEHTL (SEQ ID NO: 2) and KPRPDVTNEL (SEQ ID NO: 4) and HELGFKVVL (SEQ ID NO: 6).

In some embodiments, the vector comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, wherein the at least three cancer-specific CD8+ T cell epitopes are SPSSASLAL (SEQ ID NO: 3) and KPRPDVTNEL (SEQ ID NO: 4) and KEFAFLEHSL (SEQ ID NO: 5). In some embodiments, the vector comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes wherein the at least two cancer-specific CD8+ T cell epitopes are SPSSASLAL (SEQ ID NO: 3) and KPRPDVTNEL (SEQ ID NO: 4) and KEFAFLEHSL (SEQ ID NO: 5).

In some embodiments, the vector comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, wherein the at least three cancer-specific CD8+ T cell epitopes are SPSSASLAL (SEQ ID NO: 3) and KPRPDVTNEL (SEQ ID NO: 4) and HELGFKVVL (SEQ ID NO: 6). In some embodiments, the vector comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes wherein the at least two cancer-specific CD8+ T cell epitopes are SPSSASLAL (SEQ ID NO: 3) and KPRPDVTNEL (SEQ ID NO: 4) and HELGFKVVL (SEQ ID NO: 6).

In some embodiments, the vector comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, wherein the at least three cancer-specific CD8+ T cell epitopes are SPSSASLAL (SEQ ID NO: 3) and KEFAFLEHSL (SEQ ID NO: 5) and HELGFKVVL (SEQ ID NO: 6). In some embodiments, the vector comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes wherein the at least two cancer-specific CD8+ T cell epitopes are SPSSASLAL (SEQ ID NO: 3) and KEFAFLEHSL (SEQ ID NO: 5) and HELGFKVVL (SEQ ID NO: 6).

In some embodiments, the vector comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, wherein the at least three cancer-specific CD8+ T cell epitopes are KPRPDVTNEL (SEQ ID NO: 4) and KEFAFLEHSL (SEQ ID NO: 5) and HELGFKVVL (SEQ ID NO: 6). In some embodiments, the vector comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes wherein the at least two cancer-specific CD8+T cell epitopes are KPRPDVTNEL (SEQ ID NO: 4) and KEFAFLEHSL (SEQ ID NO: 5) and HELGFKVVL (SEQ ID NO: 6).

In some embodiments, the vector comprises: (i) a recombinant polypeptide comprising at least four cancer-specific CD8+ T cell epitopes, or (ii) a polynucleotide encoding a recombinant polypeptide comprising at least four cancer-specific CD8+ T cell epitopes, wherein the at least four cancer-specific CD8+ T cell epitopes are GVYDGREHTV (SEQ ID NO: 1) and KLVELEHTL (SEQ ID NO: 2) and SPSSASLAL (SEQ ID NO: 3) and KPRPDVTNEL (SEQ ID NO: 4). In some embodiments, the vector comprises: (i) a recombinant polypeptide consisting of at least four cancer-specific CD8+ T cell epitopes, or (ii) a polynucleotide encoding a recombinant polypeptide consisting of at least four cancer-specific CD8+ T cell epitopes, wherein the at least four cancer-specific CD8+ T cell epitopes are GVYDGREHTV (SEQ ID NO: 1) and KLVELEHTL (SEQ ID NO: 2) and SPSSASLAL (SEQ ID NO: 3) and KPRPDVTNEL (SEQ ID NO: 4).

In some embodiments, the vector comprises: (i) a recombinant polypeptide comprising at least four cancer-specific CD8+ T cell epitopes, or (ii) a polynucleotide encoding a recombinant polypeptide comprising at least four cancer-specific CD8+ T cell epitopes, wherein the at least four cancer-specific CD8+ T cell epitopes are GVYDGREHTV (SEQ ID NO: 1) and KLVELEHTL (SEQ ID NO: 2) and SPSSASLAL (SEQ ID NO: 3) and KEFAFLEHSL (SEQ ID NO: 5). In some embodiments, the vector comprises: (i) a recombinant polypeptide consisting of at least four cancer-specific CD8+ T cell epitopes, or (ii) a polynucleotide encoding a recombinant polypeptide consisting of at least four cancer-specific CD8+ T cell epitopes, wherein the at least four cancer-specific CD8+ T cell epitopes are GVYDGREHTV (SEQ ID NO: 1) and KLVELEHTL (SEQ ID NO: 2) and SPSSASLAL (SEQ ID NO: 3) and KEFAFLEHSL (SEQ ID NO: 5).

In some embodiments, the vector comprises: (i) a recombinant polypeptide comprising at least four cancer-specific CD8+ T cell epitopes, or (ii) a polynucleotide encoding a recombinant polypeptide comprising at least four cancer-specific CD8+ T cell epitopes, wherein the at least four cancer-specific CD8+ T cell epitopes are GVYDGREHTV (SEQ ID NO: 1) and KLVELEHTL (SEQ ID NO: 2) and SPSSASLAL (SEQ ID NO: 3) and HELGFKVVL (SEQ ID NO: 6). In some embodiments, the vector comprises: (i) a recombinant polypeptide consisting of at least four cancer-specific CD8+ T cell epitopes, or (ii) a polynucleotide encoding a recombinant polypeptide consisting of at least four cancer-specific CD8+ T cell epitopes, NO: 1) and KLVELEHTL (SEQ ID NO: 2) and SPSSASLAL (SEQ ID NO: 3) and HELGFKVVL (SEQ ID NO: 6).

In some embodiments, the vector comprises: (i) a recombinant polypeptide comprising at least four cancer-specific CD8+ T cell epitopes, or (ii) a polynucleotide encoding a recombinant polypeptide comprising at least four cancer-specific CD8+ T cell epitopes, wherein the at least four cancer-specific CD8+ T cell epitopes are GVYDGREHTV (SEQ ID NO: 1) and SPSSASLAL (SEQ ID NO: 3) and KPRPDVTNEL (SEQ ID NO: 4) and KEFAFLEHSL (SEQ ID NO: 5). In some embodiments, the vector comprises: (i) a recombinant polypeptide consisting of at least four cancer-specific CD8+ T cell epitopes, or (ii) a polynucleotide encoding a recombinant polypeptide consisting of at least four cancer-specific CD8+ T cell epitopes, wherein the at least four cancer-specific CD8+ T cell epitopes are GVYDGREHTV (SEQ ID NO: 1) and SPSSASLAL (SEQ ID NO: 3) and KPRPDVTNEL (SEQ ID NO: 4) and KEFAFLEHSL (SEQ ID NO: 5).

In some embodiments, the vector comprises: (i) a recombinant polypeptide comprising at least four cancer-specific CD8+ T cell epitopes, or (ii) a polynucleotide encoding a recombinant polypeptide comprising at least four cancer-specific CD8+ T cell epitopes, wherein the at least four cancer-specific CD8+ T cell epitopes are GVYDGREHTV (SEQ ID NO: 1) and SPSSASLAL (SEQ ID NO: 3) and KPRPDVTNEL (SEQ ID NO: 4) and HELGFKVVL (SEQ ID NO: 6). In some embodiments, the vector comprises: (i) a recombinant polypeptide consisting of at least four cancer-specific CD8+ T cell epitopes, or (ii) a polynucleotide encoding a recombinant polypeptide consisting of at least four cancer-specific CD8+ T cell epitopes, wherein the at least four cancer-specific CD8+ T cell epitopes are GVYDGREHTV (SEQ ID NO: 1) and SPSSASLAL (SEQ ID NO: 3) and KPRPDVTNEL (SEQ ID NO: 4) and HELGFKVVL (SEQ ID NO: 6).

In some embodiments, the vector comprises: (i) a recombinant polypeptide comprising at least four cancer-specific CD8+ T cell epitopes, or (ii) a polynucleotide encoding a recombinant polypeptide comprising at least four cancer-specific CD8+ T cell epitopes, wherein the at least four cancer-specific CD8+ T cell epitopes are GVYDGREHTV (SEQ ID NO: 1) and KPRPDVTNEL (SEQ ID NO: 4) and KEFAFLEHSL (SEQ ID NO: 5) and HELGFKVVL (SEQ ID NO: 6). In some embodiments, the vector comprises: (i) a recombinant polypeptide consisting of at least four cancer-specific CD8+ T cell epitopes, or (ii) a polynucleotide encoding a recombinant polypeptide consisting of at least four cancer-specific CD8+ T cell epitopes, NO: 1) and KPRPDVTNEL (SEQ ID NO: 4) and KEFAFLEHSL (SEQ ID NO: 5) and HELGFKVVL (SEQ ID NO: 6).

In some embodiments, the vector comprises: (i) a recombinant polypeptide comprising at least four cancer-specific CD8+ T cell epitopes, or (ii) a polynucleotide encoding a recombinant polypeptide comprising at least four cancer-specific CD8+ T cell epitopes, wherein the at least four cancer-specific CD8+ T cell epitopes are KLVELEHTL (SEQ ID NO: 2) and SPSSASLAL (SEQ ID NO: 3) and KPRPDVTNEL (SEQ ID NO: 4) and KEFAFLEHSL (SEQ ID NO: 5). In some embodiments, the vector comprises: (i) a recombinant polypeptide consisting of at least four cancer-specific CD8+ T cell epitopes, or (ii) a polynucleotide encoding a recombinant polypeptide consisting of at least four cancer-specific CD8+ T cell epitopes, wherein the at least four cancer-specific CD8+ T cell epitopes are KLVELEHTL (SEQ ID NO: 2) and SPSSASLAL (SEQ ID NO: 3) and KPRPDVTNEL (SEQ ID NO: 4) and KEFAFLEHSL (SEQ ID NO: 5).

In some embodiments, the vector comprises: (i) a recombinant polypeptide comprising at least four cancer-specific CD8+ T cell epitopes, or (ii) a polynucleotide encoding a recombinant polypeptide comprising at least four cancer-specific CD8+ T cell epitopes, wherein the at least four cancer-specific CD8+ T cell epitopes KLVELEHTL (SEQ ID NO: 2) and SPSSASLAL (SEQ ID NO: 3) and KPRPDVTNEL (SEQ ID NO: 4) and HELGFKVVL (SEQ ID NO: 6). In some embodiments, the vector comprises: (i) a recombinant polypeptide consisting of at least four cancer-specific CD8+ T cell epitopes, or (ii) a polynucleotide encoding a recombinant polypeptide consisting of at least four cancer-specific CD8+ T cell epitopes, wherein the at least four cancer-specific CD8+ T cell epitopes are KLVELEHTL (SEQ ID NO: 2) and SPSSASLAL (SEQ ID NO: 3) and KPRPDVTNEL (SEQ ID NO: 4) and HELGFKVVL (SEQ ID NO: 6).

In some embodiments, the vector comprises: (i) a recombinant polypeptide comprising at least four cancer-specific CD8+ T cell epitopes, or (ii) a polynucleotide encoding a recombinant polypeptide comprising at least four cancer-specific CD8+ T cell epitopes, wherein the at least four cancer-specific CD8+ T cell epitopes KLVELEHTL (SEQ ID NO: 2) and KPRPDVTNEL (SEQ ID NO: 4) and KEFAFLEHSL (SEQ ID NO: 5) and HELGFKVVL (SEQ ID NO: 6). In some embodiments, the vector comprises: (i) a recombinant polypeptide consisting of at least four cancer-specific CD8+ T cell epitopes, or (ii) a polynucleotide encoding a recombinant polypeptide consisting of at least four cancer-specific CD8+ T cell epitopes, wherein the at least four cancer-specific CD8+ T cell epitopes are KLVELEHTL (SEQ ID NO: 2) and KPRPDVTNEL (SEQ ID NO: 4) and KEF AFLEHSL (SEQ ID NO: 5) and HELGFKVVL (SEQ ID NO: 6).

In some embodiments, the vector comprises: (i) a recombinant polypeptide comprising at least four cancer-specific CD8+ T cell epitopes, or (ii) a polynucleotide encoding a recombinant polypeptide comprising at least four cancer-specific CD8+ T cell epitopes, wherein the at least four cancer-specific CD8+ T cell epitopes SPSSASLAL (SEQ ID NO: 3) and KPRPDVTNEL (SEQ ID NO: 4) and KEFAFLEHSL (SEQ ID NO: 5) and HELGFKVVL (SEQ ID NO: 6). In some embodiments, the vector comprises: (i) a recombinant polypeptide consisting of at least four cancer-specific CD8+ T cell epitopes, or (ii) a polynucleotide encoding a recombinant polypeptide consisting of at least four cancer-specific CD8+ T cell epitopes, wherein the at least four cancer-specific CD8+ T cell epitopes are SPSSASLAL (SEQ ID NO: 3) and KPRPDVTNEL (SEQ ID NO: 4) and KEF AFLEHSL (SEQ ID NO: 5) and HELGFKVVL (SEQ ID NO: 6).

In some embodiments, the vector comprises: (i) a recombinant polypeptide comprising at least five cancer-specific CD8+ T cell epitopes, or (ii) a polynucleotide encoding a recombinant polypeptide comprising at least five cancer-specific CD8+ T cell epitopes, wherein the at least five cancer-specific CD8+ T cell epitopes are GVYDGREHTV (SEQ ID NO: 1) and KLVELEHTL (SEQ ID NO: 2) and SPSSASLAL (SEQ ID NO: 3) and KPRPDVTNEL (SEQ ID NO: 4) and KEFAFLEHSL (SEQ ID NO: 5). In some embodiments, the vector comprises: (i) a recombinant polypeptide consisting of at least five cancer-specific CD8+ T cell epitopes, or (ii) a polynucleotide encoding a recombinant polypeptide consisting of at least five cancer-specific CD8+ T cell epitopes, wherein the at least five cancer-specific CD8+ T cell epitopes are GVYDGREHTV (SEQ ID NO: 1) and KLVELEHTL (SEQ ID NO: 2) and SPSSASLAL (SEQ ID NO: 3) and KPRPDVTNEL (SEQ ID NO: 4) and KEFAFLEHSL (SEQ ID NO: 5).

In some embodiments, the vector comprises: (i) a recombinant polypeptide comprising at least five cancer-specific CD8+ T cell epitopes, or (ii) a polynucleotide encoding a recombinant polypeptide comprising at least five cancer-specific CD8+ T cell epitopes, wherein the at least five cancer-specific CD8+ T cell epitopes are GVYDGREHTV (SEQ ID NO: 1) and KLVELEHTL (SEQ ID NO: 2) and SPSSASLAL (SEQ ID NO: 3) and KPRPDVTNEL (SEQ ID NO: 4) and HELGFKVVL (SEQ ID NO: 6). In some embodiments, the vector comprises: (i) a recombinant polypeptide consisting of at least five cancer-specific CD8+ T cell epitopes, or (ii) a polynucleotide encoding a recombinant polypeptide consisting of at least five cancer-specific CD8+ T cell epitopes, wherein the at least five cancer-specific CD8+ T cell epitopes are GVYDGREHTV (SEQ ID NO: 1) and KLVELEHTL (SEQ ID NO: 2) and SPSSASLAL (SEQ ID NO: 3) and KPRPDVTNEL (SEQ ID NO: 4) and HELGFKVVL (SEQ ID NO: 6).

In some embodiments, the vector comprises: (i) a recombinant polypeptide comprising at least five cancer-specific CD8+ T cell epitopes, or (ii) a polynucleotide encoding a recombinant polypeptide comprising at least five cancer-specific CD8+ T cell epitopes, wherein the at least five cancer-specific CD8+ T cell epitopes are GVYDGREHTV (SEQ ID NO: 1) and SPSSASLAL (SEQ ID NO: 3) and KPRPDVTNEL (SEQ ID NO: 4) and KEFAFLEHSL (SEQ ID NO: 5) and HELGFKVVL (SEQ ID NO: 6). In some embodiments, the vector comprises: (i) a recombinant polypeptide consisting of at least five cancer-specific CD8+ T cell epitopes, or (ii) a polynucleotide encoding a recombinant polypeptide consisting of at least five cancer-specific CD8+ T cell epitopes, wherein the at least five cancer-specific CD8+ T cell epitopes are GVYDGREHTV (SEQ ID NO: 1) and SPSSASLAL (SEQ ID NO: 3) and KPRPDVTNEL (SEQ ID NO: 4) and KEFAFLEHSL (SEQ ID NO: 5) and HELGFKVVL (SEQ ID NO: 6).

In some embodiments, the vector comprises: (i) a recombinant polypeptide comprising at least five cancer-specific CD8+ T cell epitopes, or (ii) a polynucleotide encoding a recombinant polypeptide comprising at least five cancer-specific CD8+ T cell epitopes, wherein the at least five cancer-specific CD8+ T cell epitopes are KLVELEHTL (SEQ ID NO: 2) and SPSSASLAL (SEQ ID NO: 3) and KPRPDVTNEL (SEQ ID NO: 4) and KEFAFLEHSL (SEQ ID NO: 5) and HELGFKVVL (SEQ ID NO: 6). In some embodiments, the vector comprises: (i) a recombinant polypeptide consisting of at least five cancer-specific CD8+ T cell epitopes, or (ii) a polynucleotide encoding a recombinant polypeptide consisting of at least five cancer-specific CD8+ T cell epitopes, wherein the at least five cancer-specific CD8+ T cell epitopes are KLVELEHTL (SEQ ID NO: 2) and SPSSASLAL (SEQ ID NO: 3) and KPRPDVTNEL (SEQ ID NO: 4) and KEFAFLEHSL (SEQ ID NO: 5) and HELGFKVVL (SEQ ID NO: 6).

In some embodiments, the vector comprises: (i) a recombinant polypeptide comprising at least five cancer-specific CD8+ T cell epitopes, or (ii) a polynucleotide encoding a recombinant polypeptide comprising at least five cancer-specific CD8+ T cell epitopes, wherein the at least five cancer-specific CD8+ T cell epitopes are GVYDGREHTV (SEQ ID NO: 1) and KLVELEHTL (SEQ ID NO: 2) and KPRPDVTNEL (SEQ ID NO: 4) and KEFAFLEHSL (SEQ ID NO: 5) and HELGFKVVL (SEQ ID NO: 6). In some embodiments, the vector comprises: (i) a recombinant polypeptide consisting of at least five cancer-specific CD8+ T cell epitopes, or (ii) a polynucleotide encoding a recombinant polypeptide consisting of at least five cancer-specific CD8+ T cell epitopes, wherein the at least five cancer-specific CD8+ T cell epitopes are GVYDGREHTV (SEQ ID NO: 1) and KLVELEHTL (SEQ ID NO: 2) and KPRPDVTNEL (SEQ ID NO: 4) and KEFAFLEHSL (SEQ ID NO: 5) and HELGFKVVL (SEQ ID NO: 6).

In some embodiments, the vector comprises: (i) a recombinant polypeptide comprising at least five cancer-specific CD8+ T cell epitopes, or (ii) a polynucleotide encoding a recombinant polypeptide comprising at least five cancer-specific CD8+ T cell epitopes, wherein the at least five cancer-specific CD8+ T cell epitopes are GVYDGREHTV (SEQ ID NO: 1) and KLVELEHTL (SEQ ID NO: 2) and KPRPDVTNEL (SEQ ID NO: 4) and KEFAFLEHSL (SEQ ID NO: 5) and HELGFKVVL (SEQ ID NO: 6). In some embodiments, the vector comprises: (i) a recombinant polypeptide consisting of at least five cancer-specific CD8+ T cell epitopes, or (ii) a polynucleotide encoding a recombinant polypeptide consisting of at least five cancer-specific CD8+ T cell epitopes, wherein the at least five cancer-specific CD8+ T cell epitopes are GVYDGREHTV (SEQ ID NO: 1) and KLVELEHTL (SEQ ID NO: 2) and KPRPDVTNEL (SEQ ID NO: 4) and KEFAFLEHSL (SEQ ID NO: 5) and HELGFKVVL (SEQ ID NO: 6).

In some embodiments, the vector comprises: (i) a recombinant polypeptide comprising at least five cancer-specific CD8+ T cell epitopes, or (ii) a polynucleotide encoding a recombinant polypeptide comprising at least five cancer-specific CD8+ T cell epitopes, wherein the at least five cancer-specific CD8+ T cell epitopes are GVYDGREHTV (SEQ ID NO: 1) and KLVELEHTL (SEQ ID NO: 2) and SPSSASLAL (SEQ ID NO: 3) and KEFAFLEHSL (SEQ ID NO: 5) and HELGFKVVL (SEQ ID NO: 6). In some embodiments, the vector comprises: (i) a recombinant polypeptide consisting of at least five cancer-specific CD8+ T cell epitopes, or (ii) a polynucleotide encoding a recombinant polypeptide consisting of at least five cancer-specific CD8+ T cell epitopes, wherein the at least five cancer-specific CD8+ T cell epitopes are GVYDGREHTV (SEQ ID NO: 1) and KLVELEHTL (SEQ ID NO: 2) and SPSSASLAL (SEQ ID NO: 3) and KEFAFLEHSL (SEQ ID NO: 5) and HELGFKVVL (SEQ ID NO: 6).

In some embodiments, the vector comprises: (i) a recombinant polypeptide comprising at least six cancer-specific CD8+ T cell epitopes, or (ii) a polynucleotide encoding a recombinant polypeptide comprising at least six cancer-specific CD8+ T cell epitopes, wherein the at least six cancer-specific CD8+ T cell epitopes are GVYDGREHTV (SEQ ID NO: 1) and KLVELEHTL (SEQ ID NO: 2) and SPSSASLAL (SEQ ID NO: 3) and KPRPDVTNEL (SEQ ID NO: 4) and KEFAFLEHSL (SEQ ID NO: 5) and HELGFKVVL (SEQ ID NO: 6). In some embodiments, the vector comprises: (i) a recombinant polypeptide comprising at least six cancer-specific CD8+ T cell epitopes, or (ii) a polynucleotide encoding a recombinant polypeptide comprising at least six cancer-specific CD8+ T cell epitopes, wherein the at least six cancer-specific CD8+ T cell epitopes are GVYDGREHTV (SEQ ID NO: 1) and KLVELEHTL (SEQ ID NO: 2) and SPSSASLAL (SEQ ID NO: 3) and KPRPDVTNEL (SEQ ID NO: 4) and KEFAFLEHSL (SEQ ID NO: 5) and HELGFKVVL (SEQ ID NO: 6).

In some embodiments, the vector comprises a sequence with at least 90% sequence identity to SEQ ID NO: 44 and a sequence with at least 90% sequence identity to SEQ ID NO: 45. In some embodiments, the vector further comprises a sequence with at least 90% sequence identity to SEQ ID NO: 47 and a sequence with at least 90% sequence identity to SEQ ID NO: 48.

Cancer specific CD8+ T cell epitopes may be determined using techniques know in the art such as proteomics approaches, mass spectrometry approaches, genomic approaches, transcriptome analysis, bioinformatics approaches and in silico methods. It would be possible for the skilled person to select an appropriate epitope to be encoded within the vector of the present disclosure.

Methods of introducing and expressing genes into a cell are known in the art. In the context of a vector, the vector can be readily introduced into a host cell, e.g., mammalian, bacterial, yeast, or insect cell by any method in the art. For example, the vector can be transferred into a host cell by physical, chemical, or biological means.

Physical methods for introducing a polynucleotide into a host cell include calcium phosphate precipitation, lipofection, particle bombardment, microinjection, electroporation, polymer encapsulation, peptide mediated transfection, or biolistic particle delivery systems such as “gene guns” and the like. Methods for producing cells comprising vectors and/or exogenous nucleic acids are well-known in the art. See, for example, Sambrook et al., 2012, Molecular Cloning: A Laboratory Manual, volumes 1-4, Cold Spring Harbor Press, NY). A non-limiting example for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like (see, e.g., U.S. Pat. Nos. 5,350,674 and 5,585,362.

Chemical means for introducing a polynucleotide into a host cell include colloidal dispersion systems, such as macromolecule complexes, nanocapsules, nanoparticles, lipid nanoparticle conjugates, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system.

Regardless of the method used to introduce exogenous nucleic acids into a host cell or otherwise expose a cell to the inhibitor of the present disclosure, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and western blots) or by assays described herein to identify agents falling within the scope of the present disclosure.

### Cells and Populations of Cells

In one aspect, the present disclosure provides a cell comprising (i) a recombinant polypeptide comprising a single epitope, e.g., a single T cell epitope, e.g., a single cancer-specific CD8+ and/or CD4+ epitope, e.g., a single cancer-specific CD8+ epitope, comprising an amino acid sequence as set forth in any of SEQ ID NOs: 1-35, or (ii) a polynucleotide encoding a recombinant polypeptide comprising a single epitope, e.g., a single T cell epitope, e.g., a single cancer-specific CD8+ and/or CD4+ epitope, e.g., a single cancer-specific CD8+ epitope comprising an amino acid sequence as set forth in any of SEQ ID NOs: 1-35. In some embodiments, the cell can be an antigen presenting cell (APC). In some embodiments, the cell can be a T cell stimulated with an APC described herein. In some embodiments, the cell comprises a CD8+ T cell. In some embodiments, the cell comprises a CD4+ T cell. In some embodiments, the cell is an inflating memory T cell, e.g., an inflating memory CD8+ T cell.

In one aspect, the present disclosure provides a population of cells wherein a cell in the population comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-35, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-35. In some embodiments, the population of cells comprises a plurality of APCs. In some embodiments, the plurality of APCs comprise (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-35, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-35. In some embodiments, the population of cells comprises a plurality of T cells stimulated with an APC described herein. In some embodiments, the population of cells comprises a plurality of CD8+ T cells. In some embodiments, the population of cells comprises a plurality of CD4+ T cells. In some embodiments, the population of cells comprises a plurality of inflating memory T cells, e.g., inflating memory CD8+ T cells.

Inflating memory T cells described herein can be characterized by the presences of specific markers and cell surface markers. Methods to identify and quantify these markers are well known in the art. Examples of suitable methods include but are not limited to affinity-based separation methods, magnetic cell sorting techniques, fluorescence-based cell sorting techniques such as FACS (fluorescence activated cell sorting). The inflating memory CD8+ T cells can be characterized by the presence of a number of markers, examples include but are not limited to CX3CR1, KLRG-1, CD44. The inflating memory CD8+ T cells can also be characterized by the low expression of a number of markers, example include but are not limited to CD62L, CD27, CD127. The term “low expression” may refer to cells wherein there is no expression of the markers, it may also refer to cells wherein there is low expression of the markers relative to other cells in the sample. In some embodiments, the population of cells comprises a plurality of inflating memory T cells, e.g., a plurality of inflating memory CD8+ T cells, e.g., a plurality of inflating memory CD8+ T cells wherein an inflating memory CD8+ T cell of the population expresses one or more of CX3CR1, KLRG-1, CD44 and/or have low expression of one or more of CD62L, CD27, CD127.

In some embodiments, the inflating memory CD8+ T cells described herein are characterized by markers selected from the group comprising CX3CR1+, KLRG-1+, CD44+, CD62L-, CCR7-, CD45RA+/− wherein the designation (+) indicates the presence of the marker, and the designation (−) indicates low expression or no expression of the marker; wherein the (−) designation means low expression this may be further indicated by “(low)”. In some embodiments, the inflating memory CD8+ T cells may be characterized by one or more markers selected from the group comprising CX3CR1+, KLRG-1+, CD44+, CD62L-, CCR7-, CD45RA+/−, CD27-(low), and/or CD127-(low). In some embodiments, the inflating memory CD8+ T cells may be characterized by the phenotype CX3CR1+, KLRG-1+, CD44+, CD62L-, CCR7-, CD45RA+/−. The inflating memory CD8+ T cells may be characterized by the phenotype CX3CR1+, KLRG-1+, CD44+, CD62L-, CCR7-, CD45RA+/−, CD27-(low), CD127-(low). The CD8+ T cells produced in an inflating memory response may have a number of other characteristics. For example, the cells may comprise a transcriptional profile driven by Tbx21 (also referred to as T-bet). These cells show a sustained expression of Tbx21. The cells may also show a sustained expression of E2f2 a transcription factor generally involved in cell growth and proliferation. The cells may also lack expression or have low expression of the transcription factor Eomes. In some embodiments, the cell comprising a recombinant polypeptide comprising a single epitope, e.g., a single T cell epitope, e.g., a single cancer-specific CD8+ and/or CD4+ epitope, e.g., a single cancer-specific CD8+ epitope, comprises a transcriptional profile driven by Tbx21. In some embodiments, the cell comprising a recombinant polypeptide comprising a single epitope, e.g., a single T cell epitope, e.g., a single cancer-specific CD8+ and/or CD4+ epitope, e.g., a single cancer-specific CD8+ epitope, comprises a sustained expression of E2f2. In some embodiments, the cell comprising a single epitope, e.g., a single T cell epitope, e.g., a single cancer-specific CD8+ and/or CD4+ epitope, e.g., a single cancer-specific CD8+ epitope comprises a low expression of the transcription factor Eomes.

The inflating memory CD8+ T cells may not demonstrate classical contraction after exposure to an antigen. During classical memory evolution after exposure to an antigen, the cells form a contracted central memory pool which makes up<1% of total circulating CD8+ T cells. However, inflating memory cells are maintained as large pools of cells which circulate in the blood. As such, the resulting inflating memory CD8+ T cells form approximately 0.01% to approximately 20% of total CD8+ T cells. In some embodiments, the population of inflating memory CD8+ T cells comprises 0.01%, 0.02%, 0.03%, 0.05%, 0.07%, 0.09%, 0.1%, 0.3%, 0.5%, 0.7%, 0.9%, 1%, 2%, 3%, 4%, 5%, 6%, 7%, 8%, 9%, 10%, 11%, 12%, 13%, 14%, 15%, 16%, 17%, 18%, 19%, or 20% of total CD8+ T cells. In some embodiments, the population of inflating memory CD8+ T cells comprises approximately 0.1% to approximately 20%, approximately 0.05% to approximately 20%, approximately 0.5% to approximately 20%, approximately 1% to approximately 20%, approximately 2% to approximately 20%, approximately 3% to approximately 20%, approximately 5% to approximately 20%, approximately 8% to approximately 20%, approximately 10% to approximately 20%, approximately 12% to approximately 20%, approximately 15% to approximately 20%, or approximately 18% to approximately 20% of total CD8+ T cells. In some embodiments, the population of inflating memory CD8+ T cells comprises approximately 12% to approximately 20% of total CD8+ T cells.

In some cases, the population of inflating memory CD8+ T cells can retain their effector memory phenotype. The resulting inflating memory CD8+ T cells can retain their memory effector phenotype for a prolonged period, wherein the effector phenotype is characterized by CD44+ and/or CD62L-. The inflating memory CD8+ T cells may retain their memory effector phenotype for up to 60 days post exposure to the vector of the present disclosure. In some embodiments, the population of inflating memory CD8+ T cells may retain their memory effector phenotype for up to 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours or longer post exposure to the vector of the present disclosure. In some embodiments, the population of inflating memory CD8+ T cells may retain their memory effector phenotype for up to 1 day, 2 days, 3 days, 4 days, 5 days, 6 days, 7 days, 8 days, 9 days, 10 days, 11 days, 12 days, 13 days, 15 days, 17 days, 20 days, 25 days, 30 days, 35 days, 40 days, 50 days, 55 days, 60 days or longer post exposure to the vector of the present disclosure.

The inflating memory CD8+ T cells may lack markers of exhaustion. T cell exhaustion can occur from excessive TCR (T cell receptor) stimulation. Markers of T cell exhaustion can include but are not limited to upregulation of markers such as PD-1, Tim-3, or Lag-3. As such, in some embodiments, the inflating memory CD8+ T cells may lack or demonstrate low expression of markers selected from the group consisting of, but not limited to, PD-1, Tim-3, and/or Lag-3.

In some embodiments, the T cells describe herein can demonstrate a distinct transcriptional profile from both central memory and exhausted memory T cell subsets. In some embodiments, the T cells describe herein can demonstrate features such as enhanced redox resilience which may be due to intrinsically lower levels of reactive oxygen species and resilience to oxidative stress. In some embodiments, the transcriptional profile is driven by the transcription factor Tbx21 with minimal contribution from Eomes. This results in a CD8+ T cell phenotype that is long lived, and present in the peripheral organs in high numbers whilst retaining effector function. In some embodiments, the antigen-specific inflating memory CD8+ T cells develop through one or more of processing, presentation and co-stimulation conditions. In some embodiments, the processing of the epitope occurs independently of the proteasome or immunoproteasome and optionally presentation by a non-hematopoietic unconventional APC during the later stages can help to preserve this phenotype. In some embodiments, using a vector which encodes (i) a recombinant polypeptide comprising one or more epitopes, e.g., one or more T cell epitopes, e.g., one or more cancer-specific CD8+ epitopes, e.g., one or more cancer-specific CD8+ epitopes, each comprising an amino acid sequence as set forth in any of SEQ ID NOs: 1-35, or (ii) a polynucleotide encoding a recombinant polypeptide comprising one or more epitopes, e.g., one or more T cell epitopes, e.g., one or more cancer-specific CD8+ epitopes, e.g., one or more cancer-specific CD8+ epitopes, each comprising an amino acid sequence as set forth in any of SEQ ID NOs: 1-35, the antigen processing requirements can be bypassed. In some embodiments, using a vector which encodes (i) a recombinant polypeptide comprising one or more epitopes, e.g., one or more T cell epitopes, e.g., one or more cancer-specific CD8+ epitopes, e.g., one or more cancer-specific CD8+ epitopes, each comprising an amino acid sequence as set forth in any of SEQ ID NOs: 1-35, or (ii) a polynucleotide encoding a recombinant polypeptide comprising one or more epitopes, e.g., one or more T cell epitopes, e.g., one or more cancer-specific CD8+ epitopes, e.g., one or more cancer-specific CD8+ epitopes, each comprising an amino acid sequence as set forth in any of SEQ ID NOs: 1-35, induces an inflating memory response.

### Compositions

In one aspect, the present disclosure provides a composition, e.g., a therapeutic composition. In some embodiments, the composition, e.g., the therapeutic composition induces an inflating memory CD8+ T cell response (i.e., a sustained, functional, durable CD8+ T cell response). In some embodiments, the resulting pool of CD8+ T cells are able to resist exhaustion which can occur due to prolonged TCR stimulation.

In some embodiments, the therapeutic composition comprises: (i) a recombinant polypeptide comprising a cancer-specific CD8+ T cell epitope, or (ii) a polynucleotide encoding a recombinant polypeptide comprising a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is GVYDGREHTV (SEQ ID NO: 1). In some embodiments, the therapeutic composition comprises: (i) a recombinant polypeptide consisting of a cancer-specific CD8+ T cell epitope, or (ii) a polynucleotide encoding a recombinant polypeptide consisting of a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is GVYDGREHTV (SEQ ID NO: 1). In some embodiments, the therapeutic composition comprises: (i) a recombinant polypeptide comprising a cancer-specific CD8+ T cell epitope, or (ii) a polynucleotide encoding a recombinant polypeptide comprising a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is GVYDGREHTV (SEQ ID NO: 1) and wherein the composition is for use in treating cancer is a subject that expresses an MHC encoded by an HLA-A*02:01 allele. In some embodiments, the therapeutic composition comprises: (i) a recombinant polypeptide consisting of a cancer-specific CD8+ T cell epitope, or (ii) a polynucleotide encoding a recombinant polypeptide consisting of a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is GVYDGREHTV (SEQ ID NO: 1) and wherein the composition is for use in treating cancer is a subject that expresses an MHC encoded by an HLA-A*02:01 allele.

In some embodiments, the therapeutic composition comprises: (i) a recombinant polypeptide comprising a cancer-specific CD8+ T cell epitope, or (ii) a polynucleotide encoding a recombinant polypeptide comprising a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is KLVELEHTL (SEQ ID NO: 2). In some embodiments, the therapeutic composition comprises: (i) a recombinant polypeptide consisting of a cancer-specific CD8+ T cell epitope, or (ii) a polynucleotide encoding a recombinant polypeptide consisting of a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is KLVELEHTL (SEQ ID NO: 2). In some embodiments, the therapeutic composition comprises: (i) a recombinant polypeptide comprising a cancer-specific CD8+ T cell epitope, or (ii) a polynucleotide encoding a recombinant polypeptide comprising a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is KLVELEHTL (SEQ ID NO: 2) and wherein the composition is for use in treating cancer is a subject that expresses an MHC encoded by an HLA-A*02:01 allele. In some embodiments, the therapeutic composition comprises: (i) a recombinant polypeptide consisting of a cancer-specific CD8+ T cell epitope, or (ii) a polynucleotide encoding a recombinant polypeptide consisting of a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is KLVELEHTL (SEQ ID NO: 2) and wherein the composition is for use in treating cancer is a subject that expresses an MHC encoded by an HLA-A*02:01 allele.

In some embodiments, the therapeutic composition comprises: (i) a recombinant a recombinant polypeptide comprising a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is SPSSASLAL (SEQ ID NO: 3). In some embodiments, the therapeutic composition comprises: (i) a recombinant polypeptide consisting of a cancer-specific CD8+ T cell epitope, or (ii) a polynucleotide encoding a recombinant polypeptide consisting of a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is SPSSASLAL (SEQ ID NO: 3). In some embodiments, the therapeutic composition comprises: (i) a recombinant polypeptide comprising a cancer-specific CD8+ T cell epitope, or (ii) a polynucleotide encoding a recombinant polypeptide comprising a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is SPSSASLAL (SEQ ID NO: 3) and wherein the composition is for use in treating cancer is a subject that expresses an MHC encoded by an HLA-B*07:02 allele and/or an HLA-B*35:03 allele. In some embodiments, the therapeutic composition comprises: (i) a recombinant polypeptide consisting of a cancer-specific CD8+ T cell epitope, or (ii) a polynucleotide encoding a recombinant polypeptide consisting of a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is SPSSASLAL (SEQ ID NO: 3) and wherein the composition is for use in treating cancer is a subject that expresses an MHC encoded by an HLA-B*07:02 allele and/or an HLA-B*35:03 allele.

In some embodiments, the therapeutic composition comprises: (i) a recombinant polypeptide comprising a cancer-specific CD8+ T cell epitope, or (ii) a polynucleotide encoding a recombinant polypeptide comprising a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is KPRPDVTNEL (SEQ ID NO: 4). In some embodiments, the therapeutic composition comprises: (i) a recombinant polypeptide consisting of a cancer-specific CD8+ T cell epitope, or (ii) a polynucleotide encoding a recombinant polypeptide consisting of a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is KPRPDVTNEL (SEQ ID NO: 4). In some embodiments, the therapeutic composition comprises: (i) a recombinant polypeptide comprising a cancer-specific CD8+ T cell epitope, or (ii) a polynucleotide encoding a recombinant polypeptide comprising a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is KPRPDVTNEL (SEQ ID NO: 4) and wherein the composition is for use in treating cancer is a subject that expresses an MHC encoded by an HLA-B*07:02 allele, an HLA-B*35:03 allele, an HLA-C*07:02 allele, or any combination thereof. In some embodiments, the therapeutic composition comprises: (i) a recombinant polypeptide consisting of a cancer-specific CD8+ T cell epitope, or (ii) a polynucleotide encoding a recombinant polypeptide consisting of a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is KPRPDVTNEL (SEQ ID NO: 4) and wherein the composition is for use in treating cancer is a subject that expresses an MHC encoded by an HLA-B*07:02 allele, an HLA-B*35:03 allele, an HLA-C*07:02 allele, or any combination thereof.

In some embodiments, the therapeutic composition comprises: (i) a recombinant polypeptide comprising a cancer-specific CD8+ T cell epitope, or (ii) a polynucleotide encoding a recombinant polypeptide comprising a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is KEFAFLEHSL (SEQ ID NO: 5). In some embodiments, the therapeutic composition comprises: (i) a recombinant polypeptide consisting of a cancer-specific CD8+ T cell epitope, or (ii) a polynucleotide encoding a recombinant polypeptide consisting of a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is KEFAFLEHSL (SEQ ID NO: 5). In some embodiments, the therapeutic composition comprises: (i) a recombinant polypeptide comprising a cancer-specific CD8+ T cell epitope, or (ii) a polynucleotide encoding a recombinant polypeptide comprising a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is KEFAFLEHSL (SEQ ID NO: 5) and wherein the composition is for use in treating cancer is a subject that expresses an MHC encoded by an HLA-B*40:01 allele, an HLA-B*40:02 allele, an HLA-B*44:02 allele, or any combination thereof. In some embodiments, the therapeutic composition comprises: (i) a recombinant polypeptide comprising a cancer-specific CD8+ T cell epitope, or (ii) a polynucleotide encoding a recombinant polypeptide comprising a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is KEFAFLEHSL (SEQ ID NO: 5) and wherein the composition is for use in treating cancer is a subject that expresses an MHC encoded by an HLA-B*44:02 allele. In some embodiments, the therapeutic composition comprises: (i) a recombinant polypeptide consisting of a cancer-specific CD8+ T cell epitope, or (ii) a polynucleotide encoding a recombinant polypeptide consisting of a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is KEFAFLEHSL (SEQ ID NO: 5) and wherein the composition is for use in treating cancer is a subject that expresses an MHC encoded by an HLA-B*40:01 allele, an HLA-B*40:02 allele, an HLA-B*44:02 allele, or any combination thereof. In some embodiments, the therapeutic composition comprises: (i) a recombinant polypeptide consisting of a cancer-specific CD8+ T cell epitope, or (ii) a polynucleotide encoding a recombinant polypeptide consisting of a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is KEFAFLEHSL (SEQ ID NO: 5) and wherein the composition is for use in treating cancer is a subject that expresses an MHC encoded by an HLA-B*44:02 allele.

In some embodiments, the therapeutic composition comprises: (i) a recombinant polypeptide comprising a cancer-specific CD8+ T cell epitope, or (ii) a polynucleotide encoding a recombinant polypeptide comprising a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is HELGFKVVL (SEQ ID NO: 6). In some embodiments, the therapeutic composition comprises: (i) a recombinant polypeptide consisting of a cancer-specific CD8+ T cell epitope, or (ii) a polynucleotide encoding a recombinant polypeptide consisting of a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is HELGFKVVL (SEQ ID NO: 6). In some embodiments, the therapeutic composition comprises: (i) a recombinant polypeptide comprising a cancer-specific CD8+ T cell epitope, or (ii) a polynucleotide encoding a recombinant polypeptide comprising a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is HELGFKVVL (SEQ ID NO: 6) and wherein the composition is for use in treating cancer is a subject that expresses an MHC encoded by an HLA-B*18:01 allele. In some embodiments, the therapeutic composition comprises: (i) a recombinant polypeptide comprising a cancer-specific CD8+ T cell epitope, or (ii) a polynucleotide encoding a recombinant polypeptide comprising a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is HELGFKVVL (SEQ ID NO: 6) and wherein the composition is for use in treating cancer is a subject that expresses an MHC encoded by an HLA-B*40:01 allele. In some embodiments, the therapeutic composition comprises: (i) a recombinant polypeptide comprising a cancer-specific CD8+ T cell epitope, or (ii) a polynucleotide encoding a recombinant polypeptide comprising a cancer-specific CD8+ T cell epitope (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is HELGFKVVL (SEQ ID NO: 6) and wherein the composition is for use in treating cancer is a subject that expresses an MHC encoded by an HLA-B*44:02 allele. In some embodiments, the therapeutic composition comprises: (i) a recombinant polypeptide consisting of a cancer-specific CD8+ T cell epitope, or (ii) a polynucleotide encoding a recombinant polypeptide consisting of a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is HELGFKVVL (SEQ ID NO: 6) and wherein the composition is for use in treating cancer is a subject that expresses an MHC encoded by an HLA-B*18:01 allele. In some embodiments, the therapeutic composition comprises: (i) a recombinant polypeptide consisting of a cancer-specific CD8+ T cell epitope, or (ii) a polynucleotide encoding a recombinant polypeptide consisting of a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is HELGFKVVL (SEQ ID NO: 6) and wherein the composition is for use in treating cancer is a subject that expresses an MHC encoded by an HLA-B*40:01 allele. In some embodiments, the therapeutic composition comprises: (i) a recombinant polypeptide consisting of a cancer-specific CD8+ T cell epitope, or (ii) a polynucleotide encoding a recombinant polypeptide consisting of a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is HELGFKVVL (SEQ ID NO: 6) and wherein the composition is for use in treating cancer is a subject that expresses an MHC encoded by an HLA-B*44:02 allele. In some embodiments, the therapeutic composition comprises: (i) a recombinant polypeptide consisting of a cancer-specific CD8+ T cell epitope, or (ii) a polynucleotide encoding a recombinant polypeptide consisting of a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is HELGFKVVL (SEQ ID NO: 6) and wherein the composition is for use in treating cancer is a subject that expresses an MHC encoded by an HLA-B*40:02 allele.

In some embodiments, the therapeutic composition comprises: (i) a recombinant polypeptide comprising a cancer-specific CD8+ T cell epitope, or (ii) a polynucleotide encoding a recombinant polypeptide comprising a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is NLRPPTQEL (SEQ ID NO: 10). In some embodiments, the therapeutic composition comprises: (i) a recombinant polypeptide consisting of a cancer-specific CD8+ T cell epitope, or (ii) a polynucleotide encoding a recombinant polypeptide consisting of a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is NLRPPTQEL (SEQ ID NO: 10). In some embodiments, the therapeutic composition comprises: (i) a recombinant polypeptide comprising a cancer-specific CD8+ T cell epitope, or (ii) a polynucleotide encoding a recombinant polypeptide comprising a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is NLRPPTQEL (SEQ ID NO: 10) and wherein the composition is for use in treating cancer is a subject that expresses an MHC encoded by an HLA-B*07:02 allele, an HLA-B*35:03 allele, an HLA-C*07:02 allele, an HLA-C*03:03 allele, or any combination thereof. In some embodiments, the therapeutic composition comprises: (i) a recombinant polypeptide consisting of a cancer-specific CD8+ T cell epitope, or (ii) a polynucleotide encoding a recombinant polypeptide consisting of a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is NLRPPTQEL (SEQ ID NO: 10) and wherein the composition is for use in treating cancer is a subject that expresses an MHC encoded by an HLA-B*07:02 allele, an HLA-B*35:03 allele, an HLA-C*07:02 allele, an HLA-C*03:03 allele, or any combination thereof.

In some embodiments, the therapeutic composition comprises: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are GVYDGREHTV (SEQ ID NO: 1) and KLVELEHTL (SEQ ID NO: 2). In some embodiments, the therapeutic composition comprises: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are GVYDGREHTV (SEQ ID NO: 1) and KLVELEHTL (SEQ ID NO: 2). In some embodiments, the therapeutic composition comprises: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are GVYDGREHTV (SEQ ID NO: 1) and KLVELEHTL (SEQ ID NO: 2) and wherein the composition is for use in treating cancer is a subject that expresses an MHC encoded by an HLA-A*02:01 allele. In some embodiments, the therapeutic composition comprises: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are GVYDGREHTV (SEQ ID NO: 1) and KLVELEHTL (SEQ ID NO: 2) and wherein the composition is for use in treating cancer is a subject that expresses an MHC encoded by an HLA-A*02:01 allele.

In some embodiments, the therapeutic composition comprises: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are GVYDGREHTV (SEQ ID NO: 1) and SPSSASLAL (SEQ ID NO: 3). In some embodiments, the therapeutic composition comprises: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are GVYDGREHTV (SEQ ID NO: 1) and SPSSASLAL (SEQ ID NO: 3). In some embodiments, the therapeutic composition comprises: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are GVYDGREHTV (SEQ ID NO: 1) and SPSSASLAL (SEQ ID NO: 3) and wherein the composition is for use in treating cancer is a subject that expresses an MHC encoded by an HLA-A*02:01 allele, an HLA-B*07:02 allele, an HLA-B*35:03 allele, or any combination thereof. In some embodiments, the therapeutic composition comprises: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are GVYDGREHTV (SEQ ID NO: 1) and SPSSASLAL (SEQ ID NO: 3) and wherein the composition is for use in treating cancer is a subject that expresses an MHC encoded by an HLA-A*02:01 allele, an HLA-B*07:02 allele, an HLA-B*35:03 allele, or any combination thereof.

In some embodiments, the therapeutic composition comprises: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are GVYDGREHTV (SEQ ID NO: 1) and KPRPDVTNEL (SEQ ID NO: 4). In some embodiments, the therapeutic composition comprises: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are GVYDGREHTV (SEQ ID NO: 1) and KPRPDVTNEL (SEQ ID NO: 4). In some embodiments, the therapeutic composition comprises: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are GVYDGREHTV (SEQ ID NO: 1) and KPRPDVTNEL (SEQ ID NO: 4) and wherein the composition is for use in treating cancer is a subject that expresses an MHC encoded by an HLA-A*02:01 allele, an HLA-B*07:02 allele, an HLA-B*35:03 allele, an HLA-C*07:02 allele, or any combination thereof. In some embodiments, the therapeutic composition comprises: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are GVYDGREHTV (SEQ ID NO: 1) and KPRPDVTNEL (SEQ ID NO: 4) and wherein the composition is for use in treating cancer is a subject that expresses an MHC encoded by an HLA-A*02:01 allele, an HLA-B*07:02 allele, an HLA-B*35:03 allele, an HLA-C*07:02 allele, or any combination thereof.

In some embodiments, the therapeutic composition comprises: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are GVYDGREHTV (SEQ ID NO: 1) and KEFAFLEHSL (SEQ ID NO: 5). In some embodiments, the therapeutic composition comprises: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are GVYDGREHTV (SEQ ID NO: 1) and KEFAFLEHSL (SEQ ID NO: 5). In some embodiments, the therapeutic composition comprises: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are GVYDGREHTV (SEQ ID NO: 1) and KEFAFLEHSL (SEQ ID NO: 5) and wherein the composition is for use in treating cancer is a subject that expresses an MHC encoded by an HLA-A*02:01 allele, an HLA-B*40:01 allele, an HLA-B*44:02 allele, an HLA-B*40:02 allele, or any combination thereof. In some embodiments, the therapeutic composition comprises: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are GVYDGREHTV (SEQ ID NO: 1) and KEFAFLEHSL (SEQ ID NO: 5) and wherein the composition is for use in treating cancer is a subject that expresses an MHC encoded by an HLA-A*02:01 allele, an HLA-B*40:01 allele, an HLA-B*44:02 allele, an HLA-B*40:02 allele, or any combination thereof.

In some embodiments, the therapeutic composition comprises: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are GVYDGREHTV (SEQ ID NO: 1) and HELGFKVVL (SEQ ID NO: 6). In some embodiments, the therapeutic composition comprises: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are GVYDGREHTV (SEQ ID NO: 1) and HELGFKVVL (SEQ ID NO: 6). In some embodiments, the therapeutic composition comprises: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are GVYDGREHTV (SEQ ID NO: 1) and HELGFKVVL (SEQ ID NO: 6) and wherein the composition is for use in treating cancer is a subject that expresses an MHC encoded by an HLA-A*02:01 allele, an HLA-B*18:01 allele an HLA-B*40:01 allele, an HLA-B*44:02 allele, an HLA-B*40:02 allele, or any combination thereof. In some embodiments, the therapeutic composition comprises: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are GVYDGREHTV (SEQ ID NO: 1) and HELGFKVVL (SEQ ID NO: 6) and wherein the composition is for use in treating cancer is a subject that expresses an MHC encoded by an HLA-A*02:01 allele, an HLA-B*18:01 allele, an HLA-B*40:01 allele, an HLA-B*44:02 allele, an HLA-B*40:02 allele, or any combination thereof.

In some embodiments, the therapeutic composition comprises: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KLVELEHTL (SEQ ID NO: 2) and SPSSASLAL (SEQ ID NO: 3). In some embodiments, the therapeutic composition comprises: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KLVELEHTL (SEQ ID NO: 2) and SPSSASLAL (SEQ ID NO: 3). In some embodiments, the therapeutic composition comprises: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KLVELEHTL (SEQ ID NO: 2) and SPSSASLAL (SEQ ID NO: 3) and wherein the composition is for use in treating cancer is a subject that expresses an MHC encoded by an HLA-A*02:01 allele, an HLA-B*07:02 allele, an HLA-B*35:03 allele, or any combination thereof. In some embodiments, the therapeutic composition comprises: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KLVELEHTL (SEQ ID NO: 2) and SPSSASLAL (SEQ ID NO: 3) and wherein the composition is for use in treating cancer is a subject that expresses an MHC encoded by an HLA-A*02:01 allele, an HLA-B*07:02 allele, an HLA-B*35:03 allele, or any combination thereof.

In some embodiments, the therapeutic composition comprises: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KLVELEHTL (SEQ ID NO: 2) and KPRPDVTNEL (SEQ ID NO: 4). In some embodiments, the therapeutic composition comprises: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KLVELEHTL (SEQ ID NO: 2) and KPRPDVTNEL (SEQ ID NO: 4). In some embodiments, the therapeutic composition comprises: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KLVELEHTL (SEQ ID NO: 2) and KPRPDVTNEL (SEQ ID NO: 4) and wherein the composition is for use in treating cancer is a subject that expresses an MHC encoded by an HLA-A*02:01 allele, an HLA-B*07:02 allele, an HLA-B*35:03 allele, an HLA-C*07:02 allele, or any combination thereof. In some embodiments, the therapeutic composition comprises: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KLVELEHTL (SEQ ID NO: 2) and KPRPDVTNEL (SEQ ID NO: 4) and wherein the composition is for use in treating cancer is a subject that expresses an MHC encoded by an HLA-A*02:01 allele, an HLA-B*07:02 allele, an HLA-B*35:03 allele, an HLA-C*07:02 allele, or any combination thereof.

In some embodiments, the therapeutic composition comprises: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KLVELEHTL (SEQ ID NO: 2) and KEFAFLEHSL (SEQ ID NO: 5). In some embodiments, the therapeutic composition comprises: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KLVELEHTL (SEQ ID NO: 2) and KEFAFLEHSL (SEQ ID NO: 5). In some embodiments, the therapeutic composition comprises: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KLVELEHTL (SEQ ID NO: 2) and KEF AFLEHSL (SEQ ID NO: 5) and wherein the composition is for use in treating cancer is a subject that expresses an MHC encoded by an HLA-A*02:01 allele, an HLA-B*40:01 allele, an HLA-B*44:02 allele, an HLA-B*40:02 allele, or any combination thereof. In some embodiments, the therapeutic composition comprises: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KLVELEHTL (SEQ ID NO: 2) and KEFAFLEHSL (SEQ ID NO: 5) and wherein the composition is for use in treating cancer is a subject that expresses an MHC encoded by an HLA-A*02:01 allele, an HLA-B*40:01 allele, an HLA-B*44:02 allele, an HLA-B*40:02 allele, or any combination thereof.

In some embodiments, the therapeutic composition comprises: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KLVELEHTL (SEQ ID NO: 2) and HELGFKVVL (SEQ ID NO: 6). In some embodiments, the therapeutic composition comprises: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KLVELEHTL (SEQ ID NO: 2) and HELGFKVVL (SEQ ID NO: 6). In some embodiments, the therapeutic composition comprises: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KLVELEHTL (SEQ ID NO: 2) and HELGFKVVL (SEQ ID NO: 6) and wherein the composition is for use in treating cancer is a subject that expresses an MHC encoded by an HLA-A*02:01 allele, an HLA-B*18:01 allele, an HLA-B*40:01 allele, an HLA-B*44:02 allele, an HLA-B*40:02 allele, or any combination thereof. In some embodiments, the therapeutic composition comprises: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KLVELEHTL (SEQ ID NO: 2) and HELGFKVVL (SEQ ID NO: 6) and wherein the composition is for use in treating cancer is a subject that expresses an MHC encoded by an HLA-A*02:01 allele, an HLA-B*18:01 allele, an HLA-B*40:01 allele, an HLA-B*44:02 allele, an HLA-B*40:02 allele, or any combination thereof.

In some embodiments, the therapeutic composition comprises: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are SPSSASLAL (SEQ ID NO: 3) and KPRPDVTNEL (SEQ ID NO: 4). In some embodiments, the therapeutic composition comprises: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are SPSSASLAL (SEQ ID NO: 3) and KPRPDVTNEL (SEQ ID NO: 4). In some embodiments, the therapeutic composition comprises: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are SPSSASLAL (SEQ ID NO: 3) and KPRPDVTNEL (SEQ ID NO: 4) and wherein the composition is for use in treating cancer is a subject that expresses an MHC encoded by an HLA-B*07:02 allele, an HLA-B*35:03 allele, an HLA-C*07:02 allele, or any combination thereof. In some embodiments, the therapeutic composition comprises: (i) a recombinant polypeptide consisting of at least two cancer-specific CD8+ T cell epitopes or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are SPSSASLAL (SEQ ID NO: 3) and KPRPDVTNEL (SEQ ID NO: 4) and wherein the composition is for use in treating cancer is a subject that expresses an MHC encoded by an HLA-B*07:02 allele, an HLA-B*35:03 allele, an HLA-C*07:02 allele, or any combination thereof.

In some embodiments, the therapeutic composition comprises: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are SPSSASLAL (SEQ ID NO: 3) and KEF AFLEHSL (SEQ ID NO: 5). In some embodiments, the therapeutic composition comprises: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are SPSSASLAL (SEQ ID NO: 3) and KEFAFLEHSL (SEQ ID NO: 5). In some embodiments, the therapeutic composition comprises: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are SPSSASLAL (SEQ ID NO: 3) and KEFAFLEHSL (SEQ ID NO: 5) and wherein the composition is for use in treating cancer is a subject that expresses an MHC encoded by an HLA-B*07:02 allele, an HLA-B*35:03 allele, an HLA-B*40:01 allele, an HLA-B*44:02 allele, an HLA-B*40:02 allele, or any combination thereof. In some embodiments, the therapeutic composition comprises: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are SPSSASLAL (SEQ ID NO: 3) and KEFAFLEHSL (SEQ ID NO: 5) and wherein the composition is for use in treating cancer is a subject that expresses an MHC encoded by an HLA-B*07:02 allele, an HLA-B*35:03 allele, an HLA-B*40:01 allele, an HLA-B*44:02 allele, an HLA-B*40:02 allele, or any combination thereof.

In some embodiments, the therapeutic composition comprises: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are SPSSASLAL (SEQ ID NO: 3) and HELGFKVVL (SEQ ID NO: 6). In some embodiments, the therapeutic composition comprises: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are SPSSASLAL (SEQ ID NO: 3) and HELGFKVVL (SEQ ID NO: 6). In some embodiments, the therapeutic composition comprises: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are SPSSASLAL (SEQ ID NO: 3) and HELGFKVVL (SEQ ID NO: 6) and wherein the composition is for use in treating cancer is a subject that expresses an MHC encoded by an HLA-B*07:02 allele, an HLA-B*35:03 allele, an HLA-B*18:01 allele, an HLA-B*40:01 allele, an HLA-B*44:02 allele, an HLA-B*40:02 allele, or any combination thereof. In some embodiments, the therapeutic composition comprises: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are SPSSASLAL (SEQ ID NO: 3) and HELGFKVVL (SEQ ID NO: 6) and wherein the composition is for use in treating cancer is a subject that expresses an MHC encoded by an HLA-B*07:02 allele, an HLA-B*35:03 allele, an HLA-B*18:01 allele, an HLA-B*40:01 allele, an HLA-B*44:02 allele, an HLA-B*40:02 allele, or any combination thereof.

In some embodiments, the therapeutic composition comprises: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KPRPDVTNEL (SEQ ID NO: 4) and KEFAFLEHSL (SEQ ID NO: 5). In some embodiments, the therapeutic composition comprises: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KPRPDVTNEL (SEQ ID NO: 4) and KEFAFLEHSL (SEQ ID NO: 5). In some embodiments, the therapeutic composition comprises: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KPRPDVTNEL (SEQ ID NO: 4) and KEFAFLEHSL (SEQ ID NO: 5) and wherein the composition is for use in treating cancer is a subject that expresses an MHC encoded by an HLA-B*07:02 allele, an HLA-B*40:01 allele, an HLA-B*44:02 allele, an HLA-B*35:03 allele, an HLA-C*07:02 allele, an HLA-B*40:02 allele, or any combination thereof. In some embodiments, the therapeutic composition comprises: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KPRPDVTNEL (SEQ ID NO: 4) and KEF AFLEHSL (SEQ ID NO: 5) and wherein the composition is for use in treating cancer is a subject that expresses an MHC encoded by an HLA-B*07:02 allele, an HLA-B*40:01 allele, an HLA-B*44:02 allele, an HLA-B*35:03 allele, an HLA-C*07:02 allele, an HLA-B*40:02 allele, or any combination thereof.

In some embodiments, the therapeutic composition comprises: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KPRPDVTNEL (SEQ ID NO: 4) and HELGFKVVL (SEQ ID NO: 6). In some embodiments, the therapeutic composition comprises: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KPRPDVTNEL (SEQ ID NO: 4) and HELGFKVVL (SEQ ID NO: 6). In some embodiments, the therapeutic composition comprises: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KPRPDVTNEL (SEQ ID NO: 4) and HELGFKVVL (SEQ ID NO: 6) and wherein the composition is for use in treating cancer is a subject that expresses an MHC encoded by an HLA-B*07:02 allele, an HLA-B*35:03 allele, an HLA-C*07:02 allele, an HLA-B*18:01 allele, an HLA-B*40:01 allele, an HLA-B*44:02 allele, an HLA-B*40:02 allele, or any combination thereof. In some embodiments, the therapeutic composition comprises: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KPRPDVTNEL (SEQ ID NO: 4) and HELGFKVVL (SEQ ID NO: 6) and wherein the composition is for use in treating cancer is a subject that expresses an MHC encoded by an HLA-B*07:02 allele, an HLA-B*35:03 allele, an HLA-C*07:02 allele, an HLA-B*18:01 allele, an HLA-B*40:01 allele, an HLA-B*44:02 allele, an HLA-B*40:02 allele, or any combination thereof.

In some embodiments, the therapeutic composition comprises: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KEFAFLEHSL (SEQ ID NO: 5) and HELGFKVVL (SEQ ID NO: 6). In some embodiments, the therapeutic composition comprises: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KEFAFLEHSL (SEQ ID NO: 5) and HELGFKVVL (SEQ ID NO: 6). In some embodiments, the therapeutic composition comprises: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KEFAFLEHSL (SEQ ID NO: 5) and HELGFKVVL (SEQ ID NO: 6) and wherein the composition is for use in treating cancer is a subject that expresses an MHC encoded by an HLA-B*18:01 allele, an HLA-B*40:01 allele, an HLA-B*44:02 allele, an HLA-B*40:02 allele, or any combination thereof. In some embodiments, the therapeutic composition comprises: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KEFAFLEHSL (SEQ ID NO: 5) and HELGFKVVL (SEQ ID NO: 6) and wherein the composition is for use in treating cancer is a subject that expresses an MHC encoded by an HLA-B*18:01 allele, an HLA-B*40:01 allele, an HLA-B*44:02 allele, an HLA-B*40:02 allele, or any combination thereof.

In some embodiments, the therapeutic composition comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KLVELEHTL (SEQ ID NO: 2) and SPSSASLAL (SEQ ID NO: 3). In some embodiments, the therapeutic composition comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KLVELEHTL (SEQ ID NO: 2) and SPSSASLAL (SEQ ID NO: 3). In some embodiments, the therapeutic composition comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KLVELEHTL (SEQ ID NO: 2) and SPSSASLAL (SEQ ID NO: 3) and wherein the composition is for use in treating cancer is a subject that expresses an MHC encoded by an HLA-A*02:01 allele, an HLA-B*07:02 allele, an HLA-B*35:03 allele, or any combination thereof. In some embodiments, the therapeutic composition comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KLVELEHTL (SEQ ID NO: 2) and SPSSASLAL (SEQ ID NO: 3) and wherein the composition is for use in treating cancer is a subject that expresses an MHC encoded by an HLA-A*02:01 allele, an HLA-B*07:02 allele, an HLA-B*35:03 allele, or any combination thereof.

In some embodiments, the therapeutic composition comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KLVELEHTL (SEQ ID NO: 2) and KPRPDVTNEL (SEQ ID NO: 4). In some embodiments, the therapeutic composition comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KLVELEHTL (SEQ ID NO: 2) and KPRPDVTNEL (SEQ ID NO: 4). In some embodiments, the therapeutic composition comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KLVELEHTL (SEQ ID NO: 2) and KPRPDVTNEL (SEQ ID NO: 4) and wherein the composition is for use in treating cancer is a subject that expresses an MHC encoded by an HLA-A*02:01 allele, an HLA-B*07:02 allele, an HLA-B*35:03 allele, an HLA-C*07:02 allele, or any combination thereof. In some embodiments, the therapeutic composition comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KLVELEHTL (SEQ ID NO: 2) and KPRPDVTNEL (SEQ ID NO: 4) and wherein the composition is for use in treating cancer is a subject that expresses an MHC encoded by an HLA-A*02:01 allele, an HLA-B*07:02 allele, an HLA-B*35:03 allele, an HLA-C*07:02 allele, or any combination thereof.

In some embodiments, the therapeutic composition comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KLVELEHTL (SEQ ID NO: 2) and KEFAFLEHSL (SEQ ID NO: 5). In some embodiments, the therapeutic composition comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KLVELEHTL (SEQ ID NO: 2) and KEFAFLEHSL (SEQ ID NO: 5). In some embodiments, the therapeutic composition comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KLVELEHTL (SEQ ID NO: 2) and KEFAFLEHSL (SEQ ID NO: 5) and wherein the composition is for use in treating cancer is a subject that expresses an MHC encoded by an HLA-A*02:01 allele, an HLA-B*40:01 allele, an HLA-B*44:02 allele, an HLA-B*40:02 allele, or any combination thereof. In some embodiments, the therapeutic composition comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KLVELEHTL (SEQ ID NO: 2) and KEFAFLEHSL (SEQ ID NO: 5) and wherein the composition is for use in treating cancer is a subject that expresses an MHC encoded by an HLA-A*02:01 allele, an HLA-B*40:01 allele, an HLA-B*44:02 allele, an HLA-B*40:02 allele, or any combination thereof.

In some embodiments, the therapeutic composition comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KLVELEHTL (SEQ ID NO: 2) and HELGFKVVL (SEQ ID NO: 6). In some embodiments, the therapeutic composition comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KLVELEHTL (SEQ ID NO: 2) and HELGFKVVL (SEQ ID NO: 6). In some embodiments, the therapeutic composition comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KLVELEHTL (SEQ ID NO: 2) and HELGFKVVL (SEQ ID NO: 6) and wherein the composition is for use in treating cancer is a subject that expresses an MHC encoded by an HLA-A*02:01 allele, an HLA-B*18:01 allele, an HLA-B*40:01 allele, an HLA-B*44:02 allele, an HLA-B*40:02 allele, or any combination thereof. In some embodiments, the therapeutic composition comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KLVELEHTL (SEQ ID NO: 2) and HELGFKVVL (SEQ ID NO: 6) and wherein the composition is for use in treating cancer is a subject that expresses an MHC encoded by an HLA-A*02:01 allele, an HLA-B*18:01 allele, an HLA-B*40:01 allele, an HLA-B*44:02 allele, an HLA-B*40:02 allele, or any combination thereof.

In some embodiments, the therapeutic composition comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and SPSSASLAL (SEQ ID NO: 3) and KPRPDVTNEL (SEQ ID NO: 4). In some embodiments, the therapeutic composition comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and SPSSASLAL (SEQ ID NO: 3) and KPRPDVTNEL (SEQ ID NO: 4). In some embodiments, the therapeutic composition comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and SPSSASLAL (SEQ ID NO: 3) and KPRPDVTNEL (SEQ ID NO: 4) and wherein the composition is for use in treating cancer is a subject that expresses an MHC encoded by an HLA-A*02:01 allele, an HLA-B*07:02 allele, an HLA-B*35:03 allele, an HLA-C*07:02 allele, or any combination thereof. In some embodiments, the therapeutic composition comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and SPSSASLAL (SEQ ID NO: 3) and KPRPDVTNEL (SEQ ID NO: 4) and wherein the composition is for use in treating cancer is a subject that expresses an MHC encoded by an HLA-A*02:01 allele, an HLA-B*07:02 allele, an HLA-B*35:03 allele, an HLA-C*07:02 allele, or any combination thereof.

In some embodiments, the therapeutic composition comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and SPSSASLAL (SEQ ID NO: 3) and KEFAFLEHSL (SEQ ID NO: 5). In some embodiments, the therapeutic composition comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and SPSSASLAL (SEQ ID NO: 3) and KEFAFLEHSL (SEQ ID NO: 5). In some embodiments, the therapeutic composition comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and SPSSASLAL (SEQ ID NO: 3) and KEFAFLEHSL (SEQ ID NO: 5) and wherein the composition is for use in treating cancer is a subject that expresses an MHC encoded by an HLA-A*02:01 allele, an HLA-B*07:02 allele, an HLA-B*35:03 allele, an HLA-B*40:01 allele, an HLA-B*44:02 allele, an HLA-B*40:02 allele, or any combination thereof. In some embodiments, the therapeutic composition comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and SPSSASLAL (SEQ ID NO: 3) and KEF AFLEHSL (SEQ ID NO: 5) and wherein the composition is for use in treating cancer is a subject that expresses an MHC encoded by an HLA-A*02:01 allele, an HLA-B*07:02 allele, an HLA-B*35:03 allele, an HLA-B*40:01 allele, an HLA-B*44:02 allele, an HLA-B*40:02 allele, or any combination thereof.

In some embodiments, the therapeutic composition comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and SPSSASLAL (SEQ ID NO: 3) and HELGFKVVL (SEQ ID NO: 6). In some embodiments, the therapeutic composition comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and SPSSASLAL (SEQ ID NO: 3) and HELGFKVVL (SEQ ID NO: 6). In some embodiments, the therapeutic composition comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and SPSSASLAL (SEQ ID NO: 3) and HELGFKVVL (SEQ ID NO: 6) and wherein the composition is for use in treating cancer is a subject that expresses an MHC encoded by an HLA-A*02:01 allele, an HLA-B*07:02 allele, an HLA-B*35:03 allelle, an HLA-B*18:01 allele, an HLA-B*40:01 allele, an HLA-B*44:02 allele, an HLA-B*40:02 allele, or any combination thereof. In some embodiments, the therapeutic composition comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and SPSSASLAL (SEQ ID NO: 3) and HELGFKVVL (SEQ ID NO: 6) and wherein the composition is for use in treating cancer is a subject that expresses an MHC encoded by an HLA-A*02:01 allele, an HLA-B*07:02 allele, an HLA-B*35:03 allelle, an HLA-B*18:01 allele, an HLA-B*40:01 allele, an HLA-B*44:02 allele, an HLA-B*40:02 allele, or any combination thereof.

In some embodiments, the therapeutic composition comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KPRPDVTNEL (SEQ ID NO: 4) and KEFAFLEHSL (SEQ ID NO: 5). In some embodiments, the therapeutic composition comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KPRPDVTNEL (SEQ ID NO: 4) and KEFAFLEHSL (SEQ ID NO: 5). In some embodiments, the therapeutic composition comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KPRPDVTNEL (SEQ ID NO: 4) and KEFAFLEHSL (SEQ ID NO: 5) and wherein the composition is for use in treating cancer is a subject that expresses an MHC encoded by an HLA-A*02:01 allele, an HLA-B*07:02 allele, an HLA-B*35:03 allele, an HLA-C*07:02 allele, an HLA-B*40:01 allele, an HLA-B*44:02 allele, an HLA-B*40:02 allele, or any combination thereof. In some embodiments, the therapeutic composition comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KPRPDVTNEL (SEQ ID NO: 4) and KEFAFLEHSL (SEQ ID NO: 5) and wherein the composition is for use in treating cancer is a subject that expresses an MHC encoded by an HLA-A*02:01 allele, an HLA-B*07:02 allele, an HLA-B*35:03 allele, an HLA-C*07:02 allele, an HLA-B*40:01 allele, an HLA-B*44:02 allele, an HLA-B*40:02 allele, or any combination thereof.

In some embodiments, the therapeutic composition comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KPRPDVTNEL (SEQ ID NO: 4) and HELGFKVVL (SEQ ID NO: 6). In some embodiments, the therapeutic composition comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KPRPDVTNEL (SEQ ID NO: 4) and HELGFKVVL (SEQ ID NO: 6). In some embodiments, the therapeutic composition comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KPRPDVTNEL (SEQ ID NO: 4) and HELGFKVVL (SEQ ID NO: 6) and wherein the composition is for use in treating cancer is a subject that expresses an MHC encoded by an HLA-A*02:01 allele, an HLA-B*07:02 allele, an HLA-B*35:03 allele, an HLA-C*07:02 allele, an HLA-B*40:01 allele, an HLA-B*44:02 allele, an HLA-B*40:02 allele, an HLA-B*18:01 allele, or any combination thereof. In some embodiments, the therapeutic composition comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KPRPDVTNEL (SEQ ID NO: 4) and HELGFKVVL (SEQ ID NO: 6) and wherein the composition is for use in treating cancer is a subject that expresses an MHC encoded by an HLA-A*02:01 allele, an HLA-B*07:02 allele, an HLA-B*35:03 allele, an HLA-C*07:02 allele, an HLA-B*40:01 allele, an HLA-B*44:02 allele, an HLA-B*40:02 allele, an HLA-B*18:01 allele, or any combination thereof.

In some embodiments, the therapeutic composition comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KLVELEHTL (SEQ ID NO: 2) and SPSSASLAL (SEQ ID NO: 3) and KPRPDVTNEL (SEQ ID NO: 4). In some embodiments, the therapeutic composition comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KLVELEHTL (SEQ ID NO: 2) and SPSSASLAL (SEQ ID NO: 3) and KPRPDVTNEL (SEQ ID NO: 4). In some embodiments, the therapeutic composition comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KLVELEHTL (SEQ ID NO: 2) and SPSSASLAL (SEQ ID NO: 3) and KPRPDVTNEL (SEQ ID NO: 4) L and wherein the composition is for use in treating cancer is a subject that expresses an MHC encoded by an HLA-A*02:01 allele, an HLA-B*07:02 allele, an HLA-B*35:03 allele, an HLA-C*07:02 allele, or any combination thereof. In some embodiments, the therapeutic composition comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KLVELEHTL (SEQ ID NO: 2) and SPSSASLAL (SEQ ID NO: 3) and KPRPDVTNEL (SEQ ID NO: 4) and wherein the composition is for use in treating cancer is a subject that expresses an MHC encoded by an HLA-A*02:01 allele, an HLA-B*07:02 allele, an HLA-B*35:03 allele, an HLA-C*07:02 allele, or any combination thereof.

In some embodiments, the therapeutic composition comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KLVELEHTL (SEQ ID NO: 2) and SPSSASLAL (SEQ ID NO: 3) and KEFAFLEHSL (SEQ ID NO: 5). In some embodiments, the therapeutic composition comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KLVELEHTL (SEQ ID NO: 2) and SPSSASLAL (SEQ ID NO: 3) and KEFAFLEHSL (SEQ ID NO: 5). In some embodiments, the therapeutic composition comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KLVELEHTL (SEQ ID NO: 2) and SPSSASLAL (SEQ ID NO: 3) and KEFAFLEHSL (SEQ ID NO: 5) and wherein the composition is for use in treating cancer is a subject that expresses an MHC encoded by an HLA-A*02:01 allele, an HLA-B*07:02 allele, an HLA-B*35:03 allele, an HLA-B*40:01 allele, an HLA-B*44:02 allele, an HLA-B*40:02 allele, or any combination thereof. In some embodiments, the therapeutic composition comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KLVELEHTL (SEQ ID NO: 2) and SPSSASLAL (SEQ ID NO: 3) and KEFAFLEHSL (SEQ ID NO: 5) and wherein the composition is for use in treating cancer is a subject that expresses an MHC encoded by an HLA-A*02:01 allele, an HLA-B*07:02 allele, an HLA-B*35:03 allele, an HLA-B*40:01 allele, an HLA-B*44:02 allele, an HLA-B*40:02 allele, or any combination thereof.

In some embodiments, the therapeutic composition comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KLVELEHTL (SEQ ID NO: 2) and SPSSASLAL (SEQ ID NO: 3) and HELGFKVVL (SEQ ID NO: 6). In some embodiments, the therapeutic composition comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KLVELEHTL (SEQ ID NO: 2) and SPSSASLAL (SEQ ID NO: 3) and HELGFKVVL (SEQ ID NO: 6). In some embodiments, the therapeutic composition comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KLVELEHTL (SEQ ID NO: 2) and SPSSASLAL (SEQ ID NO: 3) and HELGFKVVL (SEQ ID NO: 6) and wherein the composition is for use in treating cancer is a subject that expresses an MHC encoded by an HLA-A*02:01 allele, an HLA-B*07:02 allele, an HLA-B*35:03 allelle, an HLA-B*18:01 allele, an HLA-B*40:01 allele, an HLA-B*44:02 allele, an HLA-B*40:02 allele, or any combination thereof. In some embodiments, the therapeutic composition comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KLVELEHTL (SEQ ID NO: 2) and SPSSASLAL (SEQ ID NO: 3) and HELGFKVVL (SEQ ID NO: 6) and wherein the composition is for use in treating cancer is a subject that expresses an MHC encoded by an HLA-A*02:01 allele, an HLA-B*07:02 allele, an HLA-B*35:03 allelle, an HLA-B*18:01 allele, an HLA-B*40:01 allele, an HLA-B*44:02 allele, an HLA-B*40:02 allele, or any combination thereof.

In some embodiments, the therapeutic composition comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KLVELEHTL (SEQ ID NO: 2) and KPRPDVTNEL (SEQ ID NO: 4) and KEFAFLEHSL (SEQ ID NO: 5). In some embodiments, the therapeutic composition comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KLVELEHTL (SEQ ID NO: 2) and KPRPDVTNEL (SEQ ID NO: 4) and KEFAFLEHSL (SEQ ID NO: 5). In some embodiments, the therapeutic composition comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KLVELEHTL (SEQ ID NO: 2) and KPRPDVTNEL (SEQ ID NO: 4) and KEFAFLEHSL (SEQ ID NO: 5) and wherein the composition is for use in treating cancer is a subject that expresses an MHC encoded by an HLA-A*02:01 allele, an HLA-B*07:02 allele, an HLA-B*35:03 allele, an HLA-C*07:02 allele, an HLA-B*40:01 allele, an HLA-B*44:02 allele, an HLA-B*40:02 allele, or any combination thereof. In some embodiments, the therapeutic composition comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KLVELEHTL (SEQ ID NO: 2) and KPRPDVTNEL (SEQ ID NO: 4) and KEFAFLEHSL (SEQ ID NO: 5) and wherein the composition is for use in treating cancer is a subject that expresses an MHC encoded by an HLA-A*02:01 allele, an HLA-B*07:02 allele, an HLA-B*35:03 allele, an HLA-C*07:02 allele, an HLA-B*40:01 allele, an HLA-B*44:02 allele, an HLA-B*40:02 allele, or any combination thereof.

In some embodiments, the therapeutic composition comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KLVELEHTL (SEQ ID NO: 2) and KPRPDVTNEL (SEQ ID NO: 4) and HELGFKVVL (SEQ ID NO: 6). In some embodiments, the therapeutic composition comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KLVELEHTL (SEQ ID NO: 2) and KPRPDVTNEL (SEQ ID NO: 4) and HELGFKVVL (SEQ ID NO: 6). In some embodiments, the therapeutic composition comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KLVELEHTL (SEQ ID NO: 2) and KPRPDVTNEL (SEQ ID NO: 4) and HELGFKVVL (SEQ ID NO: 6) and wherein the composition is for use in treating cancer is a subject that expresses an MHC encoded by an HLA-A*02:01 allele, an HLA-B*07:02 allele, an HLA-B*35:03 allele, an HLA-C*07:02 allele, an HLA-B*40:01 allele, an HLA-B*44:02 allele, an HLA-B*40:02 allele, an HLA-B*18:01 allele, or any combination thereof. In some embodiments, the therapeutic composition comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KLVELEHTL (SEQ ID NO: 2) and KPRPDVTNEL (SEQ ID NO: 4) and HELGFKVVL (SEQ ID NO: 6) and wherein the composition is for use in treating cancer is a subject that expresses an MHC encoded by an HLA-A*02:01 allele, an HLA-B*07:02 allele, an HLA-B*35:03 allele, an HLA-C*07:02 allele, an HLA-B*40:01 allele, an HLA-B*44:02 allele, an HLA-B*40:02 allele, an HLA-B*18:01 allele, or any combination thereof.

In some embodiments, the therapeutic composition comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KLVELEHTL (SEQ ID NO: 2) and KEFAFLEHSL (SEQ ID NO: 5) and HELGFKVVL (SEQ ID NO: 6). In some embodiments, the therapeutic composition comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KLVELEHTL (SEQ ID NO: 2) and KEFAFLEHSL (SEQ ID NO: 5) and HELGFKVVL (SEQ ID NO: 6). In some embodiments, the therapeutic composition comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KLVELEHTL (SEQ ID NO: 2) and KEFAFLEHSL (SEQ ID NO: 5) and HELGFKVVL (SEQ ID NO: 6) and wherein the composition is for use in treating cancer is a subject that expresses an MHC encoded by an HLA-A*02:01 allele, an HLA-B*18:01 allele, an HLA-B*40:01 allele, an HLA-B*44:02 allele, an HLA-B*40:02 allele, or any combination thereof. In some embodiments, the therapeutic composition comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KLVELEHTL (SEQ ID NO: 2) and KEFAFLEHSL (SEQ ID NO: 5) and HELGFKVVL (SEQ ID NO: 6) and wherein the composition is for use in treating cancer is a subject that expresses an MHC encoded by an HLA-A*02:01 allele, an HLA-B*18:01 allele, an HLA-B*40:01 allele, an HLA-B*44:02 allele, an HLA-B*40:02 allele, or any combination thereof.

In some embodiments, the therapeutic composition comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are SPSSASLAL (SEQ ID NO: 3) and KPRPDVTNEL (SEQ ID NO: 4) and KEFAFLEHSL (SEQ ID NO: 5). In some embodiments, the therapeutic composition comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are SPSSASLAL (SEQ ID NO: 3) and KPRPDVTNEL (SEQ ID NO: 4) and KEFAFLEHSL (SEQ ID NO: 5). In some embodiments, the therapeutic composition comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are SPSSASLAL (SEQ ID NO: 3) and KPRPDVTNEL (SEQ ID NO: 4) and KEFAFLEHSL (SEQ ID NO: 5) and wherein the composition is for use in treating cancer is a subject that expresses an MHC encoded by an HLA-B*07:02 allele, an HLA-B*35:03 allele, an HLA-C*07:02 allele, an HLA-B*40:01 allele, an HLA-B*44:02 allele, an HLA-B*40:02 allele, or any combination thereof. In some embodiments, the therapeutic composition comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are SPSSASLAL (SEQ ID NO: 3) and KPRPDVTNEL (SEQ ID NO: 4) and KEFAFLEHSL (SEQ ID NO: 5) and wherein the composition is for use in treating cancer is a subject that expresses an MHC encoded by an HLA-B*07:02 allele, an HLA-B*35:03 allele, an HLA-C*07:02 allele, an HLA-B*40:01 allele, an HLA-B*44:02 allele, an HLA-B*40:02 allele, or any combination thereof.

In some embodiments, the therapeutic composition comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are SPSSASLAL (SEQ ID NO: 3) and KPRPDVTNEL (SEQ ID NO: 4) and HELGFKVVL (SEQ ID NO: 6). In some embodiments, the therapeutic composition comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are SPSSASLAL (SEQ ID NO: 3) and KPRPDVTNEL (SEQ ID NO: 4) and HELGFKVVL (SEQ ID NO: 6). In some embodiments, the therapeutic composition comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are SPSSASLAL (SEQ ID NO: 3) and KPRPDVTNEL (SEQ ID NO: 4) and HELGFKVVL (SEQ ID NO: 6) and wherein the composition is for use in treating cancer is a subject that expresses an MHC encoded by an HLA-B*07:02 allele, an HLA-B*35:03 allele, an HLA-C*07:02 allele, an HLA-B*40:01 allele, an HLA-B*44:02 allele, an HLA-B*40:02 allele, an HLA-B*18:01 allele, or any combination thereof. In some embodiments, the therapeutic composition comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are SPSSASLAL (SEQ ID NO: 3) and KPRPDVTNEL (SEQ ID NO: 4) and HELGFKVVL (SEQ ID NO: 6) and wherein the composition is for use in treating cancer is a subject that expresses an MHC encoded by an HLA-HLA-B*44:02 allele, an HLA-B*40:02 allele, an HLA-B*18:01 allele, or any combination thereof.

In some embodiments, the therapeutic composition comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are SPSSASLAL (SEQ ID NO: 3) and KEFAFLEHSL (SEQ ID NO: 5) and HELGFKVVL (SEQ ID NO: 6). In some embodiments, the therapeutic composition comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are SPSSASLAL (SEQ ID NO: 3) and KEFAFLEHSL (SEQ ID NO: 5) and HELGFKVVL (SEQ ID NO: 6). In some embodiments, the therapeutic composition comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are SPSSASLAL (SEQ ID NO: 3) and KEFAFLEHSL (SEQ ID NO: 5) and HELGFKVVL (SEQ ID NO: 6) and wherein the composition is for use in treating cancer is a subject that expresses an MHC encoded by an HLA-B*07:02 allele, an HLA-B*35:03 allele, an HLA-B*40:01 allele, an HLA-B*44:02 allele, an HLA-B*40:02 allele, an HLA-B*18:01 allele, or any combination thereof. In some embodiments, the therapeutic composition comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are SPSSASLAL (SEQ ID NO: 3) and KEFAFLEHSL (SEQ ID NO: 5) and HELGFKVVL (SEQ ID NO: 6) and wherein the composition is for use in treating cancer is a subject that expresses an MHC encoded by an HLA-B*07:02 allele, an HLA-B*35:03 allele, an HLA-B*40:01 allele, an HLA-B*44:02 allele, an HLA-B*40:02 allele, an HLA-B*18:01 allele, or any combination thereof.

In some embodiments, the therapeutic composition comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KPRPDVTNEL (SEQ ID NO: 4) and KEFAFLEHSL (SEQ ID NO: 5) and HELGFKVVL (SEQ ID NO: 6). In some embodiments, the therapeutic composition comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KPRPDVTNEL (SEQ ID NO: 4) and KEFAFLEHSL (SEQ ID NO: 5) and HELGFKVVL (SEQ ID NO: 6). In some embodiments, the therapeutic composition comprises: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KPRPDVTNEL (SEQ ID NO: 4) and KEFAFLEHSL (SEQ ID NO: 5) and HELGFKVVL (SEQ ID NO: 6) and wherein the composition is for use in treating cancer is a subject that expresses an MHC encoded by an HLA-B*07:02 allele, an HLA-B*35:03 allele, an HLA-C*07:02 allele, an HLA-B*40:01 allele, an HLA-B*44:02 allele, an HLA-B*40:02 allele, an HLA-B*18:01 allele, or any combination thereof. In some embodiments, the therapeutic composition comprises: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KPRPDVTNEL (SEQ ID NO: 4) and KEFAFLEHSL (SEQ ID NO: 5) and HELGFKVVL (SEQ ID NO: 6) and wherein the composition is for use in treating cancer is a subject that expresses an MHC encoded by an HLA-HLA-B*44:02 allele, an HLA-B*40:02 allele, an HLA-B*18:01 allele, or any combination thereof.

In some embodiments, the therapeutic composition comprises: (i) a recombinant polypeptide comprising at least four cancer-specific CD8+ T cell epitopes, or (ii) a polynucleotide encoding a recombinant polypeptide comprising at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are GVYDGREHTV (SEQ ID NO: 1) and KLVELEHTL (SEQ ID NO: 2) and SPSSASLAL (SEQ ID NO: 3) and KPRPDVTNEL (SEQ ID NO: 4). In some embodiments, the therapeutic composition comprises: (i) a recombinant polypeptide consisting of at least four cancer-specific CD8+ T cell epitopes, or (ii) a polynucleotide encoding a recombinant polypeptide consisting of at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are GVYDGREHTV (SEQ ID NO: 1) and KLVELEHTL (SEQ ID NO: 2) and SPSSASLAL (SEQ ID NO: 3) and KPRPDVTNEL (SEQ ID NO: 4). In some embodiments, the therapeutic composition comprises: (i) a recombinant polypeptide comprising at least four cancer-specific CD8+ T cell epitopes, or (ii) a polynucleotide encoding a recombinant polypeptide comprising at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are GVYDGREHTV (SEQ ID NO: 1) and KLVELEHTL (SEQ ID NO: 2) and SPSSASLAL (SEQ ID NO: 3) and KPRPDVTNEL (SEQ ID NO: 4) and wherein the composition is for use in treating cancer is a subject that expresses an MHC encoded by an HLA-A*02:01 allele, an HLA-B*07:02 allele, an HLA-B*35:03 allele, an HLA-C*07:02 allele, or any combination thereof. In some embodiments, the therapeutic composition comprises: (i) a recombinant polypeptide consisting of at least four cancer-specific CD8+ T cell epitopes, or (ii) a polynucleotide encoding a recombinant polypeptide consisting of at four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are GVYDGREHTV (SEQ ID NO: 1) and KLVELEHTL (SEQ ID NO: 2) and SPSSASLAL (SEQ ID NO: 3) and KPRPDVTNEL (SEQ ID NO: 4) and wherein the composition is for use in treating cancer is a subject that expresses an MHC encoded by an HLA-A*02:01 allele, an HLA-B*07:02 allele, an HLA-B*35:03 allele, an HLA-C*07:02 allele, or any combination thereof.

In some embodiments, the therapeutic composition comprises: (i) a recombinant polypeptide comprising at least four cancer-specific CD8+ T cell epitopes, or (ii) a polynucleotide encoding a recombinant polypeptide comprising at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are GVYDGREHTV (SEQ ID NO: 1) and KLVELEHTL (SEQ ID NO: 2) and SPSSASLAL (SEQ ID NO: 3) and KEFAFLEHSL (SEQ ID NO: 5). In some embodiments, the therapeutic composition comprises: (i) a recombinant polypeptide consisting of at least four cancer-specific CD8+ T cell epitopes, or (ii) a polynucleotide encoding a recombinant polypeptide consisting of at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are GVYDGREHTV (SEQ ID NO: 1) and KLVELEHTL (SEQ ID NO: 2) and SPSSASLAL (SEQ ID NO: 3) and KEFAFLEHSL (SEQ ID NO: 5). In some embodiments, the therapeutic composition comprises: (i) a recombinant polypeptide comprising at least four cancer-specific CD8+ T cell epitopes, or (ii) a polynucleotide encoding a recombinant polypeptide comprising at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are GVYDGREHTV (SEQ ID NO: 1) and KLVELEHTL (SEQ ID NO: 2) and SPSSASLAL (SEQ ID NO: 3) and KEFAFLEHSL (SEQ ID NO: 5) and wherein the composition is for use in treating cancer is a subject that expresses an MHC encoded by an HLA-A*02:01 allele, an HLA-B*07:02 allele, an HLA-B*35:03 allele, an HLA-B*40:01 allele, an HLA-B*44:02 allele, an HLA-B*40:02 allele, or any combination thereof. In some embodiments, the therapeutic composition comprises: (i) a recombinant polypeptide consisting of at least four cancer-specific CD8+ T cell epitopes, or (ii) a polynucleotide encoding a recombinant polypeptide consisting of at four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are GVYDGREHTV (SEQ ID NO: 1) and KLVELEHTL (SEQ ID NO: 2) and SPSSASLAL (SEQ ID NO: 3) and KEFAFLEHSL (SEQ ID NO: 5) and wherein the composition is for use in treating cancer is a subject that expresses an MHC encoded by an HLA-A*02:01 allele, an HLA-B*07:02 allele, an HLA-B*35:03 allele, an HLA-B*40:01 allele, an HLA-B*44:02 allele, an HLA-B*40:02 allele, or any combination thereof.

In some embodiments, the therapeutic composition comprises: (i) a recombinant polypeptide comprising at least four cancer-specific CD8+ T cell epitopes, or (ii) a polynucleotide encoding a recombinant polypeptide comprising at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are GVYDGREHTV (SEQ ID NO: 1) and KLVELEHTL (SEQ ID NO: 2) and SPSSASLAL (SEQ ID NO: 3) and HELGFKVVL (SEQ ID NO: 6). In some embodiments, the therapeutic composition comprises: (i) a recombinant polypeptide consisting of at least four cancer-specific CD8+ T cell epitopes, or (ii) a polynucleotide encoding a recombinant polypeptide consisting of at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are GVYDGREHTV (SEQ ID NO: 1) and KLVELEHTL (SEQ ID NO: 2) and SPSSASLAL (SEQ ID NO: 3) and HELGFKVVL (SEQ ID NO: 6). In some embodiments, the therapeutic composition comprises: (i) a recombinant polypeptide comprising at least four cancer-specific CD8+ T cell epitopes, or (ii) a polynucleotide encoding a recombinant polypeptide comprising at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are GVYDGREHTV (SEQ ID NO: 1) and KLVELEHTL (SEQ ID NO: 2) and SPSSASLAL (SEQ ID NO: 3) and HELGFKVVL (SEQ ID NO: 6) and wherein the composition is for use in treating cancer is a subject that expresses an MHC encoded by an HLA-A*02:01 allele, an HLA-B*07:02 allele, an HLA-B*35:03 allelle, an HLA-B*18:01 allele, an HLA-B*40:01 allele, an HLA-B*44:02 allele, an HLA-B*40:02 allele, or any combination thereof. In some embodiments, the therapeutic composition comprises: (i) a recombinant polypeptide consisting of at least four cancer-specific CD8+ T cell epitopes, or (ii) a polynucleotide encoding a recombinant polypeptide consisting of at four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are GVYDGREHTV (SEQ ID NO: 1) and KLVELEHTL (SEQ ID NO: 2) and SPSSASLAL (SEQ ID NO: 3) and HELGFKVVL (SEQ ID NO: 6) and wherein the composition is for use in treating cancer is a subject that expresses an MHC encoded by an HLA-A*02:01 allele, an HLA-B*07:02 allele, an HLA-B*35:03 allelle, an HLA-B*18:01 allele, an HLA-B*40:01 allele, an HLA-B*44:02 allele, an HLA-B*40:02 allele, or any combination thereof.

In some embodiments, the therapeutic composition comprises: (i) a recombinant polypeptide comprising at least four cancer-specific CD8+ T cell epitopes, or (ii) a polynucleotide encoding a recombinant polypeptide comprising at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are GVYDGREHTV (SEQ ID NO: 1) and SPSSASLAL (SEQ ID NO: 3) and KPRPDVTNEL (SEQ ID NO: 4) and KEFAFLEHSL (SEQ ID NO: 5). In some embodiments, the therapeutic composition comprises: (i) a recombinant polypeptide consisting of at least four cancer-specific CD8+ T cell epitopes, or (ii) a polynucleotide encoding a recombinant polypeptide consisting of at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are GVYDGREHTV (SEQ ID NO: 1) and SPSSASLAL (SEQ ID NO: 3) and KPRPDVTNEL (SEQ ID NO: 4) and KEFAFLEHSL (SEQ ID NO: 5). In some embodiments, the therapeutic composition comprises: (i) a recombinant polypeptide comprising at least four cancer-specific CD8+ T cell epitopes, or (ii) a polynucleotide encoding a recombinant polypeptide comprising at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are GVYDGREHTV (SEQ ID NO: 1) and SPSSASLAL (SEQ ID NO: 3) and KPRPDVTNEL (SEQ ID NO: 4) and KEFAFLEHSL (SEQ ID NO: 5) and wherein the composition is for use in treating cancer is a subject that expresses an MHC encoded by an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, or any combination thereof. In some embodiments, the therapeutic composition comprises: (i) a recombinant polypeptide consisting of at least four cancer-specific CD8+ T cell epitopes, or (ii) a polynucleotide encoding a recombinant polypeptide consisting of at four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are GVYDGREHTV (SEQ ID NO: 1) and SPSSASLAL (SEQ ID NO: 3) and KPRPDVTNEL (SEQ ID NO: 4) and KEFAFLEHSL (SEQ ID NO: 5) and wherein the composition is for use in treating cancer is a subject that expresses an MHC encoded by an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, or any combination thereof.

In some embodiments, the therapeutic composition comprises: (i) a recombinant polypeptide comprising at least four cancer-specific CD8+ T cell epitopes, or (ii) a polynucleotide encoding a recombinant polypeptide comprising at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are GVYDGREHTV (SEQ ID NO: 1) and SPSSASLAL (SEQ ID NO: 3) and KPRPDVTNEL (SEQ ID NO: 4) and HELGFKVVL (SEQ ID NO: 6). In some embodiments, the therapeutic composition comprises: (i) a recombinant polypeptide consisting of at least four cancer-specific CD8+ T cell epitopes, or (ii) a polynucleotide encoding a recombinant polypeptide consisting of at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are GVYDGREHTV (SEQ ID NO: 1) and SPSSASLAL (SEQ ID NO: 3) and KPRPDVTNEL (SEQ ID NO: 4) and HELGFKVVL (SEQ ID NO: 6). In some embodiments, the therapeutic composition comprises: (i) a recombinant polypeptide comprising at least four cancer-specific CD8+ T cell epitopes, or (ii) a polynucleotide encoding a recombinant polypeptide comprising at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are GVYDGREHTV (SEQ ID NO: 1) and SPSSASLAL (SEQ ID NO: 3) and KPRPDVTNEL (SEQ ID NO: 4) and HELGFKVVL (SEQ ID NO: 6) and wherein the composition is for use in treating cancer is a subject that expresses an MHC encoded by an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, an HLA-B*18:01 allele, or any combination thereof. In some embodiments, the therapeutic composition comprises: (i) a recombinant polypeptide consisting of at least four cancer-specific CD8+ T cell epitopes, or (ii) a polynucleotide encoding a recombinant polypeptide consisting of at four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are GVYDGREHTV (SEQ ID NO: 1) and SPSSASLAL (SEQ ID NO: 3) and KPRPDVTNEL (SEQ ID NO: 4) and HELGFKVVL (SEQ ID NO: 6) and wherein the composition is for use in treating cancer is a subject that expresses an MHC encoded by an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, an HLA-B*18:01 allele, or any combination thereof.

In some embodiments, the therapeutic composition comprises: (i) a recombinant polypeptide comprising at least four cancer-specific CD8+ T cell epitopes, or (ii) a polynucleotide encoding a recombinant polypeptide comprising at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are GVYDGREHTV (SEQ ID NO: 1) and KPRPDVTNEL (SEQ ID NO: 4) and KEFAFLEHSL (SEQ ID NO: 5) and HELGFKVVL (SEQ ID NO: 6). In some embodiments, the therapeutic composition comprises: (i) a recombinant polypeptide consisting of at least four cancer-specific CD8+ T cell epitopes, or (ii) a polynucleotide encoding a recombinant polypeptide consisting of at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are GVYDGREHTV (SEQ ID NO: 1) and KPRPDVTNEL (SEQ ID NO: 4) and KEFAFLEHSL (SEQ ID NO: 5) and HELGFKVVL (SEQ ID NO: 6). In some embodiments, the therapeutic composition comprises: (i) a recombinant polypeptide comprising at least four cancer-specific CD8+ T cell epitopes, or (ii) a polynucleotide encoding a recombinant polypeptide comprising at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are GVYDGREHTV (SEQ ID NO: 1) and KPRPDVTNEL (SEQ ID NO: 4) and KEFAFLEHSL (SEQ ID NO: 5) and HELGFKVVL (SEQ ID NO: 6) and wherein the composition is for use in treating cancer is a subject that expresses an MHC encoded by an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, an HLA-B*18:01 allele, or any combination thereof. In some embodiments, the therapeutic composition comprises: (i) a recombinant polypeptide consisting of at least four cancer-specific CD8+ T cell epitopes, or (ii) a polynucleotide encoding a recombinant polypeptide consisting of at four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are GVYDGREHTV (SEQ ID NO: 1) and KPRPDVTNEL (SEQ ID NO: 4) and KEFAFLEHSL (SEQ ID NO: 5) and HELGFKVVL (SEQ ID NO: 6) and wherein the composition is for use in treating cancer is a subject that expresses an MHC encoded by an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, an HLA-B*18:01 allele, or any combination thereof.

In some embodiments, the therapeutic composition comprises: (i) a recombinant polypeptide comprising at least four cancer-specific CD8+ T cell epitopes, or (ii) a polynucleotide encoding a recombinant polypeptide comprising at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are KLVELEHTL (SEQ ID NO: 2) and SPSSASLAL (SEQ ID NO: 3) and KPRPDVTNEL (SEQ ID NO: 4) and KEFAFLEHSL (SEQ ID NO: 5). In some embodiments, the therapeutic composition comprises: (i) a recombinant polypeptide consisting of at least four cancer-specific CD8+ T cell epitopes, or (ii) a polynucleotide encoding a recombinant polypeptide consisting of at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are KLVELEHTL (SEQ ID NO: 2) and SPSSASLAL (SEQ ID NO: 3) and KPRPDVTNEL (SEQ ID NO: 4) and KEFAFLEHSL (SEQ ID NO: 5). In some embodiments, the therapeutic composition comprises: (i) a recombinant polypeptide comprising at least four cancer-specific CD8+ T cell epitopes, or (ii) a polynucleotide encoding a recombinant polypeptide comprising at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are KLVELEHTL (SEQ ID NO: 2) and SPSSASLAL (SEQ ID NO: 3) and KPRPDVTNEL (SEQ ID NO: 4) and KEFAFLEHSL (SEQ ID NO: 5) and wherein the composition is for use in treating cancer is a subject that expresses an MHC encoded by an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, or any combination thereof. In some embodiments, the therapeutic composition comprises: (i) a recombinant polypeptide consisting of at least four cancer-specific CD8+ T cell epitopes, or (ii) a polynucleotide encoding a recombinant polypeptide consisting of at four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are KLVELEHTL (SEQ ID NO: 2) and SPSSASLAL (SEQ ID NO: 3) and KPRPDVTNEL (SEQ ID NO: 4) and KEFAFLEHSL (SEQ ID NO: 5) and wherein the composition is for use in treating cancer is a subject that expresses an MHC encoded by an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, or any combination thereof.

In some embodiments, the therapeutic composition comprises: (i) a recombinant polypeptide comprising at least four cancer-specific CD8+ T cell epitopes, or (ii) a polynucleotide encoding a recombinant polypeptide comprising at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are KLVELEHTL (SEQ ID NO: 2) and SPSSASLAL (SEQ ID NO: 3) and KPRPDVTNEL (SEQ ID NO: 4) and HELGFKVVL (SEQ ID NO: 6). In some embodiments, the therapeutic composition comprises: (i) a recombinant polypeptide consisting of at least four cancer-specific CD8+ T cell epitopes, or (ii) a polynucleotide encoding a recombinant polypeptide consisting of at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are KLVELEHTL (SEQ ID NO: 2) and SPSSASLAL (SEQ ID NO: 3) and KPRPDVTNEL (SEQ ID NO: 4) and HELGFKVVL (SEQ ID NO: 6). In some embodiments, the therapeutic composition comprises: (i) a recombinant polypeptide comprising at least four cancer-specific CD8+ T cell epitopes, or (ii) a polynucleotide encoding a recombinant polypeptide comprising at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are KLVELEHTL (SEQ ID NO: 2) and SPSSASLAL (SEQ ID NO: 3) and KPRPDVTNEL (SEQ ID NO: 4) and HELGFKVVL (SEQ ID NO: 6) and wherein the composition is for use in treating cancer is a subject that expresses an MHC encoded by an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, an HLA-B*18:01 allele, or any combination thereof. In some embodiments, the therapeutic composition comprises: (i) a recombinant polypeptide consisting of at least four cancer-specific CD8+ T cell epitopes, or (ii) a polynucleotide encoding a recombinant polypeptide consisting of at four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are KLVELEHTL (SEQ ID NO: 2) and SPSSASLAL (SEQ ID NO: 3) and KPRPDVTNEL (SEQ ID NO: 4) and HELGFKVVL (SEQ ID NO: 6) and wherein the composition is for use in treating cancer is a subject that expresses an MHC encoded by an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, an HLA-B*18:01 allele, or any combination thereof.

In some embodiments, the therapeutic composition comprises: (i) a recombinant polypeptide comprising at least four cancer-specific CD8+ T cell epitopes, or (ii) a polynucleotide encoding a recombinant polypeptide comprising at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are KLVELEHTL (SEQ ID NO: 2) and KPRPDVTNEL (SEQ ID NO: 4) and KEFAFLEHSL (SEQ ID NO: 5) and HELGFKVVL (SEQ ID NO: 6). In some embodiments, the therapeutic composition comprises: (i) a recombinant polypeptide consisting of at least four cancer-specific CD8+ T cell epitopes, or (ii) a polynucleotide encoding a recombinant polypeptide consisting of at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are KLVELEHTL (SEQ ID NO: 2) and KPRPDVTNEL (SEQ ID NO: 4) and KEFAFLEHSL (SEQ ID NO: 5) and HELGFKVVL (SEQ ID NO: 6). In some embodiments, the therapeutic composition comprises: (i) a recombinant polypeptide comprising at least four cancer-specific CD8+ T cell epitopes, or (ii) a polynucleotide encoding a recombinant polypeptide comprising at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are KLVELEHTL (SEQ ID NO: 2) and KPRPDVTNEL (SEQ ID NO: 4) and KEFAFLEHSL (SEQ ID NO: 5) and HELGFKVVL (SEQ ID NO: 6) and wherein the composition is for use in treating cancer is a subject that expresses an MHC encoded by an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, an HLA-B*18:01 allele, or any combination thereof. In some embodiments, the therapeutic composition comprises: (i) a recombinant polypeptide consisting of at least four cancer-specific CD8+ T cell epitopes, or (ii) a polynucleotide encoding a recombinant polypeptide consisting of at four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are KLVELEHTL (SEQ ID NO: 2) and KPRPDVTNEL (SEQ ID NO: 4) and KEFAFLEHSL (SEQ ID NO: 5) and HELGFKVVL (SEQ ID NO: 6) and wherein the composition is for use in treating cancer is a subject that expresses an MHC encoded by an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, an HLA-B*18:01 allele, or any combination thereof.

In some embodiments, the therapeutic composition comprises: (i) a recombinant polypeptide comprising at least four cancer-specific CD8+ T cell epitopes, or (ii) a polynucleotide encoding a recombinant polypeptide comprising at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are SPSSASLAL (SEQ ID NO: 3) and KPRPDVTNEL (SEQ ID NO: 4) and KEFAFLEHSL (SEQ ID NO: 5) and HELGFKVVL (SEQ ID NO: 6). In some embodiments, the therapeutic composition comprises: (i) a recombinant polypeptide consisting of at least four cancer-specific CD8+ T cell epitopes, or (ii) a polynucleotide encoding a recombinant polypeptide consisting of at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are SPSSASLAL (SEQ ID NO: 3) and KPRPDVTNEL (SEQ ID NO: 4) and KEFAFLEHSL (SEQ ID NO: 5) and HELGFKVVL (SEQ ID NO: 6). In some embodiments, the therapeutic composition comprises: (i) a recombinant polypeptide comprising at least four cancer-specific CD8+ T cell epitopes, or (ii) a polynucleotide encoding a recombinant polypeptide comprising at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are SPSSASLAL (SEQ ID NO: 3) and KPRPDVTNEL (SEQ ID NO: 4) and KEFAFLEHSL (SEQ ID NO: 5) and HELGFKVVL (SEQ ID NO: 6) and wherein the composition is for use in treating cancer is a subject that expresses an MHC encoded by an HLA-B*07:02 allele, an HLA-B*35:03 allele, an HLA-C*07:02 allele, an HLA-B*40:01 allele, an HLA-B*40:02 allele, an HLA-B*44:02 allele, an HLA-B*18:01 allele, or any combination thereof. In some embodiments, the therapeutic composition comprises: (i) a recombinant polypeptide consisting of at least four cancer-specific CD8+ T cell epitopes, or (ii) a polynucleotide encoding a recombinant polypeptide consisting of at four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are SPSSASLAL (SEQ ID NO: 3) and KPRPDVTNEL (SEQ ID NO: 4) and KEFAFLEHSL (SEQ ID NO: 5) and HELGFKVVL (SEQ ID NO: 6) and wherein the composition is for use in treating cancer is a subject that expresses an MHC encoded by an HLA-B*07:02 allele, an HLA-B*35:03 allele, an HLA-C*07:02 allele, an HLA-B*40:01 allele, an HLA-B*40:02 allele, an HLA-B*44:02 allele, an HLA-B*18:01 allele, or any combination thereof.

In some embodiments, the therapeutic composition comprises: (i) a recombinant polypeptide comprising at least five cancer-specific CD8+ T cell epitopes, or (ii) a polynucleotide encoding a recombinant polypeptide comprising at least five cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least five cancer-specific CD8+ T cell epitopes are GVYDGREHTV (SEQ ID NO: 1) and KLVELEHTL (SEQ ID NO: 2) and SPSSASLAL (SEQ ID NO: 3) and KPRPDVTNEL (SEQ ID NO: 4) and KEFAFLEHSL (SEQ ID NO: 5). In some embodiments, the therapeutic composition comprises: (i) a recombinant polypeptide consisting of at least five cancer-specific CD8+ T cell epitopes, or (ii) a polynucleotide encoding a recombinant polypeptide consisting of at least five cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least five cancer-specific CD8+ T cell epitopes are GVYDGREHTV (SEQ ID NO: 1) and KLVELEHTL (SEQ ID NO: 2) and SPSSASLAL (SEQ ID NO: 3) and KPRPDVTNEL (SEQ ID NO: 4) and KEFAFLEHSL (SEQ ID NO: 5). In some embodiments, the therapeutic composition comprises: (i) a recombinant polypeptide comprising at least five cancer-specific CD8+ T cell epitopes, or (ii) a polynucleotide encoding a recombinant polypeptide comprising at least five cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least five cancer-specific CD8+ T cell epitopes are GVYDGREHTV (SEQ ID NO: 1) and KLVELEHTL (SEQ ID NO: 2) and SPSSASLAL (SEQ ID NO: 3) and KPRPDVTNEL (SEQ ID NO: 4) and KEFAFLEHSL (SEQ ID NO: 5) and wherein the composition is for use in treating cancer is a subject that expresses an MHC encoded by an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, or any combination thereof. In some embodiments, the therapeutic composition comprises: (i) a recombinant polypeptide consisting of at least five cancer-specific CD8+ T cell epitopes, or (ii) a polynucleotide encoding a recombinant polypeptide consisting of at five cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least five cancer-specific CD8+ T cell epitopes are GVYDGREHTV (SEQ ID NO: 1) and KLVELEHTL (SEQ ID NO: 2) and SPSSASLAL (SEQ ID NO: 3) and KPRPDVTNEL (SEQ ID NO: 4) and KEFAFLEHSL (SEQ ID NO: 5) and wherein the composition is for use in treating cancer is a subject that expresses an MHC encoded by an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, or any combination thereof.

In some embodiments, the therapeutic composition comprises: (i) a recombinant polypeptide comprising at least five cancer-specific CD8+ T cell epitopes, or (ii) a polynucleotide encoding a recombinant polypeptide comprising at least five cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least five cancer-specific CD8+ T cell epitopes are GVYDGREHTV (SEQ ID NO: 1) and KLVELEHTL (SEQ ID NO: 2) and SPSSASLAL (SEQ ID NO: 3) and KPRPDVTNEL (SEQ ID NO: 4) and HELGFKVVL (SEQ ID NO: 6). In some embodiments, the therapeutic composition comprises: (i) a recombinant polypeptide consisting of at least five cancer-specific CD8+ T cell epitopes, or (ii) a polynucleotide encoding a recombinant polypeptide consisting of at least five cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least five cancer-specific CD8+ T cell epitopes are GVYDGREHTV (SEQ ID NO: 1) and KLVELEHTL (SEQ ID NO: 2) and SPSSASLAL (SEQ ID NO: 3) and KPRPDVTNEL (SEQ ID NO: 4) and HELGFKVVL (SEQ ID NO: 6). In some embodiments, the therapeutic composition comprises: (i) a recombinant polypeptide comprising at least five cancer-specific CD8+ T cell epitopes, or (ii) a polynucleotide encoding a recombinant polypeptide comprising at least five cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least five cancer-specific CD8+ T cell epitopes are GVYDGREHTV (SEQ ID NO: 1) and KLVELEHTL (SEQ ID NO: 2) and SPSSASLAL (SEQ ID NO: 3) and KPRPDVTNEL (SEQ ID NO: 4) and HELGFKVVL (SEQ ID NO: 6) and wherein the composition is for use in treating cancer is a subject that expresses an MHC encoded by an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, an HLA-B*18:01 allele, or any combination thereof. In some embodiments, the therapeutic composition comprises: (i) a recombinant polypeptide consisting of at least five cancer-specific CD8+ T cell epitopes, or (ii) a polynucleotide encoding a recombinant polypeptide consisting of at five cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least five cancer-specific CD8+ T cell epitopes are GVYDGREHTV (SEQ ID NO: 1) and KLVELEHTL (SEQ ID NO: 2) and SPSSASLAL (SEQ ID NO: 3) and KPRPDVTNEL (SEQ ID NO: 4) and HELGFKVVL (SEQ ID NO: 6) and wherein the composition is for use in treating cancer is a subject that expresses an MHC encoded by an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, an HLA-B*18:01 allele, or any combination thereof.

In some embodiments, the therapeutic composition comprises: (i) a recombinant polypeptide comprising at least five cancer-specific CD8+ T cell epitopes, or (ii) a polynucleotide encoding a recombinant polypeptide comprising at least five cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least five cancer-specific CD8+ T cell epitopes are GVYDGREHTV (SEQ ID NO: 1) and SPSSASLAL (SEQ ID NO: 3) and KPRPDVTNEL (SEQ ID NO: 4) and KEFAFLEHSL (SEQ ID NO: 5) and HELGFKVVL (SEQ ID NO: 6). In some embodiments, the therapeutic composition comprises: (i) a recombinant polypeptide consisting of at least five cancer-specific CD8+ T cell epitopes, or (ii) a polynucleotide encoding a recombinant polypeptide consisting of at least five cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least five cancer-specific CD8+ T cell epitopes are GVYDGREHTV (SEQ ID NO: 1) and SPSSASLAL (SEQ ID NO: 3) and KPRPDVTNEL (SEQ ID NO: 4) and KEFAFLEHSL (SEQ ID NO: 5) and HELGFKVVL (SEQ ID NO: 6). In some embodiments, the therapeutic composition comprises: (i) a recombinant polypeptide comprising at least five cancer-specific CD8+ T cell epitopes, or (ii) a polynucleotide encoding a recombinant polypeptide comprising at least five cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least five cancer-specific CD8+ T cell epitopes are GVYDGREHTV (SEQ ID NO: 1) and SPSSASLAL (SEQ ID NO: 3) and KPRPDVTNEL (SEQ ID NO: 4) and KEFAFLEHSL (SEQ ID NO: 5) and HELGFKVVL (SEQ ID NO: 6) and wherein the composition is for use in treating cancer is a subject that expresses an MHC encoded by an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, an HLA-B*18:01 allele, or any combination thereof. In some embodiments, the therapeutic composition comprises: (i) a recombinant polypeptide consisting of at least five cancer-specific CD8+ T cell epitopes, or (ii) a polynucleotide encoding a recombinant polypeptide consisting of at five cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least five cancer-specific CD8+ T cell epitopes are GVYDGREHTV (SEQ ID NO: 1) and SPSSASLAL (SEQ ID NO: 3) and KPRPDVTNEL (SEQ ID NO: 4) and KEFAFLEHSL (SEQ ID NO: 5) and HELGFKVVL (SEQ ID NO: 6) and wherein the composition is for use in treating cancer is a subject that expresses an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, an HLA-B*18:01 allele, or any combination thereof.

In some embodiments, the therapeutic composition comprises: (i) a recombinant polypeptide comprising at least five cancer-specific CD8+ T cell epitopes, or (ii) a polynucleotide encoding a recombinant polypeptide comprising at least five cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least five cancer-specific CD8+ T cell epitopes are KLVELEHTL (SEQ ID NO: 2) and SPSSASLAL (SEQ ID NO: 3) and KPRPDVTNEL (SEQ ID NO: 4) and KEFAFLEHSL (SEQ ID NO: 5) and HELGFKVVL (SEQ ID NO: 6). In some embodiments, the therapeutic composition comprises: (i) a recombinant polypeptide consisting of at least five cancer-specific CD8+ T cell epitopes, or (ii) a polynucleotide encoding a recombinant polypeptide consisting of at least five cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least five cancer-specific CD8+ T cell epitopes are KLVELEHTL (SEQ ID NO: 2) and SPSSASLAL (SEQ ID NO: 3) and KPRPDVTNEL (SEQ ID NO: 4) and KEFAFLEHSL (SEQ ID NO: 5) and HELGFKVVL (SEQ ID NO: 6). In some embodiments, the therapeutic composition comprises: (i) a recombinant polypeptide comprising at least five cancer-specific CD8+ T cell epitopes, or (ii) a polynucleotide encoding a recombinant polypeptide comprising at least five cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least five cancer-specific CD8+ T cell epitopes are KLVELEHTL (SEQ ID NO: 2) and SPSSASLAL (SEQ ID NO: 3) and KPRPDVTNEL (SEQ ID NO: 4) and KEFAFLEHSL (SEQ ID NO: 5) and HELGFKVVL (SEQ ID NO: 6) and wherein the composition is for use in treating cancer is a subject that expresses an MHC encoded by an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, an HLA-B*18:01 allele, or any combination thereof. In some embodiments, the therapeutic composition comprises: (i) a recombinant polypeptide consisting of at least five cancer-specific CD8+ T cell epitopes, or (ii) a polynucleotide encoding a recombinant polypeptide consisting of at five cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least five cancer-specific CD8+ T cell epitopes are KLVELEHTL (SEQ ID NO: 2) and SPSSASLAL (SEQ ID NO: 3) and KPRPDVTNEL (SEQ ID NO: 4) and KEFAFLEHSL (SEQ ID NO: 5) and HELGFKVVL (SEQ ID NO: 6) and wherein the composition is for use in treating cancer is a subject that expresses an MHC encoded by an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, an HLA-B*18:01 allele, or any combination thereof.

In some embodiments, the therapeutic composition comprises: (i) a recombinant polypeptide comprising at least five cancer-specific CD8+ T cell epitopes, or (ii) a polynucleotide encoding a recombinant polypeptide comprising at least five cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least five cancer-specific CD8+ T cell epitopes are GVYDGREHTV (SEQ ID NO: 1) and KLVELEHTL (SEQ ID NO: 2) and KPRPDVTNEL (SEQ ID NO: 4) and KEFAFLEHSL (SEQ ID NO: 5) and HELGFKVVL (SEQ ID NO: 6). In some embodiments, the therapeutic composition comprises: (i) a recombinant polypeptide consisting of at least five cancer-specific CD8+ T cell epitopes, or (ii) a polynucleotide encoding a recombinant polypeptide consisting of at least five cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least five cancer-specific CD8+ T cell epitopes are GVYDGREHTV (SEQ ID NO: 1) and KLVELEHTL (SEQ ID NO: 2) and KPRPDVTNEL (SEQ ID NO: 4) and KEFAFLEHSL (SEQ ID NO: 5) and HELGFKVVL (SEQ ID NO: 6). In some embodiments, the therapeutic composition comprises: (i) a recombinant polypeptide comprising at least five cancer-specific CD8+ T cell epitopes, or (ii) a polynucleotide encoding a recombinant polypeptide comprising at least five cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least five cancer-specific CD8+ T cell epitopes are GVYDGREHTV (SEQ ID NO: 1) and KLVELEHTL (SEQ ID NO: 2) and KPRPDVTNEL (SEQ ID NO: 4) and KEFAFLEHSL (SEQ ID NO: 5) and HELGFKVVL (SEQ ID NO: 6) and wherein the composition is for use in treating cancer is a subject that expresses an MHC encoded by an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, an HLA-B*18:01 allele, or any combination thereof. In some embodiments, the therapeutic composition comprises: (i) a recombinant polypeptide consisting of at least five cancer-specific CD8+ T cell epitopes, or (ii) a polynucleotide encoding a recombinant polypeptide consisting of at five cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least five cancer-specific CD8+ T cell epitopes are GVYDGREHTV (SEQ ID NO: 1) and KLVELEHTL (SEQ ID NO: 2) and KPRPDVTNEL (SEQ ID NO: 4) and KEFAFLEHSL (SEQ ID NO: 5) and HELGFKVVL (SEQ ID NO: 6) and wherein the composition is for use in treating cancer is a subject that expresses an MHC encoded by an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, an HLA-B*18:01 allele, or any combination thereof.

In some embodiments, the therapeutic composition comprises: (i) a recombinant polypeptide comprising at least five cancer-specific CD8+ T cell epitopes, or (ii) a polynucleotide encoding a recombinant polypeptide comprising at least five cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least five cancer-specific CD8+ T cell epitopes are GVYDGREHTV (SEQ ID NO: 1) and KLVELEHTL (SEQ ID NO: 2) and SPSSASLAL (SEQ ID NO: 3) and KEFAFLEHSL (SEQ ID NO: 5) and HELGFKVVL (SEQ ID NO: 6). In some embodiments, the therapeutic composition comprises: (i) a recombinant polypeptide consisting of at least five cancer-specific CD8+ T cell epitopes, or (ii) a polynucleotide encoding a recombinant polypeptide consisting of at least five cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least five cancer-specific CD8+ T cell epitopes are GVYDGREHTV (SEQ ID NO: 1) and KLVELEHTL (SEQ ID NO: 2) and SPSSASLAL (SEQ ID NO: 3) and KEFAFLEHSL (SEQ ID NO: 5) and HELGFKVVL (SEQ ID NO: 6). In some embodiments, the therapeutic composition comprises: (i) a recombinant polypeptide comprising at least five cancer-specific CD8+ T cell epitopes, or (ii) a polynucleotide encoding a recombinant polypeptide comprising at least five cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least five cancer-specific CD8+ T cell epitopes are GVYDGREHTV (SEQ ID NO: 1) and KLVELEHTL (SEQ ID NO: 2) and SPSSASLAL (SEQ ID NO: 3) and KEFAFLEHSL (SEQ ID NO: 5) and HELGFKVVL (SEQ ID NO: 6) and wherein the composition is for use in treating cancer is a subject that expresses an MHC encoded by an HLA-A*02:01 allele, an HLA-B*07:02 allele, an HLA-B*35:03 allele, an HLA-B*40:01 allele, an HLA-B*40:02 allele, an HLA-B*44:02 allele, an HLA-B*18:01 allele, or any combination thereof. In some embodiments, the therapeutic composition comprises: (i) a recombinant polypeptide consisting of at least five cancer-specific CD8+ T cell epitopes, or (ii) a polynucleotide encoding a recombinant polypeptide consisting of at five cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least five cancer-specific CD8+ T cell epitopes are GVYDGREHTV (SEQ ID NO: 1) and KLVELEHTL (SEQ ID NO: 2) and SPSSASLAL (SEQ ID NO: 3) and KEFAFLEHSL (SEQ ID NO: 5) and HELGFKVVL (SEQ ID NO: 6) and wherein the composition is for use in treating cancer is a subject that expresses an MHC encoded by an HLA-A*02:01 allele, an HLA-B*07:02 allele, an HLA-B*35:03 allele, an HLA-B*40:01 allele, an HLA-B*40:02 allele, an HLA-B*44:02 allele, an HLA-B*18:01 allele, or any combination thereof.

In some embodiments, the therapeutic composition comprises: (i) a recombinant polypeptide comprising at least six cancer-specific CD8+ T cell epitopes, or (ii) a polynucleotide encoding a recombinant polypeptide comprising at least six cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least six cancer-specific CD8+ T cell epitopes are GVYDGREHTV (SEQ ID NO: 1) and KLVELEHTL (SEQ ID NO: 2) and SPSSASLAL (SEQ ID NO: 3) and KPRPDVTNEL (SEQ ID NO: 4) and KEFAFLEHSL (SEQ ID NO: 5) and HELGFKVVL (SEQ ID NO: 6). In some embodiments, the therapeutic composition comprises: (i) a recombinant polypeptide consisting of at least six cancer-specific CD8+ T cell epitopes, or (ii) a polynucleotide encoding a recombinant polypeptide consisting of at least six cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least six cancer-specific CD8+ T cell epitopes are GVYDGREHTV (SEQ ID NO: 1) and KLVELEHTL (SEQ ID NO: 2) and SPSSASLAL (SEQ ID NO: 3) and KPRPDVTNEL (SEQ ID NO: 4) and KEFAFLEHSL (SEQ ID NO: 5) and HELGFKVVL (SEQ ID NO: 6). In some embodiments, the therapeutic composition comprises: (i) a recombinant polypeptide comprising at least six cancer-specific CD8+ T cell epitopes, or (ii) a polynucleotide encoding a recombinant polypeptide comprising at least six cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least six cancer-specific CD8+ T cell epitopes are GVYDGREHTV (SEQ ID NO: 1) and KLVELEHTL (SEQ ID NO: 2) and SPSSASLAL (SEQ ID NO: 3) and KPRPDVTNEL (SEQ ID NO: 4) and KEFAFLEHSL (SEQ ID NO: 5) and HELGFKVVL (SEQ ID NO: 6) and wherein the composition is for use in treating cancer is a subject that expresses an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, an HLA-B*18:01 allele, or any combination thereof. In some embodiments, the therapeutic composition comprises: (i) a recombinant polypeptide consisting of at least six cancer-specific CD8+ T cell epitopes, or (ii) a polynucleotide encoding a recombinant polypeptide consisting of at six cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least six cancer-specific CD8+ T cell epitopes are GVYDGREHTV (SEQ ID NO: 1) and KLVELEHTL (SEQ ID NO: 2) and SPSSASLAL (SEQ ID NO: 3) and KPRPDVTNEL (SEQ ID NO: 4) and KEFAFLEHSL (SEQ ID NO: 5) and HELGFKVVL (SEQ ID NO: 6) and wherein the composition is for use in treating cancer is a subject that expresses an MHC encoded by an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, an HLA-B*18:01 allele, or any combination thereof.

In some embodiments, the therapeutic composition comprises: (i) a recombinant polypeptide comprising one, two, three, four, five, six, or more cancer-specific CD8+ T cell epitopes, or (ii) a polynucleotide encoding a recombinant polypeptide comprising one, two, three, four, five, six, or more cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the one, two, three, four, five, six, or more cancer-specific CD8+ T cell epitopes comprise a cancer-specific CD8+ T cell epitope or plurality of cancer-specific CD8+ T cell epitopes described herein and wherein the composition is for use in treating cancer is a subject that expresses an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, an HLA-B*18:01 allele, an HLA-A*03:01 allele, an HLA-A*31:01 allele, an HLA-A*11:01 allele, an HLA-C*03:03 allele, an HLA-C*03:04 allele, an HLA-B*15:01 allele, an HLA-B*08:01 allele, an HLA-A*02:05 allele, or any combination thereof.

In some embodiments, the therapeutic composition comprises more than one vectors, e.g., at least 2, at least 3, at least 4, at least 5, or at least 6 vectors, wherein the first, second, third, fourth, fifth, and/or the sixth vector is a vector described herein.

In some embodiment, the first vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-35, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-35 and the second vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-35, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-35.

In some embodiment, the first vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-35, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-35, the second vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-35, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-35, and the third vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-35, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-35.

In some embodiment, the first vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-35, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-35, the second vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-35, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-35, and the third vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-35, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-35, and the fourth vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-35, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-35.

In some embodiment, the first vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-35, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-35, the second vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-35, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-35, and the third vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-35, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-35, and the fourth vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-35, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-35, and the fifth vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-35, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-35.

In some embodiment, the first vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-35, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-35, the second vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-35, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-35, and the third vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-35, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-35, and the fourth vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-35, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-35, and the fifth vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-35, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-35, and the sixth vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-35, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-35.

In some embodiment, the first vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-6, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-6, and the second vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-6, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-6.

In some embodiment, the first vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-6, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-6, the second vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-6, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-6, and the third vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-6, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-6.

In some embodiment, the first vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-6, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-6, the second vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-6, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-6, and the third vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-6, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-6, and the fourth vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-6, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-6.

In some embodiment, the first vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-6, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-6, the second vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-6, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-6, and the third vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-6, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-6, and the fourth vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-6, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-6, and the fifth vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-6, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-6.

In some embodiment, the first vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-6, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-6, the second vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-6, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-6, and the third vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-6, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-6, and the fourth vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-6, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-6, and the fifth vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-6, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-6, and the sixth vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-6, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 1-6.

In some embodiment, the first vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 7-12, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 7-12, and the second vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 7-12, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 7-12.

In some embodiment, the first vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 7-12, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 7-12, the second vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 7-12, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 7-12, and the third vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 7-12, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 7-12.

In some embodiment, the first vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 7-12, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 7-12, the second vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 7-12, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 7-12, and the third vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 7-12, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 7-12, and the fourth vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 7-12, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 7-12.

In some embodiment, the first vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 7-12, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 7-12, the second vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 7-12, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 7-12, and the third vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 7-12, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 7-12, and the fourth vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 7-12, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 7-12, and the fifth vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 7-12, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 7-12.

In some embodiment, the first vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 7-12, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 7-12, the second vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 7-12, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 7-12, and the third vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 7-12, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 7-12, and the fourth vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 7-12, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 7-12, and the fifth vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 7-12, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 7-12, and the sixth vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 7-12, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 7-12.

In some embodiment, the first vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 13-18, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 13-18, and the second vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 13-18, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 13-18.

In some embodiment, the first vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 13-18, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 13-18, the second vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 13-18, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 13-18, and the third vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 13-18, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 13-18.

In some embodiment, the first vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 13-18, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 13-18, the second vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 13-18, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 13-18, and the third vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 13-18, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 13-18, and the fourth vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 13-18, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 13-18.

In some embodiment, the first vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 13-18, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 13-18, the second vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 13-18, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 13-18, and the third vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 13-18, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 13-18, and the fourth vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 13-18, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 13-18, and the fifth vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 13-18, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 13-18.

In some embodiment, the first vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 13-18, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 13-18, the second vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 13-18, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 13-18, and the third vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 13-18, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 13-18, and the fourth vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 13-18, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 13-18, and the fifth vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 13-18, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 13-18, and the sixth vector comprises (i) a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 13-18, or (ii) a polynucleotide encoding a recombinant polypeptide comprising a single epitope, e.g., a single T cell epitope, e.g., a single cancer-specific CD8+ epitope, e.g., a single cancer-specific CD8+ epitope comprising an amino acid sequence as set forth in any of SEQ ID NOs: 13-18.

In some embodiments, the composition, e.g., the therapeutic composition is capable of inducing production of an inflated memory CD8+ T cell response that is capable of controlling tumor growth in the subject greater than 1, day, 2 days, 5 days, 10 days, 15 days, 20 days, 25 days, 30 days, 40 days, 45 days, 50 days, 55 days, 60 days, or longer after the composition is administered.

In some embodiments, the composition, e.g., the therapeutic composition is an immunogenic composition. The immunogenic composition may further comprise one or more additional active ingredients, pharmaceutically acceptable carrier, diluent, excipient or adjuvant.

The immunogenic composition comprising a vector according to the disclosure may be used in combination with at least one other immunogenic composition comprising a vector according to the disclosure, wherein each vector encodes an identical cancer specific CD8+ T cell epitope. The immunogenic composition comprising a vector according to the disclosure may be used in combination with at least one other immunogenic composition comprising a vector according to the disclosure, wherein each vector encodes a different cancer specific CD8+ T cell epitope. In some embodiments, the immunogenic composition comprising a first vector according to the disclosure may be administered separately, sequentially or simultaneously with an immunogenic composition comprising a second vector according to the disclosure. In some embodiments, the immunogenic composition comprising a first vector according to the disclosure may be administered separately, sequentially or simultaneously with an immunogenic composition comprising a second vector described herein and an immunogenic composition comprising a third vector described herein. In some embodiments, the immunogenic composition comprising a first vector according to the disclosure may be administered separately, sequentially or simultaneously with an immunogenic composition comprising a second vector, a third vector, and a fourth vector described herein. In some embodiments, the immunogenic composition comprising a first vector according to the disclosure may be administered separately, sequentially or simultaneously with an immunogenic composition comprising a second vector, a third vector, a fourth vector, and a fifth vector described herein. In some embodiments, the immunogenic composition comprising a first vector according to the disclosure may be administered separately, sequentially or simultaneously with an immunogenic composition comprising a second vector, a third vector, a fourth vector, a fifth vector, and a sixth vector described herein. In some embodiments, the immunogenic composition comprising a first vector according to the disclosure may be administered separately, sequentially or simultaneously with an immunogenic composition comprising a second vector, a third vector, a fourth vector, a fifth vector, a sixth vector, and a seventh vector described herein. In some embodiments, the immunogenic composition comprising a first vector according to the disclosure may be administered separately, sequentially or simultaneously with an immunogenic composition comprising a second vector, a third vector, a fourth vector, a fifth vector, a sixth vector, a seventh vector, and an eighth vector described herein.

The use of a cocktail of vectors encoding different epitopes may result in a stronger immune response, further there may be a synergistic effect which enhances the immune response. In some embodiments, the immunogenic composition comprises a cocktail of vectors encoding different epitopes, e.g., cancer-specific CD8+ epitopes.

The composition can be in the form of a liquid, e.g., a solution, emulsion, or suspension. The liquid compositions of the disclosure, whether they are solutions, suspensions or other like form, can also include one or more of the following: sterile diluents such as water, saline solution, preferably physiological saline, Ringer's solution, isotonic sodium chloride, fixed oils such as synthetic mono or digylcerides, polyethylene glycols, glycerin, or other solvents; antibacterial agents such as benzyl alcohol or methyl paraben; and agents for the adjustment of tonicity such as sodium chloride or dextrose. The composition can be enclosed in an ampoule, a disposable syringe or a multiple-dose vial made of glass, plastic or other material.

The compositions can be prepared using methodology well known in the pharmaceutical art. For example, a composition intended to be administered by injection can be prepared by combining a vector of the present disclosure with water so as to form a solution. A surfactant can be added to facilitate the formation of a homogeneous solution or suspension.

In some embodiments, the disclosure provides a host cell, comprising one or more recombinant polypeptides, polynucleotides, vectors and/or the compositions described herein. Host cells are genetically engineered (transduced or transformed or transfected) with the vectors of this disclosure which can be, for example, a cloning vector or an expression vector. The vector can be, for example, in the form of a plasmid, a viral particle, a phage, etc. The host cell genetically engineered (transduced or transformed or transfected) with one or more vectors, e.g., one or more vectors disclosed herein. The host cell may be used to produce an adenoviral stock. The engineered host cells can be cultured in conventional nutrient media modified as appropriate for activating promoters, selecting transformants or amplifying the polynucleotides. The culture conditions, such as temperature, pH and the like, are those previously used with the host cell selected for expression and will be apparent to a person skilled in the arts.

Suitable host cells for expression of a polypeptide include prokaryotes, yeast, insect or higher eukaryotic cells under the control of appropriate promoters. Prokaryotes include gram negative or gram positive organisms, for example E. coli or bacilli. Higher eukaryotic cells include established cell lines of mammalian origin. Cell-free translation systems could also be employed. Appropriate cloning and expression vectors for use with bacterial, fungal, yeast, and mammalian cellular hosts are well known in the art (see Pouwels et al., Cloning Vectors: A Laboratory Manual, Elsevier, N.Y., 1985). In some embodiments, the host cell can be mammalian for example human or mouse.

### Pharmaceutical Compositions

In one aspect, the disclosure provides a pharmaceutical composition comprising one or more recombinant polypeptides, polynucleotides, vectors, cells, or compositions disclosed herein and optionally further comprising a pharmaceutically acceptable carrier, excipient or additive. The term “pharmaceutical composition”, as used herein, refers to a composition formulated for pharmaceutical use. Pharmaceutical compositions comprises an immunologically effective amount of one or more recombinant polypeptides, polynucleotides, vectors, cells, or compositions disclosed herein, and optionally one or more other components which are pharmaceutically acceptable. In some embodiments, a pharmaceutical composition disclosed herein can contain at least one, at least 2, at least 3, at least, 4, at least 5, or 6 epitopes, e.g., T cell epitopes, e.g., cancer-specific CD8+ and/or CD4+ epitopes, e.g., cancer specific CD8+ T cell epitopes. In some embodiments, a pharmaceutical composition disclosed herein can contain 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 epitopes, e.g., T cell epitopes, e.g., cancer-specific CD8+ and/or CD4+ epitopes, e.g., cancer specific CD8+ T cell epitopes. When administered as a combination, the recombinant polypeptides, polynucleotides, vectors, cells, or compositions can be formulated as separate compositions that are given at the same time or different times, or the recombinant polypeptides, polynucleotides, vectors, cells, or compositions can be given as a single composition.

In some embodiments, the pharmaceutical composition comprises additional agents, e.g., for specific delivery, increasing half-life, or other therapeutic compounds. In some embodiments, the pharmaceutical composition may comprise one or more of dimethylsulfoxide (DMSO), dextrose, water, succinate, poly I: poly C, poly-L-lysine, carboxymethylcellulose, and/or chloride.

In some embodiments, a pharmaceutically acceptable carrier comprises any vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.)

Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanthin; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier,” “pharmaceutically acceptable carrier” or the like are used interchangeably herein.

Pharmaceutical compositions can comprise one or more pH buffering compounds to maintain the pH of the formulation at a predetermined level that reflects physiological pH, such as in the range of about 5.0 to about 8.0. The pH buffering compound used in the aqueous liquid formulation can be an amino acid or mixture of amino acids, such as histidine or a mixture of amino acids such as histidine and glycine. Alternatively, the pH buffering compound is preferably an agent which maintains the pH of the formulation at a predetermined level, such as in the range of about 5.0 to about 8.0, and which does not chelate calcium ions. Illustrative examples of such pH buffering compounds include, but are not limited to, imidazole and acetate ions. The pH buffering compound may be present in any amount suitable to maintain the pH of the formulation at a predetermined level.

Pharmaceutical compositions can also contain one or more osmotic modulating agents, i.e., a compound that modulates the osmotic properties (e.g., tonicity, osmolality, and/or osmotic pressure) of the formulation to a level that is acceptable to the blood stream and blood cells of recipient individuals. The osmotic modulating agent can be an agent that does not chelate calcium ions. The osmotic modulating agent can be any compound known or available to those skilled in the art that modulates the osmotic properties of the formulation. One skilled in the art may empirically determine the suitability of a given osmotic modulating agent for use in the inventive formulation. Illustrative examples of suitable types of osmotic modulating agents include, but are not limited to, salts, such as sodium chloride and sodium acetate; sugars, such as sucrose, dextrose, and mannitol; amino acids, such as glycine; and mixtures of one or more of these agents and/or types of agents. The osmotic modulating agent(s) may be present in any concentration sufficient to modulate the osmotic properties of the formulation.

The pharmaceutical composition comprising one or more recombinant polypeptides, polynucleotides, vectors, cells, or compositions disclosed herein may be used in vitro, ex-vivo, or in vivo. In some embodiments, the pharmaceutical composition is formulated for delivery to a subject, e.g., for inducing an immune response.

Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration. In some embodiments, the pharmaceutical composition described herein is administered locally to a diseased site (e.g., tumor site). In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber. In other embodiments, the pharmaceutical composition described herein is delivered in a controlled release system. In one embodiment, a pump can be used. In another embodiment, polymeric materials can be used. In some embodiments, the pharmaceutical composition is formulated in accordance with routine procedures as a composition adapted for intravenous or subcutaneous administration to a subject, e.g., a human. In some embodiments, pharmaceutical composition for administration by injection are solutions in sterile isotonic use as solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachets indicating the quantity of active agent. Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

A pharmaceutical composition for systemic administration can be a liquid, e.g., sterile saline, viralsterile saline, lactated Ringer's or Hank's solution. In addition, the pharmaceutical composition can be in solid forms and re-dissolved or suspended immediately prior to use. Lyophilized forms are also contemplated. The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Compounds can be entrapped in “stabilized plasmid-lipid particles” (SPLP) containing the fusogenic lipid dioleoylphosphatidylethanolamine (DOPE), low levels (5-10 mol %) of cationic lipid, and stabilized by a polyethyleneglycol (PEG) coating. Positively charged lipids such as N41-(2,3-dioleoyloxi) propy 1]-N,N,N-trimethyl-amoniummethylsulfate, or “DOTAP,” are particularly preferred for such particles and vesicles.

The pharmaceutical composition described herein can be administered or packaged as a unit dose, for example, in reference to a pharmaceutical composition to physically discrete units suitable as unitary dosage for the subject, each unit containing a predetermined quantity of active material calculated to produce the desired therapeutic effect in association with the required diluent, i.e., carrier, or vehicle. Further, the pharmaceutical composition can be provided as a pharmaceutical kit comprising (a) a container containing a compound of the invention in lyophilized form and (b) a second container containing a pharmaceutically acceptable diluent (e.g., sterile used for reconstitution or dilution of the lyophilized compound of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

Formulation of the pharmaceutical compositions described herein can be prepared by any method known or hereafter developed in the art of pharmacology. In general, such preparatory methods include the step of bringing the active ingredient(s) into association with an excipient and/or one or more other accessory ingredients, and then, if necessary and/or desirable, shaping and/or packaging the product into a desired single- or multi-dose unit. Pharmaceutical formulations can additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants, and the like, as suited to the particular dosage form desired.

A formulation for intravenous administration of the recombinant polypeptides, polynucleotides, vectors, cells, or compositions of the disclosure may be in the form of a sterile injectable aqueous or non-aqueous (e.g., oleaginous) solution or suspension. The sterile injectable preparation may also be in a sterile injectable solution or suspension in a non-toxic parenterally-acceptable diluent or solvent, for example, a solution in 1,3-butanediol. Among the acceptable vehicles and solvents that may be employed are water, phosphate buffer solution, Ringer's solution and isotonic sodium chloride solution. In addition, sterile, fixed oils may be employed as a solvent or suspending medium. For this purpose, any bland fixed oil may be employed, including synthetic mono- or diglycerides. In addition, fatty acids such as oleic acid may be used in the preparation of the intravenous formulation of the disclosure.

In some embodiments, the composition or pharmaceutical composition comprises at least two vectors as described herein. In some embodiments, the vectors are identical, e.g., both adenoviral vectors. In some embodiments, the vectors are different; e.g., an adenoviral vector and an AAV. In some embodiments, the vectors may be provided as separate medicaments for administration at the same time or at different times to a subject in need thereof.

In some embodiments, wherein the composition comprises at least two vectors as described herein, the vectors may be provided as separate medicaments for administration at different times. When administered separately and at different times, the composition comprising either vector may be administered first. In some embodiments, the compositions can be administered on the same day or on different days. In some embodiments, the compositions can be administered using the same schedule or at different schedules during the treatment cycle.

Alternatively, wherein the composition comprises at least two vectors as described herein, the administration of the vectors may be performed simultaneously. Wherein simultaneous administration is used the vectors may be formulated as separate pharmaceutical compositions. In some embodiments, the at least two vectors may be formulated as a single pharmaceutical composition. In some embodiments, the at least three vectors may be formulated as a single pharmaceutical composition. In some embodiments, the at least four vectors may be formulated as a single pharmaceutical composition. In some embodiments, the at least five vectors may be formulated as a single pharmaceutical composition. In some embodiments, the at least six vectors may be formulated as a single pharmaceutical composition. In some embodiments, the at least seven vectors may be formulated as a single pharmaceutical composition. In some embodiments, the at least eight vectors may be formulated as a single pharmaceutical composition. In some embodiments, the composition comprises all vectors that are different. In some embodiments, the composition comprises at least two identical vectors. In some embodiments, the composition comprises at least three identical vectors. In some embodiments, the composition comprises at least four identical vectors. In some embodiments, the composition comprises at least five identical vectors. In some embodiments, the composition comprises at least six identical vectors.

In some embodiments, the recombinant polypeptides, polynucleotides, vectors, cells, compositions, pharmaceutical compositions, or formulations disclosed herein induce an inflating memory T cell response, e.g., an inflating memory CD8+ T cell response in the subject. In some embodiments, the inflating memory T cell response, e.g., an inflating memory CD8+ T cell response, targets cancer cells. In some embodiments, recombinant polypeptides, polynucleotides, vectors, cells, compositions, pharmaceutical compositions, or formulations disclosed herein may comprise of one or a variety of epitopes (e.g., one or a variety of epitopes described herein) targeted to an HLA type encoding an MHC of a subject's cancer cells. In some embodiments, this immunotherapy prevents T cell exhaustion while specifically targeting engineered T cells to the patient's specific cancer HLA type to ensure an effective immune response.

### Methods of Producing Compositions

In one aspect, the present disclosure provides a method of producing the composition described herein comprising (i) synthesizing a nucleotide sequence encoding a single cancer-specific CD8+ T cell epitope, e.g., a single cancer-specific CD8+ T cell epitope described herein, as a sense and antisense primer; (ii) cloning the nucleotide sequence encoding the single cancer-specific CD8+ T cell epitope, e.g., the single cancer-specific CD8+ T cell epitope into a first plasmid; and (iii) cloning a sequence comprising the nucleotide sequence encoding the single cancer-specific CD8+ T cell epitope, e.g., the single cancer-specific CD8+ T cell epitope from a first plasmid into a second vector, e.g., a second vector comprising adenoviral DNA. Suitable cloning methods are known within the art, examples of cloning methods include but are not limited to, restriction ligations methods, Gateway cloning, Gibson assembly, ligation independent cloning. The person skilled in the art will be able to determine a suitable method to clone the sequence into the plasmid. The cloning method to introduce the nucleic acid sequence encoding epitope sequence into the first plasmid may be the same or different from the cloning method used to. In some embodiments, the cloning method to introduce the nucleic acid sequence encoding epitope sequence into the first plasmid is selected from restriction ligations methods, Gateway cloning, Gibson assembly, ligation independent cloning. In some embodiments, the cloning method to introduce the nucleic acid sequence encoding epitope into the second plasmid comprising the adenoviral DNA is selected from restriction ligations methods, Gateway cloning, Gibson assembly, ligation independent cloning. In some embodiments, the second vector encodes an adenoviral vector, e.g., an adenoviral vector described herein, e.g., an adhu5 adenoviral vector.

In some embodiments, the present disclosure relates to a method for inducing a T cell immune response in an animal against a cancer-specific epitope, e.g., a cancer-specific CD8+ epitope, the method comprising contacting a cell with a recombinant polypeptide, polynucleotide, vector, cell, composition, pharmaceutical composition, or a formulation as described herein.

The cell may be contacted with the recombinant polypeptide, polynucleotide, vector, cell, composition, pharmaceutical composition, or the formulation in an in vitro manner, in an ex vivo manner, or in an in vivo manner. Wherein the cells are contacted with the recombinant polypeptide, polynucleotide, vector, cell, composition, pharmaceutical composition, or the formulation either in vitro or ex vivo, the cells may then be administered to a subject. The T cell immune response may comprise an inflating memory CD8+ T cell response.

### Methods of Treatment

In some aspects, the present disclosure provides a method of treating cancer in a subject in need thereof comprising administering one or more recombinant polypeptides, polynucleotides, vectors, cells, compositions, pharmaceutical compositions, or formulations disclosed herein.

In some embodiments, the method of treating cancer in a subject in need thereof comprises administering to the subject a composition comprising: (i) a recombinant polypeptide comprising a cancer-specific CD8+ T cell epitope, or (ii) a polynucleotide encoding a recombinant polypeptide comprising a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is GVYDGREHTV (SEQ ID NO: 1). In some embodiments, the method of treating cancer in a subject in need thereof comprises administering to the subject a composition comprising: (i) a recombinant polypeptide consisting of a cancer-specific CD8+ T cell epitope, or (ii) a polynucleotide encoding a recombinant polypeptide consisting of a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is GVYDGREHTV (SEQ ID NO: 1). In some embodiments, the method of treating cancer in a subject in need thereof comprises administering to the subject a composition comprising: (i) a recombinant polypeptide comprising a cancer-specific CD8+ T cell epitope, or (ii) a polynucleotide encoding a recombinant polypeptide comprising a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is GVYDGREHTV (SEQ ID NO: 1) and wherein the subject expresses an MHC encoded by an HLA-A*02:01 allele. In some embodiments, the method of treating cancer in a subject in need thereof comprises administering to the subject a composition comprising: (i) a recombinant polypeptide consisting of a cancer-specific CD8+ T cell epitope, or (ii) a polynucleotide encoding a recombinant polypeptide consisting of a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is GVYDGREHTV (SEQ ID NO: 1) and wherein the subject expresses an MHC encoded by an HLA-A*02:01 allele.

In some embodiments, the method of treating cancer in a subject in need thereof comprises administering to the subject a composition comprising: (i) a recombinant polypeptide comprising a cancer-specific CD8+ T cell epitope, or (ii) a polynucleotide encoding a recombinant polypeptide comprising a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is KLVELEHTL (SEQ ID NO: 2). In some embodiments, the method of treating cancer in a subject in need thereof comprises administering to the subject a composition comprising: (i) a recombinant polypeptide consisting of a cancer-specific CD8+ T cell epitope, or (ii) a polynucleotide encoding a recombinant polypeptide consisting of a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is KLVELEHTL (SEQ ID NO: 2). In some embodiments, the method of treating cancer in a subject in need thereof comprises administering to the subject a composition comprising: (i) a recombinant polypeptide comprising a cancer-specific CD8+ T cell epitope, or (ii) a polynucleotide encoding a recombinant polypeptide comprising a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is KLVELEHTL (SEQ ID NO: 2) and wherein the subject expresses an MHC encoded by an HLA-A*02:01 allele. In some embodiments, the method of treating cancer in a subject in need thereof comprises administering to the subject a composition comprising: (i) a recombinant polypeptide consisting of a cancer-specific CD8+ T cell epitope, or (ii) a polynucleotide encoding a recombinant polypeptide consisting of a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is KLVELEHTL (SEQ ID NO: 2) and wherein the subject expresses an MHC encoded by an HLA-A*02:01 allele.

In some embodiments, the method of treating cancer in a subject in need thereof comprises administering to the subject a composition comprising: (i) a recombinant polypeptide comprising a cancer-specific CD8+ T cell epitope, or (ii) a polynucleotide encoding a recombinant polypeptide comprising a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is SPSSASLAL (SEQ ID NO: 3). In some embodiments, the method of treating cancer in a subject in need thereof comprises administering to the subject a composition comprising: (i) a recombinant polypeptide consisting of a cancer-specific CD8+ T cell epitope, or (ii) a polynucleotide encoding a recombinant polypeptide consisting of a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is SPSSASLAL (SEQ ID NO: 3). In some embodiments, the method of treating cancer in a subject in need thereof comprises administering to the subject a composition comprising: (i) a recombinant polypeptide comprising a cancer-specific CD8+ T cell epitope, or (ii) a polynucleotide encoding a recombinant polypeptide comprising a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is SPSSASLAL (SEQ ID NO: 3) and wherein the subject expresses an MHC encoded by an HLA-B*07:02 allele and/or an HLA-B*35:03 allele. In some embodiments, the method of treating cancer in a subject in need thereof comprises administering to the subject a composition comprising: (i) a recombinant polypeptide consisting of a cancer-specific CD8+ T cell epitope, or (ii) a polynucleotide encoding a recombinant polypeptide consisting of a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is SPSSASLAL (SEQ ID NO: 3) and wherein the subject expresses an MHC encoded by an HLA-B*07:02 allele and/or an HLA-B*35:03 allele.

In some embodiments, the method of treating cancer in a subject in need thereof comprises administering to the subject a composition comprising: (i) a recombinant polypeptide comprising a cancer-specific CD8+ T cell epitope, or (ii) a polynucleotide encoding a recombinant polypeptide comprising a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is KPRPDVTNEL (SEQ ID NO: 4). In some embodiments, the method of treating cancer in a subject in need thereof comprises administering to the subject a composition comprising: (i) a recombinant polypeptide consisting of a cancer-specific CD8+ T cell epitope, or (ii) a polynucleotide encoding a recombinant polypeptide consisting of a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is KPRPDVTNEL (SEQ ID NO: 4). In some embodiments, the method of treating cancer in a subject in need thereof comprises administering to the subject a composition comprising: (i) a recombinant polypeptide comprising a cancer-specific CD8+ T cell epitope, or (ii) a polynucleotide encoding a recombinant polypeptide comprising a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is KPRPDVTNEL (SEQ ID NO: 4) and wherein the subject expresses an MHC encoded by an HLA-B*07:02 allele, an HLA-B*35:03 allele, an HLA-C*07:02 allele, or any combination thereof. In some embodiments, the method of treating cancer in a subject in need thereof comprises administering to the subject a composition comprising: (i) a recombinant polypeptide consisting of a cancer-specific CD8+ T cell epitope, or (ii) a polynucleotide encoding a recombinant polypeptide consisting of a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is KPRPDVTNEL (SEQ ID NO: 4) and wherein the subject expresses an MHC encoded by an HLA-B*07:02 allele, an HLA-B*35:03 allele, an HLA-C*07:02 allele, or any combination thereof.

In some embodiments, the method of treating cancer in a subject in need thereof comprises administering to the subject a composition comprising: (i) a recombinant polypeptide comprising a cancer-specific CD8+ T cell epitope, or (ii) a polynucleotide encoding a recombinant polypeptide comprising a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is KEFAFLEHSL (SEQ ID NO: 5). In some embodiments, the method of treating cancer in a subject in need thereof comprises administering to the subject a composition comprising: (i) a recombinant polypeptide consisting of a cancer-specific CD8+ T cell epitope, or (ii) a polynucleotide encoding a recombinant polypeptide consisting of a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is KEFAFLEHSL (SEQ ID NO: 5). In some embodiments, the method of treating cancer in a subject in need thereof comprises administering to the subject a composition comprising: (i) a recombinant polypeptide comprising a cancer-specific CD8+ T cell epitope, or (ii) a polynucleotide encoding a recombinant polypeptide comprising a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is KEFAFLEHSL (SEQ ID NO: 5) and wherein the subject expresses an MHC encoded by an HLA-B*40:01 allele. In some embodiments, the method of treating cancer in a subject in need thereof comprises administering to the subject a composition comprising: (i) a recombinant polypeptide comprising a cancer-specific CD8+ T cell epitope, or (ii) a polynucleotide encoding a recombinant polypeptide comprising a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is KEFAFLEHSL (SEQ ID NO: 5) and wherein the subject expresses an MHC encoded by an HLA-B*40:01 allele, an HLA-B*40:02 allele, an HLA-B*44:02 allele, or any combination thereof. In some embodiments, the method of treating cancer in a subject in need thereof comprises administering to the subject a composition comprising: (i) a recombinant polypeptide consisting of a cancer-specific CD8+ T cell epitope, or (ii) a polynucleotide encoding a recombinant polypeptide consisting of a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is KEFAFLEHSL (SEQ ID NO: 5) and wherein the subject expresses an MHC encoded by an HLA-B*40:01 allele. In some embodiments, the method of treating cancer in a subject in need thereof comprises administering to the subject a composition comprising: (i) a recombinant polypeptide consisting of a cancer-specific CD8+ T cell epitope, or (ii) a polynucleotide encoding a recombinant polypeptide consisting of a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is KEFAFLEHSL (SEQ ID NO: 5) and wherein the subject expresses an MHC encoded by an HLA-B*44:02 allele.

In some embodiments, the method of treating cancer in a subject in need thereof comprises administering to the subject a composition comprising: (i) a recombinant polypeptide comprising a cancer-specific CD8+ T cell epitope, or (ii) a polynucleotide encoding a recombinant polypeptide comprising a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is HELGFKVVL (SEQ ID NO: 6). In some embodiments, the method of treating cancer in a subject in need thereof comprises administering to the subject a composition comprising: (i) a recombinant polypeptide consisting of a cancer-specific CD8+ T cell epitope, or (ii) a polynucleotide encoding a recombinant polypeptide consisting of a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is HELGFKVVL (SEQ ID NO: 6). In some embodiments, the method of treating cancer in a subject in need thereof comprises administering to the subject a composition comprising: (i) a recombinant polypeptide comprising a cancer-specific CD8+ T cell epitope, or (ii) a polynucleotide encoding a recombinant polypeptide comprising a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is HELGFKVVL (SEQ ID NO: 6) and wherein the subject expresses an MHC encoded by an HLA-B*18:01 allele. In some embodiments, the method of treating cancer in a subject in need thereof comprises administering to the subject a composition comprising: (i) a recombinant polypeptide comprising a cancer-specific CD8+ T cell epitope, or (ii) a polynucleotide encoding a recombinant polypeptide comprising a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is HELGFKVVL (SEQ ID NO: 6) and wherein the subject expresses an MHC encoded by an HLA-B*40:01 allele. In some embodiments, the method of treating cancer in a subject in need thereof comprises administering to the subject a composition comprising: (i) a recombinant polypeptide comprising a cancer-specific CD8+ T cell epitope, or (ii) a polynucleotide encoding a recombinant polypeptide comprising a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is HELGFKVVL (SEQ ID NO: 6) and wherein the subject expresses an MHC encoded by an HLA-B*44:02 allele. In some embodiments, the method of treating cancer in a subject in need thereof comprises administering to the subject a composition comprising: (i) a recombinant polypeptide consisting of a cancer-specific CD8+ T cell epitope, or (ii) a polynucleotide encoding a recombinant polypeptide consisting of a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is HELGFKVVL (SEQ ID NO: 6) and wherein the subject expresses an MHC encoded by an HLA-B*18:01 allele. In some embodiments, the method of treating cancer in a subject in need thereof comprises administering to the subject a composition comprising: (i) a recombinant polypeptide consisting of a cancer-specific CD8+ T cell epitope, or (ii) a polynucleotide encoding a recombinant polypeptide consisting of a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is HELGFKVVL (SEQ ID NO: 6) and wherein the subject expresses an MHC encoded by an HLA-B*40:01 allele. In some embodiments, the method of treating cancer in a subject in need thereof comprises administering to the subject a composition comprising: (i) a recombinant polypeptide consisting of a cancer-specific CD8+ T cell epitope, or (ii) a polynucleotide encoding a recombinant polypeptide consisting of a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is HELGFKVVL (SEQ ID NO: 6) and wherein the subject expresses an MHC encoded by an HLA-B*44:02 allele. In some embodiments, the therapeutic composition comprises: (i) a recombinant polypeptide consisting of a cancer-specific CD8+ T cell epitope, or (ii) a polynucleotide encoding a recombinant polypeptide consisting of a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is HELGFKVVL (SEQ ID NO: 6) and wherein the composition is for use in treating cancer is a subject that expresses an MHC encoded by an HLA-B*40:02 allele.

In some embodiments, the method of treating cancer in a subject in need thereof comprises administering: (i) a recombinant polypeptide comprising a cancer-specific CD8+ T cell epitope, or (ii) a polynucleotide encoding a recombinant polypeptide comprising a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is NLRPPTQEL (SEQ ID NO: 10). In some embodiments, the method of treating cancer in a subject in need thereof comprises administering: (i) a recombinant polypeptide consisting of a cancer-specific CD8+ T cell epitope, or (ii) a polynucleotide encoding a recombinant polypeptide consisting of a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is NLRPPTQEL (SEQ ID NO: 10). In some embodiments, the method of treating cancer in a subject in need thereof comprises administering: (i) a recombinant polypeptide comprising a cancer-specific CD8+ T cell epitope, or (ii) a polynucleotide encoding a recombinant polypeptide comprising a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is NLRPPTQEL (SEQ ID NO: 10) and wherein the composition is for use in treating cancer is a subject that expresses an MHC encoded by an HLA-B*07:02 allele, an HLA-B*35:03 allele, an HLA-C*07:02 allele, an HLA-C*03:03 allele, or any combination thereof. In some embodiments, the method of treating cancer in a subject in need thereof comprises administering: (i) a recombinant polypeptide consisting of a cancer-specific CD8+ T cell epitope, or (ii) a polynucleotide encoding a recombinant polypeptide consisting of a cancer-specific CD8+ T cell epitope, (iii) a vector comprising (i) or (ii), (iv) antigen presenting cells (APCs) comprising (i), (ii), or (iii), or (v) T cells stimulated with APCs comprising (i), (ii), or (iii); wherein the cancer-specific CD8+ T cell epitope is NLRPPTQEL (SEQ ID NO: 10) and wherein the composition is for use in treating cancer is a subject that expresses an MHC encoded by an HLA-B*07:02 allele, an HLA-B*35:03 allele, an HLA-C*07:02 allele, an HLA-C*03:03 allele, or any combination thereof.

In some embodiments, the method of treating cancer in a subject in need thereof comprises administering: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are GVYDGREHTV (SEQ ID NO: 1) and KLVELEHTL (SEQ ID NO: 2). In some embodiments, the method of treating cancer in a subject in need thereof comprises administering: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are GVYDGREHTV (SEQ ID NO: 1) and KLVELEHTL (SEQ ID NO: 2). In some embodiments, the method of treating cancer in a subject in need thereof comprises administering: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are GVYDGREHTV (SEQ ID NO: 1) and KLVELEHTL (SEQ ID NO: 2) and wherein the subject expresses an MHC encoded by an HLA-A*02:01 allele. In some embodiments, the method of treating cancer in a subject in need thereof comprises administering: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are GVYDGREHTV (SEQ ID NO: 1) and KLVELEHTL (SEQ ID NO: 2) and wherein the subject expresses an MHC encoded by an HLA-A*02:01 allele.

In some embodiments, the method of treating cancer in a subject in need thereof comprises administering: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are GVYDGREHTV (SEQ ID NO: 1) and SPSSASLAL (SEQ ID NO: 3). In some embodiments, the method of treating cancer in a subject in need thereof comprises administering: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are GVYDGREHTV (SEQ ID NO: 1) and SPSSASLAL (SEQ ID NO: 3). In some embodiments, the method of treating cancer in a subject in need thereof comprises administering: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are GVYDGREHTV (SEQ ID NO: 1) and SPSSASLAL (SEQ ID NO: 3) and wherein the subject expresses an MHC encoded by an HLA-A*02:01 allele, an HLA-B*07:02 allele, an HLA-B*35:03 allele, or any combination thereof. In some embodiments, the method of treating cancer in a subject in need thereof comprises administering: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are GVYDGREHTV (SEQ ID NO: 1) and SPSSASLAL (SEQ ID NO: 3) and wherein the subject expresses an MHC encoded by an HLA-A*02:01 allele, an HLA-B*07:02 allele, an HLA-B*35:03 allele, or any combination thereof.

In some embodiments, the method of treating cancer in a subject in need thereof comprises administering: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are GVYDGREHTV (SEQ ID NO: 1) and KPRPDVTNEL (SEQ ID NO: 4). In some embodiments, the method of treating cancer in a subject in need thereof comprises administering: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are GVYDGREHTV (SEQ ID NO: 1) and KPRPDVTNEL (SEQ ID NO: 4). In some embodiments, the method of treating cancer in a subject in need thereof comprises administering: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are GVYDGREHTV (SEQ ID NO: 1) and KPRPDVTNEL (SEQ ID NO: 4) and wherein the subject expresses an MHC encoded by an HLA-A*02:01 allele, an HLA-B*07:02 allele, an HLA-B*35:03 allele, an HLA-C*07:02 allele, or any combination thereof. In some embodiments, the method of treating cancer in a subject in need thereof comprises administering: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are GVYDGREHTV (SEQ ID NO: 1) and KPRPDVTNEL (SEQ ID NO: 4) and wherein the subject expresses an MHC encoded by an HLA-A*02:01 allele, an HLA-B*07:02 allele, an HLA-B*35:03 allele, an HLA-C*07:02 allele, or any combination thereof.

In some embodiments, the method of treating cancer in a subject in need thereof comprises administering: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are GVYDGREHTV (SEQ ID NO: 1) and KEFAFLEHSL (SEQ ID NO: 5). In some embodiments, the method of treating cancer in a subject in need thereof comprises administering: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are GVYDGREHTV (SEQ ID NO: 1) and KEFAFLEHSL (SEQ ID NO: 5). In some embodiments, the method of treating cancer in a subject in need thereof comprises administering: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are GVYDGREHTV (SEQ ID NO: 1) and KEFAFLEHSL (SEQ ID NO: 5) and wherein the subject expresses an MHC encoded by an HLA-A*02:01 allele, an HLA-B*40:01 allele, an HLA-B*44:02 allele, an HLA-B*40:02 allele, or any combination thereof. In some embodiments, the method of treating cancer in a subject in need thereof comprises administering: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are GVYDGREHTV (SEQ ID NO: 1) and KEFAFLEHSL (SEQ ID NO: 5) and wherein the subject expresses an MHC encoded by an HLA-A*02:01 allele, an HLA-B*40:01 allele, an HLA-B*44:02 allele, an HLA-B*40:02 allele, or any combination thereof.

In some embodiments, the method of treating cancer in a subject in need thereof comprises administering: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are GVYDGREHTV (SEQ ID NO: 1) and HELGFKVVL (SEQ ID NO: 6). In some embodiments, the method of treating cancer in a subject in need thereof comprises administering: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are GVYDGREHTV (SEQ ID NO: 1) and HELGFKVVL (SEQ ID NO: 6). In some embodiments, the method of treating cancer in a subject in need thereof comprises administering: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are GVYDGREHTV (SEQ ID NO: 1) and HELGFKVVL (SEQ ID NO: 6) and wherein the subject expresses an MHC encoded by an HLA-A*02:01 allele, an HLA-B*18:01 allele an HLA-B*40:01 allele, an HLA-B*44:02 allele, an HLA-B*40:02 allele, or any combination thereof. In some embodiments, the method of treating cancer in a subject in need thereof comprises administering: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are GVYDGREHTV (SEQ ID NO: 1) and HELGFKVVL (SEQ ID NO: 6) and wherein the subject expresses an MHC encoded by an HLA-A*02:01 allele, an HLA-B*18:01 allele an HLA-B*40:01 allele, an HLA-B*44:02 allele, an HLA-B*40:02 allele, or any combination thereof.

In some embodiments, the method of treating cancer in a subject in need thereof comprises administering: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KLVELEHTL (SEQ ID NO: 2) and SPSSASLAL (SEQ ID NO: 3). In some embodiments, the method of treating cancer in a subject in need thereof comprises administering: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KLVELEHTL (SEQ ID NO: 2) and SPSSASLAL (SEQ ID NO: 3). In some embodiments, the method of treating cancer in a subject in need thereof comprises administering: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KLVELEHTL (SEQ ID NO: 2) and SPSSASLAL (SEQ ID NO: 3) and wherein the subject expresses an MHC encoded by an HLA-A*02:01 allele, an HLA-B*07:02 allele, an HLA-B*35:03 allele, or any combination thereof. In some embodiments, the method of treating cancer in a subject in need thereof comprises administering: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KLVELEHTL (SEQ ID NO: 2) and SPSSASLAL (SEQ ID NO: 3) and wherein the subject expresses an MHC encoded by an HLA-A*02:01 allele, an HLA-B*07:02 allele, an HLA-B*35:03 allele, or any combination thereof.

In some embodiments, the method of treating cancer in a subject in need thereof comprises administering: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KLVELEHTL (SEQ ID NO: 2) and KPRPDVTNEL (SEQ ID NO: 4). In some embodiments, the method of treating cancer in a subject in need thereof comprises administering: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KLVELEHTL (SEQ ID NO: 2) and KPRPDVTNEL (SEQ ID NO: 4). In some embodiments, the method of treating cancer in a subject in need thereof comprises administering: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KLVELEHTL (SEQ ID NO: 2) and KPRPDVTNEL (SEQ ID NO: 4) and wherein the subject expresses an MHC encoded by an HLA-A*02:01 allele, an HLA-B*07:02 allele, an HLA-B*35:03 allele, an HLA-C*07:02 allele, or any combination thereof. In some embodiments, the method of treating cancer in a subject in need thereof comprises administering: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KLVELEHTL (SEQ ID NO: 2) and KPRPDVTNEL (SEQ ID NO: 4) and wherein the subject expresses an MHC encoded by an HLA-A*02:01 allele, an HLA-B*07:02 allele, an HLA-B*35:03 allele, an HLA-C*07:02 allele, or any combination thereof.

In some embodiments, the method of treating cancer in a subject in need thereof comprises administering: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KLVELEHTL (SEQ ID NO: 2) and KEFAFLEHSL (SEQ ID NO: 5). In some embodiments, the method of treating cancer in a subject in need thereof comprises administering: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KLVELEHTL (SEQ ID NO: 2) and KEFAFLEHSL (SEQ ID NO: 5). In some embodiments, the method of treating cancer in a subject in need thereof comprises administering: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KLVELEHTL (SEQ ID NO: 2) and KEFAFLEHSL (SEQ ID NO: 5) and wherein the subject expresses an MHC encoded by an HLA-A*02:01 allele, an HLA-B*40:01 allele, an HLA-B*44:02 allele, an HLA-B*40:02 allele, or any combination thereof. In some embodiments, the method of treating cancer in a subject in need thereof comprises administering: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KLVELEHTL (SEQ ID NO: 2) and KEFAFLEHSL (SEQ ID NO: 5) and wherein the subject expresses an MHC encoded by an HLA-A*02:01 allele, an HLA-B*40:01 allele, an HLA-B*44:02 allele, an HLA-B*40:02 allele, or any combination thereof.

In some embodiments, the method of treating cancer in a subject in need thereof comprises administering: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KLVELEHTL (SEQ ID NO: 2) and HELGFKVVL (SEQ ID NO: 6). In some embodiments, the method of treating cancer in a subject in need thereof comprises administering: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KLVELEHTL (SEQ ID NO: 2) and HELGFKVVL (SEQ ID NO: 6). In some embodiments, the method of treating cancer in a subject in need thereof comprises administering: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KLVELEHTL (SEQ ID NO: 2) and HELGFKVVL (SEQ ID NO: 6) and wherein the subject expresses an MHC encoded by an HLA-A*02:01 allele, an HLA-B*18:01 allele, an HLA-B*40:01 allele, an HLA-B*44:02 allele, an HLA-B*40:02 allele, or any combination thereof. In some embodiments, the method of treating cancer in a subject in need thereof comprises administering: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KLVELEHTL (SEQ ID NO: 2) and HELGFKVVL (SEQ ID NO: 6) and wherein the subject expresses an MHC encoded by an HLA-A*02:01 allele, an HLA-B*18:01 allele, an HLA-B*40:01 allele, an HLA-B*44:02 allele, an HLA-B*40:02 allele, or any combination thereof.

In some embodiments, the method of treating cancer in a subject in need thereof comprises administering: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are SPSSASLAL (SEQ ID NO: 3) and KPRPDVTNEL (SEQ ID NO: 4). In some embodiments, the method of treating cancer in a subject in need thereof comprises administering: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are SPSSASLAL (SEQ ID NO: 3) and KPRPDVTNEL (SEQ ID NO: 4). In some embodiments, the method of treating cancer in a subject in need thereof comprises administering: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are SPSSASLAL (SEQ ID NO: 3) and KPRPDVTNEL (SEQ ID NO: 4) and wherein the subject expresses an MHC encoded by an HLA-B*07:02 allele, an HLA-B*35:03 allele, an HLA-C*07:02 allele, or any combination thereof. In some embodiments, the method of treating cancer in a subject in need thereof comprises administering: (i) a recombinant polypeptide consisting of at least two cancer-specific CD8+ T cell epitopes or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are SPSSASLAL (SEQ ID NO: 3) and KPRPDVTNEL (SEQ ID NO: 4) and wherein the subject expresses an MHC encoded by an HLA-B*07:02 allele, an HLA-B*35:03 allele, an HLA-C*07:02 allele, or any combination thereof.

In some embodiments, the method of treating cancer in a subject in need thereof comprises administering: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are SPSSASLAL (SEQ ID NO: 3) and KEFAFLEHSL (SEQ ID NO: 5). In some embodiments, the method of treating cancer in a subject in need thereof comprises administering: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are SPSSASLAL (SEQ ID NO: 3) and KEFAFLEHSL (SEQ ID NO: 5). In some embodiments, the method of treating cancer in a subject in need thereof comprises administering: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are SPSSASLAL (SEQ ID NO: 3) and KEFAFLEHSL (SEQ ID NO: 5) and wherein the subject expresses an MHC encoded by an HLA-B*07:02 allele, an HLA-B*35:03 allele, an HLA-B*40:01 allele, an HLA-B*44:02 allele, an HLA-B*40:02 allele, or any combination thereof. In some embodiments, the method of treating cancer in a subject in need thereof comprises administering: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are SPSSASLAL (SEQ ID NO: 3) and KEFAFLEHSL (SEQ ID NO: 5) and wherein the subject expresses an MHC encoded by an HLA-B*07:02 allele, an HLA-B*35:03 allele, an HLA-B*40:01 allele, an HLA-B*44:02 allele, an HLA-B*40:02 allele, or any combination thereof.

In some embodiments, the method of treating cancer in a subject in need thereof comprises administering: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are SPSSASLAL (SEQ ID NO: 3) and HELGFKVVL (SEQ ID NO: 6). In some embodiments, the method of treating cancer in a subject in need thereof comprises administering: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are SPSSASLAL (SEQ ID NO: 3) and HELGFKVVL (SEQ ID NO: 6). In some embodiments, the method of treating cancer in a subject in need thereof comprises administering: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are SPSSASLAL (SEQ ID NO: 3) and HELGFKVVL (SEQ ID NO: 6) and wherein the subject expresses an MHC encoded by an HLA-B*07:02 allele, an HLA-B*35:03 allele, an HLA-B*18:01 allele, an HLA-B*40:01 allele, an HLA-B*44:02 allele, an HLA-B*40:02 allele, or any combination thereof. In some embodiments, the method of treating cancer in a subject in need thereof comprises administering: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are SPSSASLAL (SEQ ID NO: 3) and HELGFKVVL (SEQ ID NO: 6) and wherein the subject expresses an MHC encoded by an HLA-B*07:02 allele, an HLA-B*35:03 allele, an HLA-B*18:01 allele, an HLA-B*40:01 allele, an HLA-B*44:02 allele, an HLA-B*40:02 allele, or any combination thereof.

In some embodiments, the method of treating cancer in a subject in need thereof comprises administering: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KPRPDVTNEL (SEQ ID NO: 4) and KEFAFLEHSL (SEQ ID NO: 5). In some embodiments, the method of treating cancer in a subject in need thereof comprises administering: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KPRPDVTNEL (SEQ ID NO: 4) and KEFAFLEHSL (SEQ ID NO: 5). In some embodiments, the method of treating cancer in a subject in need thereof comprises administering: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KPRPDVTNEL (SEQ ID NO: 4) and KEF AFLEHSL (SEQ ID NO: 5) and wherein the subject expresses an MHC encoded by an HLA-B*07:02 allele, an HLA-B*40:01 allele, an HLA-B*44:02 allele, an HLA-B*35:03 allele, an HLA-C*07:02 allele, an HLA-B*40:02 allele, or any combination thereof. In some embodiments, the method of treating cancer in a subject in need thereof comprises administering: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KPRPDVTNEL (SEQ ID NO: 4) and KEFAFLEHSL (SEQ ID NO: 5) and wherein the subject expresses an HLA-B*07:02 allele, an HLA-B*40:01 allele, an HLA-B*44:02 allele, an HLA-B*35:03 allele, an HLA-C*07:02 allele, an HLA-B*40:02 allele, or any combination thereof.

In some embodiments, the method of treating cancer in a subject in need thereof comprises administering: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KPRPDVTNEL (SEQ ID NO: 4) and HELGFKVVL (SEQ ID NO: 6). In some embodiments, the method of treating cancer in a subject in need thereof comprises administering: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KPRPDVTNEL (SEQ ID NO: 4) and HELGFKVVL (SEQ ID NO: 6). In some embodiments, the method of treating cancer in a subject in need thereof comprises administering: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KPRPDVTNEL (SEQ ID NO: 4) and HELGFKVVL (SEQ ID NO: 6) and wherein the subject expresses an MHC encoded by an HLA-B*07:02 allele, an HLA-B*35:03 allele, an HLA-C*07:02 allele, an HLA-B*18:01 allele, an HLA-B*40:01 allele, an HLA-B*44:02 allele, an HLA-B*40:02 allele, or any combination thereof. In some embodiments, the method of treating cancer in a subject in need thereof comprises administering: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KPRPDVTNEL (SEQ ID NO: 4) and HELGFKVVL (SEQ ID NO: 6) and wherein the subject expresses an MHC encoded by an HLA-B*07:02 allele, an HLA-B*35:03 allele, an HLA-C*07:02 allele, an HLA-B*18:01 allele, an HLA-B*40:01 allele, an HLA-B*44:02 allele, an HLA-B*40:02 allele, or any combination thereof.

In some embodiments, the method of treating cancer in a subject in need thereof comprises administering: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KEFAFLEHSL (SEQ ID NO: 5) and HELGFKVVL (SEQ ID NO: 6). In some embodiments, the method of treating cancer in a subject in need thereof comprises administering: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KEFAFLEHSL (SEQ ID NO: 5) and HELGFKVVL (SEQ ID NO: 6). In some embodiments, the method of treating cancer in a subject in need thereof comprises administering: (i) a recombinant polypeptide comprising at least two cancer-specific CD8+ T cell epitopes, or (ii) a polynucleotide encoding a recombinant polypeptide comprising at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KEFAFLEHSL (SEQ ID NO: 5) and HELGFKVVL (SEQ ID NO: 6) and wherein the subject expresses an MHC encoded by an HLA-B*18:01 allele, an HLA-B*40:01 allele, an HLA-B*44:02 allele, an HLA-B*40:02 allele, or any combination thereof. In some embodiments, the method of treating cancer in a subject in need thereof comprises administering: (i) a recombinant polypeptide consisting of at least two cancer-specific CD8+ T cell epitopes, or (ii) a polynucleotide encoding a recombinant polypeptide consisting of at least two cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least two cancer-specific CD8+ T cell epitopes are KEFAFLEHSL (SEQ ID NO: 5) and HELGFKVVL (SEQ ID NO: 6) and wherein the subject expresses an MHC encoded by an HLA-B*18:01 allele, an HLA-B*40:01 allele, an HLA-B*44:02 allele, an HLA-B*40:02 allele, or any combination thereof.

In some embodiments, the method of treating cancer in a subject in need thereof comprises administering: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KLVELEHTL (SEQ ID NO: 2) and SPSSASLAL (SEQ ID NO: 3). In some embodiments, the method of treating cancer in a subject in need thereof comprises administering: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KLVELEHTL (SEQ ID NO: 2) and SPSSASLAL (SEQ ID NO: 3). In some embodiments, the method of treating cancer in a subject in need thereof comprises administering: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KLVELEHTL (SEQ ID NO: 2) and SPSSASLAL (SEQ ID NO: 3) and wherein the subject expresses an MHC encoded by an HLA-A*02:01 allele, an HLA-B*07:02 allele, an HLA-B*35:03 allele, or any combination thereof. In some embodiments, the method of treating cancer in a subject in need thereof comprises administering: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KLVELEHTL (SEQ ID NO: 2) and SPSSASLAL (SEQ ID NO: 3) and wherein the subject expresses an MHC encoded by an HLA-A*02:01 allele, an HLA-B*07:02 allele, an HLA-B*35:03 allele, or any combination thereof.

In some embodiments, the method of treating cancer in a subject in need thereof comprises administering: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KLVELEHTL (SEQ ID NO: 2) and KPRPDVTNEL (SEQ ID NO: 4). In some embodiments, the method of treating cancer in a subject in need thereof comprises administering: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KLVELEHTL (SEQ ID NO: 2) and KPRPDVTNEL (SEQ ID NO: 4). In some embodiments, the method of treating cancer in a subject in need thereof comprises administering: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KLVELEHTL (SEQ ID NO: 2) and KPRPDVTNEL (SEQ ID NO: 4) and wherein the subject expresses an MHC encoded by an HLA-A*02:01 allele, an HLA-B*07:02 allele, an HLA-B*35:03 allele, an HLA-C*07:02 allele, or any combination thereof. In some embodiments, the method of treating cancer in a subject in need thereof comprises administering: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KLVELEHTL (SEQ ID NO: 2) and KPRPDVTNEL (SEQ ID NO: 4) and wherein the subject expresses an MHC encoded by an HLA-A*02:01 allele, an HLA-B*07:02 allele, an HLA-B*35:03 allele, an HLA-C*07:02 allele, or any combination thereof.

In some embodiments, the method of treating cancer in a subject in need thereof comprises administering: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KLVELEHTL (SEQ ID NO: 2) and KEFAFLEHSL (SEQ ID NO: 5). In some embodiments, the method of treating cancer in a subject in need thereof comprises administering: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KLVELEHTL (SEQ ID NO: 2) and KEFAFLEHSL (SEQ ID NO: 5). In some embodiments, the method of treating cancer in a subject in need thereof comprises administering: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KLVELEHTL (SEQ ID NO: 2) and KEFAFLEHSL (SEQ ID NO: 5) and wherein the subject expresses an MHC encoded by an HLA-A*02:01 allele, an HLA-B*40:01 allele, an HLA-B*44:02 allele, an HLA-B*40:02 allele, or any combination thereof. In some embodiments, the method of treating cancer in a subject in need thereof comprises administering: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KLVELEHTL (SEQ ID NO: 2) and KEFAFLEHSL (SEQ ID NO: 5) and wherein the subject expresses an MHC encoded by an HLA-A*02:01 allele, an HLA-B*40:01 allele, an HLA-B*44:02 allele, an HLA-B*40:02 allele, or any combination thereof.

In some embodiments, the method of treating cancer in a subject in need thereof comprises administering: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KLVELEHTL (SEQ ID NO: 2) and HELGFKVVL (SEQ ID NO: 6). In some embodiments, the method of treating cancer in a subject in need thereof comprises administering: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KLVELEHTL (SEQ ID NO: 2) and HELGFKVVL (SEQ ID NO: 6). In some embodiments, the method of treating cancer in a subject in need thereof comprises administering: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KLVELEHTL (SEQ ID NO: 2) and HELGFKVVL (SEQ ID NO: 6) and wherein the subject expresses an MHC encoded by an HLA-A*02:01 allele, an HLA-B*18:01 allele, an HLA-B*40:01 allele, an HLA-B*44:02 allele, an HLA-B*40:02 allele, or any combination thereof. In some embodiments, the method of treating cancer in a subject in need thereof comprises administering: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KLVELEHTL (SEQ ID NO: 2) and HELGFKVVL (SEQ ID NO: 6) and wherein the subject expresses an MHC encoded by an HLA-A*02:01 allele, an HLA-B*18:01 allele, an HLA-B*40:01 allele, an HLA-B*44:02 allele, an HLA-B*40:02 allele, or any combination thereof.

In some embodiments, the method of treating cancer in a subject in need thereof comprises administering: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and SPSSASLAL (SEQ ID NO: 3) and KPRPDVTNEL (SEQ ID NO: 4). In some embodiments, the method of treating cancer in a subject in need thereof comprises administering: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and SPSSASLAL (SEQ ID NO: 3) and KPRPDVTNEL (SEQ ID NO: 4). In some embodiments, the method of treating cancer in a subject in need thereof comprises administering: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and SPSSASLAL (SEQ ID NO: 3) and KPRPDVTNEL (SEQ ID NO: 4) and wherein the subject expresses an MHC encoded by an HLA-A*02:01 allele, an HLA-B*07:02 allele, an HLA-B*35:03 allele, an HLA-C*07:02 allele, or any combination thereof. In some embodiments, the method of treating cancer in a subject in need thereof comprises administering: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and SPSSASLAL (SEQ ID NO: 3) and KPRPDVTNEL (SEQ ID NO: 4) and wherein the subject expresses an MHC encoded by an HLA-A*02:01 allele, an HLA-B*07:02 allele, an HLA-B*35:03 allele, an HLA-C*07:02 allele, or any combination thereof.

In some embodiments, the method of treating cancer in a subject in need thereof comprises administering: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and SPSSASLAL (SEQ ID NO: 3) and KEFAFLEHSL (SEQ ID NO: 5). In some embodiments, the method of treating cancer in a subject in need thereof comprises administering: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and SPSSASLAL (SEQ ID NO: 3) and KEFAFLEHSL (SEQ ID NO: 5). In some embodiments, the method of treating cancer in a subject in need thereof comprises administering: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and SPSSASLAL (SEQ ID NO: 3) and KEFAFLEHSL (SEQ ID NO: 5) and wherein the subject expresses an MHC encoded by an HLA-A*02:01 allele, an HLA-B*07:02 allele, an HLA-B*35:03 allele, an HLA-B*40:01 allele, an HLA-B*44:02 allele, an HLA-B*40:02 allele, or any combination thereof. In some embodiments, the method of treating cancer in a subject in need thereof comprises administering: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and SPSSASLAL (SEQ ID NO: 3) and KEFAFLEHSL (SEQ ID NO: 5) and wherein the subject expresses an MHC encoded by an HLA-A*02:01 allele, an HLA-B*07:02 allele, an HLA-B*35:03 allele, an HLA-B*40:01 allele, an HLA-B*44:02 allele, an HLA-B*40:02 allele, or any combination thereof.

In some embodiments, the method of treating cancer in a subject in need thereof comprises administering: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and SPSSASLAL (SEQ ID NO: 3) and HELGFKVVL (SEQ ID NO: 6). In some embodiments, the method of treating cancer in a subject in need thereof comprises administering: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and SPSSASLAL (SEQ ID NO: 3) and HELGFKVVL (SEQ ID NO: 6). In some embodiments, the method of treating cancer in a subject in need thereof comprises administering: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and SPSSASLAL (SEQ ID NO: 3) and HELGFKVVL (SEQ ID NO: 6) and wherein the subject expresses an MHC encoded by an HLA-A*02:01 allele, an HLA-B*07:02 allele, an HLA-B*35:03 allelle, an HLA-B*18:01 allele, an HLA-B*40:01 allele, an HLA-B*44:02 allele, an HLA-B*40:02 allele, or any combination thereof. In some embodiments, the method of treating cancer in a subject in need thereof comprises administering: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and SPSSASLAL (SEQ ID NO: 3) and HELGFKVVL (SEQ ID NO: 6) and wherein the subject expresses an MHC encoded by an HLA-A*02:01 allele, an HLA-B*07:02 allele, an HLA-B*35:03 allelle, an HLA-B*18:01 allele, an HLA-B*40:01 allele, an HLA-B*44:02 allele, an HLA-B*40:02 allele, or any combination thereof.

In some embodiments, the method of treating cancer in a subject in need thereof comprises administering: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KPRPDVTNEL (SEQ ID NO: 4) and KEFAFLEHSL (SEQ ID NO: 5). In some embodiments, the method of treating cancer in a subject in need thereof comprises administering: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KPRPDVTNEL (SEQ ID NO: 4) and KEFAFLEHSL (SEQ ID NO: 5). In some embodiments, the method of treating cancer in a subject in need thereof comprises administering: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KPRPDVTNEL (SEQ ID NO: 4) and KEFAFLEHSL (SEQ ID NO: 5) and wherein the subject expresses an MHC encoded by an HLA-A*02:01 allele, an HLA-B*07:02 allele, an HLA-B*35:03 allele, an HLA-C*07:02 allele, an HLA-B*40:01 allele, an HLA-B*44:02 allele, an HLA-B*40:02 allele, or any combination thereof. In some embodiments, the method of treating cancer in a subject in need thereof comprises administering: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KPRPDVTNEL (SEQ ID NO: 4) and KEFAFLEHSL (SEQ ID NO: 5) and wherein the subject expresses an MHC encoded by an HLA-A*02:01 allele, an HLA-B*07:02 allele, an HLA-B*35:03 allele, an HLA-C*07:02 allele, an HLA-B*40:01 allele, an HLA-B*44:02 allele, an HLA-B*40:02 allele, or any combination thereof.

In some embodiments, the method of treating cancer in a subject in need thereof comprises administering: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KPRPDVTNEL (SEQ ID NO: 4) and HELGFKVVL (SEQ ID NO: 6). In some embodiments, the method of treating cancer in a subject in need thereof comprises administering: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KPRPDVTNEL (SEQ ID NO: 4) and HELGFKVVL (SEQ ID NO: 6). In some embodiments, the method of treating cancer in a subject in need thereof comprises administering: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KPRPDVTNEL (SEQ ID NO: 4) and HELGFKVVL (SEQ ID NO: 6) and wherein the subject expresses an MHC encoded by an HLA-A*02:01 allele, an HLA-B*07:02 allele, an HLA-B*35:03 allele, an HLA-C*07:02 allele, an HLA-B*40:01 allele, an HLA-B*44:02 allele, an HLA-B*40:02 allele, an HLA-B*18:01 allele, or any combination thereof. In some embodiments, the method of treating cancer in a subject in need thereof comprises administering: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KPRPDVTNEL (SEQ ID NO: 4) and HELGFKVVL (SEQ ID NO: 6) and wherein the subject expresses an MHC encoded by an HLA-A*02:01 allele, an HLA-B*07:02 allele, an HLA-B*35:03 allele, an HLA-C*07:02 allele, an HLA-B*40:01 allele, an HLA-B*44:02 allele, an HLA-B*40:02 allele, an HLA-B*18:01 allele, or any combination thereof.

In some embodiments, the method of treating cancer in a subject in need thereof comprises administering: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KPRPDVTNEL (SEQ ID NO: 4) and KEFAFLEHSL (SEQ ID NO: 5). In some embodiments, the method of treating cancer in a subject in need thereof comprises administering: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KEFAFLEHSL (SEQ ID NO: 5) and HELGFKVVL (SEQ ID NO: 6). In some embodiments, the method of treating cancer in a subject in need thereof comprises administering: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KEFAFLEHSL (SEQ ID NO: 5) and HELGFKVVL (SEQ ID NO: 6) and wherein the subject expresses an MHC encoded by an HLA-A*02:01 allele, an HLA-B*40:01 allele, an HLA-B*44:02 allele, an HLA-B*40:02 allele, an HLA-B*18:01 allele, or any combination thereof. In some embodiments, the method of treating cancer in a subject in need thereof comprises administering: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are GVYDGREHTV (SEQ ID NO: 1) and KEFAFLEHSL (SEQ ID NO: 5) and HELGFKVVL (SEQ ID NO: 6) and wherein the subject expresses an MHC encoded by an HLA-A*02:01 allele, an HLA-B*40:01 allele, an HLA-B*44:02 allele, an HLA-B*40:02 allele, an HLA-B*18:01 allele, or any combination thereof.

In some embodiments, the method of treating cancer in a subject in need thereof comprises administering: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KLVELEHTL (SEQ ID NO: 2) and SPSSASLAL (SEQ ID NO: 3) and KPRPDVTNEL (SEQ ID NO: 4). In some embodiments, the method of treating cancer in a subject in need thereof comprises administering: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KLVELEHTL (SEQ ID NO: 2) and SPSSASLAL (SEQ ID NO: 3) and KPRPDVTNEL (SEQ ID NO: 4). In some embodiments, the method of treating cancer in a subject in need thereof comprises administering: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KLVELEHTL (SEQ ID NO: 2) and SPSSASLAL (SEQ ID NO: 3) and KPRPDVTNEL (SEQ ID NO: 4) and wherein the subject expresses an MHC encoded by an HLA-A*02:01 allele, an HLA-B*07:02 allele, an HLA-B*35:03 allele, an HLA-C*07:02 allele, or any combination thereof. In some embodiments, the method of treating cancer in a subject in need thereof comprises administering: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KLVELEHTL (SEQ ID NO: 2) and SPSSASLAL (SEQ ID NO: 3) and KPRPDVTNEL (SEQ ID NO: 4) and wherein the subject expresses an MHC encoded by an HLA-A*02:01 allele, an HLA-B*07:02 allele, an HLA-B*35:03 allele, an HLA-C*07:02 allele, or any combination thereof.

In some embodiments, the method of treating cancer in a subject in need thereof comprises administering: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KLVELEHTL (SEQ ID NO: 2) and SPSSASLAL (SEQ ID NO: 3) and KEFAFLEHSL (SEQ ID NO: 5). In some embodiments, the method of treating cancer in a subject in need thereof comprises administering: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KLVELEHTL (SEQ ID NO: 2) and SPSSASLAL (SEQ ID NO: 3) and KEFAFLEHSL (SEQ ID NO: 5). In some embodiments, the method of treating cancer in a subject in need thereof comprises administering: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KLVELEHTL (SEQ ID NO: 2) and SPSSASLAL (SEQ ID NO: 3) and KEFAFLEHSL (SEQ ID NO: 5) and wherein the subject expresses an MHC encoded by an HLA-A*02:01 allele, an HLA-B*07:02 allele, an HLA-B*35:03 allele, an HLA-B*40:01 allele, an HLA-B*44:02 allele, an HLA-B*40:02 allele, or any combination thereof. In some embodiments, the method of treating cancer in a subject in need thereof comprises administering: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KLVELEHTL (SEQ ID NO: 2) and SPSSASLAL (SEQ ID NO: 3) and KEFAFLEHSL (SEQ ID NO: 5) and wherein the subject expresses an MHC encoded by an HLA-A*02:01 allele, an HLA-B*07:02 allele, an HLA-B*35:03 allele, an HLA-B*40:01 allele, an HLA-B*44:02 allele, an HLA-B*40:02 allele, or any combination thereof.

In some embodiments, the method of treating cancer in a subject in need thereof comprises administering: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KLVELEHTL (SEQ ID NO: 2) and SPSSASLAL (SEQ ID NO: 3) and HELGFKVVL (SEQ ID NO: 6). In some embodiments, the method of treating cancer in a subject in need thereof comprises administering: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KLVELEHTL (SEQ ID NO: 2) and SPSSASLAL (SEQ ID NO: 3) and HELGFKVVL (SEQ ID NO: 6). In some embodiments, the method of treating cancer in a subject in need thereof comprises administering: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KLVELEHTL (SEQ ID NO: 2) and SPSSASLAL (SEQ ID NO: 3) and HELGFKVVL (SEQ ID NO: 6) and wherein the subject expresses an MHC encoded by an HLA-A*02:01 allele, an HLA-B*07:02 allele, an HLA-B*35:03 allelle, an HLA-B*18:01 allele, an HLA-B*40:01 allele, an HLA-B*44:02 allele, an HLA-B*40:02 allele, or any combination thereof. In some embodiments, the method of treating cancer in a subject in need thereof comprises administering: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KLVELEHTL (SEQ ID NO: 2) and SPSSASLAL (SEQ ID NO: 3) and HELGFKVVL (SEQ ID NO: 6) and wherein the subject expresses an MHC encoded by an HLA-A*02:01 allele, an HLA-B*07:02 allele, an HLA-B*35:03 allelle, an HLA-B*18:01 allele, an HLA-B*40:01 allele, an HLA-B*44:02 allele, an HLA-B*40:02 allele, or any combination thereof.

In some embodiments, the method of treating cancer in a subject in need thereof comprises administering: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KLVELEHTL (SEQ ID NO: 2) and KPRPDVTNEL (SEQ ID NO: 4) and KEFAFLEHSL (SEQ ID NO: 5). In some embodiments, the method of treating cancer in a subject in need thereof comprises administering: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KLVELEHTL (SEQ ID NO: 2) and KPRPDVTNEL (SEQ ID NO: 4) and KEFAFLEHSL (SEQ ID NO: 5). In some embodiments, the method of treating cancer in a subject in need thereof comprises administering: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KLVELEHTL (SEQ ID NO: 2) and KPRPDVTNEL (SEQ ID NO: 4) and KEFAFLEHSL (SEQ ID NO: 5) and wherein the subject expresses an MHC encoded by an HLA-A*02:01 allele, an HLA-B*07:02 allele, an HLA-B*35:03 allele, an HLA-C*07:02 allele, an HLA-B*40:01 allele, an HLA-B*44:02 allele, an HLA-B*40:02 allele, or any combination thereof. In some embodiments, the method of treating cancer in a subject in need thereof comprises administering: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KLVELEHTL (SEQ ID NO: 2) and KPRPDVTNEL (SEQ ID NO: 4) and KEFAFLEHSL (SEQ ID NO: 5) and wherein the subject expresses an MHC encoded by an HLA-A*02:01 allele, an HLA-B*07:02 allele, an HLA-B*35:03 allele, an HLA-C*07:02 allele, an HLA-B*40:01 allele, an HLA-B*44:02 allele, an HLA-B*40:02 allele, or any combination thereof.

In some embodiments, the method of treating cancer in a subject in need thereof comprises administering: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KLVELEHTL (SEQ ID NO: 2) and KPRPDVTNEL (SEQ ID NO: 4) and HELGFKVVL (SEQ ID NO: 6). In some embodiments, the method of treating cancer in a subject in need thereof comprises administering: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KLVELEHTL (SEQ ID NO: 2) and KPRPDVTNEL (SEQ ID NO: 4) and HELGFKVVL (SEQ ID NO: 6). In some embodiments, the method of treating cancer in a subject in need thereof comprises administering: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KLVELEHTL (SEQ ID NO: 2) and KPRPDVTNEL (SEQ ID NO: 4) and HELGFKVVL (SEQ ID NO: 6) and wherein the subject expresses an MHC encoded by an HLA-A*02:01 allele, an HLA-B*07:02 allele, an HLA-B*35:03 allele, an HLA-C*07:02 allele, an HLA-B*40:01 allele, an HLA-B*44:02 allele, an HLA-B*40:02 allele, an HLA-B*18:01 allele, or any combination thereof. In some embodiments, the method of treating cancer in a subject in need thereof comprises administering: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KLVELEHTL (SEQ ID NO: 2) and KPRPDVTNEL (SEQ ID NO: 4) and HELGFKVVL (SEQ ID NO: 6) and wherein the subject expresses an MHC encoded by an HLA-A*02:01 allele, an HLA-B*07:02 allele, an HLA-B*35:03 allele, an HLA-C*07:02 allele, an HLA-B*40:01 allele, an HLA-B*44:02 allele, an HLA-B*40:02 allele, an HLA-B*18:01 allele, or any combination thereof.

In some embodiments, the method of treating cancer in a subject in need thereof comprises administering: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KLVELEHTL (SEQ ID NO: 2) and KEFAFLEHSL (SEQ ID NO: 5) and HELGFKVVL (SEQ ID NO: 6). In some embodiments, the method of treating cancer in a subject in need thereof comprises administering: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KLVELEHTL (SEQ ID NO: 2) and KEFAFLEHSL (SEQ ID NO: 5) and HELGFKVVL (SEQ ID NO: 6). In some embodiments, the method of treating cancer in a subject in need thereof comprises administering: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KLVELEHTL (SEQ ID NO: 2) and KEFAFLEHSL (SEQ ID NO: 5) and HELGFKVVL (SEQ ID NO: 6) and wherein the subject expresses an MHC encoded by an HLA-A*02:01 allele, an HLA-B*18:01 allele, an HLA-B*40:01 allele, an HLA-B*44:02 allele, an HLA-B*40:02 allele, or any combination thereof. In some embodiments, the method of treating cancer in a subject in need thereof comprises administering: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KLVELEHTL (SEQ ID NO: 2) and KEFAFLEHSL (SEQ ID NO: 5) and HELGFKVVL (SEQ ID NO: 6) and wherein the subject expresses an MHC encoded by an HLA-A*02:01 allele, an HLA-B*18:01 allele, an HLA-B*40:01 allele, an HLA-B*44:02 allele, an HLA-B*40:02 allele, or any combination thereof.

In some embodiments, the method of treating cancer in a subject in need thereof comprises administering: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are SPSSASLAL (SEQ ID NO: 3) and KPRPDVTNEL (SEQ ID NO: 4) and KEFAFLEHSL (SEQ ID NO: 5). In some embodiments, the method of treating cancer in a subject in need thereof comprises administering: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are SPSSASLAL (SEQ ID NO: 3) and KPRPDVTNEL (SEQ ID NO: 4) and KEFAFLEHSL (SEQ ID NO: 5). In some embodiments, the method of treating cancer in a subject in need thereof comprises administering: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are SPSSASLAL (SEQ ID NO: 3) and KPRPDVTNEL (SEQ ID NO: 4) and KEFAFLEHSL (SEQ ID NO: 5) and wherein the subject expresses an MHC encoded by an HLA-B*07:02 allele, an HLA-B*35:03 allele, an HLA-C*07:02 allele, an HLA-B*40:01 allele, an HLA-B*44:02 allele, an HLA-B*40:02 allele, or any combination thereof. In some embodiments, the method of treating cancer in a subject in need thereof comprises administering: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are SPSSASLAL (SEQ ID NO: 3) and KPRPDVTNEL (SEQ ID NO: 4) and KEF AFLEHSL (SEQ ID NO: 5) and wherein the subject expresses an MHC encoded by an HLA-B*07:02 allele, an HLA-B*35:03 allele, an or any combination thereof.

In some embodiments, the method of treating cancer in a subject in need thereof comprises administering: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are SPSSASLAL (SEQ ID NO: 3) and KPRPDVTNEL (SEQ ID NO: 4) and HELGFKVVL (SEQ ID NO: 6). In some embodiments, the method of treating cancer in a subject in need thereof comprises administering: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are SPSSASLAL (SEQ ID NO: 3) and KPRPDVTNEL (SEQ ID NO: 4) and HELGFKVVL (SEQ ID NO: 6). In some embodiments, the method of treating cancer in a subject in need thereof comprises administering: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are SPSSASLAL (SEQ ID NO: 3) and KPRPDVTNEL (SEQ ID NO: 4) and HELGFKVVL (SEQ ID NO: 6) and wherein the subject expresses an MHC encoded by an HLA-B*07:02 allele, an HLA-B*35:03 allele, an HLA-C*07:02 allele, an HLA-B*40:01 allele, an HLA-B*44:02 allele, an HLA-B*40:02 allele, an HLA-B*18:01 allele, or any combination thereof. In some embodiments, the method of treating cancer in a subject in need thereof comprises administering: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are SPSSASLAL (SEQ ID NO: 3) and KPRPDVTNEL (SEQ ID NO: 4) and HELGFKVVL (SEQ ID NO: 6) and wherein the subject expresses an MHC encoded by an HLA-B*07:02 allele, an HLA-B*35:03 allele, an an HLA-B*18:01 allele, or any combination thereof.

In some embodiments, the method of treating cancer in a subject in need thereof comprises administering: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are SPSSASLAL (SEQ ID NO: 3) and KEFAFLEHSL (SEQ ID NO: 5) and HELGFKVVL (SEQ ID NO: 6). In some embodiments, the method of treating cancer in a subject in need thereof comprises administering: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are SPSSASLAL (SEQ ID NO: 3) and KEFAFLEHSL (SEQ ID NO: 5) and HELGFKVVL (SEQ ID NO: 6). In some embodiments, the method of treating cancer in a subject in need thereof comprises administering: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are SPSSASLAL (SEQ ID NO: 3) and KEFAFLEHSL (SEQ ID NO: 5) and HELGFKVVL (SEQ ID NO: 6) and wherein the subject expresses an MHC encoded by an HLA-B*07:02 allele, an HLA-B*35:03 allele, an HLA-B*40:01 allele, an HLA-B*44:02 allele, an HLA-B*40:02 allele, an HLA-B*18:01 allele, or any combination thereof. In some embodiments, the method of treating cancer in a subject in need thereof comprises administering: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are SPSSASLAL (SEQ ID NO: 3) and KEFAFLEHSL (SEQ ID NO: 5) and HELGFKVVL (SEQ ID NO: 6) and wherein the subject expresses an MHC encoded by an HLA-B*07:02 allele, an HLA-B*35:03 allele, an HLA-B*40:01 allele, an HLA-B*44:02 allele, an HLA-B*40:02 allele, an HLA-B*18:01 allele, or any combination thereof.

In some embodiments, the method of treating cancer in a subject in need thereof comprises administering: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KPRPDVTNEL (SEQ ID NO: 4) and KEFAFLEHSL (SEQ ID NO: 5) and HELGFKVVL (SEQ ID NO: 6). In some embodiments, the method of treating cancer in a subject in need thereof comprises administering: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KPRPDVTNEL (SEQ ID NO: 4) and KEFAFLEHSL (SEQ ID NO: 5) and HELGFKVVL (SEQ ID NO: 6). In some embodiments, the method of treating cancer in a subject in need thereof comprises administering: (i) a recombinant polypeptide comprising at least three cancer-specific CD8+ T cell epitopes, or (ii) a polynucleotide encoding a recombinant polypeptide comprising at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KPRPDVTNEL (SEQ ID NO: 4) and KEFAFLEHSL (SEQ ID NO: 5) and HELGFKVVL (SEQ ID NO: 6) and wherein the subject expresses an MHC encoded by an HLA-B*07:02 allele, an HLA-B*35:03 allele, an HLA-C*07:02 allele, an HLA-B*40:01 allele, an HLA-B*44:02 allele, an HLA-B*40:02 allele, an HLA-B*18:01 allele, or any combination thereof. In some embodiments, the method of treating cancer in a subject in need thereof comprises administering: (i) a recombinant polypeptide consisting of at least three cancer-specific CD8+ T cell epitopes, or (ii) a polynucleotide encoding a recombinant polypeptide consisting of at least three cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least three cancer-specific CD8+ T cell epitopes are KPRPDVTNEL (SEQ ID NO: 4) and KEFAFLEHSL (SEQ ID NO: 5) and HELGFKVVL (SEQ ID NO: 6) and wherein the subject expresses an MHC encoded by an HLA-B*07:02 allele, an HLA-B*35:03 allele, an an HLA-B*18:01 allele, or any combination thereof.

In some embodiments, the method of treating cancer in a subject in need thereof comprises administering: (i) a recombinant polypeptide comprising at least four cancer-specific CD8+ T cell epitopes, or (ii) a polynucleotide encoding a recombinant polypeptide comprising at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are GVYDGREHTV (SEQ ID NO: 1) and KLVELEHTL (SEQ ID NO: 2) and SPSSASLAL (SEQ ID NO: 3) and KPRPDVTNEL (SEQ ID NO: 4). In some embodiments, the method of treating cancer in a subject in need thereof comprises administering: (i) a recombinant polypeptide consisting of at least four cancer-specific CD8+ T cell epitopes, or (ii) a polynucleotide encoding a recombinant polypeptide consisting of at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are GVYDGREHTV (SEQ ID NO: 1) and KLVELEHTL (SEQ ID NO: 2) and SPSSASLAL (SEQ ID NO: 3) and KPRPDVTNEL (SEQ ID NO: 4). In some embodiments, the method of treating cancer in a subject in need thereof comprises administering: (i) a recombinant polypeptide comprising at least four cancer-specific CD8+ T cell epitopes, or (ii) a polynucleotide encoding a recombinant polypeptide comprising at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are GVYDGREHTV (SEQ ID NO: 1) and KLVELEHTL (SEQ ID NO: 2) and SPSSASLAL (SEQ ID NO: 3) and KPRPDVTNEL (SEQ ID NO: 4) and wherein the subject expresses an MHC encoded by an HLA-A*02:01 allele, an HLA-B*07:02 allele, an HLA-B*35:03 allele, an HLA-C*07:02 allele, or any combination thereof. In some embodiments, the method of treating cancer in a subject in need thereof comprises administering: (i) a recombinant polypeptide consisting of at least four cancer-specific CD8+ T cell epitopes, or (ii) a polynucleotide encoding a recombinant polypeptide consisting of at four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are GVYDGREHTV (SEQ ID NO: 1) and KLVELEHTL (SEQ ID NO: 2) and SPSSASLAL (SEQ ID NO: 3) and KPRPDVTNEL (SEQ ID NO: 4) and wherein the subject expresses an MHC encoded by an HLA-A*02:01 allele, an HLA-B*07:02 allele, an HLA-B*35:03 allele, an HLA-C*07:02 allele, or any combination thereof.

In some embodiments, the method of treating cancer in a subject in need thereof comprises administering: (i) a recombinant polypeptide comprising at least four cancer-specific CD8+ T cell epitopes, or (ii) a polynucleotide encoding a recombinant polypeptide comprising at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are GVYDGREHTV (SEQ ID NO: 1) and KLVELEHTL (SEQ ID NO: 2) and SPSSASLAL (SEQ ID NO: 3) and KEFAFLEHSL (SEQ ID NO: 5). In some embodiments, the method of treating cancer in a subject in need thereof comprises administering: (i) a recombinant polypeptide consisting of at least four cancer-specific CD8+ T cell epitopes, or (ii) a polynucleotide encoding a recombinant polypeptide consisting of at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are GVYDGREHTV (SEQ ID NO: 1) and KLVELEHTL (SEQ ID NO: 2) and SPSSASLAL (SEQ ID NO: 3) and KEFAFLEHSL (SEQ ID NO: 5). In some embodiments, the method of treating cancer in a subject in need thereof comprises administering: (i) a recombinant polypeptide comprising at least four cancer-specific CD8+ T cell epitopes, or (ii) a polynucleotide encoding a recombinant polypeptide comprising at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are GVYDGREHTV (SEQ ID NO: 1) and KLVELEHTL (SEQ ID NO: 2) and SPSSASLAL (SEQ ID NO: 3) and KEFAFLEHSL (SEQ ID NO: 5) and wherein the subject expresses an MHC encoded by an HLA-A*02:01 allele, an HLA-B*07:02 allele, an HLA-B*35:03 allele, an HLA-B*40:01 allele, an HLA-B*44:02 allele, an HLA-B*40:02 allele, or any combination thereof. In some embodiments, the method of treating cancer in a subject in need thereof comprises administering: (i) a recombinant polypeptide consisting of at least four cancer-specific CD8+ T cell epitopes, or (ii) a polynucleotide encoding a recombinant polypeptide consisting of at four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); NO: 1) and KLVELEHTL (SEQ ID NO: 2) and SPSSASLAL (SEQ ID NO: 3) and KEFAFLEHSL (SEQ ID NO: 5) and wherein the subject expresses an MHC encoded by an HLA-A*02:01 allele, an HLA-B*07:02 allele, an HLA-B*35:03 allele, an HLA-B*40:01 allele, an HLA-B*44:02 allele, an HLA-B*40:02 allele, or any combination thereof.

In some embodiments, the method of treating cancer in a subject in need thereof comprises administering: (i) a recombinant polypeptide comprising at least four cancer-specific CD8+ T cell epitopes, or (ii) a polynucleotide encoding a recombinant polypeptide comprising at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are GVYDGREHTV (SEQ ID NO: 1) and KLVELEHTL (SEQ ID NO: 2) and SPSSASLAL (SEQ ID NO: 3) and HELGFKVVL (SEQ ID NO: 6). In some embodiments, the method of treating cancer in a subject in need thereof comprises administering: (i) a recombinant polypeptide consisting of at least four cancer-specific CD8+ T cell epitopes, or (ii) a polynucleotide encoding a recombinant polypeptide consisting of at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are GVYDGREHTV (SEQ ID NO: 1) and KLVELEHTL (SEQ ID NO: 2) and SPSSASLAL (SEQ ID NO: 3) and HELGFKVVL (SEQ ID NO: 6). In some embodiments, the method of treating cancer in a subject in need thereof comprises administering: (i) a recombinant polypeptide comprising at least four cancer-specific CD8+ T cell epitopes, or (ii) a polynucleotide encoding a recombinant polypeptide comprising at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are GVYDGREHTV (SEQ ID NO: 1) and KLVELEHTL (SEQ ID NO: 2) and SPSSASLAL (SEQ ID NO: 3) and HELGFKVVL (SEQ ID NO: 6) and wherein the subject expresses an MHC encoded by an HLA-A*02:01 allele, an HLA-B*07:02 allele, an HLA-B*35:03 allelle, an HLA-B*18:01 allele, an HLA-B*40:01 allele, an HLA-B*44:02 allele, an HLA-B*40:02 allele, or any combination thereof. In some embodiments, the method of treating cancer in a subject in need thereof comprises administering: (i) a recombinant polypeptide consisting of at least four cancer-specific CD8+ T cell epitopes, or (ii) a polynucleotide encoding a recombinant polypeptide consisting of at four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are GVYDGREHTV (SEQ ID NO: 1) and KLVELEHTL (SEQ ID NO: 2) and SPSSASLAL (SEQ ID NO: 3) and HELGFKVVL (SEQ ID NO: 6) and wherein the subject expresses an MHC encoded by an HLA-A*02:01 allele, an HLA-B*07:02 allele, an HLA-B*35:03 allelle, an HLA-B*18:01 allele, an HLA-B*40:01 allele, an HLA-B*44:02 allele, an HLA-B*40:02 allele, or any combination thereof.

In some embodiments, the method of treating cancer in a subject in need thereof comprises administering: (i) a recombinant polypeptide comprising at least four cancer-specific CD8+ T cell epitopes, or (ii) a polynucleotide encoding a recombinant polypeptide comprising at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are GVYDGREHTV (SEQ ID NO: 1) and SPSSASLAL (SEQ ID NO: 3) and KPRPDVTNEL (SEQ ID NO: 4) and KEFAFLEHSL (SEQ ID NO: 5). In some embodiments, the method of treating cancer in a subject in need thereof comprises administering: (i) a recombinant polypeptide consisting of at least four cancer-specific CD8+ T cell epitopes, or (ii) a polynucleotide encoding a recombinant polypeptide consisting of at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are GVYDGREHTV (SEQ ID NO: 1) and SPSSASLAL (SEQ ID NO: 3) and KPRPDVTNEL (SEQ ID NO: 4) and KEFAFLEHSL (SEQ ID NO: 5). In some embodiments, the method of treating cancer in a subject in need thereof comprises administering: (i) a recombinant polypeptide comprising at least four cancer-specific CD8+ T cell epitopes, or (ii) a polynucleotide encoding a recombinant polypeptide comprising at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are GVYDGREHTV (SEQ ID NO: 1) and SPSSASLAL (SEQ ID NO: 3) and KPRPDVTNEL (SEQ ID NO: 4) and KEFAFLEHSL (SEQ ID NO: 5) and wherein the subject expresses an MHC encoded by an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, or any combination thereof. In some embodiments, the method of treating cancer in a subject in need thereof comprises administering: (i) a recombinant polypeptide consisting of at least four cancer-specific CD8+ T cell epitopes, or (ii) a polynucleotide encoding a recombinant polypeptide consisting of at four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are GVYDGREHTV (SEQ ID NO: 1) and SPSSASLAL (SEQ ID NO: 3) and KPRPDVTNEL (SEQ ID NO: 4) and KEFAFLEHSL (SEQ ID NO: 5) and wherein the subject expresses an MHC encoded by an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, or any combination thereof.

In some embodiments, the method of treating cancer in a subject in need thereof comprises administering: (i) a recombinant polypeptide comprising at least four cancer-specific CD8+ T cell epitopes, or (ii) a polynucleotide encoding a recombinant polypeptide comprising at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are GVYDGREHTV (SEQ ID NO: 1) and SPSSASLAL (SEQ ID NO: 3) and KPRPDVTNEL (SEQ ID NO: 4) and HELGFKVVL (SEQ ID NO: 6). In some embodiments, the method of treating cancer in a subject in need thereof comprises administering: (i) a recombinant polypeptide consisting of at least four cancer-specific CD8+ T cell epitopes, or (ii) a polynucleotide encoding a recombinant polypeptide consisting of at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are GVYDGREHTV (SEQ ID NO: 1) and SPSSASLAL (SEQ ID NO: 3) and KPRPDVTNEL (SEQ ID NO: 4) and HELGFKVVL (SEQ ID NO: 6). In some embodiments, the method of treating cancer in a subject in need thereof comprises administering: (i) a recombinant polypeptide comprising at least four cancer-specific CD8+ T cell epitopes, or (ii) a polynucleotide encoding a recombinant polypeptide comprising at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are GVYDGREHTV (SEQ ID NO: 1) and SPSSASLAL (SEQ ID NO: 3) and KPRPDVTNEL (SEQ ID NO: 4) and HELGFKVVL (SEQ ID NO: 6) and wherein the subject expresses an MHC encoded by an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, an HLA-B*18:01 allele, or any combination thereof. In some embodiments, the method of treating cancer in a subject in need thereof comprises administering: (i) a recombinant polypeptide consisting of at least four cancer-specific CD8+ T cell epitopes, or (ii) a polynucleotide encoding a recombinant polypeptide consisting of at four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are GVYDGREHTV (SEQ ID NO: 1) and SPSSASLAL (SEQ ID NO: 3) and KPRPDVTNEL (SEQ ID NO: 4) and HELGFKVVL (SEQ ID NO: 6) and wherein the subject expresses an MHC encoded by an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, an HLA-B*18:01 allele, or any combination thereof.

In some embodiments, the method of treating cancer in a subject in need thereof comprises administering: (i) a recombinant polypeptide comprising at least four cancer-specific CD8+ T cell epitopes, or (ii) a polynucleotide encoding a recombinant polypeptide comprising at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are GVYDGREHTV (SEQ ID NO: 1) and SPSSASLAL (SEQ ID NO: 3) and KEFAFLEHSL (SEQ ID NO: 5) and HELGFKVVL (SEQ ID NO: 6). In some embodiments, the method of treating cancer in a subject in need thereof comprises administering: (i) a recombinant polypeptide consisting of at least four cancer-specific CD8+ T cell epitopes, or (ii) a polynucleotide encoding a recombinant polypeptide consisting of at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are GVYDGREHTV (SEQ ID NO: 1) and SPSSASLAL (SEQ ID NO: 3) and KEFAFLEHSL (SEQ ID NO: 5) and HELGFKVVL (SEQ ID NO: 6). In some embodiments, the method of treating cancer in a subject in need thereof comprises administering: (i) a recombinant polypeptide comprising at least four cancer-specific CD8+ T cell epitopes, or (ii) a polynucleotide encoding a recombinant polypeptide comprising at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are GVYDGREHTV (SEQ ID NO: 1) and SPSSASLAL (SEQ ID NO: 3) and KEFAFLEHSL (SEQ ID NO: 5) and HELGFKVVL (SEQ ID NO: 6) and wherein the subject expresses an MHC encoded by an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, an HLA-B*18:01 allele, or any combination thereof. In some embodiments, the method of treating cancer in a subject in need thereof comprises administering: (i) a recombinant polypeptide consisting of at least four cancer-specific CD8+ T cell epitopes, or (ii) a polynucleotide encoding a recombinant polypeptide consisting of at four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are GVYDGREHTV (SEQ ID NO: 1) and SPSSASLAL (SEQ ID NO: 3) and KEFAFLEHSL (SEQ ID NO: 5) and HELGFKVVL (SEQ ID NO: 6) and wherein the subject expresses an MHC encoded by an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, an HLA-B*18:01 allele, or any combination thereof.

In some embodiments, the method of treating cancer in a subject in need thereof comprises administering: (i) a recombinant polypeptide comprising at least four cancer-specific CD8+ T cell epitopes, or (ii) a polynucleotide encoding a recombinant polypeptide comprising at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are GVYDGREHTV (SEQ ID NO: 1) and KPRPDVTNEL (SEQ ID NO: 4) and KEFAFLEHSL (SEQ ID NO: 5) and HELGFKVVL (SEQ ID NO: 6). In some embodiments, the method of treating cancer in a subject in need thereof comprises administering: (i) a recombinant polypeptide consisting of at least four cancer-specific CD8+ T cell epitopes, or (ii) a polynucleotide encoding a recombinant polypeptide consisting of at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are GVYDGREHTV (SEQ ID NO: 1) and KPRPDVTNEL (SEQ ID NO: 4) and KEFAFLEHSL (SEQ ID NO: 5) and HELGFKVVL (SEQ ID NO: 6). In some embodiments, the method of treating cancer in a subject in need thereof comprises administering: (i) a recombinant polypeptide comprising at least four cancer-specific CD8+ T cell epitopes, or (ii) a polynucleotide encoding a recombinant polypeptide comprising at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are GVYDGREHTV (SEQ ID NO: 1) and KPRPDVTNEL (SEQ ID NO: 4) and KEFAFLEHSL (SEQ ID NO: 5) and HELGFKVVL (SEQ ID NO: 6) and wherein the subject expresses an MHC encoded by an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, an HLA-B*18:01 allele, or any combination thereof. In some embodiments, the method of treating cancer in a subject in need thereof comprises administering: (i) a recombinant polypeptide consisting of at least four cancer-specific CD8+ T cell epitopes, or (ii) a polynucleotide encoding a recombinant polypeptide consisting of at four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are GVYDGREHTV (SEQ ID NO: 1) and KPRPDVTNEL (SEQ ID NO: 4) and KEFAFLEHSL (SEQ ID NO: 5) and HELGFKVVL (SEQ ID NO: 6) and wherein the subject expresses an MHC encoded by an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, an HLA-B*18:01 allele, or any combination thereof.

In some embodiments, the method of treating cancer in a subject in need thereof comprises administering: (i) a recombinant polypeptide comprising at least four cancer-specific CD8+ T cell epitopes, or (ii) a polynucleotide encoding a recombinant polypeptide comprising at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are KLVELEHTL (SEQ ID NO: 2) and SPSSASLAL (SEQ ID NO: 3) and KPRPDVTNEL (SEQ ID NO: 4) and KEFAFLEHSL (SEQ ID NO: 5). In some embodiments, the method of treating cancer in a subject in need thereof comprises administering: (i) a recombinant polypeptide consisting of at least four cancer-specific CD8+ T cell epitopes, or (ii) a polynucleotide encoding a recombinant polypeptide consisting of at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are KLVELEHTL (SEQ ID NO: 2) and SPSSASLAL (SEQ ID NO: 3) and KPRPDVTNEL (SEQ ID NO: 4) and KEFAFLEHSL (SEQ ID NO: 5). In some embodiments, the method of treating cancer in a subject in need thereof comprises administering: (i) a recombinant polypeptide comprising at least four cancer-specific CD8+ T cell epitopes, or (ii) a polynucleotide encoding a recombinant polypeptide comprising at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are KLVELEHTL (SEQ ID NO: 2) and SPSSASLAL (SEQ ID NO: 3) and KPRPDVTNEL (SEQ ID NO: 4) and KEFAFLEHSL (SEQ ID NO: 5) and wherein the subject expresses an MHC encoded by an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, or any combination thereof. In some embodiments, the method of treating cancer in a subject in need thereof comprises administering: (i) a recombinant polypeptide consisting of at least four cancer-specific CD8+ T cell epitopes, or (ii) a polynucleotide encoding a recombinant polypeptide consisting of at four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are KLVELEHTL (SEQ ID NO: 2) and SPSSASLAL (SEQ ID NO: 3) and KPRPDVTNEL (SEQ ID NO: 4) and KEFAFLEHSL (SEQ ID NO: 5) and wherein the subject expresses an MHC encoded by an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, or any combination thereof.

In some embodiments, the method of treating cancer in a subject in need thereof comprises administering: (i) a recombinant polypeptide comprising at least four cancer-specific CD8+ T cell epitopes, or (ii) a polynucleotide encoding a recombinant polypeptide comprising at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are KLVELEHTL (SEQ ID NO: 2) and SPSSASLAL (SEQ ID NO: 3) and KPRPDVTNEL (SEQ ID NO: 4) and HELGFKVVL (SEQ ID NO: 6). In some embodiments, the method of treating cancer in a subject in need thereof comprises administering: (i) a recombinant polypeptide consisting of at least four cancer-specific CD8+ T cell epitopes, or (ii) a polynucleotide encoding a recombinant polypeptide consisting of at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are KLVELEHTL (SEQ ID NO: 2) and SPSSASLAL (SEQ ID NO: 3) and KPRPDVTNEL (SEQ ID NO: 4) and HELGFKVVL (SEQ ID NO: 6). In some embodiments, the method of treating cancer in a subject in need thereof comprises administering: (i) a recombinant polypeptide comprising at least four cancer-specific CD8+ T cell epitopes, or (ii) a polynucleotide encoding a recombinant polypeptide comprising at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are KLVELEHTL (SEQ ID NO: 2) and SPSSASLAL (SEQ ID NO: 3) and KPRPDVTNEL (SEQ ID NO: 4) and HELGFKVVL (SEQ ID NO: 6) and wherein the subject expresses an MHC encoded by an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, an HLA-B*18:01 allele, or any combination thereof. In some embodiments, the method of treating cancer in a subject in need thereof comprises administering: (i) a recombinant polypeptide consisting of at least four cancer-specific CD8+ T cell epitopes, or (ii) a polynucleotide encoding a recombinant polypeptide consisting of at four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are KLVELEHTL (SEQ ID NO: 2) and SPSSASLAL (SEQ ID NO: 3) and KPRPDVTNEL (SEQ ID NO: 4) and HELGFKVVL (SEQ ID NO: 6) and wherein the subject expresses an MHC encoded by an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, an HLA-B*18:01 allele, or any combination thereof.

In some embodiments, the method of treating cancer in a subject in need thereof comprises administering: (i) a recombinant polypeptide comprising at least four cancer-specific CD8+ T cell epitopes, or (ii) a polynucleotide encoding a recombinant polypeptide comprising at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are KLVELEHTL (SEQ ID NO: 2) and KPRPDVTNEL (SEQ ID NO: 4) and KEFAFLEHSL (SEQ ID NO: 5) and HELGFKVVL (SEQ ID NO: 6). In some embodiments, the method of treating cancer in a subject in need thereof comprises administering: (i) a recombinant polypeptide consisting of at least four cancer-specific CD8+ T cell epitopes, or (ii) a polynucleotide encoding a recombinant polypeptide consisting of at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are KLVELEHTL (SEQ ID NO: 2) and KPRPDVTNEL (SEQ ID NO: 4) and KEFAFLEHSL (SEQ ID NO: 5) and HELGFKVVL (SEQ ID NO: 6). In some embodiments, the method of treating cancer in a subject in need thereof comprises administering: (i) a recombinant polypeptide comprising at least four cancer-specific CD8+ T cell epitopes, or (ii) a polynucleotide encoding a recombinant polypeptide comprising at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are KLVELEHTL (SEQ ID NO: 2) and KPRPDVTNEL (SEQ ID NO: 4) and KEFAFLEHSL (SEQ ID NO: 5) and HELGFKVVL (SEQ ID NO: 6) and wherein the subject expresses an MHC encoded by an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, an HLA-B*18:01 allele, or any combination thereof. In some embodiments, the method of treating cancer in a subject in need thereof comprises administering: (i) a recombinant polypeptide consisting of at least four cancer-specific CD8+ T cell epitopes, or (ii) a polynucleotide encoding a recombinant polypeptide consisting of at four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are KLVELEHTL (SEQ ID NO: 2) and KPRPDVTNEL (SEQ ID NO: 4) and KEFAFLEHSL (SEQ ID NO: 5) and HELGFKVVL (SEQ ID NO: 6) and wherein the subject expresses an MHC encoded by an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, an HLA-B*18:01 allele, or any combination thereof.

In some embodiments, the method of treating cancer in a subject in need thereof comprises administering: (i) a recombinant polypeptide comprising at least four cancer-specific CD8+ T cell epitopes, or (ii) a polynucleotide encoding a recombinant polypeptide comprising at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are SPSSASLAL (SEQ ID NO: 3) and KPRPDVTNEL (SEQ ID NO: 4) and KEFAFLEHSL (SEQ ID NO: 5) and HELGFKVVL (SEQ ID NO: 6). In some embodiments, the method of treating cancer in a subject in need thereof comprises administering: (i) a recombinant polypeptide consisting of at least four cancer-specific CD8+ T cell epitopes, or (ii) a polynucleotide encoding a recombinant polypeptide consisting of at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are SPSSASLAL (SEQ ID NO: 3) and KPRPDVTNEL (SEQ ID NO: 4) and KEFAFLEHSL (SEQ ID NO: 5) and HELGFKVVL (SEQ ID NO: 6). In some embodiments, the method of treating cancer in a subject in need thereof comprises administering: (i) a recombinant polypeptide comprising at least four cancer-specific CD8+ T cell epitopes, or (ii) a polynucleotide encoding a recombinant polypeptide comprising at least four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are SPSSASLAL (SEQ ID NO: 3) and KPRPDVTNEL (SEQ ID NO: 4) and KEFAFLEHSL (SEQ ID NO: 5) and HELGFKVVL (SEQ ID NO: 6) and wherein the subject expresses an MHC encoded by an HLA-B*07:02 allele, an HLA-B*35:03 allele, an HLA-C*07:02 allele, an HLA-B*40:01 allele, an HLA-B*40:02 allele, an HLA-B*44:02 allele, an HLA-B*18:01 allele, or any combination thereof. In some embodiments, the method of treating cancer in a subject in need thereof comprises administering: (i) a recombinant polypeptide consisting of at least four cancer-specific CD8+ T cell epitopes, or (ii) a polynucleotide encoding a recombinant polypeptide consisting of at four cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least four cancer-specific CD8+ T cell epitopes are SPSSASLAL (SEQ ID NO: 3) and KPRPDVTNEL (SEQ ID NO: 4) and KEFAFLEHSL (SEQ ID NO: 5) and HELGFKVVL (SEQ ID NO: 6) and wherein the subject expresses an MHC encoded by an HLA-B*07:02 allele, an HLA-B*35:03 allele, an HLA-C*07:02 allele, an HLA-B*40:01 allele, an HLA-B*40:02 allele, an HLA-B*44:02 allele, an HLA-B*18:01 allele, or any combination thereof.

In some embodiments, the method of treating cancer in a subject in need thereof comprises administering: (i) a recombinant polypeptide comprising at least five cancer-specific CD8+ T cell epitopes, or (ii) a polynucleotide encoding a recombinant polypeptide comprising at least five cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least five cancer-specific CD8+ T cell epitopes are GVYDGREHTV (SEQ ID NO: 1) and KLVELEHTL (SEQ ID NO: 2) and SPSSASLAL (SEQ ID NO: 3) and KPRPDVTNEL (SEQ ID NO: 4) and KEFAFLEHSL (SEQ ID NO: 5). In some embodiments, the method of treating cancer in a subject in need thereof comprises administering: (i) a recombinant polypeptide consisting of at least five cancer-specific CD8+ T cell epitopes, or (ii) a polynucleotide encoding a recombinant polypeptide consisting of at least five cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least five cancer-specific CD8+ T cell epitopes are GVYDGREHTV (SEQ ID NO: 1) and KLVELEHTL (SEQ ID NO: 2) and SPSSASLAL (SEQ ID NO: 3) and KPRPDVTNEL (SEQ ID NO: 4) and KEFAFLEHSL (SEQ ID NO: 5). In some embodiments, the method of treating cancer in a subject in need thereof comprises administering: (i) a recombinant polypeptide comprising at least five cancer-specific CD8+ T cell epitopes, or (ii) a polynucleotide encoding a recombinant polypeptide comprising at least five cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least five cancer-specific CD8+ T cell epitopes are GVYDGREHTV (SEQ ID NO: 1) and KLVELEHTL (SEQ ID NO: 2) and SPSSASLAL (SEQ ID NO: 3) and KPRPDVTNEL (SEQ ID NO: 4) and KEFAFLEHSL (SEQ ID NO: 5) and wherein the subject expresses an MHC encoded by an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, or any combination thereof. In some embodiments, the method of treating cancer in a subject in need thereof comprises administering: (i) a recombinant polypeptide consisting of at least five cancer-specific CD8+ T cell epitopes, or (ii) a polynucleotide encoding a recombinant polypeptide consisting of at five cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least five cancer-specific CD8+ T cell epitopes are GVYDGREHTV (SEQ ID NO: 1) and KLVELEHTL (SEQ ID NO: 2) and SPSSASLAL (SEQ ID NO: 3) and KPRPDVTNEL (SEQ ID NO: 4) and KEFAFLEHSL (SEQ ID NO: 5) and wherein the subject expresses an MHC encoded by an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, or any combination thereof.

In some embodiments, the method of treating cancer in a subject in need thereof comprises administering: (i) a recombinant polypeptide comprising at least five cancer-specific CD8+ T cell epitopes, or (ii) a polynucleotide encoding a recombinant polypeptide comprising at least five cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least five cancer-specific CD8+ T cell epitopes are GVYDGREHTV (SEQ ID NO: 1) and KLVELEHTL (SEQ ID NO: 2) and SPSSASLAL (SEQ ID NO: 3) and KPRPDVTNEL (SEQ ID NO: 4) and HELGFKVVL (SEQ ID NO: 6). In some embodiments, the method of treating cancer in a subject in need thereof comprises administering: (i) a recombinant polypeptide consisting of at least five cancer-specific CD8+ T cell epitopes, or (ii) a polynucleotide encoding a recombinant polypeptide consisting of at least five cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least five cancer-specific CD8+ T cell epitopes are GVYDGREHTV (SEQ ID NO: 1) and KLVELEHTL (SEQ ID NO: 2) and SPSSASLAL (SEQ ID NO: 3) and KPRPDVTNEL (SEQ ID NO: 4) and HELGFKVVL (SEQ ID NO: 6). In some embodiments, the method of treating cancer in a subject in need thereof comprises administering: (i) a recombinant polypeptide comprising at least five cancer-specific CD8+ T cell epitopes, or (ii) a polynucleotide encoding a recombinant polypeptide comprising at least five cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least five cancer-specific CD8+ T cell epitopes are GVYDGREHTV (SEQ ID NO: 1) and KLVELEHTL (SEQ ID NO: 2) and SPSSASLAL (SEQ ID NO: 3) and KPRPDVTNEL (SEQ ID NO: 4) and HELGFKVVL (SEQ ID NO: 6) and wherein the subject expresses an MHC encoded by an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, an HLA-B*18:01 allele, or any combination thereof. In some embodiments, the method of treating cancer in a subject in need thereof comprises administering: (i) a recombinant polypeptide consisting of at least five cancer-specific CD8+ T cell epitopes, or (ii) a polynucleotide encoding a recombinant polypeptide consisting of at five cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least five cancer-specific CD8+ T cell epitopes are GVYDGREHTV (SEQ ID NO: 1) and KLVELEHTL (SEQ ID NO: 2) and SPSSASLAL (SEQ ID NO: 3) and KPRPDVTNEL (SEQ ID NO: 4) and HELGFKVVL (SEQ ID NO: 6) and wherein the subject expresses an MHC encoded by an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, an HLA-B*18:01 allele, or any combination thereof.

In some embodiments, the method of treating cancer in a subject in need thereof comprises administering: (i) a recombinant polypeptide comprising at least five cancer-specific CD8+ T cell epitopes, or (ii) a polynucleotide encoding a recombinant polypeptide comprising at least five cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least five cancer-specific CD8+ T cell epitopes are GVYDGREHTV (SEQ ID NO: 1) and SPSSASLAL (SEQ ID NO: 3) and KPRPDVTNEL (SEQ ID NO: 4) and KEFAFLEHSL (SEQ ID NO: 5) and HELGFKVVL (SEQ ID NO: 6). In some embodiments, the method of treating cancer in a subject in need thereof comprises administering: (i) a recombinant polypeptide consisting of at least five cancer-specific CD8+ T cell epitopes, or (ii) a polynucleotide encoding a recombinant polypeptide consisting of at least five cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least five cancer-specific CD8+ T cell epitopes are GVYDGREHTV (SEQ ID NO: 1) and SPSSASLAL (SEQ ID NO: 3) and KPRPDVTNEL (SEQ ID NO: 4) and KEFAFLEHSL (SEQ ID NO: 5) and HELGFKVVL (SEQ ID NO: 6). In some embodiments, the method of treating cancer in a subject in need thereof comprises administering: (i) a recombinant polypeptide comprising at least five cancer-specific CD8+ T cell epitopes, or (ii) a polynucleotide encoding a recombinant polypeptide comprising at least five cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least five cancer-specific CD8+ T cell epitopes are GVYDGREHTV (SEQ ID NO: 1) and SPSSASLAL (SEQ ID NO: 3) and KPRPDVTNEL (SEQ ID NO: 4) and KEFAFLEHSL (SEQ ID NO: 5) and HELGFKVVL (SEQ ID NO: 6) and wherein the subject expresses an MHC encoded by an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, an HLA-B*18:01 allele, or any combination thereof. In some embodiments, the method of treating cancer in a subject in need thereof comprises administering: (i) a recombinant polypeptide consisting of at least five cancer-specific CD8+ T cell epitopes, or (ii) a polynucleotide encoding a recombinant polypeptide consisting of at five cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least five cancer-specific CD8+ T cell epitopes are GVYDGREHTV (SEQ ID NO: 1) and SPSSASLAL (SEQ ID NO: 3) and KPRPDVTNEL (SEQ ID NO: 4) and KEFAFLEHSL (SEQ ID NO: 5) and HELGFKVVL (SEQ ID NO: 6) and wherein the subject expresses an MHC encoded by an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, an HLA-B*18:01 allele, or any combination thereof.

In some embodiments, the method of treating cancer in a subject in need thereof comprises administering: (i) a recombinant polypeptide comprising at least five cancer-specific CD8+ T cell epitopes, or (ii) a polynucleotide encoding a recombinant polypeptide comprising at least five cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least five cancer-specific CD8+ T cell epitopes are KLVELEHTL (SEQ ID NO: 2) and SPSSASLAL (SEQ ID NO: 3) and KPRPDVTNEL (SEQ ID NO: 4) and KEFAFLEHSL (SEQ ID NO: 5) and HELGFKVVL (SEQ ID NO: 6). In some embodiments, the method of treating cancer in a subject in need thereof comprises administering: (i) a recombinant polypeptide consisting of at least five cancer-specific CD8+ T cell epitopes, or (ii) a polynucleotide encoding a recombinant polypeptide consisting of at least five cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least five cancer-specific CD8+ T cell epitopes are KLVELEHTL (SEQ ID NO: 2) and SPSSASLAL (SEQ ID NO: 3) and KPRPDVTNEL (SEQ ID NO: 4) and KEFAFLEHSL (SEQ ID NO: 5) and HELGFKVVL (SEQ ID NO: 6). In some embodiments, the method of treating cancer in a subject in need thereof comprises administering: (i) a recombinant polypeptide comprising at least five cancer-specific CD8+ T cell epitopes, or (ii) a polynucleotide encoding a recombinant polypeptide comprising at least five cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least five cancer-specific CD8+ T cell epitopes are KLVELEHTL (SEQ ID NO: 2) and SPSSASLAL (SEQ ID NO: 3) and KPRPDVTNEL (SEQ ID NO: 4) and KEFAFLEHSL (SEQ ID NO: 5) and HELGFKVVL (SEQ ID NO: 6) and wherein the subject expresses an MHC encoded by an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, an HLA-B*18:01 allele, or any combination thereof. In some embodiments, the method of treating cancer in a subject in need thereof comprises administering: (i) a recombinant polypeptide consisting of at least five cancer-specific CD8+ T cell epitopes, or (ii) a polynucleotide encoding a recombinant polypeptide consisting of at five cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least five cancer-specific CD8+ T cell epitopes are KLVELEHTL (SEQ ID NO: 2) and SPSSASLAL (SEQ ID NO: 3) and KPRPDVTNEL (SEQ ID NO: 4) and KEFAFLEHSL (SEQ ID NO: 5) and HELGFKVVL (SEQ ID NO: 6) and wherein the subject expresses an MHC encoded by an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, an HLA-B*18:01 allele, or any combination thereof.

In some embodiments, the method of treating cancer in a subject in need thereof comprises administering: (i) a recombinant polypeptide comprising at least five cancer-specific CD8+ T cell epitopes, or (ii) a polynucleotide encoding a recombinant polypeptide comprising at least five cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least five cancer-specific CD8+ T cell epitopes are GVYDGREHTV (SEQ ID NO: 1) and KLVELEHTL (SEQ ID NO: 2) and KPRPDVTNEL (SEQ ID NO: 4) and KEFAFLEHSL (SEQ ID NO: 5) and HELGFKVVL (SEQ ID NO: 6). In some embodiments, the method of treating cancer in a subject in need thereof comprises administering: (i) a recombinant polypeptide consisting of at least five cancer-specific CD8+ T cell epitopes, or (ii) a polynucleotide encoding a recombinant polypeptide consisting of at least five cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least five cancer-specific CD8+ T cell epitopes are GVYDGREHTV (SEQ ID NO: 1) and KLVELEHTL (SEQ ID NO: 2) and KPRPDVTNEL (SEQ ID NO: 4) and KEFAFLEHSL (SEQ ID NO: 5) and HELGFKVVL (SEQ ID NO: 6). In some embodiments, the method of treating cancer in a subject in need thereof comprises administering: (i) a recombinant polypeptide comprising at least five cancer-specific CD8+ T cell epitopes, or (ii) a polynucleotide encoding a recombinant polypeptide comprising at least five cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least five cancer-specific CD8+ T cell epitopes are GVYDGREHTV (SEQ ID NO: 1) and KLVELEHTL (SEQ ID NO: 2) and KPRPDVTNEL (SEQ ID NO: 4) and KEFAFLEHSL (SEQ ID NO: 5) and HELGFKVVL (SEQ ID NO: 6) and wherein the subject expresses an MHC encoded by an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, an HLA-B*18:01 allele, or any combination thereof. In some embodiments, the method of treating cancer in a subject in need thereof comprises administering: (i) a recombinant polypeptide consisting of at least five cancer-specific CD8+ T cell epitopes, or (ii) a polynucleotide encoding a recombinant polypeptide consisting of at five cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least five cancer-specific CD8+ T cell epitopes are GVYDGREHTV (SEQ ID NO: 1) and KLVELEHTL (SEQ ID NO: 2) and KPRPDVTNEL (SEQ ID NO: 4) and KEF AFLEHSL (SEQ ID NO: 5) and HELGFKVVL (SEQ ID NO: 6) and wherein the subject expresses an MHC encoded by an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, an HLA-B*18:01 allele, or any combination thereof.

In some embodiments, the method of treating cancer in a subject in need thereof comprises administering: (i) a recombinant polypeptide comprising at least five cancer-specific CD8+ T cell epitopes, or (ii) a polynucleotide encoding a recombinant polypeptide comprising at least five cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least five cancer-specific CD8+ T cell epitopes are GVYDGREHTV (SEQ ID NO: 1) and KLVELEHTL (SEQ ID NO: 2) and SPSSASLAL (SEQ ID NO: 3) and KEFAFLEHSL (SEQ ID NO: 5) and HELGFKVVL (SEQ ID NO: 6). In some embodiments, the method of treating cancer in a subject in need thereof comprises administering: (i) a recombinant polypeptide consisting of at least five cancer-specific CD8+ T cell epitopes, or (ii) a polynucleotide encoding a recombinant polypeptide consisting of at least five cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least five cancer-specific CD8+ T cell epitopes are GVYDGREHTV (SEQ ID NO: 1) and KLVELEHTL (SEQ ID NO: 2) and SPSSASLAL (SEQ ID NO: 3) and KEFAFLEHSL (SEQ ID NO: 5) and HELGFKVVL (SEQ ID NO: 6). In some embodiments, the method of treating cancer in a subject in need thereof comprises administering: (i) a recombinant polypeptide comprising at least five cancer-specific CD8+ T cell epitopes, or (ii) a polynucleotide encoding a recombinant polypeptide comprising at least five cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least five cancer-specific CD8+ T cell epitopes are GVYDGREHTV (SEQ ID NO: 1) and KLVELEHTL (SEQ ID NO: 2) and SPSSASLAL (SEQ ID NO: 3) and KEFAFLEHSL (SEQ ID NO: 5) and HELGFKVVL (SEQ ID NO: 6) and wherein the subject expresses an MHC encoded by an HLA-A*02:01 allele, an HLA-B*07:02 allele, an HLA-B*35:03 allele, an HLA-B*40:01 allele, an HLA-B*40:02 allele, an HLA-B*44:02 allele, an HLA-B*18:01 allele, or any combination thereof. In some embodiments, the method of treating cancer in a subject in need thereof comprises administering: (i) a recombinant polypeptide consisting of at least five cancer-specific CD8+ T cell epitopes, or (ii) a polynucleotide encoding a recombinant polypeptide consisting of at five cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least five cancer-specific CD8+ T cell epitopes are GVYDGREHTV (SEQ ID NO: 1) and KLVELEHTL (SEQ ID NO: 2) and SPSSASLAL (SEQ ID NO: 3) and KEFAFLEHSL (SEQ ID NO: 5) and HELGFKVVL (SEQ ID NO: 6) and wherein the subject expresses an MHC encoded by an HLA-A*02:01 allele, an HLA-B*07:02 allele, an HLA-B*35:03 allele, an HLA-B*40:01 allele, an HLA-B*40:02 allele, an HLA-B*44:02 allele, an HLA-B*18:01 allele, or any combination thereof.

In some embodiments, the method of treating cancer in a subject in need thereof comprises administering: (i) a recombinant polypeptide comprising at least six cancer-specific CD8+ T cell epitopes, or (ii) a polynucleotide encoding a recombinant polypeptide comprising at least six cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least six cancer-specific CD8+ T cell epitopes are GVYDGREHTV (SEQ ID NO: 1) and KLVELEHTL (SEQ ID NO: 2) and SPSSASLAL (SEQ ID NO: 3) and KPRPDVTNEL (SEQ ID NO: 4) and KEFAFLEHSL (SEQ ID NO: 5) and HELGFKVVL (SEQ ID NO: 6). In some embodiments, the method of treating cancer in a subject in need thereof comprises administering: (i) a recombinant polypeptide consisting of at least six cancer-specific CD8+ T cell epitopes, or (ii) a polynucleotide encoding a recombinant polypeptide consisting of at least six cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least six cancer-specific CD8+ T cell epitopes are GVYDGREHTV (SEQ ID NO: 1) and KLVELEHTL (SEQ ID NO: 2) and SPSSASLAL (SEQ ID NO: 3) and KPRPDVTNEL (SEQ ID NO: 4) and KEFAFLEHSL (SEQ ID NO: 5) and HELGFKVVL (SEQ ID NO: 6). In some embodiments, the method of treating cancer in a subject in need thereof comprises administering: (i) a recombinant polypeptide comprising at least six cancer-specific CD8+ T cell epitopes, or (ii) a polynucleotide encoding a recombinant polypeptide comprising at least six cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least six cancer-specific CD8+ T cell epitopes are GVYDGREHTV (SEQ ID NO: 1) and KLVELEHTL (SEQ ID NO: 2) and SPSSASLAL (SEQ ID NO: 3) and KPRPDVTNEL (SEQ ID NO: 4) and KEFAFLEHSL (SEQ ID NO: 5) and HELGFKVVL (SEQ ID NO: 6) and wherein the subject expresses an MHC encoded by an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, an HLA-B*18:01 allele, or any combination thereof. In some embodiments, the method of treating cancer in a subject in need thereof comprises administering: (i) a recombinant polypeptide consisting of at least six cancer-specific CD8+ T cell epitopes, or (ii) a polynucleotide encoding a recombinant polypeptide consisting of at six cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the at least six cancer-specific CD8+ T cell epitopes are GVYDGREHTV (SEQ ID NO: 1) and KLVELEHTL (SEQ ID NO: 2) and SPSSASLAL (SEQ ID NO: 3) and KPRPDVTNEL (SEQ ID NO: 4) and KEF AFLEHSL (SEQ ID NO: 5) and HELGFKVVL (SEQ ID NO: 6) and wherein the subject expresses an MHC encoded by an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, an HLA-B*18:01 allele, or any combination thereof.

In some embodiments, the method of treating cancer in a subject in need thereof comprises administering: (i) a recombinant polypeptide comprising one, two, three, four, five, six, or more cancer-specific CD8+ T cell epitopes, or (ii) a polynucleotide encoding a recombinant polypeptide comprising one, two, three, four, five, six, or more cancer-specific CD8+ T cell epitopes, (iii) a vector comprising (i) or (ii), (iv) antigen presenting cells (APCs) comprising (i), (ii), or (iii), or (v) T cells stimulated with APCs comprising (i), (ii), or (iii); wherein the one, two, three, four, five, six, or more cancer-specific CD8+ T cell epitopes comprise a cancer-specific CD8+ T cell epitope or plurality of cancer-specific CD8+ T cell epitopes described herein and wherein the composition is for use in treating cancer is a subject that expresses an HLA-A*02:01 allele, an HLA-B*07:02 allele, an HLA-B*35:03 allele, an HLA-C*07:02 allele, an HLA-B*40:01 allele, an HLA-B*40:02 allele, an HLA-B*44:02 allele, an HLA-B*18:01 allele, an HLA-A*03:01 allele, an HLA-A*31:01 allele, an HLA-A*11:01 allele, an HLA-C*03:03 allele, an HLA-C*03:04 allele, an HLA-B*15:01 allele, an HLA-B*08:01 allele, an HLA-A*02:05 allele, or any combination thereof.

In some embodiments, the cancer is a colorectal cancer, prostate cancer, esophageal cancer, liver cancer, renal cancer, lung cancer, bladder cancer, breast cancer, pancreatic cancer, brain cancer, hepatocellular cancer, lymphoma, leukaemia, gastric cancer, cervical cancer, ovarian cancer, thyroid cancer, melanoma, carcinoma, head and neck cancer, skin cancer, nasopharyngeal cancer, Epstein Barr driven cancers, Human Papilloma virus driven cancers or soft tissue sarcoma. In some embodiments, the cancer is a primary cancer. In some embodiments, the cancer is a relapsed and/or a refractory cancer.

In some instances, the subject expresses an MHC encoded by an HLA-A*02:01 allele and is administered a composition comprising a cancer-specific CD8+ T cell epitope selected from GVYDGREHTV (SEQ ID NO: 1) and/or KLVELEHTL (SEQ ID NO: 2).

In some instances, the subject expresses an MHC encoded by an HLA-B*07:02 allele and is administered a composition comprising a cancer-specific CD8+ T cell epitope selected from SPSSASLAL (SEQ ID NO: 3) and/or KPRPDVTNEL (SEQ ID NO: 4). In some instances, the subject expresses an MHC encoded by an HLA-B*07:02 allele and is administered a composition comprising a cancer-specific CD8+ T cell epitope selected from SPSSASLAL (SEQ ID NO: 3), KPRPDVTNEL (SEQ ID NO: 4), NLRPPTQEL (SEQ ID NO: 10), LRPPTQEL (SEQ ID NO: 12), and/or RPASPRPAP (SEQ ID NO: 13). In some instances, the subject expresses an MHC encoded by an HLA-B*07:02 allele and is administered a composition comprising a cancer-specific CD8+ T cell epitope selected from SPSSASLAL (SEQ ID NO: 3), KPRPDVTNEL (SEQ ID NO: 4), NLRPPTQEL (SEQ ID NO: 10), RPASPRPAP (SEQ ID NO: 13), GPRPSPTRSV (SEQ ID NO: 20), SPRSPSPSL (SEQ ID NO: 22), VPQEAVRAPL (SEQ ID NO: 23), IYPFINSH (SEQ ID NO: 24), and/or SPSSASLTL (SEQ ID NO: 25).

In some instances, the subject expresses an MHC encoded by an HLA-B*35:03 allele and is administered a composition comprising a cancer-specific CD8+ T cell epitope selected from SPSSASLAL (SEQ ID NO: 3) and/or KPRPDVTNEL (SEQ ID NO: 4). In some instances, the subject expresses an MHC encoded by an HLA-B*35:03 allele and is administered a composition comprising a cancer-specific CD8+ T cell epitope selected from SPSSASLAL (SEQ ID NO: 3), KPRPDVTNEL (SEQ ID NO: 4), NLRPPTQEL (SEQ ID NO: 10), LRPPTQEL (SEQ ID NO: 12), and/or RPASPRPAP (SEQ ID NO: 13). In some instances, the subject expresses an MHC encoded by an HLA-B*35:03 allele and is administered a composition comprising a cancer-specific CD8+ T cell epitope selected from SPSSASLAL (SEQ ID NO: 3), KPRPDVTNEL (SEQ ID NO: 4), NLRPPTQEL (SEQ ID NO: 10), RPASPRPAP (SEQ ID NO: 13), GPRPSPTRSV (SEQ ID NO: 20), SPRSPSPSL (SEQ ID NO: 22), VPQEAVRAPL (SEQ ID NO: 23), IYPFINSH (SEQ ID NO: 24), and/or SPSSASLTL (SEQ ID NO: 25).

In some instances, the subject expresses an MHC encoded by an HLA-B*40:01 allele and is administered a composition comprising a cancer-specific CD8+ T cell epitope selected from KEFAFLEHSL (SEQ ID NO: 5) and/or HELGFKVVL (SEQ ID NO: 6).

In some instances, the subject expresses an MHC encoded by an HLA-B*40:02 allele and is administered a composition comprising a cancer-specific CD8+ T cell epitope selected from KEFAFLEHSL (SEQ ID NO: 5) and/or HELGFKVVL (SEQ ID NO: 6).

In some instances, the subject expresses an MHC encoded by an HLA-B*44:02 allele and is administered a composition comprising a cancer-specific CD8+ T cell epitope selected from KEFAFLEHSL (SEQ ID NO: 5) and/or HELGFKVVL (SEQ ID NO: 6).

In some instances, the subject expresses an MHC encoded by an HLA-B*18:01 allele and is administered a composition comprising a cancer-specific CD8+ T cell epitope HELGFKVVL (SEQ ID NO: 6).

In some instances, the subject expresses an MHC encoded by an HLA-A*03:01 allele and is administered a composition comprising a cancer-specific CD8+ T cell epitope selected from LKPDHIQR (SEQ ID NO: 7), SLFGARPGR (SEQ ID NO: 8) and/or KTEVHGRLK (SEQ ID NO: 9). In some instances, the subject expresses an MHC encoded by an HLA-A*03:01 allele and is administered a composition comprising a cancer-specific CD8+ T cell epitope selected from LKPDHIQR (SEQ ID NO: 7), RVSLPKLGYK (SEQ ID NO: 11), SLFGARPGR (SEQ ID NO: 8) and/or KTEVHGRLK (SEQ ID NO: 9). In some instances, the subject expresses an MHC encoded by an HLA-A*03:01 allele and is administered a composition comprising a cancer-specific CD8+ T cell epitope selected from LKPDHIQR (SEQ ID NO: 7), RVSLPKLGYK (SEQ ID NO: 11), SLFGARPGR (SEQ ID NO: 8), KTEVHGRLK (SEQ ID NO: 9), RLHSFTLRQK (SEQ ID NO: 17), RVFTSSLKTK (SEQ ID NO: 18), RVLAKGLAK (SEQ ID NO: 19), KTPLHTLLK (SEQ ID NO: 16) and/or SLYQTIRLK (SEQ ID NO: 15).

In some instances, the subject expresses an MHC encoded by an HLA-A*11:01 allele and is administered a composition comprising a cancer-specific CD8+ T cell epitope selected from LKPDHIQR (SEQ ID NO: 7), SLFGARPGR (SEQ ID NO: 8) and/or KTEVHGRLK (SEQ ID NO: 9). In some instances, the subject expresses an MHC encoded by an HLA-A*03:01 allele and is administered a composition comprising a cancer-specific CD8+ T cell epitope selected from LKPDHIQR (SEQ ID NO: 7), RVSLPKLGYK (SEQ ID NO: 11), SLFGARPGR (SEQ ID NO: 8) and/or KTEVHGRLK (SEQ ID NO: 9). In some instances, the subject expresses an MHC encoded by an HLA-A*11:01 allele and is administered a composition comprising a cancer-specific CD8+ T cell epitope selected from LKPDHIQR (SEQ ID NO: 7), RVSLPKLGYK (SEQ ID NO: 11), SLFGARPGR (SEQ ID NO: 8), KTEVHGRLK (SEQ ID NO: 9), RLHSFTLRQK (SEQ ID NO: 17), RVFTSSLKTK (SEQ ID NO: 18), RVLAKGLAK (SEQ ID NO: 19), KTPLHTLLK (SEQ ID NO: 16) and/or SLYQTIRLK (SEQ ID NO: 15).

In some instances, the subject expresses an MHC encoded by an HLA-A*31:01 allele and is administered a composition comprising the cancer-specific CD8+ T cell epitope SLFGARPGR (SEQ ID NO: 8).

In some instances, the subject expresses an MHC encoded by an HLA-A*15:01 allele and is administered a composition comprising a cancer-specific CD8+ T cell epitope GQHLHLETF (SEQ ID NO: 21).

In some instances, the subject expresses an MHC encoded by an HLA-B*08:01 allele and is administered a composition comprising a cancer-specific CD8+ T cell epitope selected from LNKVKTSL (SEQ ID NO: 26) and/or NLKTHLRL (SEQ ID NO: 27). In some instances, the subject expresses an MHC encoded by an HLA-C*07:01 allele and is administered a composition comprising a cancer-specific CD8+ T cell epitope FRGVFVHRY (SEQ ID NO: 28). In some instances, the subject expresses an MHC encoded by an HLA-C*07:02 allele and is administered a composition comprising a cancer-specific CD8+ T cell epitope LRPPTQEL (SEQ ID NO: 12) or IYPFINSH (SEQ ID NO: 24). In some instances, the subject expresses an MHC encoded by an HLA-B*35:01 allele and is administered a composition comprising a cancer-specific CD8+ T cell epitope TSGPVTEKY (SEQ ID NO: 29). In some instances, the subject expresses an MHC encoded by an HLA-A*02:05 allele and is administered a composition comprising a cancer-specific CD8+ T cell epitope VVAAHLAGA (SEQ ID NO: 30).

In some instances, the subject expresses an MHC encoded by an HLA-A*02:01 allele, and/or an HLA-B*07:02 allele and is administered a composition comprising a cancer-specific CD8+ T cell epitope selected from GVYDGREHTV (SEQ ID NO: 1), KLVELEHTL (SEQ ID NO: 2), SPSSASLAL (SEQ ID NO: 3) and/or KPRPDVTNEL (SEQ ID NO: 4).

In some instances, the subject expresses an MHC encoded by an HLA-A*02:01 allele, and is administered a composition comprising a cancer-specific CD8+ T cell epitope selected from GVYDGREHTV (SEQ ID NO: 1), KLVELEHTL (SEQ ID NO: 2), KEFAFLEHSL (SEQ ID NO: 5) and/or HELGFKVVL (SEQ ID NO: 6).

In some instances, the subject expresses an MHC encoded by an HLA-B*07:02 allele, and is administered a composition comprising a cancer-specific CD8+ T cell epitope selected from SPSSASLAL (SEQ ID NO: 3), KPRPDVTNEL (SEQ ID NO: 4), KEFAFLEHSL (SEQ ID NO: 5) and/or HELGFKVVL (SEQ ID NO: 6).

In some instances, the subject expresses an MHC encoded by an HLA-A*02:01 allele, an HLA-B*07:02 allele, and is administered a composition comprising a cancer-specific CD8+ T cell epitope selected from GVYDGREHTV (SEQ ID NO: 1), KLVELEHTL (SEQ ID NO: 2), SPSSASLAL (SEQ ID NO: 3), KPRPDVTNEL (SEQ ID NO: 4), KEFAFLEHSL (SEQ ID NO: 5) and/or HELGFKVVL (SEQ ID NO: 6).

In some instances, the subject expresses an MHC encoded by an HLA-B*07:02 allele and is administered (i) a recombinant polypeptide comprising SEQ ID NO: 13, or (ii) a polynucleotide encoding a recombinant polypeptide comprising SEQ ID NO: 13, or (iii) a vector comprising (i) or (ii).

In some instances, the subject expresses an MHC encoded by an HLA-C*03:04 allele and is administered (i) a recombinant polypeptide comprising SEQ ID NO: 14, or (ii) a polynucleotide encoding a recombinant polypeptide comprising SEQ ID NO: 14, or (iii) a vector comprising (i) or (ii).

In some instances, the subject expresses an MHC encoded by an HLA-A*02:01 allele and/or an MHC encoded by an HLA-C*03:04 allele and is administered a drug product comprising (i) a first recombinant polypeptide comprising SEQ ID NO: 13, or (ii) a first polynucleotide encoding a recombinant polypeptide comprising SEQ ID NO: 13, or (iii) a first vector comprising (i) or (ii) and (iv) a second recombinant polypeptide comprising SEQ ID NO: 14, or (v) a second polynucleotide encoding a recombinant polypeptide comprising SEQ ID NO: 14 or (vi) a second vector comprising (iv) or (v).

In some instances, the subject expresses an MHC encoded by an HLA-A*03:01allele and is administered (i) a recombinant polypeptide comprising SEQ ID NO: 15, or (ii) a polynucleotide encoding a recombinant polypeptide comprising SEQ ID NO: 15, or (iii) a vector comprising (i) or (ii).

In some instances, the subject expresses an MHC encoded by an HLA-A*03:01 allele and is administered (i) a recombinant polypeptide comprising SEQ ID NO: 16, or (ii) a polynucleotide encoding a recombinant polypeptide comprising SEQ ID NO: 16, or (iii) a vector comprising (i) or (ii).

In some instances, the subject expresses an MHC encoded by an HLA-A*03:01 allele and is administered a drug product comprising (i) a first recombinant polypeptide comprising SEQ ID NO: 15, or (ii) a first polynucleotide encoding a recombinant polypeptide comprising SEQ ID NO: 15, or (iii) a first vector comprising (i) or (ii) and (iv) a second recombinant polypeptide comprising SEQ ID NO: 16, or (v) a second polynucleotide encoding a recombinant polypeptide comprising SEQ ID NO: 16, or (vi) a second vector comprising (iv) or (v).

In some instances, the subject expresses an MHC encoded by an HLA-A*03:01 allele and is administered (i) a recombinant polypeptide comprising SEQ ID NO: 17, or (ii) a polynucleotide encoding a recombinant polypeptide comprising SEQ ID NO: 17, or (iii) a vector comprising (i) or (ii).

In some instances, the subject expresses an MHC encoded by an HLA-A*03:01 allele and is administered (i) a recombinant polypeptide comprising SEQ ID NO: 18, or (ii) a polynucleotide encoding a recombinant polypeptide comprising SEQ ID NO: 18, or (iii) a vector comprising (i) or (ii).

In some instances, the subject expresses an MHC encoded by an HLA-A*03:01 and is administered a drug product comprising (i) a first recombinant polypeptide comprising SEQ ID NO: 17, or (ii) a first polynucleotide encoding a recombinant polypeptide comprising SEQ ID NO: 17, or (iii) a first vector comprising (i) or (ii) and (iv) a second recombinant polypeptide comprising SEQ ID NO: 18, or (v) a second polynucleotide encoding a recombinant polypeptide comprising SEQ ID NO: 18, or (vi) a second vector comprising (iv) or (v).

In some instances, the subject expresses an MHC encoded by an HLA-A*02:01 allele, an MHC encoded by an HLA-C*03:04 allele, and an MHC encoded by an HLA-A*03:01 allele and is administered a first drug product comprising (i) a first recombinant polypeptide comprising SEQ ID NO: 13, or (ii) a first polynucleotide encoding a recombinant polypeptide comprising SEQ ID NO: 13, or (iii) a first vector comprising (i) or (ii) and (iv) a second recombinant polypeptide comprising SEQ ID NO: 14, or (v) a second polynucleotide encoding a recombinant polypeptide comprising SEQ ID NO: 14, or (vi) a second vector comprising (iv) or (iv) and a second drug product comprising (i) a third recombinant polypeptide comprising SEQ ID NO: 15, or (ii) a third polynucleotide encoding a recombinant polypeptide comprising SEQ ID NO: 15, or (iii) a third vector comprising (i) or (ii) and (iv) a fourth recombinant polypeptide comprising SEQ ID NO: 16, or (v) a fourth polynucleotide encoding a recombinant polypeptide comprising SEQ ID NO: 16, or (vi) a fourth vector comprising (iv) or (v).

In some instances, the subject expresses an MHC encoded by an HLA-A*02:01 allele, an MHC encoded by an HLA-C*03:04 allele, and an MHC encoded by an HLA-A*03:01 allele, and is administered a first drug product comprising (i) a first recombinant polypeptide comprising SEQ ID NO: 13, or (ii) a first polynucleotide encoding a recombinant polypeptide comprising SEQ ID NO: 13, or (iii) a first vector comprising (i) or (ii) and a (iv) a second recombinant polypeptide comprising SEQ ID NO: 14, or (v) a second polynucleotide encoding a recombinant polypeptide comprising SEQ ID NO: 14, or (vi) a second vector comprising (iv) or (v) and a second drug product comprising drug product comprising (i) a third recombinant polypeptide comprising SEQ ID NO: 17, or (ii) a third polynucleotide encoding a recombinant polypeptide comprising SEQ ID NO: 17, or (iii) a third vector comprising (i) or (ii) and (iv) a fourth recombinant polypeptide comprising SEQ ID NO: 18, or (v) a fourth polynucleotide encoding a recombinant polypeptide comprising SEQ ID NO: 18, or (vi) a fourth vector comprising (iv) or (v).

In some instances, the subject expresses an MHC encoded by an HLA-A*03:01 allele, and is administered a first drug product comprising (i) a first recombinant polypeptide comprising SEQ ID NO: 15, or (ii) a first polynucleotide encoding a recombinant polypeptide comprising SEQ ID NO: 15, or (iii) a first vector comprising (i) or (ii) and a (iv) a second recombinant polypeptide comprising SEQ ID NO: 16, or (v) a second polynucleotide encoding a recombinant polypeptide comprising SEQ ID NO: 16, or (vi) a second vector comprising (iv) or (v) and a second drug product comprising (i) a third recombinant polypeptide comprising SEQ ID NO: 17, or (ii) a third polynucleotide encoding a recombinant polypeptide comprising SEQ ID NO: 17, or (iii) a third vector comprising (i) or (ii) and (iv) a fourth recombinant polypeptide comprising SEQ ID NO: 18, or (v) a fourth polynucleotide encoding a recombinant polypeptide comprising SEQ ID NO: 18, or (vi) a fourth vector comprising (iv) or (v).

In some instances, the subject expresses an MHC encoded by an HLA-A*02:01 allele, an MHC encoded by an HLA-C*03:04 allele, and an MHC encoded by an HLA-A*03:01 allele, and is administered a first drug product comprising (i) a first recombinant polypeptide comprising SEQ ID NO: 13, or (ii) a first polynucleotide encoding a recombinant polypeptide comprising SEQ ID NO: 13, or (iii) a first vector comprising (i) or (ii) and (iv) a second recombinant polypeptide comprising SEQ ID NO: 14, or (v) a second polynucleotide encoding a recombinant polypeptide comprising SEQ ID NO: 14, or (vi) a second vector comprising (iv) or (iv); a second drug product comprising (i) a third recombinant polypeptide comprising SEQ ID NO: 15, or (ii) a third polynucleotide encoding a recombinant polypeptide comprising SEQ ID NO: 15, or (iii) a third vector comprising (i) or (ii) and (iv) a fourth recombinant polypeptide comprising SEQ ID NO: 16, or (v) a fourth polynucleotide encoding a recombinant polypeptide comprising SEQ ID NO: 16, or (vi) a fourth vector comprising (iv) or (v); and a third drug product comprising (i) a fifth recombinant polypeptide comprising SEQ ID NO: 17, or (ii) a fifth polynucleotide encoding a recombinant polypeptide comprising SEQ ID NO: 17, or (iii) a fifth vector comprising (i) or (ii) and (iv) a sixth recombinant polypeptide comprising SEQ ID NO: 18, or (v) a sixth polynucleotide encoding a recombinant polypeptide comprising SEQ ID NO: 18, or (vi) a sixth vector comprising (iv) or (v).

In some instances, the subject expresses an MHC encoded by an HLA-A*03:01 allele and is administered (i) a recombinant polypeptide comprising SEQ ID NO: 7, or (ii) a polynucleotide encoding a recombinant polypeptide comprising SEQ ID NO: 7, or (iii) a vector comprising (i) or (ii).

In some instances, the subject expresses an MHC encoded by an HLA-A*03:01 allele and is administered (i) a recombinant polypeptide comprising SEQ ID NO: 8, or (ii) a polynucleotide encoding a recombinant polypeptide comprising SEQ ID NO: 8, or (iii) a vector comprising (i) or (ii).

In some instances, the subject expresses an MHC encoded by an HLA-A*03:01 allele and is administered a drug product comprising (i) a first recombinant polypeptide comprising SEQ ID NO: 7, or (ii) a first polynucleotide encoding a recombinant polypeptide comprising SEQ ID NO: 7, or (iii) a first vector comprising (i) or (ii) and (iv) a second recombinant polypeptide comprising SEQ ID NO: 8, or (v) a second polynucleotide encoding a recombinant polypeptide comprising SEQ ID NO: 8 or (vi) a second vector comprising (iv) or (v).

In some instances, the subject expresses an MHC encoded by an HLA-A*03:01 allele and is administered (i) a recombinant polypeptide comprising SEQ ID NO: 9, or (ii) a polynucleotide encoding a recombinant polypeptide comprising SEQ ID NO: 9, or (iii) a vector comprising (i) or (ii).

In some instances, the subject expresses an MHC encoded by an HLA-B*07:02 allele and is administered (i) a recombinant polypeptide comprising SEQ ID NO: 10, or (ii) a polynucleotide encoding a recombinant polypeptide comprising SEQ ID NO: 10, or (iii) a vector comprising (i) or (ii).

In some instances, the subject expresses an MHC encoded by an HLA-A*03:01 allele and an MHC encoded by an HLA-B*07:02 allele and is administered a drug product comprising (i) a first recombinant polypeptide comprising SEQ ID NO: 9, or (ii) a first polynucleotide encoding a recombinant polypeptide comprising SEQ ID NO: 9, or (iii) a first vector comprising (i) or (ii) and (iv) a second recombinant polypeptide comprising SEQ ID NO: 10, or (v) a second polynucleotide encoding a recombinant polypeptide comprising SEQ ID NO: 10, or (vi) a second vector comprising (iv) or (v).

In some instances, the subject expresses an MHC encoded by an HLA-A*03:01 allele and is administered (i) a recombinant polypeptide comprising SEQ ID NO: 11, or (ii) a polynucleotide encoding a recombinant polypeptide comprising SEQ ID NO: 11, or (iii) a vector comprising (i) or (ii).

In some instances, the subject expresses an MHC encoded by an HLA-C*07:02 allele and is administered (i) a recombinant polypeptide comprising SEQ ID NO: 12, or (ii) a polynucleotide encoding a recombinant polypeptide comprising SEQ ID NO: 12, or (iii) a vector comprising (i) or (ii).

In some instances, the subject expresses an MHC encoded by an HLA-A*03:01 allele and an MHC encoded by an HLA-C*07:02 allele and is administered a drug product comprising (i) a first recombinant polypeptide comprising SEQ ID NO: 11, or (ii) a first polynucleotide encoding a recombinant polypeptide comprising SEQ ID NO: 11, or (iii) a first vector comprising (i) or (ii) and (iv) a second recombinant polypeptide comprising SEQ ID NO: 12, or (v) a second polynucleotide encoding a recombinant polypeptide comprising SEQ ID NO: 12, or (vi) a second vector comprising (iv) or (v).

In some instances, the subject expresses an MHC encoded by an HLA-A*03:01 allele and an MHC encoded by an HLA-B*07:02 allele, and an MHC encoded by an HLA-A*03:01 allele and is administered a first drug product comprising (i) a first recombinant polypeptide comprising SEQ ID NO: 7, or (ii) a first polynucleotide encoding a recombinant polypeptide comprising SEQ ID NO: 7, or (iii) a first vector comprising (i) or (ii) and (iv) a second recombinant polypeptide comprising SEQ ID NO: 8, or (v) a second polynucleotide encoding a recombinant polypeptide comprising SEQ ID NO: 8, or (vi) a second vector comprising (iv) or (iv) and a second drug product comprising (i) a third recombinant polypeptide comprising SEQ ID NO: 9, or (ii) a third polynucleotide encoding a recombinant polypeptide comprising SEQ ID NO: 9, or (iii) a third vector comprising (i) or (ii) and (iv) a fourth recombinant polypeptide comprising SEQ ID NO: 10, or (v) a fourth polynucleotide encoding a recombinant polypeptide comprising SEQ ID NO: 10, or (vi) a fourth vector comprising (iv) or (v).

In some instances, the subject expresses an MHC encoded by an HLA-A*03:01 allele, and an MHC encoded by an HLA-C*07:02 allele and is administered a first drug product comprising (i) a first recombinant polypeptide comprising SEQ ID NO: 7, or (ii) a first polynucleotide encoding a recombinant polypeptide comprising SEQ ID NO: 7, or (iii) a first vector comprising (i) or (ii) and a (iv) a second recombinant polypeptide comprising SEQ ID NO: 8, or (v) a second polynucleotide encoding a recombinant polypeptide comprising SEQ ID NO: 8, or (vi) a second vector comprising (iv) or (v) and a second drug product comprising drug product comprising (i) a third recombinant polypeptide comprising SEQ ID NO: 11, or (ii) a third polynucleotide encoding a recombinant polypeptide comprising SEQ ID NO: 11, or (iii) a third vector comprising (i) or (ii) and (iv) a fourth recombinant polypeptide comprising SEQ ID NO: 12, or (v) a fourth polynucleotide encoding a recombinant polypeptide comprising SEQ ID NO: 12, or (vi) a fourth vector comprising (iv) or (v).

In some instances, the subject expresses an MHC encoded by an HLA-A*03:01 allele, an MHC encoded by an HLA-B*07:02 allele, and an MHC encoded by an HLA-C*07:02 allele and is administered a first drug product comprising (i) a first recombinant polypeptide comprising SEQ ID NO: 9, or (ii) a first polynucleotide encoding a recombinant polypeptide comprising SEQ ID NO: 9, or (iii) a first vector comprising (i) or (ii) and a (iv) a second recombinant polypeptide comprising SEQ ID NO: 10, or (v) a second polynucleotide encoding a recombinant polypeptide comprising SEQ ID NO: 10, or (vi) a second vector comprising (iv) or (v) and a second drug product comprising (i) a third recombinant polypeptide comprising SEQ ID NO: 11, or (ii) a third polynucleotide encoding a recombinant polypeptide comprising SEQ ID NO: 11, or (iii) a third vector comprising (i) or (ii) and (iv) a fourth recombinant polypeptide comprising SEQ ID NO: 12, or (v) a fourth polynucleotide encoding a recombinant polypeptide comprising SEQ ID NO: 12, or (vi) a fourth vector comprising (iv) or (v). In some instances, the subject expresses an MHC encoded by an HLA-A*03:01 allele, an MHC encoded by an HLA-B*07:02 allele, and an MHC encoded by an HLA-C*07:02 allele, and is administered a first drug product comprising (i) a first recombinant polypeptide comprising SEQ ID NO: 7, or (ii) a first polynucleotide encoding a recombinant polypeptide comprising SEQ ID NO: 7, or (iii) a first vector comprising (i) or (ii) and (iv) a second recombinant polypeptide comprising SEQ ID NO: 8, or (v) a second polynucleotide encoding a recombinant polypeptide comprising SEQ ID NO: 8, or (vi) a second vector comprising (iv) or (iv); a second drug product comprising (i) a third recombinant polypeptide comprising SEQ ID NO: 9, or (ii) a third polynucleotide encoding a recombinant polypeptide comprising SEQ ID NO: 9, or (iii) a third vector comprising (i) or (ii) and (iv) a fourth recombinant polypeptide comprising SEQ ID NO: 10, or (v) a fourth polynucleotide encoding a recombinant polypeptide comprising SEQ ID NO: 10, or (vi) a fourth vector comprising (iv) or (v); and a third drug product comprising (i) a fifth recombinant polypeptide comprising SEQ ID NO: 11, or (ii) a fifth polynucleotide encoding a recombinant polypeptide comprising SEQ ID NO: 11, or (iii) a fifth vector comprising (i) or (ii) and (iv) a sixth recombinant polypeptide comprising SEQ ID NO: 12, or (v) a sixth polynucleotide encoding a recombinant polypeptide comprising SEQ ID NO: 12, or (vi) a sixth vector comprising (iv) or (v).

In some instances, the subject expresses an MHC encoded by an HLA-A*02:01 allele and is administered (i) a recombinant polypeptide comprising SEQ ID NO: 1, or (ii) a polynucleotide encoding a recombinant polypeptide comprising SEQ ID NO: 1, or (iii) a vector comprising (i) or (ii).

In some instances, the subject expresses an MHC encoded by an HLA-A*02:01 allele and is administered (i) a recombinant polypeptide comprising SEQ ID NO: 2, or (ii) a polynucleotide encoding a recombinant polypeptide comprising SEQ ID NO: 2, or (iii) a vector comprising (i) or (ii).

In some instances, the subject expresses an MHC encoded by an HLA-A*02:01 allele and is administered a drug product comprising (i) a first recombinant polypeptide comprising SEQ ID NO: 1, or (ii) a first polynucleotide encoding a recombinant polypeptide comprising SEQ ID NO: 1, or (iii) a first vector comprising (i) or (ii) and (iv) a second recombinant polypeptide comprising SEQ ID NO: 2, or (v) a second polynucleotide encoding a recombinant polypeptide comprising SEQ ID NO: 2 or (vi) a second vector comprising (iv) or (v).

In some instances, the subject expresses an MHC encoded by an HLA-B*07:02 allele and is administered (i) a recombinant polypeptide comprising SEQ ID NO: 3, or (ii) a polynucleotide encoding a recombinant polypeptide comprising SEQ ID NO: 3, or (iii) a vector comprising (i) or (ii).

In some instances, the subject expresses an MHC encoded by an HLA-B*07:02 allele and is administered (i) a recombinant polypeptide comprising SEQ ID NO: 4, or (ii) a polynucleotide encoding a recombinant polypeptide comprising SEQ ID NO: 4, or (iii) a vector comprising (i) or (ii).

In some instances, the subject expresses an MHC encoded by an HLA-B*07:02 allele and is administered a drug product comprising (i) a first recombinant polypeptide comprising SEQ ID NO: 3, or (ii) a first polynucleotide encoding a recombinant polypeptide comprising SEQ ID NO: 3, or (iii) a first vector comprising (i) or (ii) and (iv) a second recombinant polypeptide comprising SEQ ID NO: 4, or (v) a second polynucleotide encoding a recombinant polypeptide comprising SEQ ID NO: 4, or (vi) a second vector comprising (iv) or (v).

In some instances, the subject expresses an MHC encoded by an HLA-B*40:01 allele and is administered (i) a recombinant polypeptide comprising SEQ ID NO: 5, or (ii) a polynucleotide encoding a recombinant polypeptide comprising SEQ ID NO: 5, or (iii) a vector comprising (i) or (ii).

In some instances, the subject expresses an MHC encoded by an HLA-B*40:01 allele and is administered (i) a recombinant polypeptide comprising SEQ ID NO: 6, or (ii) a polynucleotide encoding a recombinant polypeptide comprising SEQ ID NO: 6, or (iii) a vector comprising (i) or (ii).

In some instances, the subject expresses an MHC encoded by an HLA-B*40:01 allele and is administered a drug product comprising (i) a first recombinant polypeptide comprising SEQ ID NO: 5, or (ii) a first polynucleotide encoding a recombinant polypeptide comprising SEQ ID NO: 5, or (iii) a first vector comprising (i) or (ii) and (iv) a second recombinant polypeptide comprising SEQ ID NO: 6, or (v) a second polynucleotide encoding a recombinant polypeptide comprising SEQ ID NO: 6, or (vi) a second vector comprising (iv) or (v).

In some instances, the subject expresses an MHC encoded by an HLA-A*02:01 allele and an MHC encoded by an HLA-B*07:02 allele, and is administered a first drug product comprising (i) a first recombinant polypeptide comprising SEQ ID NO: 1, or (ii) a first polynucleotide encoding a recombinant polypeptide comprising SEQ ID NO: 1, or (iii) a first vector comprising (i) or (ii) and (iv) a second recombinant polypeptide comprising SEQ ID NO: 2, or (v) a second polynucleotide encoding a recombinant polypeptide comprising SEQ ID NO: 2, or (vi) a second vector comprising (iv) or (iv) and a second drug product comprising (i) a third recombinant polypeptide comprising SEQ ID NO: 3, or (ii) a third polynucleotide encoding a recombinant polypeptide comprising SEQ ID NO: 3, or (iii) a third vector comprising (i) or (ii) and (iv) a fourth recombinant polypeptide comprising SEQ ID NO: 4, or (v) a fourth polynucleotide encoding a recombinant polypeptide comprising SEQ ID NO: 4, or (vi) a fourth vector comprising (iv) or (v).

In some instances, the subject expresses an MHC encoded by an HLA-A*02:01 allele and an MHC encoded by an HLA-B*40:01 allele, and is administered a first drug product comprising (i) a first recombinant polypeptide comprising SEQ ID NO: 1, or (ii) a first polynucleotide encoding a recombinant polypeptide comprising SEQ ID NO: 1, or (iii) a first vector comprising (i) or (ii) and a (iv) a second recombinant polypeptide comprising SEQ ID NO: 2, or (v) a second polynucleotide encoding a recombinant polypeptide comprising SEQ ID NO: 2, or (vi) a second vector comprising (iv) or (v) and a second drug product comprising drug product comprising (i) a third recombinant polypeptide comprising SEQ ID NO: 5, or (ii) a third polynucleotide encoding a recombinant polypeptide comprising SEQ ID NO: 5, or (iii) a third vector comprising (i) or (ii) and (iv) a fourth recombinant polypeptide comprising SEQ ID NO: 6, or (v) a fourth polynucleotide encoding a recombinant polypeptide comprising SEQ ID NO: 6, or (vi) a fourth vector comprising (iv) or (v).

In some instances, the subject expresses an MHC encoded by an HLA-B*07:02 allele and an MHC encoded by an HLA-B*40:01 allele, and is administered a first drug product comprising (i) a first recombinant polypeptide comprising SEQ ID NO: 3, or (ii) a first polynucleotide encoding a recombinant polypeptide comprising SEQ ID NO: 3, or (iii) a first vector comprising (i) or (ii) and a (iv) a second recombinant polypeptide comprising SEQ ID NO: 4, or (v) a second polynucleotide encoding a recombinant polypeptide comprising SEQ ID NO: 4, or (vi) a second vector comprising (iv) or (v) and a second drug product comprising (i) a third recombinant polypeptide comprising SEQ ID NO: 5, or (ii) a third polynucleotide encoding a recombinant polypeptide comprising SEQ ID NO: 5, or (iii) a third vector comprising (i) or (ii) and (iv) a fourth recombinant polypeptide comprising SEQ ID NO: 6, or (v) a fourth polynucleotide encoding a recombinant polypeptide comprising SEQ ID NO: 6, or (vi) a fourth vector comprising (iv) or (v).

In some instances, the subject expresses an MHC encoded by an HLA-A*02:01 allele an MHC encoded by an HLA-B*07:02 allele, and an MHC encoded by an HLA-B*40:01 allele, and is administered a first drug product comprising (i) a first recombinant polypeptide comprising SEQ ID NO: 1, or (ii) a first polynucleotide encoding a recombinant polypeptide comprising SEQ ID NO: 1, or (iii) a first vector comprising (i) or (ii) and (iv) a second recombinant polypeptide comprising SEQ ID NO: 2, or (v) a second polynucleotide encoding a recombinant polypeptide comprising SEQ ID NO: 2, or (vi) a second vector comprising (iv) or (iv); a second drug product comprising (i) a third recombinant polypeptide comprising SEQ ID NO: 3, or (ii) a third polynucleotide encoding a recombinant polypeptide comprising SEQ ID NO: 3, or (iii) a third vector comprising (i) or (ii) and (iv) a fourth recombinant polypeptide comprising SEQ ID NO: 4, or (v) a fourth polynucleotide encoding a recombinant polypeptide comprising SEQ ID NO: 4, or (vi) a fourth vector comprising (iv) or (v); and a third drug product comprising (i) a fifth recombinant polypeptide comprising SEQ ID NO: 5, or (ii) a fifth polynucleotide encoding a recombinant polypeptide comprising SEQ ID NO: 5, or (iii) a fifth vector comprising (i) or (ii) and (iv) a sixth recombinant polypeptide comprising SEQ ID NO: 6, or (v) a sixth polynucleotide encoding a recombinant polypeptide comprising SEQ ID NO: 6, or (vi) a sixth vector comprising (iv) or (v).

The disclosure furthermore relates to a method of treating or preventing a cancer, comprising administering a therapeutically effective amount of the vector or composition according to the disclosure to a subject in need thereof. In some embodiments, the disclosure relates to the use of a vector or composition described herein in the manufacture of a medicament for the treatment or prevention of cancer. In some embodiments, the disclosure relates to the use of a vector or composition described herein in the treatment or prevention of cancer.

In one embodiment, the treatment can include administering to the subject a composition. In some cases, the composition comprises a recombinant polypeptide. In some cases, the recombinant polypeptide can include a cancer-specific CD8+ T cell epitope. In some cases, the composition can include a polynucleotide encoding a recombinant polypeptide. In some instances, the recombinant polypeptide can include a cancer-specific CD8+ T cell epitope. In some cases, the composition can include antigen presenting cells (APCs). In some instances, the APCs can include a recombinant polypeptide. In some aspects, the recombinant polypeptide can include a cancer-specific CD8+ T cell epitope. In some instances, the APCs can include a polynucleotide encoding a recombinant polypeptide. In some aspects, the polynucleotide encoding a recombinant polypeptide can include a cancer-specific CD8+ T cell epitope. In some cases, the composition can include T cells stimulated with APCs. In some instances, the APCs can include a recombinant polypeptide. In some aspects, the recombinant polypeptide can include a cancer-specific CD8+ T cell epitope. In some instances, the APCs can include a polynucleotide encoding a recombinant polypeptide. In some aspects, the polynucleotide encoding a recombinant polypeptide can include a cancer-specific CD8+ T cell epitope.

In some embodiments, a subject can express an MHC encoded by an HLA-A*02:01 allele. In some embodiments, the subject can express an MHC encoded by an HLA-A*03:01 allele. In some embodiments, the subject can express an MHC encoded by an HLA-A*31:01 allele. In some embodiments, the subject can express an MHC encoded by an HLA-B*40:01 allele. In some embodiments, the subject can express an MHC encoded by an HLA-B*44:02 allele. In some embodiments, the subject can express an MHC encoded by an HLA-B*18:01 allele. In some embodiments, the subject can express an MHC encoded by an HLA-B*07:02 allele. In some embodiments, the subject can express an MHC encoded by an HLA-A*15:01 allele. In some embodiments, the subject can express an MHC encoded by an HLA-A*03:04 allele. In some embodiments, the subject can express an MHC encoded by an HLA-C*07:02 allele. In some embodiments, the subject can express an MHC encoded by an HLA-B*07:02 allele. In some embodiments, the subject can express an MHC encoded by an HLA-B*08:01 allele. In some embodiments, the subject can express an MHC encoded by an HLA-C*07:01 allele. In some embodiments, the subject can express an MHC encoded by an HLA-B*35:01 allele. In some embodiments, the subject can express an MHC encoded by an HLA-A*02:05 allele.

In some embodiments, the subject can express an MHC encoded by an HLA-A*02:01 allele, an HLA-A*03:01 allele, an HLA-A*11:01 allele, an HLA-A*31:01 allele, an HLA-B*40:01 allele, an HLA-B*40:02 allele, an HLA-B*44:02 allele, an HLA-B*18:01 allele, an HLA-B*07:02 allele, an HLA-B*35:03 allele, an HLA-A*15:01 allele, an HLA-A*03:04 allele, an HLA-C*07:02 allele, an HLA-B*03:04 allele, an HLA-B*07:02 allele, an HLA-B*08:01 allele, an HLA-C*07:01 allele, an HLA-B*35:01 allele, an HLA-A*02:05 allele, or any combination, e.g., combination of two, e.g., combination of three thereof.

The present disclosure also provides a method of inducing an inflating memory CD8+ T cell response, comprising the step of; administering a therapeutically effective amount of the recombinant polypeptides, polynucleotides, vectors, cells (e.g., APCs, T cells), composition, pharmaceutical compositions, or formulations according to the disclosure, to a subject in need thereof optionally wherein the CD8+ T cells are characterized by one or more markers selected from the group comprising CX3CR1+, KLRG-1+, CD44+ and CD62L−.

In some embodiments, the CD8+ T cells are characterized by the phenotype CX3CR1+, KLRG-1+, CD44+, CCR7—, CD45RA+/−, and CD62L−. In some embodiments, the CD8+ T cells are further characterized by the phenotype CX3CR1+, KLRG-1+, CD44+, CD62L−, CCR7−, CD45RA+/−, CD27 (low), and CD127 (low).

In some cases, the recombinant polypeptides, polynucleotides, vectors, cells (e.g., APCs, T cells), composition, pharmaceutical compositions, or formulations described herein can be used for treating a cancer selected from colorectal cancer, prostate cancer, esophageal cancer, liver cancer, renal cancer, lung cancer, bladder cancer, breast cancer, pancreatic cancer, brain cancer, hepatocellular cancer, lymphoma, leukaemia, gastric cancer, cervical cancer, ovarian cancer, thyroid cancer, melanoma, carcinoma, head and neck cancer, skin cancer, nasopharyngeal cancer, Epstein Barr driven cancers, Human Papilloma virus driven cancers or soft tissue sarcoma. In some cases, the composition can be used for treating esophageal cancer. In some embodiments, the cancer is a primary cancer. In some embodiments, the cancer is a relapsed and/or a refractory cancer. In some embodiments, the recombinant polypeptides, polynucleotides, vectors, cells (e.g., APCs, T cells), composition, pharmaceutical compositions, or formulations described herein can be used for preventing formation of a secondary tumor. In some embodiments, the recombinant polypeptides, polynucleotides, vectors, cells (e.g., APCs, T cells), composition, pharmaceutical compositions, or formulations described herein can be used for preventing metastasis.

The recombinant polypeptides, polynucleotides, vectors, cells (e.g., APCs, T cells), compositions, pharmaceutical compositions, or formulations described herein as described herein may be administered by any convenient route. The recombinant polypeptides, polynucleotides, vectors, cells (e.g., APCs, T cells), compositions, pharmaceutical compositions, or formulations described herein may be administered by any convenient route, including but not limited to oral, topical, parenteral, sublingual, rectal, vaginal, ocular, intranasal, pulmonary, intradermal, intravitreal, intramuscular, intraperitoneal, intravenous, subcutaneous, intracerebral, transdermal, transmucosal, subcutaneous, intra-arterial, or by inhalation. Parenteral administration includes, for example, intravenous, intramuscular, intraarterial, intraperitoneal, intranasal, rectal, intravesical, intradermal, topical, or subcutaneous administration. In some embodiments, the vector or composition is administered intravenously or intramuscularly. Compositions can take the form of one or more dosage units.

In specific embodiments, it may be desirable to administer the recombinant polypeptides, polynucleotides, vectors, cells (e.g., APCs, T cells), compositions, pharmaceutical compositions, or formulations described herein locally to the area in need of treatment such at as the site of a tumor. In another embodiment it may be desirable to administer the recombinant polypeptides, polynucleotides, vectors, cells (e.g., APCs, T cells), compositions, pharmaceutical compositions, or formulations described herein by intravenous injection or infusion. The amount of the recombinant polypeptides, polynucleotides, vectors, or cells (e.g., APCs, T cells) of the present disclosure that is effective/active in the treatment of a particular disorder or condition will depend on the nature of the disorder or condition and can be determined by standard clinical techniques. In addition, in vitro or in vivo assays can optionally be employed to help identify optimal dosage ranges. The precise dose to be employed in the compositions will also depend on the route of administration, and the seriousness of the disease or disorder, and should be decided according to the judgment of the practitioner and each patient's circumstances.

The compositions, pharmaceutical compositions, or formulations described herein comprise an effective amount of the recombinant polypeptides, polynucleotides, vectors, or cells (e.g., APCs, T cells) according to the present disclosure such that a suitable dosage will be obtained. The correct dosage of the compounds will vary according to the particular formulation, the mode of administration, and its particular site, host and the disease being treated. Other factors like age, body weight, sex, diet, time of administration, rate of excretion, condition of the host, drug combinations, reaction sensitivities and severity of the disease shall be taken into account. Administration can be carried out continuously or periodically.

The recombinant polypeptides, polynucleotides, vectors, cells (e.g., APCs, T cells), compositions, pharmaceutical compositions, or formulations described herein may be administered as a single dose. The dose may be provided in a prophylactic setting or a therapeutic setting. In some embodiments, the single dose may be provided as a single dose unit further comprising one or more additional active ingredients, pharmaceutically acceptable carrier, diluent, excipient or adjuvant.

The recombinant polypeptides, polynucleotides, vectors, cells (e.g., APCs, T cells), compositions, pharmaceutical compositions, or formulations described herein may be administered as multiple doses. Wherein multiple doses are administered, one or more may be administered prophylactically or one or more may be administered therapeutically. Where multiple doses are administered, one or more may be administered prophylactically and one or more may be administered therapeutically.

Dose delays and/or dose reductions and schedule adjustments are performed as needed depending on individual patient tolerance to treatments.

In some embodiments, a single dose of the recombinant polypeptides, polynucleotides, vectors, cells (e.g., APCs, T cells), compositions, pharmaceutical compositions, or formulations described herein is administered. In some embodiments, multiple doses, (e.g., 2, 3, 4, 5, 6, 7, 8, 9, 10, or more) of the recombinant polypeptides, polynucleotides, vectors, cells (e.g., APCs, T cells), compositions, pharmaceutical compositions, or formulations described herein are administered. In some embodiments, the first dose is administered 1 day, 2 day, 3 days, 4 days, 5 days, 6 days, 7 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, or 15 weeks after the diagnostic biopsy. In some embodiments, the second dose is administered after 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks after the first dose. In some embodiments, the second dose is administered after 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, or 28 weeks after the diagnostic biopsy. In some embodiments, the third dose is administered after 1 month, 2 months, 3 months, 4 months, 5 months, 6 months or longer after the first dose.

In some embodiments, the first and the second dose comprise identical recombinant polypeptides, polynucleotides, vectors, cells (e.g., APCs, T cells), compositions, pharmaceutical compositions, or formulations described herein. In some embodiments, the first and the second dose comprise different recombinant polypeptides, polynucleotides, vectors, cells (e.g., APCs, T cells), compositions, pharmaceutical compositions, or formulations described herein. In some embodiments, the first and the second dose comprise different recombinant polypeptides, polynucleotides, vectors, cells (e.g., APCs, T cells), compositions, pharmaceutical compositions, or formulations wherein the recombinant polypeptides, polynucleotides, vectors, cells (e.g., APCs, T cells), compositions, pharmaceutical compositions, or formulations comprise different epitopes, e.g., T cell epitopes, e.g., cancer-specific CD8+ and/or CD4+ epitopes, e.g., cancer-specific CD8+ epitopes. In some embodiments, a sample (e.g., tumor sample) from the subject is analyzed for presence of T cell epitopes, e.g., cancer-specific CD8+ and/or CD4+ T cell epitopes, e.g., cancer-specific CD8+ T cell epitopes after administration of the first dose. In some embodiments, the composition of the first dose and the second dose is identical. In some embodiments, the composition of the second dose is altered based on the analysis result, e.g., to specifically target the T cell epitopes, e.g., cancer-specific CD8+ and/or CD4+ T cell epitopes, e.g., cancer-specific CD8+ T cell epitopes present in the subject sample (e.g., tumor sample).

In some embodiments, the subject is administered a second therapeutic agent before administering the first dose. In some embodiments, the second therapeutic agent is a chemotherapeutic agent. In some embodiments, the second therapeutic agent is a combination of multiple (e.g., 1, 2, 3, or 4) chemotherapeutic agents. In some embodiments, the second therapeutic agent is selected from one or more of fluorouracil, leucovorin, oxaliplatin, and docetaxel. In some embodiments, the second therapeutic agent can optionally comprise leucovorin.

In some embodiments, the second therapeutic agent is administered 1 day, 2 day, 3 days, 4 days, 5 days, 6 days, 7 days, 1 week, 2 weeks, 3 weeks, 4 weeks, 5 weeks, 6 weeks, 7 weeks, 8 weeks, 9 weeks, or 10 weeks before the first dose. In some embodiments, the second therapeutic agent is administered 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, or 28 weeks before the second dose.

In some embodiments, a second dose of the second therapeutic agent therapeutic agent is administered. In some embodiments, the second dose of the second therapeutic agent is administered after administering the first dose. In some embodiments, the second dose of the second therapeutic agent therapeutic agent is administered before administering the second dose. In some embodiments, the second dose of the second therapeutic agent is administered after administering the second dose. In some embodiments, the second dose of the second therapeutic agent is administered after 16 weeks, 17 weeks, 18 weeks, 19 weeks, 20 weeks, 21 weeks, 22 weeks, 23 weeks, or 24 weeks after the first dose. In some embodiments, the second dose of the second therapeutic agent therapeutic agent is administered 1 week, 2 week, 3 weeks, 4 weeks, 5 weeks, 6 weeks, 7 weeks, 8 weeks, 9 weeks, or 10 weeks before the second dose. In some embodiments, the second dose of the second therapeutic agent therapeutic agent is administered 1 week, 2 week, 3 weeks, 4 weeks, 5 weeks, 6 weeks, 7 weeks, 8 weeks, 9 weeks, or 10 weeks after the second dose. In some embodiments the first dose of the second therapeutic agent is administered at once. In some embodiments the second dose of the second therapeutic agent is administered at once. In some embodiments, the second dose of the second therapeutic agent administration is spread over 1 days, 2 days, 3 days, 4 days, 5 days, 6 days, 7 days, 1 week, 2 weeks, 3 weeks, 4 weeks, 5 weeks, 6 weeks, 7 weeks, or 8 weeks.

In some embodiments, the recombinant polypeptides, polynucleotides, vectors, cells (e.g., APCs, T cells), compositions, pharmaceutical compositions, or formulations described herein can be administer in a prime-boost regime. In some embodiments, the first dose is administered to prime the immune response to a target antigen (prime dose). In some embodiments, the second dose (boost dose) is administered after the first dose has been administered to prime the immune response to the target antigen. In some embodiments, the boost dose elicits an equal or greater measurable immune response compared to the response elicited by administration of the prime dose. In some embodiments, the prime dose elicits an immune response to the target antigen, and the boost dose elicits an equal or greater measurable immune response compared to the response elicited by administration of the prime dose. An “effective amount” of a “prime dose” refers to the amount of target antigen (e.g., T cell epitope, e.g., a cancer-specific CD8+ epitope described herein) which elicits a measurable immune response in a subject as compared to the immune response in the mammalian subject in the absence of administration of the antigen. An “effective amount” of a “boost dose” refers to the amount of antigen (e.g., T cell epitope, e.g., a cancer-specific CD8+ epitope described herein) which elicits an immune response to the target antigen upon administration to a subject which previously has been administered a prime dose of the target antigen.

In some embodiments, the prime dose is administered intramuscularly. In some embodiments, the prime dose is administered intravenously. In some embodiments, the boost dose is administered intramuscularly. In some embodiments, the boost dose is administered intravenously. In some embodiments, the prime dose is administered intramuscularly, and the boost dose is administered intramuscularly. In some embodiments, the prime dose is administered intravenously, and the boost dose is administered intravenously. In some embodiments, the prime dose is administered intravenously, and the boost dose is administered intramuscularly. In some embodiments, prime dose is administered intramuscularly, and the boost dose is administered intravenously.

In some embodiments, the prime dose comprises at least 1, at least 2, at least 3, at least 4, at least 5, or no more than 6 vectors. In some embodiments, the prime dose comprises 2×10¹⁰ viral particles per dose. In some embodiments, the prime dose comprises a first vector at a concentration of 1×10¹⁰, 5×10¹⁰, or 1×10¹¹ viral particles, a second vector at a concentration of 1×10¹⁰, 5×10¹⁰, or 1×10¹¹ viral particles, a third vector at a concentration of 1×10¹⁰, 5×10¹⁰, or 1×10¹¹ viral particles, a fourth vector at a concentration of 1×10¹⁰, 5×10¹⁰, or 1×10¹¹ viral particles, a fifth vector at a concentration of 1×10¹⁰, 5×10¹⁰, or 1×10¹¹ viral particles, and/or a sixth vector at a concentration of 1×10¹⁰, 5×10¹⁰, or 1×10¹¹ viral particles, optionally wherein the total concentration of viral particle in the dose is no more than 3×10¹¹ viral particles per dose. In some embodiments, the prime dose comprises a first vector at a concentration of 1×10¹¹, 2×10¹¹, or 3×10¹¹ viral particle, a second vector at a concentration of 1×10¹¹, 2×10¹¹, or 3×10¹¹ viral particle, a third vector at a concentration of 1×10¹¹, 2×10¹¹, or 3×10¹¹ viral particle, a fourth vector at a concentration of 1×10¹¹, 2×10¹¹, or 3×10¹¹ viral particle, a fifth vector at a concentration of 1×10¹¹, 2×10¹¹, or 3×10¹¹ viral particle, and/or a sixth vector at a concentration of 1×10¹¹, 2×10¹¹, or 3×10¹¹ viral particle, wherein the total concentration of viral particle in the dose is no more than 3×10¹¹ viral particle per dose.

In some embodiments, the boost dose comprises at least 1, at least 2, at least 3, at least 4, at least 5, or no more than 6 vectors. In some embodiments, the boost dose comprises 2×10¹⁰ viral particles per dose. In some embodiments, the boost dose comprises a first vector at a concentration of 1×10¹⁰, 5×10¹⁰, or 1×10¹¹ viral particles, a second vector at a concentration of 1×10¹⁰, 5×10¹⁰, or 1×10¹¹ viral particles, a third vector at a concentration of 1×10¹⁰, 5×10¹⁰, or 1×10¹¹ viral particles, a fourth vector at a concentration of 1×10¹⁰, 5×10¹⁰, or 1×10¹¹ viral particles, a fifth vector at a concentration of 1×10¹⁰, 5×10¹⁰, or 1×10¹¹ viral particles, and/or a sixth vector at a concentration of 1×10¹⁰, 5×10¹⁰, or 1×10¹¹ viral particles, optionally wherein the total concentration of viral particle in the dose is no more than 3×10¹¹ viral particles per dose. In some embodiments, the boost dose comprises a first vector at a concentration of 1×10¹¹, 2×10¹¹, or 3×10¹¹ viral particle, a second vector at a concentration of 1×10¹¹, 2×10¹¹, or 3×10¹¹ viral particle, a third vector at a concentration of 1×10¹¹, 2×10¹¹, or 3×10¹¹ viral particle, a fourth vector at a concentration of 1×10¹¹, 2×10¹¹, or 3×10¹¹ viral particle, a fifth vector at a concentration of 1×10¹¹, 2×10¹¹, or 3×10¹¹ viral particle, and/or a sixth vector at a concentration of 1×10¹¹, 2×10¹¹, or 3×10¹¹ viral particle, wherein the total concentration of viral particle in the dose is no more than 3×10¹¹ viral particle per dose.

In some embodiments, the prime dose is administered 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, or 20 weeks after the diagnostic biopsy. In some embodiments, the boost dose is administered after 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks after the prime dose. In some embodiments, the boost dose is administered 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, or 28 weeks after the diagnostic biopsy. In some embodiments, a second boost dose is administered after 1 month, 2 month, 3 month, 4 month, 5 month, 6 months or longer after the prime dose. In some embodiments, a second boost dose is administered after 1 month, 2 month, 3 month, 4 month, 5 month, 6 months or longer after the first boost dose. In some embodiments, a sample (e.g., tumor sample) from the subject is analyzed for presence of T cell epitopes, e.g., cancer-specific CD8+ T cell epitopes after administration of the prime dose. In some embodiments, the composition of the prime dose and the boost dose is identical. In some embodiments, the composition of the boost dose is altered based on the analysis result, e.g., to specifically target the T cell epitopes, e.g., cancer-specific CD8+ T cell epitopes present in the subject sample (e.g., tumor sample).

Administration of the boost dose can be initiated when a population of antigen-specific immune cells has expanded (increased in number) to at least 20% the maximal number of antigen-specific immune cells that is eventually attained; to at least 30%; to at least 40%; to at least 50%; to at least 60%; to at least 70%; to at least 80%; to at least 90%; to at least 95%; to at least 99% the maximal number of antigen-specific immune cells that is eventually attained. Additional schedules of prime-boost dosing are available, for example, the boost dose can be initiated when the population of antigen-specific cells has contracted to under 90% the maximal number of antigen-specific cells; under 80%; under 70%; under 60%; under 50%; under 40%; under 30%; under 20%; under 10%; under 5%; under 1.0%; under 0.5%; under 0.1%; under 0.05%; or under 0.01% the maximal number of antigen-specific immune cells. The antigen-specific cells can be identified as specific for an empty vector, or specific for a cancer-specific CD8+ T cell epitope expressed by a polynucleotide contained in the vector.

In some embodiments, the boost dose can enhance the prime dose immune response by at least two-fold, at times between about three- and five-fold or five-fold to ten-fold, or from ten-fold to 100-fold or greater. In some embodiments, the prime dose and boost dose have a synergistic effect on the immune response. In some embodiments, the enhanced immune response will include a T-cell response, e.g., a CD8+ T-cell response. In some embodiments, the prime dose and boost dose will break a subject's tolerogenic state towards a target cancer-specific CD8+ T cell epitope disclosed herein.

In some embodiments, the vector, e.g., the viral vector in the therapeutic composition can be administered in a dose, or dosages, where each dose comprises between 1×10³ and 7.5×10¹³ viral particles per 70 kg body weight; and so on.

In some embodiments, the vector, e.g., the viral vector in the therapeutic composition can be administered in a dose, or dosages, where each dose comprises between 1×10² and 5×10¹² viral particles per 70 kg body weight; between 1×10³ and 5×1¹¹ viral particles per 70 kg body weight; between 1×10⁴ and 5×10¹¹ viral particles per 70 kg body weight; between 1×10⁵ and 5×10¹¹ viral particles per 70 kg body weight; between 1×10⁶ and 5×10¹¹ viral particles per 70 kg body weight; between 1×10⁷ and 5×10¹¹ viral particles per 70 kg body weight; between 1×10⁸ and 5×10¹¹ viral particles per 70 kg body weight; between 1×10⁹ and 5×10¹¹ viral particles per 70 kg body weight; between 1×10¹⁰ and 5×10¹¹ viral particles per 70 kg body weight; between 1×10¹⁰ and 5×10¹¹ viral particles per 70 kg body weight; 2×10² and 5×10¹² viral particles per 70 kg body weight; between 2×10³ and 5×10¹¹ viral particles per 70 kg body weight; between 2×10⁴ and 5×10¹¹viral particles per 70 kg body weight; between 2×10⁵ and 5×10¹¹ viral particles per 70 kg body weight; between 2×10⁶ and 5×10¹¹ viral particles per 70 kg body weight; between 2×10⁷ and 5×10¹¹viral particles per 70 kg body weight; between 2×10⁸ and 5×10¹¹ viral particles per 70 kg body weight; between 2×10⁹ and 5×10¹¹ viral particles per 70 kg body weight; between 2×10¹⁰ and 5×10¹¹ viral particles per 70 kg body weight; between 2×10¹¹ and 5×10¹¹ viral particles per 70 kg body weight, wet weight; 1×10² and 5×10¹² viral particles per 70 kg body weight; between 1×10³ and 5×10¹¹ viral particles per 70 kg body weight; between 1×10⁴ and 4×10¹¹ viral particles per 70 kg body weight; between 1×10⁵ and 4×10¹¹ viral particles per 70 kg body weight; between 1×10⁶ and 4×10¹¹ viral particles per 70 kg body weight; between 1×10⁷ and 4×10¹¹ viral particles per 70 kg body weight; between 1×10⁸ and 4×10¹¹ viral particles per 70 kg body weight; between 1×10⁹ and 4×10¹¹ viral particles per 70 kg body weight; between 1×10¹⁰ and 4×10¹¹ viral particles per 70 kg body weight; between 1×10¹¹ and 4×10¹¹ viral particles per 70 kg body weight; 1×10⁵ and 3×10¹⁵ viral particles per 70 kg body weight; between 1×10⁶ and 3×10¹⁵ viral particles per 70 kg body weight; between 1×10⁷ and 3×10¹⁵ viral particles per 70 kg body weight; between 1×10⁸ and 3×10¹⁵ viral particles per 70 kg body weight; between 1×10⁸ and 3×10¹⁵ viral particles per 70 kg body weight; between 1×10⁹ and 3×10¹⁵ viral particles per 70 kg body weight; between 1×10⁹ and 3×10¹⁵ viral particles per 70 kg body weight; between 1×10¹⁰ and 3×10¹⁵ viral particles per 70 kg body weight; between 1×10¹¹ and 3×10¹⁵ viral particles per 70 kg body weight; 1×10⁵ and 3×10¹¹ viral particles per 70 kg body weight; between 1×10⁶ and 3×10¹¹ viral particles per 70 kg body weight; between 1×10⁷ and 3×10¹¹viral particles per 70 kg body weight; between 1×10⁸ and 3×10¹¹ viral particles per 70 kg body weight; between 1×10⁸ and 3×10¹¹ viral particles per 70 kg body weight; between 1×10⁹ and 3×10¹¹ viral particles per 70 kg body weight; between 1×10⁹ and 3×10¹¹viral particles per 70 kg body weight; between 1×10¹⁰ and 3×10¹¹ viral particles per 70 kg body weight; or between 1×10¹¹ and 3×10¹¹viral particles per 70 kg body weight; and so on.

In some embodiments, a vaccine comprise a modality. A modality can comprise an inactivated vaccine, a live-attneuated vaccine, a messenger RNA (mRNA) vaccine, a peptide vaccine, a polysaccharide-based vaccine, a toxoid vaccine, or a viral (e.g., viral vector) vaccine. In some cases, a vaccine may be a viral vaccine, a mRNA vaccine, or a peptide vaccine. In some cases, a prime dose (e.g., first dose) and a boost dose (e.g., second dose) described herein may comprise the same vaccine modality. In some embodiments, administration of the prime dose (e.g., first dose) and boost dose (e.g., second dose) of the same modality can be referred to as homologous vaccine administration (e.g., homologous prime-boost). In some cases, a prime dose (e.g., first dose) and a boost dose (e.g., second dose) described herein may comprise different vaccine modalities. In some embodiments, administration of the prime dose (e.g., first dose) and boost dose (e.g., second dose) of different modalities can be referred to as heterologous vaccine administration (e.g., heterologous prime-boost).

In some cases, a prime dose (e.g., first dose) may be a viral (e.g., an adenoviral) vaccine and a boost dose (e.g., second dose) may be a viral (e.g., an adenoviral) vaccine. In some cases, a prime dose (e.g., first dose) may be a viral (e.g., an adenoviral) vaccine and a boost dose (e.g., second dose) may be a mRNA vaccine. In some cases, a prime dose (e.g., first dose) may be a viral (e.g., an adenoviral) vaccine and a boost dose (e.g., second dose) may be a peptide-based vaccine. In some cases, a prime dose (e.g., first dose) may be a mRNA vaccine and a boost dose (e.g., second dose) may be a mRNA vaccine. In some cases, a prime dose (e.g., first dose) may be a mRNA vaccine and a boost dose (e.g., second dose) may be a peptide-based vaccine. In some cases, a prime dose (e.g., first dose) may be a mRNA vaccine and a boost dose (e.g., second dose) may be a viral (e.g., adenoviral) vaccine. In some cases, a prime dose (e.g., first dose) may be a peptide-based vaccine and a boost dose (e.g., second dose) may be a viral (e.g., adenoviral) vaccine. In some cases, a prime dose (e.g., first dose) may be a peptide-based vaccine and a boost dose (e.g., second dose) may be a mRNA vaccine. In some cases, a prime dose (e.g., first dose) may be a peptide-based vaccine and a boost dose (e.g., second dose) may be a peptide-based vaccine.

In some cases, homologous administration may result in a greater T cell response, as measured by ELISpot assay. An ELISpot assay can quantify a number of spot-forming cells (e.g., cytokine and/or antibody secreting cells). A greater number of spot-forming cells (SFCs) may indicate increased cytokine production and/or a greater T cell response. A number of SFCs may be measured following a prime dose (e.g., first dose), a boost dose (e.g., second dose), or any combination thereof. In some cases, SFCs from a sample may be measured on a day of prime dose (e.g., first dose) administration. In some cases, SFCs from a sample may be measured at least about, at most about, or about 1, 2, 3, 4, 5, 6, 7, 8, 9, 10, 15, 20, 25, 30, 35, 40, 45, 50, 75, 100, 120, 150, or 200 days following a prime dose (e.g., first dose) administration, or a range between any of those two values. In some cases, SFCs from a sample may be measured on a day of boost dose (e.g., second dose) administration. In some cases, SFCs from a sample may be measured at least about, at most about, or about 1, 2, 3, 4, 5, 6, 7, 8, 9, 10, 15, 20, 25, 30, 35, 40, 45, 50, 75, 100, 120, 150, or 200 days following a boost dose (e.g., second dose) administration, or a range between any of those two values.

In some embodiments, homologous administration of prime dose and boost dose (e.g., viral-viral administration, mRNA-mRNA administration, or peptide-peptide administration) can result in at least about 1, at least about 5, at least about 10, at least about 20, at least about 50, at least about 100, at least about 250, at least about 500, at least about 750, at least about 1,000, at least about 2,000, at least about 3,000, at least about 4,000, at least about 5,000, at least about 6,000, at least about 7,000, at least about 8,000, at least about 9,000, at least about 10,000, at least about 25,000, at least about 50,000, or greater than about 50,000 SFCs per million peripheral blood mononuclear cells (PBMCs) at a timepoint post prime dose vaccination. In some embodiments, homologous administration of prime dose and boost dose (e.g., viral-viral administration, mRNA-mRNA administration, or peptide-peptide administration) can result in at most about 50,000, at most about 25,000, at most about 10,000, at most about 9,000, at most about 8,000, at most about 7,000, at most about 6,000, at most about 5,000, at most about 4,000, at most about 3,000, at most about 2,000, at most about 1,000, at most about 750, at most about 500, at most about 250, at most about 100, at most about 50, at most about 20, at most about 10, at most about 15, at most about 1, or less than about 1 SFC(s) per million PBMCs at a timepoint post prime dose vaccination. In some embodiments, homologous administration of prime dose and boost dose (e.g., viral-viral administration, mRNA-mRNA administration, or peptide-peptide administration) can result in from about 1 to about 50,000 SFCs per million peripheral blood mononuclear cells (PBMCs) at a timepoint post prime dose vaccination. In some embodiments, homologous administration of prime dose and boost dose (e.g., viral-viral administration, mRNA-mRNA administration, or peptide-peptide administration) can result in from about 1 to about 5, about 1 to about 10, about 1 to about 50, about 1 to about 100, about 1 to about 500, about 1 to about 1,000, about 1 to about 2,500, about 1 to about 5,000, about 1 to about 10,000, about 1 to about 25,000, about 1 to about 50,000, about 5 to about 10, about 5 to about 50, about 5 to about 100, about 5 to about 500, about 5 to about 1,000, about 5 to about 2,500, about 5 to about 5,000, about 5 to about 10,000, about 5 to about 25,000, about 5 to about 50,000, about 10 to about 50, about 10 to about 100, about 10 to about 500, about 10 to about 1,000, about 10 to about 2,500, about 10 to about 5,000, about 10 to about 10,000, about 10 to about 25,000, about 10 to about 50,000, about 50 to about 100, about 50 to about 500, about 50 to about 1,000, about 50 to about 2,500, about 50 to about 5,000, about 50 to about 10,000, about 50 to about 25,000, about 50 to about 50,000, about 100 to about 500, about 100 to about 1,000, about 100 to about 2,500, about 100 to about 5,000, about 100 to about 10,000, about 100 to about 25,000, about 100 to about 50,000, about 500 to about 1,000, about 500 to about 2,500, about 500 to about 5,000, about 500 to about 10,000, about 500 to about 25,000, about 500 to about 50,000, about 1,000 to about 2,500, about 1,000 to about 5,000, about 1,000 to about 10,000, about 1,000 to about 25,000, about 1,000 to about 50,000, about 2,500 to about 5,000, about 2,500 to about 10,000, about 2,500 to about 25,000, about 2,500 to about 50,000, about 5,000 to about 10,000, about 5,000 to about 25,000, about 5,000 to about 50,000, about 10,000 to about 25,000, about 10,000 to about 50,000, or about 25,000 to about 50,000 SFCs per million peripheral blood mononuclear cells (PBMCs) at a timepoint post prime dose vaccination.

In some embodiments, homologous administration of prime dose and boost dose (e.g., viral-viral administration, mRNA-mRNA administration, or peptide-peptide administration) can result in at least about 1, at least about 5, at least about 10, at least about 20, at least about 50, at least about 100, at least about 250, at least about 500, at least about 750, at least about 1,000, at least about 2,000, at least about 3,000, at least about 4,000, at least about 5,000, at least about 6,000, at least about 7,000, at least about 8,000, at least about 9,000, at least about 10,000, at least about 25,000, at least about 50,000, or greater than about 50,000 SFCs per million peripheral blood mononuclear cells (PBMCs) at a timepoint post boost dose vaccination. In some embodiments, homologous administration of prime dose and boost dose (e.g., viral-viral administration, mRNA-mRNA administration, or peptide-peptide administration) can result in at most about 50,000, at most about 25,000, at most about 10,000, at most about 9,000, at most about 8,000, at most about 7,000, at most about 6,000, at most about 5,000, at most about 4,000, at most about 3,000, at most about 2,000, at most about 1,000, at most about 750, at most about 500, at most about 250, at most about 100, at most about 50, at most about 20, at most about 10, at most about 15, at most about 1, or less than about 1 SFC(s) per million PBMCs at a timepoint post boost dose vaccination. In some embodiments, homologous administration of prime dose and boost dose (e.g., viral-viral administration, mRNA-mRNA administration, or peptide-peptide administration) can result in from about 1 to about 50,000 SFCs per million peripheral blood mononuclear cells (PBMCs) at a timepoint post prime dose vaccination. In some embodiments, homologous administration of prime dose and boost dose (e.g., viral-viral administration, mRNA-mRNA administration, or peptide-peptide administration) can result in from about 1 to about 5, about 1 to about 10, about 1 to about 50, about 1 to about 100, about 1 to about 500, about 1 to about 1,000, about 1 to about 2,500, about 1 to about 5,000, about 1 to about 10,000, about 1 to about 25,000, about 1 to about 50,000, about 5 to about 10, about 5 to about 50, about 5 to about 100, about 5 to about 500, about 5 to about 1,000, about 5 to about 2,500, about 5 to about 5,000, about 5 to about 10,000, about 5 to about 25,000, about 5 to about 50,000, about 10 to about 50, about 10 to about 100, about 10 to about 500, about 10 to about 1,000, about 10 to about 2,500, about 10 to about 5,000, about 10 to about 10,000, about 10 to about 25,000, about 10 to about 50,000, about 50 to about 100, about 50 to about 500, about 50 to about 1,000, about 50 to about 2,500, about 50 to about 5,000, about 50 to about 10,000, about 50 to about 25,000, about 50 to about 50,000, about 100 to about 500, about 100 to about 1,000, about 100 to about 2,500, about 100 to about 5,000, about 100 to about 10,000, about 100 to about 25,000, about 100 to about 50,000, about 500 to about 1,000, about 500 to about 2,500, about 500 to about 5,000, about 500 to about 10,000, about 500 to about 25,000, about 500 to about 50,000, about 1,000 to about 2,500, about 1,000 to about 5,000, about 1,000 to about 10,000, about 1,000 to about 25,000, about 1,000 to about 50,000, about 2,500 to about 5,000, about 2,500 to about 10,000, about 2,500 to about 25,000, about 2,500 to about 50,000, about 5,000 to about 10,000, about 5,000 to about 25,000, about 5,000 to about 50,000, about 10,000 to about 25,000, about 10,000 to about 50,000, or about 25,000 to about 50,000 SFCs per million peripheral blood mononuclear cells (PBMCs) at a timepoint post boost dose vaccination.

In some embodiments, heterologous administration of prime dose and boost dose (e.g., viral-mRNA administration, viral-peptide administration, mRNA-viral administration, mRNA-peptide administration, peptide-mRNA administration, or peptide-viral administration) can result in at least about 1, at least about 5, at least about 10, at least about 20, at least about 50, at least about 100, at least about 250, at least about 500, at least about 750, at least about 1,000, at least about 2,000, at least about 3,000, at least about 4,000, at least about 5,000, at least about 6,000, at least about 7,000, at least about 8,000, at least about 9,000, at least about 10,000, at least about 25,000, at least about 50,000, or greater than about 50,000 SFCs per million peripheral blood mononuclear cells (PBMCs) at a timepoint post prime dose vaccination. In some embodiments, heterologous administration of prime dose and boost dose (e.g., viral-mRNA administration, viral-peptide administration, mRNA-viral administration, mRNA-peptide administration, peptide-mRNA administration, or peptide-viral administration) can result in at most about 50,000, at most about 25,000, at most about 10,000, at most about 9,000, at most about 8,000, at most about 7,000, at most about 6,000, at most about 5,000, at most about 4,000, at most about 3,000, at most about 2,000, at most about 1,000, at most about 750, at most about 500, at most about 250, at most about 100, at most about 50, at most about 20, at most about 10, at most about 15, at most about 1, or less than about 1 SFC(s) per million PBMCs at a timepoint post prime dose vaccination. In some embodiments, heterologous administration of prime dose and boost dose (e.g., viral-mRNA administration, viral-peptide administration, mRNA-viral administration, mRNA-peptide administration, peptide-mRNA administration, or peptide-viral administration) can result in from about 1 to about 50,000 SFCs per million peripheral blood mononuclear cells (PBMCs) at a timepoint post prime dose vaccination. In some embodiments, heterologous administration of prime dose and boost dose (e.g., viral-mRNA administration, viral-peptide administration, mRNA-viral administration, mRNA-peptide administration, peptide-mRNA administration, or peptide-viral administration) can result in from about 1 to about 5, about 1 to about 10, about 1 to about 50, about 1 to about 100, about 1 to about 500, about 1 to about 1,000, about 1 to about 2,500, about 1 to about 5,000, about 1 to about 10,000, about 1 to about 25,000, about 1 to about 50,000, about 5 to about 10, about 5 to about 50, about 5 to about 100, about 5 to about 500, about 5 to about 1,000, about 5 to about 2,500, about 5 to about 5,000, about 5 to about 10,000, about 5 to about 25,000, about 5 to about 50,000, about 10 to about 50, about 10 to about 100, about 10 to about 500, about 10 to about 1,000, about 10 to about 2,500, about 10 to about 5,000, about 10 to about 10,000, about 10 to about 25,000, about 10 to about 50,000, about 50 to about 100, about 50 to about 500, about 50 to about 1,000, about 50 to about 2,500, about 50 to about 5,000, about 50 to about 10,000, about 50 to about 25,000, about 50 to about 50,000, about 100 to about 500, about 100 to about 1,000, about 100 to about 2,500, about 100 to about 5,000, about 100 to about 10,000, about 100 to about 25,000, about 100 to about 50,000, about 500 to about 1,000, about 500 to about 2,500, about 500 to about 5,000, about 500 to about 10,000, about 500 to about 25,000, about 500 to about 50,000, about 1,000 to about 2,500, about 1,000 to about 5,000, about 1,000 to about 10,000, about 1,000 to about 25,000, about 1,000 to about 50,000, about 2,500 to about 5,000, about 2,500 to about 10,000, about 2,500 to about 25,000, about 2,500 to about 50,000, about 5,000 to about 10,000, about 5,000 to about 25,000, about 5,000 to about 50,000, about 10,000 to about 25,000, about 10,000 to about 50,000, or about 25,000 to about 50,000 SFCs per million peripheral blood mononuclear cells (PBMCs) at a timepoint post prime dose vaccination.

In some embodiments, heterologous administration of prime dose and boost dose (e.g., viral-mRNA administration, viral-peptide administration, mRNA-viral administration, mRNA-peptide administration, peptide-mRNA administration, or peptide-viral administration) can result in at least about 1, at least about 5, at least about 10, at least about 20, at least about 50, at least about 100, at least about 250, at least about 500, at least about 750, at least about 1,000, at least about 2,000, at least about 3,000, at least about 4,000, at least about 5,000, at least about 6,000, at least about 7,000, at least about 8,000, at least about 9,000, at least about 10,000, at least about 25,000, at least about 50,000, or greater than about 50,000 SFCs per million peripheral blood mononuclear cells (PBMCs) at a timepoint post boost dose vaccination. In some embodiments, heterologous administration of prime dose and boost dose (e.g., viral-mRNA administration, viral-peptide administration, mRNA-viral administration, mRNA-peptide administration, peptide-mRNA administration, or peptide-viral administration) can result in at most about 50,000, at most about 25,000, at most about 10,000, at most about 9,000, at most about 8,000, at most about 7,000, at most about 6,000, at most about 5,000, at most about 4,000, at most about 3,000, at most about 2,000, at most about 1,000, at most about 750, at most about 500, at most about 250, at most about 100, at most about 50, at most about 20, at most about 10, at most about 15, at most about 1, or less than about 1 SFC(s) per million PBMCs at a timepoint post boost dose vaccination. In some embodiments, heterologous administration of prime dose and boost dose (e.g., viral-mRNA administration, viral-peptide administration, mRNA-viral administration, mRNA-peptide administration, peptide-mRNA administration, or peptide-viral administration) can result in from about 1 to about 50,000 SFCs per million peripheral blood mononuclear cells (PBMCs) at a timepoint post prime dose vaccination. In some embodiments, heterologous administration of prime dose and boost dose (e.g., viral-mRNA administration, viral-peptide administration, mRNA-viral administration, mRNA-peptide administration, peptide-mRNA administration, or peptide-viral administration) can result in from about 1 to about 5, about 1 to about 10, about 1 to about 50, about 1 to about 100, about 1 to about 500, about 1 to about 1,000, about 1 to about 2,500, about 1 to about 5,000, about 1 to about 10,000, about 1 to about 25,000, about 1 to about 50,000, about 5 to about 10, about 5 to about 50, about 5 to about 100, about 5 to about 500, about 5 to about 1,000, about 5 to about 2,500, about 5 to about 5,000, about 5 to about 10,000, about 5 to about 25,000, about 5 to about 50,000, about 10 to about 50, about 10 to about 100, about 10 to about 500, about 10 to about 1,000, about 10 to about 2,500, about 10 to about 5,000, about 10 to about 10,000, about 10 to about 25,000, about 10 to about 50,000, about 50 to about 100, about 50 to about 500, about 50 to about 1,000, about 50 to about 2,500, about 50 to about 5,000, about 50 to about 10,000, about 50 to about 25,000, about 50 to about 50,000, about 100 to about 500, about 100 to about 1,000, about 100 to about 2,500, about 100 to about 5,000, about 100 to about 10,000, about 100 to about 25,000, about 100 to about 50,000, about 500 to about 1,000, about 500 to about 2,500, about 500 to about 5,000, about 500 to about 10,000, about 500 to about 25,000, about 500 to about 50,000, about 1,000 to about 2,500, about 1,000 to about 5,000, about 1,000 to about 10,000, about 1,000 to about 25,000, about 1,000 to about 50,000, about 2,500 to about 5,000, about 2,500 to about 10,000, about 2,500 to about 25,000, about 2,500 to about 50,000, about 5,000 to about 10,000, about 5,000 to about 25,000, about 5,000 to about 50,000, about 10,000 to about 25,000, about 10,000 to about 50,000, or about 25,000 to about 50,000 SFCs per million peripheral blood mononuclear cells (PBMCs) at a timepoint post boost dose vaccination.

In the therapy of cancer, the recombinant polypeptides, polynucleotides, vectors, cells (e.g., APCs, T cells), compositions, pharmaceutical compositions, or formulations described herein, can be used in combination with existing therapies. In some embodiments, the recombinant polypeptides, polynucleotides, vectors, cells (e.g., APCs, T cells), compositions, pharmaceutical compositions, or formulations described herein is used in combination with an additional therapy or therapeutic agent (e.g., existing), for example an anti-cancer therapy, e.g., a neoadjuvant therapy. In some embodiments, the disclosure also relates to a combination therapy comprising administration of the recombinant polypeptides, polynucleotides, vectors, cells (e.g., APCs, T cells), compositions, pharmaceutical compositions, or formulations described herein and an anti-cancer therapy. The anti-cancer therapy may include one or more of therapeutic agent (e.g., a combination of different chemotherapeutic agents, antibodies), radiotherapies (y-rays, X-rays, and/or the directed delivery of radioisotopes, microwaves, UV radiation and the like), hormone therapy (steroidal, peptide, and the like), gene therapies (e.g., antisense, retroviral therapy and the like), viral therapy, RNA therapy, bone marrow transplantation, nanotherapy, and/or oncolytic drugs. Exemplary therapeutic agents include but are not limited to antibodies, antibody-drug conjugates, immunogenic vaccines, checkpoint inhibitors (e.g., immune checkpoint inhibitors, e.g., PD-1 antibodies), antineoplastic agents, immunogenic agents, attenuated cancerous cells, hormones, tumor antigens, antigen presenting cells such as dendritic cells pulsed with tumor-derived antigen or nucleic acids, immune stimulating cytokines (e.g., IL-2, IFNa2, GM-CSF), targeted small molecules and biological molecules (such as components of signal transduction pathways, e.g., modulators of tyrosine kinases and inhibitors of receptor tyrosine kinases, and agents that bind to tumor-specific antigens, including EGFR antagonists), an anti-inflammatory agent, a cytotoxic agent, a radiotoxic agent, an immunosuppressive agent and cells transfected with a gene encoding an immune stimulating cytokine (e.g., GM-CSF), chemotherapy. In some embodiments, the recombinant polypeptides, polynucleotides, vectors, cells (e.g., APCs, T cells), compositions, pharmaceutical compositions, or formulations described herein may be administered in combination with an anti-cancer therapy as described herein. In some embodiments, the recombinant polypeptides, polynucleotides, vectors, cells (e.g., APCs, T cells), compositions, pharmaceutical compositions, or formulations described herein may be administered in combination with cisplatin or histone deacetylase inhibitors. In some embodiments, the recombinant polypeptides, polynucleotides, vectors, cells (e.g., APCs, T cells), compositions, pharmaceutical compositions, or formulations described herein is used in combination with surgery. In some embodiments, the recombinant polypeptides, polynucleotides, vectors, cells (e.g., APCs, T cells), compositions, pharmaceutical compositions, or formulations described herein is administered before, during, or after surgery. In some embodiments, the recombinant polypeptides, polynucleotides, vectors, cells (e.g., APCs, T cells), compositions, pharmaceutical compositions, or formulations described herein are administered in combination with anti-diarrheal agents, anti-emetic agents, analgesics, opioids and/or non-steroidal anti-inflammatory agents. In some embodiments, the recombinant polypeptides, polynucleotides, vectors, cells (e.g., APCs, T cells), compositions, pharmaceutical compositions, or formulations described herein may be administered at the same time or at a different time as the other therapy, e.g., simultaneously, separately or sequentially.

The recombinant polypeptides, polynucleotides, vectors, cells (e.g., APCs, T cells), compositions, pharmaceutical compositions, or formulations and the additional therapy or therapeutic agent may be provided as separate medicaments for administration at the same time or at different times. In some embodiments, recombinant polypeptides, polynucleotides, vectors, cells (e.g., APCs, T cells), compositions, pharmaceutical compositions, or formulations and the additional therapy or therapeutic agent are provided as separate medicaments for administration at different times. When administered separately and at different times, either the recombinant polypeptides, polynucleotides, vectors, cells (e.g., APCs, T cells), compositions, pharmaceutical compositions, or formulations as described herein or the additional therapy or therapeutic agent may be administered first. In some embodiments, both can be administered on the same day or on different days, and they can be administered using the same schedule or at different schedules during the treatment cycle.

Alternatively, the administration of the additional therapy or therapeutic agent may be performed simultaneously with the administration of the recombinant polypeptides, polynucleotides, vectors, cells (e.g., APCs, T cells), compositions, pharmaceutical compositions, or formulations as described herein. Wherein simultaneous administration is used, the recombinant polypeptides, polynucleotides, vectors, cells (e.g., APCs, T cells), compositions, pharmaceutical compositions, or formulations described herein, and the additional therapy or therapeutic agent may be formulated as separate pharmaceutical compositions. In some embodiments, the recombinant polypeptides, polynucleotides, vectors, cells (e.g., APCs, T cells), compositions, pharmaceutical compositions, or formulations described herein, and the additional therapy or therapeutic agent may be formulated as a single pharmaceutical composition.

In some embodiments, the recombinant polypeptides, polynucleotides, vectors, cells (e.g., APCs, T cells), compositions, pharmaceutical compositions, or formulations described herein can be used in combination with an immunomodulatory agent. In some embodiments, the immunomodulatory agent may be administered simultaneously, sequentially or separately with the immunomodulatory agent. In some embodiments, the immunomodulatory agent may be an immune checkpoint inhibitor. Examples of immune checkpoint inhibitors include but are not limited to inhibitors of an immune checkpoint protein selected from the group consisting of CTLA-4, PD-1, PD-L1, PD-L2, TIM-3, LAG-3, B7-H3, B7-H4, B7-H6, A2aR, BTLA, GALS and IDO.

Certain tumor types have previously been reported to be unresponsive to anti-PD-1 and anti PD-L1 monotherapies. Immunization with a vector described herein can result in enhanced tumor control when administered in combination with a checkpoint inhibitor such as an anti-PD-L1 therapy. This has been shown effective in tumor models which are known to be unresponsive to standard checkpoint inhibitor therapy. As such, in some embodiments, the recombinant polypeptides, polynucleotides, vectors, cells (e.g., APCs, T cells), compositions, pharmaceutical compositions, or formulations described herein may be used in combination with a check point inhibitor for the treatment of checkpoint inhibitor unresponsive tumors.

The present disclosure provides methods for preventing relapse of cancer in a subject, the methods comprising administering to a subject in need thereof recombinant polypeptides, polynucleotides, vectors, cells (e.g., APCs, T cells), compositions, pharmaceutical compositions, or formulations described herein. The present disclosure also provides methods for preventing relapse of cancer in a subject, the methods comprising administering to a subject in need thereof an immunogenic composition comprising one or more of: (i) a recombinant polypeptide, (ii) a polynucleotide encoding a recombinant polypeptide, (iii) a cell, (iv) a population of cells, (v) a vector, (vi) a composition, (vii) a pharmaceutical composition, or (viii) a formulation as described herein.

The present disclosure provides methods for preventing metastasis of cancer in a subject, the methods comprising administering to a subject in need thereof recombinant polypeptides, polynucleotides, vectors, cells (e.g., APCs, T cells), compositions, pharmaceutical compositions, or formulations described herein. The present disclosure also provides methods for preventing metastasis of cancer in a subject, the methods comprising administering to a subject in need thereof an immunogenic composition comprising one or more of: (i) a recombinant polypeptide, (ii) a polynucleotide encoding a recombinant polypeptide, (iii) a cell, (iv) a population of cells, (v) a vector, (vi) a composition, (vii) a pharmaceutical composition, or (viii) a formulation as described herein.

The recombinant polypeptides, polynucleotides, vectors, cells (e.g., APCs, T cells), compositions, pharmaceutical compositions, or formulations and the immunomodulatory agent may be provided as separate medicaments for administration at the same time or at different times. In some embodiments, the vector or composition of the present disclosure and the immunomodulatory agent are provided as separate medicaments for administration at different times. When administered separately and at different times, either the vector or the immunomodulatory agent may be administered first. In some embodiments, both can be administered on the same day or on different days, and they can be administered using the same schedule or at different schedules during the treatment cycle.

Alternatively, the administration of the immunomodulatory agent may be performed simultaneously with the administration of the vector or immunogenic composition as described herein. Wherein simultaneous administration is used, the vector or immunogenic composition and the immunomodulatory agent may be formulated as separate pharmaceutical compositions. In some embodiments, the vector or immunogenic composition and the immunomodulatory agent may be formulated as a single pharmaceutical composition.

The recombinant polypeptides, polynucleotides, vectors, cells (e.g., APCs, T cells), compositions, pharmaceutical compositions, or formulations described herein can be administered prophylactically or therapeutically. The term “prophylactically” refers to administration intended to have a protective effect against disease. The term “therapeutically” refers to administration intended to have a curative effect.

Wherein the composition, pharmaceutical composition, or formulation comprises at least two vectors and wherein the vectors comprise recombinant polypeptide comprising different epitopes as disclosed herein or polynucleotides encoding different epitopes as described above, there may be synergy between the vectors. Wherein the immunogenic composition comprises at least three vectors and wherein the vectors comprise recombinant polypeptide comprising different epitopes as disclosed herein or polynucleotides encoding different epitopes as described above, there may be synergy between the vectors. Wherein the immunogenic composition comprises at least four vectors and wherein the vectors comprise recombinant polypeptide comprising different epitopes as disclosed herein or polynucleotides encoding different epitopes as described above, there may be synergy between the vectors. Wherein the immunogenic composition comprises at least five vectors and wherein the vectors comprise recombinant polypeptide comprising different epitopes as disclosed herein or polynucleotides encoding different epitopes as described above, there may be synergy between the vectors. Wherein the immunogenic composition comprises at least six vectors and wherein the vectors comprise recombinant polypeptide comprising different epitopes as disclosed herein or polynucleotides encoding different epitopes as described above, there may be synergy between the vectors. As such, each of the vectors may be administered at a sub-optimal dose. The term “sub-optimal” dose refers to a dose level that it is not intended to fully remove or eradicate the tumor, but nevertheless results in some tumor cells or tissue becoming necrotic. The skilled person will be able to determine an appropriate dose required in order to achieve this, depending on factors such as, age of the patient, status of the disease and size and location of tumor or metastases.

### Kits

In various aspects, provided herein is a kit for producing the composition described herein. In some embodiments, the kit comprises a recombinant polypeptide, a polynucleotide, a vector, a cell (e.g., APCs, T cells), a population of cells, a composition, a pharmaceutical composition, or a formulation described herein.

In some embodiments, the kit comprises a recombinant polypeptide comprising one or more cancer-specific epitopes comprising GVYDGREHTV (SEQ ID NO: 1) and KLVELEHTL (SEQ ID NO: 2) and SPSSASLAL (SEQ ID NO: 3) and KPRPDVTNEL (SEQ ID NO: 4) and KEFAFLEHSL (SEQ ID NO: 5) and HELGFKVVL (SEQ ID NO: 6) or any combination thereof. In some embodiments, the kit comprises a recombinant polypeptide comprising one or more cancer-specific epitopes comprising GVYDGREHTV (SEQ ID NO: 1) and KLVELEHTL (SEQ ID NO: 2) and SPSSASLAL (SEQ ID NO: 3) and KPRPDVTNEL (SEQ ID NO: 4) and KEFAFLEHSL (SEQ ID NO: 5), HELGFKVVL (SEQ ID NO: 6), LKPDHIQR (SEQ ID NO: 7), SLFGARPGR (SEQ ID NO: 8), KTEVHGRLK (SEQ ID NO: 9), NLRPPTQEL (SEQ ID NO: 10), RVSLPKLGYK (SEQ ID NO: 11), LRPPTQEL (SEQ ID NO: 12), and RPASPRPAP (SEQ ID NO: 13) or any combination thereof. In some embodiments, the kit comprises a recombinant polypeptide comprising one or more cancer-specific epitopes comprising GVYDGREHTV (SEQ ID NO: 1), KLVELEHTL (SEQ ID NO: 2), SPSSASLAL (SEQ ID NO: 3), KPRPDVTNEL (SEQ ID NO: 4), KEFAFLEHSL (SEQ ID NO: 5), HELGFKVVL (SEQ ID NO: 6), LKPDHIQR (SEQ ID NO: 7), SLFGARPGR (SEQ ID NO: 8), KTEVHGRLK (SEQ ID NO: 9), NLRPPTQEL (SEQ ID NO: 10), RVSLPKLGYK (SEQ ID NO: 11), LRPPTQEL (SEQ ID NO: 12), RPASPRPAP (SEQ ID NO: 13), IATKIALQM (SEQ ID NO: 14), SLYQTIRLK (SEQ ID NO: 15), KTPLHTLLK (SEQ ID NO: 16), RLHSFTLRQK (SEQ ID NO: 17), RVFTSSLKTK (SEQ ID NO: 18), RVLAKGLAK (SEQ ID NO: 19), GPRPSPTRSV (SEQ ID NO: 20), GQHLHLETF (SEQ ID NO: 21), SPRSPSPSL (SEQ ID NO: 22), VPQEAVRAPL (SEQ ID NO: 23), IYPFINSH (SEQ ID NO: 24), SPSSASLTL (SEQ ID NO: 25), LNKVKTSL (SEQ ID NO: 26), NLKTHLRL (SEQ ID NO: 27), FRGVFVHRY (SEQ ID NO: 28), TSGPVTEKY (SEQ ID NO: 29), and VVAAHLAGA (SEQ ID NO: 30) or any combination thereof. In some embodiments, the kit comprises a vector, composition, or formulation as described herein. In some embodiments, the kit is useful for producing a vector, composition, or formulation as described herein.

In some embodiments, a kit described herein includes one or more additional active ingredients, pharmaceutically acceptable carrier, diluent, excipient or adjuvant, and optionally instructions for use. The additional active agent may include checkpoint inhibitors, antineoplastic agents, immunogenic agents, attenuated cancerous cells, tumor antigens, antigen presenting cells such as dendritic cells pulsed with tumor-derived antigen or nucleic acids, immune stimulating cytokines (e.g., IL-2, IFNa2, GM-C SF), targeted small molecules and biological molecules (such as components of signal transduction pathways, e.g. modulators of tyrosine kinases and inhibitors of receptor tyrosine kinases, and agents that bind to tumor-specific antigens, including EGFR antagonists), an anti-inflammatory agent, a cytotoxic agent, a radiotoxic agent, or an immunosuppressive agent and cells transfected with a gene encoding an immune stimulating cytokine (e.g., GM-CSF).

The ability of an adjuvant to increase an immune response to an antigen is typically manifested by a significant or substantial increase in an immune-mediated reaction, or reduction in disease symptoms.

The pharmaceutical acceptable carrier, diluent, excipient or adjuvant may include; sterile diluents such as water, saline solution, preferably physiological saline, Ringer's solution, isotonic sodium chloride, fixed oils such as synthetic mono or digylcerides, polyethylene glycols, glycerin, or other solvents; antibacterial agents such as benzyl alcohol or methyl paraben; and agents for the adjustment of tonicity such as sodium chloride or dextrose.

Typically, the kits are compartmentalized for ease of use and can include one or more containers with reagents. In certain embodiments, all of the kit components are packaged together. Alternatively, one or more individual components of the kit can be provided in a separate package from the other kits components. The kits can also include instructions for using the kit components.

TABLE 10
APPENDIX
Exemplary Sequences

| | SEQ | SEQ | |
|---|---|---|---|
| Descrip- | Identi- | ID | |
| tion | fier | NO. | Sequence |
| CMV | A36 | 36 | cctttcactcattagatgcatgtcgttacataacttacggtaaatggcccgcctggctgaccgcccaac |
| promoter | | | gacccccgcccattgacgtcaataatgacgtatgttcccatagtaacgccaatagggactttccattga |
| | | | cgtcaatgggtggagtatttacggtaaactgcccacttggcagtacatcaagtgtatcatatgccaagt |
| | | | acgccccctattgacgtcaatgacggtaaatggcccgcctggcattatgcccagtacatgaccttatg |
| | | | ggactttcctacttggcagtacatctacgtattagtcatcgctattaccatggtgatgcggttttggcagt |
| | | | acatcaatgggcgtggatagcggtttgactcacggggatttccaagtctccaccccattgacgtcaat |
| | | | gggagtttgttttggcaccaaaatcaacgggactttccaaaatgtcgtaacaactccgccccattgacg |
| | | | caaatgggcggtaggcgtgtacggtgggaggtctatataagcagagctctccctatcagtgatagag |
| | | | atctccctatcagtgatagagatcgtcgacgagctcgtttagtgaaccgtcagatcgcctggagacgc |
| | | | catccacgctgttttgacctccatagaagacaccgggaccgatccagcctccggttaagcttggtacc |
| Kozak | A37 | 37 | cccgccgccacc |
| sequence | | | |
| Start codon | A38 | | atg |
| A1 Epitope | A39 | 39 | ggagtttacgatggcagagagcacaccgtc |
| codon | | | |
| Stop codon | A40 | | taa |
| Stop codon | A41 | | tga |
| Stop codon | A42 | | tag |
| BGH poly | A43 | 43 | gcggccgctcgagcatgcatctagagggccctattctatagtgtcacctaaatgctagagctcgctga |
| A sequence | | | tcagcctcgactgtgccttctagttgccagccatctgttgtttgcccctcccccgtgccttccttgaccct |
| | | | ggaaggtgccactcccactgtcctttcctaataaaatgaggaaattgcatcgcattgtctgagtaggtgt |
| | | | cattctattctggggggtggggtggggcaggacagcaagggggaggattgggaagacaatagcag |
| | | | gcatgctggggatgcggtgggctctatgg |
| 5′ | A44 | 44 | ttaattaacatcatcaataatataccttattttggattgaagccaatatgataatgagggggtggagtttgt |
| Adenoviral | | | gacgtggcgcggggcgtgggaacggggcgggtgacgtagtagtgtggcggaagtgtgatgttgc |
| nucleotide | | | aagtgtggcggaacacatgtaagcgacggatgtggcaaaagtgacgtttttggtgtgcgccggtgta |
| sequence | | | cacaggaagtgacaattttcgcgcggttttaggcggatgttgtagtaaatttgggcgtaaccgagtaag |
| excluding | | | atttggccattttcgcgggaaaactgaataagaggaagtgaaatctgaataattttgtgttactcatagc |
| the | | | gcgtaatatttgtctagggccgcggggactttgaccgtttacgtggagactcgcccaggtgtttttctca |
| minigene | | | ggtgttttccgcgttccgggtcaaagttggcgttttattattatagtcagtcgaagcttggatccggtacc |
| cassette | | | tctagaattctcgagcggccgctagcgacatcgatcacaagtttgtacaaaaaagcaggctccaccat |
| | | | ggtaactataacggtcctaaggtagcgaagaaccaattcagtcgag |
| 3′ | A45 | 45 | cttctgaggcggaaagaaccagctggggctcgaggggggatcgatccgtcgagatatctagggata |
| Adenoviral | | | acagggtaattagacccagctttcttgtacaaagtggtgatcgattcgacagatcactgaaatgtgtgg |
| nucleotide | | | gcgtggcttaagggtgggaaagaatatataaggtgggggtcttatgtagttttgtatctgttttgcagca |
| sequence | | | gccgccgccgccatgagcaccaactcgtttgatggaagcattgtgagctcatatttgacaacgcgcat |
| excluding | | | gcccccatgggccggggtgcgtcagaatgtgatgggctccagcattgatggtcgccccgtcctgcc |
| the | | | cgcaaactctactaccttgacctacgagaccgtgtctggaacgccgttggagactgcagcctccgcc |
| minigene | | | gccgcttcagccgctgcagccaccgcccgcgggattgtgactgactttgctttcctgagcccgcttgc |
| cassette | | | aagcagtgcagcttcccgttcatccgcccgcgatgacaagttgacggctcttttggcacaattggattc |
| | | | tttgacccgggaacttaatgtcgtttctcagcagctgttggatctgcgccagcaggtttctgccctgaag |
| | | | gcttcctcccctcccaatgcggtttaaaacataaataaaaaaccagactctgtttggatttggatcaagc |
| | | | aagtgtcttgctgtctttatttaggggttttgcgcgcgcggtaggcccgggaccagcggtctcggtcgt |
| | | | tgagggtcctgtgtattttttccaggacgtggtaaaggtgactctggatgttcagatacatgggcataag |
| | | | cccgtctctggggtggaggtagcaccactgcagagcttcatgctgcggggtggtgttgtagatgatc |
| | | | cagtcgtagcaggagcgctgggcgtggtgcctaaaaatgtctttcagtagcaagctgattgccaggg |
| | | | gcaggcccttggtgtaagtgtttacaaagcggttaagctgggatgggtgcatacgtggggatatgag |
| | | | atgcatcttggactgtatttttaggttggctatgttcccagccatatccctccggggattcatgttgtgcag |
| | | | aaccaccagcacagtgtatccggtgcacttgggaaatttgtcatgtagcttagaaggaaatgcgtgga |
| | | | agaacttggagacgcccttgtgacctccaagattttccatgcattcgtccataatgatggcaatgggcc |
| | | | cacgggcggcggcctgggcgaagatatttctgggatcactaacgtcatagttgtgttccaggatgag |
| | | | atcgtcataggccatttttacaaagcgcgggcggagggtgccagactgcggtataatggttccatccg |
| | | | gcccaggggcgtagttaccctcacagatttgcatttcccacgctttgagttcagatggggggatcatgt |
| | | | ctacctgcggggcgatgaagaaaacggtttccggggtaggggagatcagctgggaagaaagcag |
| | | | gttcctgagcagctgcgacttaccgcagccggtgggcccgtaaatcacacctattaccggctgcaac |
| | | | tggtagttaagagagctgcagctgccgtcatccctgagcaggggggccacttcgttaagcatgtccct |
| | | | gactcgcatgttttccctgaccaaatccgccagaaggcgctcgccgcccagcgatagcagttcttgc |
| | | | aaggaagcaaagtttttcaacggtttgagaccgtccgccgtaggcatgcttttgagcgtttgaccaagc |
| | | | agttccaggcggtcccacagctcggtcacctgctctacggcatctcgatccagcatatctcctcgtttc |
| | | | gcgggttggggcggctttcgctgtacggcagtagtcggtgctcgtccagacgggccagggtcatgt |
| | | | ctttccacgggcgcagggtcctcgtcagcgtagtctgggtcacggtgaaggggtgcgctccgggct |
| | | | gcgcgctggccagggtgcgcttgaggctggtcctgctggtgctgaagcgctgccggtcttcgccct |
| | | | gcgcgtcggccaggtagcatttgaccatggtgtcatagtccagcccctccgcggcgtggcccttggc |
| | | | gcgcagcttgcccttggaggaggcgccgcacgaggggcagtgcagacttttgagggcgtagagct |
| | | | tgggcgcgagaaataccgattccggggagtaggcatccgcgccgcaggccccgcagacggtctc |
| | | | gcattccacgagccaggtgagctctggccgttcggggtcaaaaaccaggtttcccccatgctttttgat |
| | | | gcgtttcttacctctggtttccatgagccggtgtccacgctcggtgacgaaaaggctgtccgtgtcccc |
| | | | gtatacagacttgagaggcctgtcctcgagcggtgttccgcggtcctcctcgtatagaaactcggacc |
| | | | actctgagacaaaggctcgcgtccaggccagcacgaaggaggctaagtgggaggggtagcggtc |
| | | | gttgtccactagggggtccactcgctccagggtgtgaagacacatgtcgccctcttcggcatcaagg |
| | | | aaggtgattggtttgtaggtgtaggccacgtgaccgggtgttcctgaaggggggctataaaaggggg |
| | | | tgggggcgcgttcgtcctcactctcttccgcatcgctgtctgcgagggccagctgttggggtgagtac |
| | | | tccctctgaaaagcgggcatgacttctgcgctaagattgtcagtttccaaaaacgaggaggatttgata |
| | | | ttcacctggcccgcggtgatgcctttgagggggccgcatccatctggtcagaaaagacaatctttttg |
| | | | ttgtcaagcttggtggcaaacgacccgtagagggcgttggacagcaacttggcgatggagcgcagg |
| | | | gtttggtttttgtcgcgatcggcgcgctccttggccgcgatgtttagctgcacgtattcgcgcgcaacg |
| | | | caccgccattcgggaaagacggtggtgcgctcgtcgggcaccaggtgcacgcgccaaccgcggtt |
| | | | gtgcagggtgacaaggtcaacgctggtggctacctctccgcgtaggcgctcgttggtccagcagag |
| | | | gcggccgcccttgcgcgagcagaatggcggtagggggtctagctgcgtctcgtccggggggtctg |
| | | | cgtccacggtaaagaccccgggcagcaggcgcgcgtcgaagtagtctatcttgcatccttgcaagtc |
| | | | tagcgcctgctgccatgcgcgggcggcaagcgcgcgctcgtatgggttgagtgggggaccccatg |
| | | | gcatggggtgggtgagcgcggaggcgtacatgccgcaaatgtcgtaaacgtagaggggctctctg |
| | | | agtattccaagatatgtagggtagcatcttccaccgcggatgctggcgcgcacgtaatcgtatagttcg |
| | | | tgcgagggagcgaggaggtcgggaccgaggttgctacggggggctgctctgctcggaagactat |
| | | | ctgcctgaagatggcatgtgagttggatgatatggttggacgctggaagacgttgaagctggcgtctg |
| | | | tgagacctaccgcgtcacgcacgaaggaggcgtaggagtcgcgcagcttgttgaccagctcggcg |
| | | | gtgacctgcacgtctagggcgcagtagtccagggtttccttgatgatgtcatacttatcctgtccctttttt |
| | | | ttccacagctcgcggttgaggacaaactcttcgcggtctttccagtactcttggatcggaaacccgtcg |
| | | | gcctccgaacggtaagagcctagcatgtagaactggttgacggcctggtaggcgcagcatccctttt |
| | | | ctacgggtagcgcgtatgcctgcgcggccttccggagcgaggtgtgggtgagcgcaaaggtgtcc |
| | | | ctgaccatgactttgaggtactggtatttgaagtcagtgtcgtcgcatccgccctgctcccagagcaaa |
| | | | aagtccgtgcgctttttggaacgcggatttggcagggcgaaggtgacatcgttgaagagtatctttccc |
| | | | gcgcgaggcataaagttgcgtgtgatgcggaagggtcccggcacctcggaacggttgttaattacct |
| | | | gggcggcgagcacgatctcgtcaaagccgttgatgttgtggcccacaatgtaaagttccaagaagc |
| | | | gcgggatgcccttgatggaaggcaattttttaagttcctcgtaggtgagctcttcaggggagctgagc |
| | | | ccgtgctctgaaagggcccagtctgcaagatgagggttggaagcgacgaatgagctccacaggtca |
| | | | cgggccattagcatttgcaggtggtcgcgaaaggtcctaaactggcgacctatggccattttttctggg |
| | | | gtgatgcagtagaaggtaagcgggtcttgttcccagcggtcccatccaaggttcgcggctaggtctc |
| | | | gcgcggcagtcactagaggctcatctccgccgaacttcatgaccagcatgaagggcacgagctgct |
| | | | tcccaaaggcccccatccaagtataggtctctacatcgtaggtgacaaagagacgctcggtgcgag |
| | | | gatgcgagccgatcgggaagaactggatctcccgccaccaattggaggagtggctattgatgtggt |
| | | | gaaagtagaagtccctgcgacgggccgaacactcgtgctggcttttgtaaaaacgtgcgcagtactg |
| | | | gcagcggtgcacgggctgtacatcctgcacgaggttgacctgacgaccgcgcacaaggaagcag |
| | | | agtgggaatttgagcccctcgcctggcgggtttggctggtggtcttctacttcggctgcttgtccttgac |
| | | | cgtctggctgctcgaggggagttacggtggatcggaccaccacgccgcgcgagcccaaagtccag |
| | | | atgtccgcgcgcggcggtcggagcttgatgacaacatcgcgcagatgggagctgtccatggtctgg |
| | | | agctcccgcggcgtcaggtcagggggagctcctgcaggtttacctcgcatagacgggtcagggc |
| | | | gcgggctagatccaggtgatacctaatttccaggggctggttggtggcggcgtcgatggcttgcaag |
| | | | aggccgcatccccgcggcgcgactacggtaccgcgcggcgggcggtgggccgcgggggtgtcc |
| | | | ttggatgatgcatctaaaagcggtgacgcgggcgagcccccggaggtagggggggctccggacc |
| | | | cgccgggagagggggcaggggcacgtcggcgccgcgcgcgggcaggagctggtgctgcgcgc |
| | | | gtaggttgctggcgaacgcgacgacgcggcggttgatctcctgaatctggcgcctctgcgtgaaga |
| | | | cgacgggcccggtgagcttgaacctgaaagagagttcgacagaatcaatttcggtgtcgttgacggc |
| | | | ggcctggcgcaaaatctcctgcacgtctcctgagttgtcttgataggcgatctcggccatgaactgctc |
| | | | gatctcttcctcctggagatctccgcgtccggctcgctccacggtggcggcgaggtcgttggaaatg |
| | | | cgggccatgagctgcgagaaggcgttgaggcctccctcgttccagacgcggctgtagaccacgcc |
| | | | cccttcggcatcgcgggcgcgcatgaccacctgcgcgagattgagctccacgtgccgggcgaaga |
| | | | cggcgtagtttcgcaggcgctgaaagaggtagttgagggtggtggcggtgtgttctgccacgaaga |
| | | | agtacataacccagcgtcgcaacgtggattcgttgatatcccccaaggcctcaaggcgctccatggc |
| | | | ctcgtagaagtccacggcgaagttgaaaaactgggagttgcgcgccgacacggttaactcctcctcc |
| | | | agaagacggatgagctcggcgacagtgtcgcgcacctcgcgctcaaaggctacaggggcctcttct |
| | | | tcttcttcaatctcctcttccataagggcctccccttcttcttcttctggcggcggtgggggagggggga |
| | | | cacggcggcgacgacggcgcaccgggaggcggtcgacaaagcgctcgatcatctccccgcggc |
| | | | gacggcgcatggtctcggtgacggcgcggccgttctcgcgggggcgcagttggaagacgccgcc |
| | | | cgtcatgtcccggttatgggttggcggggggctgccatgcggcagggatacggcgctaacgatgca |
| | | | tctcaacaattgttgtgtaggtactccgccgccgagggacctgagcgagtccgcatcgaccggatcg |
| | | | gaaaacctctcgagaaaggcgtctaaccagtcacagtcgcaaggtaggctgagcaccgtggggg |
| | | | cggcagcgggcggcggtcggggttgtttctggcggaggtgctgctgatgatgtaattaaagtaggc |
| | | | ggtcttgagacggcggatggtcgacagaagcaccatgtccttgggtccggcctgctgaatgcgcag |
| | | | gcggtcggccatgccccaggcttcgttttgacatcggcgcaggtctttgtagtagtcttgcatgagcct |
| | | | ttctaccggcacttcttcttctccttcctcttgtcctgcatctcttgcatctatcgctgcggcggcggcgga |
| | | | gtttggccgtaggtggcgccctcttcctcccatgcgtgtgaccccgaagcccctcatcggctgaagc |
| | | | agggctaggtcggcgacaacgcgctcggctaatatggcctgctgcacctgcgtgagggtagactgg |
| | | | aagtcatccatgtccacaaagcggtggtatgcgcccgtgttgatggtgtaagtgcagttggccataac |
| | | | ggaccagttaacggtctggtgacccggctgcgagagctcggtgtacctgagacgcgagtaagccct |
| | | | cgagtcaaatacgtagtcgttgcaagtccgcaccaggtactggtatcccaccaaaaagtgcggcgg |
| | | | cggctggcggtagaggggccagcgtagggtggccggggctccgggggcgagatcttccaacata |
| | | | aggcgatgatatccgtagatgtacctggacatccaggtgatgccggcggcggtggtggaggcgcg |
| | | | cggaaagtcgcggacgcggttccagatgttgcgcagcggcaaaaagtgctccatggtcgggacgc |
| | | | tctggccggtcaggcgcgcgcaatcgttgacgctctagaccgtgcaaaaggagagcctgtaagcg |
| | | | ggcactcttccgtggtctggtggataaattcgcaagggtatcatggcggacgaccggggttcgagcc |
| | | | ccgtatccggccgtccgccgtgatccatgcggttaccgcccgcgtgtcgaacccaggtgtgcgacg |
| | | | tcagacaacgggggagtgctccttttggcttccttccaggcgcggcggctgctgcgctagcttttttgg |
| | | | ccactggccgcgcgcagcgtaagcggttaggctggaaagcgaaagcattaagtggctcgctccctg |
| | | | tagccggagggttattttccaagggttgagtcgcgggacccccggttcgagtctcggaccggccgg |
| | | | actgcggcgaacgggggtttgcctccccgtcatgcaagaccccgcttgcaaattcctccggaaaca |
| | | | gggacgagccccttttttgcttttcccagatgcatccggtgctgcggcagatgcgcccccctcctcag |
| | | | cagcggcaagagcaagagcagcggcagacatgcagggcaccctcccctcctcctaccgcgtcag |
| | | | gaggggcgacatccgcggttgacgcggcagcagatggtgattacgaacccccgcggcgccgggc |
| | | | ccggcactacctggacttggaggagggcgagggcctggcgcggctaggagcgccctctcctgag |
| | | | cggcacccaagggtgcagctgaagcgtgatacgcgtgaggcgtacgtgccgcggcagaacctgtt |
| | | | tcgcgaccgcgagggagaggagcccgaggagatgcgggatcgaaagttccacgcagggcgcga |
| | | | gctgcggcatggcctgaatcgcgagcggttgctgcgcgaggaggactttgagcccgacgcgcgaa |
| | | | ccgggattagtcccgcgcgcgcacacgtggggccgccgacctggtaaccgcatacgagcagac |
| | | | ggtgaaccaggagattaactttcaaaaaagctttaacaaccacgtgcgtacgcttgtggcgcgcgag |
| | | | gaggtggctataggactgatgcatctgtgggactttgtaagcgcgctggagcaaaacccaaatagca |
| | | | agccgctcatggcgcagctgttccttatagtgcagcacagcagggacaacgaggcattcagggatg |
| | | | cgctgctaaacatagtagagcccgagggccgctggctgctcgatttgataaacatcctgcagagcat |
| | | | agtggtgcaggagcgcagcttgagcctggctgacaaggtggccgccatcaactattccatgcttagc |
| | | | ctgggcaagttttacgcccgcaagatataccataccccttacgttcccatagacaaggaggtaaagat |
| | | | cgaggggttctacatgcgcatggcgctgaaggtgcttaccttgagcgacgacctgggcgtttatcgc |
| | | | aacgagcgcatccacaaggccgtgagcgtgagccggcggcgcgagctcagcgaccgcgagctg |
| | | | atgcacagcctgcaaagggccctggctggcacgggcagcggcgatagagaggccgagtcctactt |
| | | | tgacgcgggcgctgacctgcgctgggccccaagccgacgcgccctggaggcagctggggccgg |
| | | | acctgggctggcggtggcacccgcgcgcgctggcaacgtcggcggcgtggaggaatatgacgag |
| | | | gacgatgagtacgagccagaggacggcgagtactaagcggtgatgtttctgatcagatgatgcaag |
| | | | acgcaacggacccggcggtgcgggcggcgctgcagagccagccgtccggccttaactccacgga |
| | | | cgactggcgccaggtcatggaccgcatcatgtcgctgactgcgcgcaatcctgacgcgttccggca |
| | | | gcagccgcaggccaaccggctctccgcaattctggaagcggtggtcccggcgcgcgcaaacccc |
| | | | acgcacgagaaggtgctggcgatcgtaaacgcgctggccgaaaacagggccatccggcccgacg |
| | | | aggccggcctggtctacgacgcgctgcttcagcgcgtggctcgttacaacagcggcaacgtgcaga |
| | | | ccaacctggaccggctggtgggggatgtgcgcgaggccgtggcgcagcgtgagcgcgcgcagc |
| | | | agcagggcaacctgggctccatggttgcactaaacgccttcctgagtacacagcccgccaacgtgc |
| | | | cgcggggacaggaggactacaccaactttgtgagcgcactgcggctaatggtgactgagacaccg |
| | | | caaagtgaggtgtaccagtctgggccagactattttttccagaccagtagacaaggcctgcagaccgt |
| | | | aaacctgagccaggctttcaaaaacttgcaggggctgtggggggtgcgggctcccacaggcgacc |
| | | | gcgcgaccgtgtctagcttgctgacgcccaactcgcgcctgttgctgctgctaatagcgcccttcacg |
| | | | gacagtggcagcgtgtcccgggacacatacctaggtcacttgctgacactgtaccgcgaggccata |
| | | | ggtcaggcgcatgtggacgagcatactttccaggagattacaagtgtcagccgcgcgctggggcag |
| | | | gaggacacgggcagcctggaggcaaccctaaactacctgctgaccaaccggcggcagaagatcc |
| | | | cctcgttgcacagtttaaacagcgaggaggagcgcattttgcgctacgtgcagcagagcgtgagcct |
| | | | taacctgatgcgcgacggggtaacgcccagcgtggcgctggacatgaccgcgcgcaacatggaa |
| | | | ccgggcatgtatgcctcaaaccggccgtttatcaaccgcctaatggactacttgcatcgcgcggccg |
| | | | ccgtgaaccccgagtatttcaccaatgccatcttgaacccgcactggctaccgccccctggtttctaca |
| | | | ccgggggattcgaggtgcccgagggtaacgatggattcctctgggacgacatagacgacagcgtgt |
| | | | tttccccgcaaccgcagaccctgctagagttgcaacagcgcgagcaggcagaggcggcgctgcga |
| | | | aaggaaagcttccgcaggccaagcagcttgtccgatctaggcgctgcggccccgcggtcagatgct |
| | | | agtagcccatttccaagcttgatagggtctcttaccagcactcgcaccacccgcccgcgcctgctgg |
| | | | gcgaggaggagtacctaaacaactcgctgctgcagccgcagcgcgaaaaaaacctgcctccggca |
| | | | tttcccaacaacgggatagagagcctagtggacaagatgagtagatggaagacgtacgcgcagga |
| | | | gcacagggacgtgccaggcccgcgcccgcccacccgtcgtcaaaggcacgaccgtcagcgggg |
| | | | tctggtgtgggaggacgatgactcggcagacgacagcagcgtcctggatttgggagggagtggca |
| | | | acccgtttgcgcaccttcgccccaggctggggagaatgttttaaaaaaaaaaaaagcatgatgcaaa |
| | | | ataaaaaactcaccaaggccatggcaccgagcgttggttttcttgtattccccttagtatgcggcgcgc |
| | | | ggcgatgtatgaggaaggtcctcctccctcctacgagagtgtggtgagcgcggcgccagtggcgg |
| | | | cggcgctgggttctcccttcgatgctcccctggacccgccgtttgtgcctccgcggtacctgcggcct |
| | | | accggggggagaaacagcatccgttactctgagttggcacccctattcgacaccacccgtgtgtacc |
| | | | tggtggacaacaagtcaacggatgtggcatccctgaactaccagaacgaccacagcaactttctgac |
| | | | cacggtcattcaaaacaatgactacagcccgggggaggcaagcacacagaccatcaatcttgacga |
| | | | ccggtcgcactggggggcgacctgaaaaccatcctgcataccaacatgccaaatgtgaacgagtt |
| | | | catgtttaccaataagtttaaggcgcgggtgatggtgtcgcgcttgcctactaaggacaatcaggtgg |
| | | | agctgaaatacgagtgggtggagttcacgctgcccgagggcaactactccgagaccatgaccatag |
| | | | accttatgaacaacgcgatcgtggagcactacttgaaagtgggcagacagaacggggttctggaaa |
| | | | gcgacatcggggtaaagtttgacacccgcaacttcagactggggtttgaccccgtcactggtcttgtc |
| | | | atgcctggggtatatacaaacgaagccttccatccagacatcattttgctgccaggatgcggggtgga |
| | | | cttcacccacagccgcctgagcaacttgttgggcatccgcaagcggcaacccttccaggagggcttt |
| | | | aggatcacctacgatgatctggagggtggtaacattcccgcactgttggatgtggacgcctaccagg |
| | | | cgagcttgaaagatgacaccgaacagggcgggggtggcgcaggcggcagcaacagcagtggca |
| | | | gcggcgcggaagagaactccaacgcggcagccgcggcaatgcagccggtggaggacatgaacg |
| | | | atcatgccattcgcggcgacacctttgccacacgggctgaggagaagcgcgctgaggccgaagca |
| | | | gcggccgaagctgccgcccccgctgcgcaacccgaggtcgagaagcctcagaagaaaccggtg |
| | | | atcaaacccctgacagaggacagcaagaaacgcagttacaacctaataagcaatgacagcaccttc |
| | | | acccagtaccgcagctggtaccttgcatacaactacggcgaccctcagaccggaatccgctcatgg |
| | | | accctgctttgcactcctgacgtaacctgcggctcggagcaggtctactggtcgttgccagacatgat |
| | | | gcaagaccccgtgaccttccgctccacgcgccagatcagcaactttccggtggtgggcgccgagct |
| | | | gttgcccgtgcactccaagagcttctacaacgaccaggccgtctactcccaactcatccgccagttta |
| | | | cctctctgacccacgtgttcaatcgctttcccgagaaccagattttggcgcgcccgccagcccccacc |
| | | | atcaccaccgtcagtgaaaacgttcctgctctcacagatcacgggacgctaccgctgcgcaacagca |
| | | | tcggaggagtccagcgagtgaccattactgacgccagacgccgcacctgcccctacgtttacaagg |
| | | | ccctgggcatagtctcgccgcgcgtcctatcgagccgcactttttgagcaagcatgtccatccttatat |
| | | | cgcccagcaataacacaggctggggcctgcgcttcccaagcaagatgtttggcggggccaagaag |
| | | | cgctccgaccaacacccagtgcgcgtgcgcgggcactaccgcgcgccctggggcgcgcacaaac |
| | | | gcggccgcactgggcgcaccaccgtcgatgacgccatcgacgcggtggtggaggaggcgcgca |
| | | | actacacgcccacgccgccaccagtgtccacagtggacgcggccattcagaccgtggtgcgcgga |
| | | | gcccggcgctatgctaaaatgaagagacggcggaggcgcgtagcacgtcgccaccgccgccgac |
| | | | ccggcactgccgcccaacgcgcggcggcggccctgcttaaccgcgcacgtcgcaccggccgac |
| | | | gggcggccatgcgggccgctcgaaggctggccgcgggtattgtcactgtgccccccaggtccagg |
| | | | cgacgagcggccgccgcagcagccgcggccattagtgctatgactcagggtcgcaggggcaacg |
| | | | tgtattgggtgcgcgactcggttagcggcctgcgcgtgcccgtgcgcacccgccccccgcgcaact |
| | | | agattgcaagaaaaaactacttagactcgtactgttgtatgtatccagcggcggcggcgcgcaacga |
| | | | agctatgtccaagcgcaaaatcaaagaagagatgctccaggtcatcgcgccggagatctatggccc |
| | | | cccgaagaaggaagagcaggattacaagccccgaaagctaaagcgggtcaaaaagaaaaagaaa |
| | | | gatgatgatgatgaacttgacgacgaggtggaactgctgcacgctaccgcgcccaggcgacgggt |
| | | | acagtggaaaggtcgacgcgtaaaacgtgttttgcgacccggcaccaccgtagtctttacgcccggt |
| | | | gagcgctccacccgcacctacaagcgcgtgtatgatgaggtgtacggcgacgaggacctgcttgag |
| | | | caggccaacgagcgcctcggggagtttgcctacggaaagcggcataaggacatgctggcgttgcc |
| | | | gctggacgagggcaacccaacacctagcctaaagcccgtaacactgcagcaggtgctgcccgcgc |
| | | | ttgcaccgtccgaagaaaagcgcggcctaaagcgcgagtctggtgacttggcacccaccgtgcag |
| | | | ctgatggtacccaagcgccagcgactggaagatgtcttggaaaaaatgaccgtggaacctgggctg |
| | | | gagcccgaggtccgcgtgcggccaatcaagcaggtggcgccgggactgggcgtgcagaccgtg |
| | | | gacgttcagatacccactaccagtagcaccagtattgccaccgccacagagggcatggagacacaa |
| | | | acgtccccggttgcctcagcggtggcggatgccgcggtgcaggcggtcgctgcggccgcgtccaa |
| | | | gacctctacggaggtgcaaacggacccgtggatgtttcgcgtttcagccccccggcgcccgcgccg |
| | | | ttcgaggaagtacggcgccgccagcgcgctactgcccgaatatgccctacatccttccattgcgcct |
| | | | acccccggctatcgtggctacacctaccgccccagaagacgagcaactacccgacgccgaaccac |
| | | | cactggaacccgccgccgccgtcgccgtcgccagcccgtgctggccccgatttccgtgcgcaggg |
| | | | tggctcgcgaaggaggcaggaccctggtgctgccaacagcgcgctaccaccccagcatcgtttaaa |
| | | | agccggtctttgtggttcttgcagatatggccctcacctgccgcctccgtttcccggtgccgggattcc |
| | | | gaggaagaatgcaccgtaggaggggcatggccggccacggcctgacgggcggcatgcgtcgtg |
| | | | cgcaccaccggcggcggcgcgcgtcgcaccgtcgcatgcgcggcggtatcctgcccctccttattc |
| | | | cactgatcgccgcggcgattggcgccgtgcccggaattgcatccgtggccttgcaggcgcagaga |
| | | | cactgattaaaaacaagttgcatgtggaaaaatcaaaataaaaagtctggactctcacgctcgcttggt |
| | | | cctgtaactattttgtagaatggaagacatcaactttgcgtctctggccccgcgacacggctcgcgccc |
| | | | gttcatgggaaactggcaagatatcggcaccagcaatatgagcggtggcgccttcagctggggctc |
| | | | gctgtggagcggcattaaaaatttcggttccaccgttaagaactatggcagcaaggcctggaacagc |
| | | | agcacaggccagatgctgagggataagttgaaagagcaaaatttccaacaaaaggtggtagatggc |
| | | | ctggcctctggcattagcggggtggtggacctggccaaccaggcagtgcaaaataagattaacagt |
| | | | aagcttgatccccgccctcccgtagaggagcctccaccggccgtggagacagtgtctccagaggg |
| | | | gcgtggcgaaaagcgtccgcgccccgacagggaagaaactctggtgacgcaaatagacgagcct |
| | | | ccctcgtacgaggaggcactaaagcaaggcctgcccaccacccgtcccatcgcgcccatggctac |
| | | | cggagtgctgggccagcacacacccgtaacgctggacctgcctccccccgccgacacccagcag |
| | | | aaacctgtgctgccaggcccgaccgccgttgttgtaacccgtcctagccgcgcgtccctgcgccgc |
| | | | gccgccagcggtccgcgatcgttgcggcccgtagccagtggcaactggcaaagcacactgaacag |
| | | | catcgtgggtctgggggtgcaatccctgaagcgccgacgatgcttctgatagctaacgtgtcgtatgt |
| | | | gtgtcatgtatgcgtccatgtcgccgccagaggagctgctgagccgccgcgcgcccgctttccaag |
| | | | atggctaccccttcgatgatgccgcagtggtcttacatgcacatctcgggccaggacgcctcggagt |
| | | | acctgagccccgggctggtgcagtttgcccgcgccaccgagacgtacttcagcctgaataacaagtt |
| | | | tagaaaccccacggtggcgcctacgcacgacgtgaccacagaccggtcccagcgtttgacgctgc |
| | | | ggttcatccctgtggaccgtgaggatactgcgtactcgtacaaggcgcggttcaccctagctgtgggt |
| | | | gataaccgtgtgctggacatggcttccacgtactttgacatccgcggcgtgctggacaggggcccta |
| | | | cttttaagccctactctggcactgcctacaacgccctggctcccaagggtgccccaaatccttgcgaat |
| | | | gggatgaagctgctactgctcttgaaataaacctagaagaagaggacgatgacaacgaagacgaag |
| | | | tagacgagcaagctgagcagcaaaaaactcacgtatttgggcaggcgccttattctggtataaatatta |
| | | | caaaggagggtattcaaataggtgtcgaaggtcaaacacctaaatatgccgataaaacatttcaacct |
| | | | gaacctcaaataggagaatctcagtggtacgaaacagaaattaatcatgcagctgggagagtcctaa |
| | | | aaaagactaccccaatgaaaccatgttacggttcatatgcaaaacccacaaatgaaaatggagggca |
| | | | aggcattcttgtaaagcaacaaaatggaaagctagaaagtcaagtggaaatgcaatttttctcaactac |
| | | | tgaggcagccgcaggcaatggtgataacttgactcctaaagtggtattgtacagtgaagatgtagata |
| | | | tagaaaccccagacactcatatttcttacatgcccactattaaggaaggtaactcacgagaactaatgg |
| | | | gccaacaatctatgcccaacaggcctaattacattgcttttagggacaattttattggtctaatgtattaca |
| | | | acagcacgggtaatatgggtgttctggcgggccaagcatcgcagttgaatgctgttgtagatttgcaa |
| | | | gacagaaacacagagctttcataccagcttttgcttgattccattggtgatagaaccaggtacttttctat |
| | | | gtggaatcaggctgttgacagctatgatccagatgttagaattattgaaaatcatggaactgaagatga |
| | | | acttccaaattactgctttccactgggaggtgtgattaatacagagactcttaccaaggtaaaacctaaa |
| | | | acaggtcaggaaaatggatgggaaaaagatgctacagaattttcagataaaaatgaaataagagttg |
| | | | gaaataattttgccatggaaatcaatctaaatgccaacctgtggagaaatttcctgtactccaacatagc |
| | | | gctgtatttgcccgacaagctaaagtacagtccttccaacgtaaaaatttctgataacccaaacaccta |
| | | | cgactacatgaacaagcgagtggtggctcccgggctagtggactgctacattaaccttggagcacgc |
| | | | tggtcccttgactatatggacaacgtcaacccatttaaccaccaccgcaatgctggcctgcgctaccg |
| | | | ctcaatgttgctgggcaatggtcgctatgtgcccttccacatccaggtgcctcagaagttctttgccatt |
| | | | aaaaacctccttctcctgccgggctcatacacctacgagtggaacttcaggaaggatgttaacatggtt |
| | | | ctgcagagctccctaggaaatgacctaagggttgacggagccagcattaagtttgatagcatttgcctt |
| | | | tacgccaccttcttccccatggcccacaacaccgcctccacgcttgaggccatgcttagaaacgaca |
| | | | ccaacgaccagtcctttaacgactatctctccgccgccaacatgctctaccctatacccgccaacgct |
| | | | accaacgtgcccatatccatcccctcccgcaactgggcggctttccgcggctgggccttcacgcgcc |
| | | | ttaagactaaggaaaccccatcactgggctcgggctacgacccttattacacctactctggctctatac |
| | | | cctacctagatggaaccttttacctcaaccacacctttaagaaggtggccattacctttgactcttctgtc |
| | | | agctggcctggcaatgaccgcctgcttacccccaacgagtttgaaattaagcgctcagttgacgggg |
| | | | agggttacaacgttgcccagtgtaacatgaccaaagactggttcctggtacaaatgctagctaactata |
| | | | acattggctaccagggcttctatatcccagagagctacaaggaccgcatgtactccttctttagaaactt |
| | | | ccagcccatgagccgtcaggtggtggatgatactaaatacaaggactaccaacaggtgggcatcct |
| | | | acaccaacacaacaactctggatttgttggctaccttgcccccaccatgcgcgaaggacaggcctac |
| | | | cctgctaacttcccctatccgcttataggcaagaccgcagttgacagcattacccagaaaaagtttcttt |
| | | | gcgatcgcaccctttggcgcatcccattctccagtaactttatgtccatgggcgcactcacagacctgg |
| | | | gccaaaaccttctctacgccaactccgcccacgcgctagacatgacttttgaggtggatcccatggac |
| | | | gagcccacccttctttatgttttgtttgaagtctttgacgtggtccgtgtgcaccagccgcaccgcggcg |
| | | | tcatcgaaaccgtgtacctgcgcacgcccttctcggccggcaacgccacaacataaagaagcaagc |
| | | | aacatcaacaacagctgccgccatgggctccagtgagcaggaactgaaagccattgtcaaagatctt |
| | | | ggttgtgggccatattttttgggcacctatgacaagcgctttccaggctttgtttctccacacaagctcgc |
| | | | ctgcgccatagtcaatacggccggtcgcgagactgggggcgtacactggatggcctttgcctggaa |
| | | | cccgcactcaaaaacatgctacctctttgagccctttggcttttctgaccagcgactcaagcaggtttac |
| | | | cagtttgagtacgagtcactcctgcgccgtagcgccattgcttcttcccccgaccgctgtataacgctg |
| | | | gaaaagtccacccaaagcgtacaggggcccaactcggccgcctgtggactattctgctgcatgtttct |
| | | | ccacgcctttgccaactggccccaaactcccatggatcacaaccccaccatgaaccttattaccggg |
| | | | gtacccaactccatgctcaacagtccccaggtacagcccaccctgcgtcgcaaccaggaacagctc |
| | | | tacagcttcctggagcgccactcgccctacttccgcagccacagtgcgcagattaggagcgccactt |
| | | | ctttttgtcacttgaaaaacatgtaaaaataatgtactagagacactttcaataaaggcaaatgcttttattt |
| | | | gtacactctcgggtgattatttacccccacccttgccgtctgcgccgtttaaaaatcaaaggggttctgc |
| | | | cgcgcatcgctatgcgccactggcagggacacgttgcgatactggtgtttagtgctccacttaaactc |
| | | | aggcacaaccatccgcggcagctcggtgaagttttcactccacaggctgcgcaccatcaccaacgc |
| | | | gtttagcaggtcgggcgccgatatcttgaagtcgcagttggggcctccgccctgcgcgcgcgagttg |
| | | | cgatacacagggttgcagcactggaacactatcagcgccgggtggtgcacgctggccagcacgct |
| | | | cttgtcggagatcagatccgcgtccaggtcctccgcgttgctcaggggaacggagtcaactttggta |
| | | | gctgccttcccaaaaagggcgcgtgcccaggctttgagttgcactcgcaccgtagtggcatcaaaag |
| | | | gtgaccgtgcccggtctgggcgttaggatacagcgcctgcataaaagccttgatctgcttaaaagcc |
| | | | acctgagcctttgcgccttcagagaagaacatgccgcaagacttgccggaaaactgattggccgga |
| | | | caggccgcgtcgtgcacgcagcaccttgcgtcggtgttggagatctgcaccacatttcggccccacc |
| | | | ggttcttcacgatcttggccttgctagactgctccttcagcgcgcgctgcccgttttcgctcgtcacatc |
| | | | catttcaatcacgtgctccttatttatcataatgcttccgtgtagacacttaagctcgccttcgatctcagc |
| | | | gcagcggtgcagccacaacgcgcagcccgtgggctcgtgatgcttgtaggtcacctctgcaaacga |
| | | | ctgcaggtacgcctgcaggaatcgccccatcatcgtcacaaaggtcttgttgctggtgaaggtcagct |
| | | | gcaacccgcggtgctcctcgttcagccaggtcttgcatacggccgccagagcttccacttggtcagg |
| | | | cagtagtttgaagttcgcctttagatcgttatccacgtggtacttgtccatcagcgcgcgcgcagcctcc |
| | | | atgcccttctcccacgcagacacgatcggcacactcagcgggttcatcaccgtaatttcactttccgct |
| | | | tcgctgggctcttcctcttcctcttgcgtccgcataccacgcgccactgggtcgtcttcattcagccgcc |
| | | | gcactgtgcgcttacctcctttgccatgcttgattagcaccggtgggttgctgaaacccaccatttgtag |
| | | | cgccacatcttctctttcttcctcgctgtccacgattacctctggtgatggcgggcgctcgggcttggga |
| | | | gaagggcgcttctttttcttcttgggcgcaatggccaaatccgccgccgaggtcgatggccggggc |
| | | | tgggtgtgcgcggcaccagcgcgtcttgtgatgagtcttcctcgtcctcggactcgatacgccgcctc |
| | | | atccgcttttttgggggcgcccggggaggcggcggcgacggggacggggacgacacgtcctcca |
| | | | tggttgggggacgtcgcgccgcaccgcgtccgcgctcgggggtggtttcgcgctgctcctcttcccg |
| | | | actggccatttccttctcctataggcagaaaaagatcatggagtcagtcgagaagaaggacagccta |
| | | | accgccccctctgagttcgccaccaccgcctccaccgatgccgccaacgcgcctaccaccttcccc |
| | | | gtcgaggcacccccgcttgaggaggaggaagtgattatcgagcaggacccaggttttgtaagcgaa |
| | | | gacgacgaggaccgctcagtaccaacagaggataaaaagcaagaccaggacaacgcagaggca |
| | | | aacgaggaacaagtcgggcggggggacgaaaggcatggcgactacctagatgtgggagacgac |
| | | | gtgctgttgaagcatctgcagcgccagtgcgccattatctgcgacgcgttgcaagagcgcagcgatg |
| | | | tgcccctcgccatagcggatgtcagccttgcctacgaacgccacctattctcaccgcgcgtaccccc |
| | | | caaacgccaagaaaacggcacatgcgagcccaacccgcgcctcaacttctaccccgtatttgccgt |
| | | | gccagaggtgcttgccacctatcacatctttttccaaaactgcaagatacccctatcctgccgtgccaa |
| | | | ccgcagccgagcggacaagcagctggccttgcggcagggcgctgtcatacctgatatcgcctcgct |
| | | | caacgaagtgccaaaaatctttgagggtcttggacgcgacgagaagcgcgcggcaaacgctctgc |
| | | | aacaggaaaacagcgaaaatgaaagtcactctggagtgttggtggaactcgagggtgacaacgcg |
| | | | cgcctagccgtactaaaacgcagcatcgaggtcacccactttgcctacccggcacttaacctacccc |
| | | | ccaaggtcatgagcacagtcatgagtgagctgatcgtgcgccgtgcgcagcccctggagagggat |
| | | | gcaaatttgcaagaacaaacagaggagggcctacccgcagttggcgacgagcagctagcgcgctg |
| | | | gcttcaaacgcgcgagcctgccgacttggaggagcgacgcaaactaatgatggccgcagtgctcgt |
| | | | taccgtggagcttgagtgcatgcagcggttctttgctgacccggagatgcagcgcaagctagaggaa |
| | | | acattgcactacacctttcgacagggctacgtacgccaggcctgcaagatctccaacgtggagctct |
| | | | gcaacctggtctcctaccttggaattttgcacgaaaaccgccttgggcaaaacgtgcttcattccacgc |
| | | | tcaagggcgaggcgcgccgcgactacgtccgcgactgcgtttacttatttctatgctacacctggcag |
| | | | acggccatgggcgtttggcagcagtgcttggaggagtgcaacctcaaggagctgcagaaactgcta |
| | | | aagcaaaacttgaaggacctatggacggccttcaacgagcgctccgtggccgcgcacctggcgga |
| | | | catcattttccccgaacgcctgcttaaaaccctgcaacagggtctgccagacttcaccagtcaaagca |
| | | | tgttgcagaactttaggaactttatcctagagcgctcaggaatcttgcccgccacctgctgtgcacttcc |
| | | | tagcgactttgtgcccattaagtaccgcgaatgccctccgccgctttggggccactgctaccttctgca |
| | | | gctagccaactaccttgcctaccactctgacataatggaagacgtgagcggtgacggtctactggagt |
| | | | gtcactgtcgctgcaacctatgcaccccgcaccgctccctggtttgcaattcgcagctgcttaacgaa |
| | | | agtcaaattatcggtacctttgagctgcagggtccctcgcctgacgaaaagtccgcggctccggggtt |
| | | | gaaactcactccggggctgtggacgtcggcttaccttcgcaaatttgtacctgaggactaccacgccc |
| | | | acgagattaggttctacgaagaccaatcccgcccgcctaatgcggagcttaccgcctgcgtcattacc |
| | | | cagggccacattcttggccaattgcaagccatcaacaaagcccgccaagagtttctgctacgaaagg |
| | | | gacggggggtttacttggacccccagtccggcgaggagctcaacccaatccccccgccgccgcag |
| | | | ccctatcagcagcagccggggcccttgcttcccaggatggcacccaaaaagaagctgcagctgc |
| | | | cgccgccacccacggacgaggaggaatactgggacagtcaggcagaggaggttttggacgagga |
| | | | ggaggaggacatgatggaagactgggagagcctagacgaggaagcttccgaggtcgaagaggtg |
| | | | tcagacgaaacaccgtcaccctcggtcgcattcccctcgccggcgccccagaaatcggcaaccggt |
| | | | tccagcatggctacaacctccgctcctcaggcgccgccggcactgcccgttcgccgacccaaccgt |
| | | | agatgggacaccactggaaccagggccggtaagtccaagcagccgccgccgttagcccaagagc |
| | | | aacaacagcgccaaggctaccgctcatggcgcgggcacaagaacgccatagttgcttgcttgcaag |
| | | | actgtgggggcaacatctccttcgcccgccgctttcttctctaccatcacggcgtggccttcccccgta |
| | | | acatcctgcattactaccgtcatctctacagcccatactgcaccggcggcagcggcagcaacagcag |
| | | | cggccacacagaagcaaaggcgaccggatagcaagactctgacaaagcccaagaaatccacagc |
| | | | ggcggcagcagcaggaggaggagcgctgcgtctggcgcccaacgaacccgtatcgacccgcga |
| | | | gcttagaaacaggatttttcccactctgtatgctatatttcaacagagcaggggccaagaacaagagct |
| | | | gaaaataaaaaacaggtctctgcgatccctcacccgcagctgcctgtatcacaaaagcgaagatcag |
| | | | cttcggcgcacgctggaagacgcggaggctctcttcagtaaatactgcgcgctgactcttaaggact |
| | | | agtttcgcgcccttttcaaatttaagcgcgaaaactacgtcatctccagcggccacacccggcgcca |
| | | | gcacctgttgtcagcgccattatgagcaaggaaattcccacgccctacatgtggagttaccagccaca |
| | | | aatgggacttgcggctggagctgcccaagactactcaacccgaataaactacatgagcgcgggacc |
| | | | ccacatgatatcccgggtcaacggaatacgcgcccaccgaaaccgaattctcctggaacaggcggc |
| | | | tattaccaccacacctcgtaataaccttaatccccgtagttggcccgctgccctggtgtaccaggaaag |
| | | | tcccgctcccaccactgtggtacttcccagagacgcccaggccgaagttcagatgactaactcaggg |
| | | | gcgcagcttgcgggcggctttcgtcacagggtgcggtcgcccgggcagggtataactcacctgaca |
| | | | atcagagggcgaggtattcagctcaacgacgagtcggtgagctcctcgcttggtctccgtccggacg |
| | | | ggacatttcagatcggcggcgccggccgctcttcattcacgcctcgtcaggcaatcctaactctgcag |
| | | | acctcgtcctctgagccgcgctctggaggcattggaactctgcaatttattgaggagtttgtgccatcg |
| | | | gtctactttaaccccttctcgggacctcccggccactatccggatcaatttattcctaactttgacgcggt |
| | | | aaaggactcggcggacggctacgactgaatgttaagtggagaggcagagcaactgcgcctgaaac |
| | | | acctggtccactgtcgccgccacaagtgctttgcccgcgactccggtgagttttgctactttgaattgcc |
| | | | cgaggatcatatcgagggcccggcgcacggcgtccggcttaccgcccagggagagcttgcccgta |
| | | | gcctgattcgggagtttacccagcgccccctgctagttgagcgggacaggggaccctgtgttctcact |
| | | | gtgatttgcaactgtcctaaccctggattacatcaagatctttgttgccatctctgtgctgagtataataaa |
| | | | tacagaaattaaaatatactggggctcctatcgccatcctgtaaacgccaccgtcttcacccgcccaa |
| | | | gcaaaccaaggcgaaccttacctggtacttttaacatctctccctctgtgatttacaacagtttcaaccca |
| | | | gacggagtgagtctacgagagaacctctccgagctcagctactccatcagaaaaaacaccaccctc |
| | | | cttacctgccgggaacgtacgagtgcgtcaccggccgctgcaccacacctaccgcctgaccgtaaa |
| | | | ccagactttttccggacagacctcaataactctgtttaccagaacaggaggtgagcttagaaaaccctt |
| | | | agggtattaggccaaaggcgcagctactgtggggtttatgaacaattcaagcaactctacgggctatt |
| | | | ctaattcaggtttctctagaaatggacggaattattacagagcagcgcctgctagaaagacgcagggc |
| | | | agcggccgagcaacagcgcatgaatcaagagctccaagacatggttaacttgcaccagtgcaaaa |
| | | | ggggtatcttttgtctggtaaagcaggccaaagtcacctacgacagtaataccaccggacaccgcctt |
| | | | agctacaagttgccaaccaagcgtcagaaattggtggtcatggtgggagaaaagcccattaccataa |
| | | | ctcagcactcggtagaaaccgaaggctgcattcactcaccttgtcaaggacctgaggatctctgcacc |
| | | | cttattaagaccctgtgcggtctcaaagatcttattccctttaactaataaaaaaaaataataaagcatca |
| | | | cttacttaaaatcagttagcaaatttctgtccagtttattcagcagcacctccttgccctcctcccagctct |
| | | | ggtattgcagcttcctcctggctgcaaactttctccacaatctaaatggaatgtcagtttcctcctgttcct |
| | | | gtccatccgcacccactatcttcatgttgttgcagatgaagcgcgcaagaccgtctgaagataccttca |
| | | | accccgtgtatccatatgacacggaaaccggtcctccaactgtgccttttcttactcctccctttgtatcc |
| | | | cccaatgggtttcaagagagtccccctggggtactctctttgcgcctatccgaacctctagttacctcca |
| | | | atggcatgcttgcgctcaaaatgggcaacggcctctctctggacgaggccggcaaccttacctccca |
| | | | aaatgtaaccactgtgagcccacctctcaaaaaaaccaagtcaaacataaacctggaaatatctgcac |
| | | | ccctcacagttacctcagaagccctaactgtggctgccgccgcacctctaatggtcgcgggcaacac |
| | | | actcaccatgcaatcacaggccccgctaaccgtgcacgactccaaacttagcattgccacccaagga |
| | | | cccctcacagtgtcagaaggaaagctagccctgcaaacatcaggccccctcaccaccaccgatagc |
| | | | agtacccttactatcactgcctcaccccctctaactactgccactggtagcttgggcattgacttgaaag |
| | | | agcccatttatacacaaaatggaaaactaggactaaagtacggggctcctttgcatgtaacagacgac |
| | | | ctaaacactttgaccgtagcaactggtccaggtgtgactattaataatacttccttgcaaactaaagttac |
| | | | tggagccttgggttttgattcacaaggcaatatgcaacttaatgtagcaggaggactaaggattgattct |
| | | | caaaacagacgccttatacttgatgttagttatccgtttgatgctcaaaaccaactaaatctaagactag |
| | | | gacagggccctctttttataaactcagcccacaacttggatattaactacaacaaaggcctttacttgttt |
| | | | acagcttcaaacaattccaaaaagcttgaggttaacctaagcactgccaaggggttgatgtttgacgct |
| | | | acagccatagccattaatgcaggagatgggcttgaatttggttcacctaatgcaccaaacacaaatcc |
| | | | cctcaaaacaaaaattggccatggcctagaatttgattcaaacaaggctatggttcctaaactaggaac |
| | | | tggccttagttttgacagcacaggtgccattacagtaggaaacaaaaataatgataagctaactttgtg |
| | | | gaccacaccagctccatctcctaactgtagactaaatgcagagaaagatgctaaactcactttggtctt |
| | | | aacaaaatgtggcagtcaaatacttgctacagtttcagttttggctgttaaaggcagtttggctccaatat |
| | | | ctggaacagttcaaagtgctcatcttattataagatttgacgaaaatggagtgctactaaacaattccttc |
| | | | ctggacccagaatattggaactttagaaatggagatcttactgaaggcacagcctatacaaacgctgtt |
| | | | ggatttatgcctaacctatcagcttatccaaaatctcacggtaaaactgccaaaagtaacattgtcagtc |
| | | | aagtttacttaaacggagacaaaactaaacctgtaacactaaccattacactaaacggtacacaggaa |
| | | | acaggagacacaactccaagtgcatactctatgtcattttcatgggactggtctggccacaactacattaa |
| | | | tgaaatatttgccacatcctcttacactttttcatacattgcccaagaataaagaatcgtttgtgttatgtt |
| | | | tcaacgtgtttatttttcaattgcagaaaatttcgaatcatttttcattcagtagtatagccccaccaccaca |
| | | | tagcttatacagatcaccgtaccttaatcaaactcacagaaccctagtattcaacctgccacctccctcc |
| | | | caacacacagagtacacagtcctttctccccggctggccttaaaaagcatcatatcatgggtaacaga |
| | | | catattcttaggtgttatattccacacggtttcctgtcgagccaaacgctcatcagtgatattaataaactc |
| | | | cccgggcagctcacttaagttcatgtcgctgtccagctgctgagccacaggctgctgtccaacttgcg |
| | | | gttgcttaacgggcggcgaaggagaagtccacgcctacatgggggtagagtcataatcgtgcatca |
| | | | ggatagggcggtggtgctgcagcagcgcgcgaataaactgctgccgccgccgctccgtcctgcag |
| | | | gaatacaacatggcagtggtctcctcagcgatgattcgcaccgcccgcagcataaggcgccttgtcc |
| | | | tccgggcacagcagcgcaccctgatctcacttaaatcagcacagtaactgcagcacagcaccacaa |
| | | | tattgttcaaaatcccacagtgcaaggcgctgtatccaaagctcatggggggaccacagaacccac |
| | | | gtggccatcataccacaagcgcaggtagattaagtggcgacccctcataaacacgctggacataaa |
| | | | cattacctcttttggcatgttgtaattcaccacctcccggtaccatataaacctctgattaaacatggcgc |
| | | | catccaccaccatcctaaaccagctggccaaaacctgcccgccggctatacactgcagggaaccgg |
| | | | gactggaacaatgacagtggagagcccaggactcgtaaccatggatcatcatgctcgtcatgatatc |
| | | | aatgttggcacaacacaggcacacgtgcatacacttcctcaggattacaagctcctcccgcgttagaa |
| | | | ccatatcccagggaacaacccattcctgaatcagcgtaaatcccacactgcagggaagacctcgca |
| | | | cgtaactcacgttgtgcattgtcaaagtgttacattcgggcagcagcggatgatcctccagtatggtag |
| | | | cgcgggtttctgtctcaaaaggaggtagacgatccctactgtacggagtgcgccgagacaaccgag |
| | | | atcgtgttggtcgtagtgtcatgccaaatggaacgccggacgtagtcatatttcctgaagcaaaacca |
| | | | ggtgcgggcgtgacaaacagatctgcgtctccggtctcgccgcttagatcgctctgtgtagtagttgta |
| | | | gtatatccactctctcaaagcatccaggcgccccctggcttcgggttctatgtaaactccttcatgcgcc |
| | | | gctgccctgataacatccaccaccgcagaataagccacacccagccaacctacacattcgttctgcg |
| | | | agtcacacacgggaggagcgggaagagctggaagaaccatgtttttttttttattccaaaagattatcc |
| | | | aaaacctcaaaatgaagatctattaagtgaacgcgctcccctccggtggcgtggtcaaactctacagc |
| | | | caaagaacagataatggcatttgtaagatgttgcacaatggcttccaaaaggcaaacggccctcacgt |
| | | | ccaagtggacgtaaaggctaaacccttcagggtgaatctcctctataaacattccagcaccttcaacc |
| | | | atgcccaaataattctcatctcgccaccttctcaatatatctctaagcaaatcccgaatattaagtccggc |
| | | | cattgtaaaaatctgctccagagcgccctccaccttcagcctcaagcagcgaatcatgattgcaaaaa |
| | | | ttcaggttcctcacagacctgtataagattcaaaagcggaacattaacaaaaataccgcgatcccgta |
| | | | ggtcccttcgcagggccagctgaacataatcgtgcaggtctgcacggaccagcgcggccacttccc |
| | | | cgccaggaaccatgacaaaagaacccacactgattatgacacgcatactcggagctatgctaacca |
| | | | gcgtagccccgatgtaagcttgttgcatgggcggcgatataaaatgcaaggtgctgctcaaaaaatc |
| | | | aggcaaagcctcgcgcaaaaaagaaagcacatcgtagtcatgctcatgcagataaaggcaggtaa |
| | | | gctccggaaccaccacagaaaaagacaccatttttctctcaaacatgtctgcgggtttctgcataaaca |
| | | | caaaataaaataacaaaaaaacatttaaacattagaagcctgtcttacaacaggaaaaacaacccttat |
| | | | aagcataagacggactacggccatgccggcgtgaccgtaaaaaaactggtcaccgtgattaaaaag |
| | | | caccaccgacagctcctcggtcatgtccggagtcataatgtaagactcggtaaacacatcaggttgat |
| | | | tcacatcggtcagtgctaaaaagcgaccgaaatagcccgggggaatacatacccgcaggcgtaga |
| | | | gacaacattacagcccccataggaggtataacaaaattaataggagagaaaaacacataaacacctg |
| | | | aaaaaccctcctgcctaggcaaaatagcaccctcccgctccagaacaacatacagcgcttccacag |
| | | | cggcagccataacagtcagccttaccagtaaaaaagaaaacctattaaaaaaacaccactcgacac |
| | | | ggcaccagctcaatcagtcacagtgtaaaaaagggccaagtgcagagcgagtatatataggactaa |
| | | | aaaatgacgtaacggttaaagtccacaaaaaacacccagaaaaccgcacgcgaacctacgcccag |
| | | | aaacgaaagccaaaaaacccacaacttcctcaaatcgtcacttccgttttcccacgttacgtcacttcc |
| | | | cattttaagaaaactacaattcccaacacatacaagttactccgccctaaaacctacgtcacccgcccc |
| | | | gttcccacgccccgcgccacgtcacaaactccaccccctcattatcatattggcttcaatccaaaataa |
| | | | ggtatattattgatgatgttaattaa |
| Ad5-A1 | A46 | 46 | ttaattaacatcatcaataatataccttattttggattgaagccaatatgataatgagggggtggagtt |
| minigene | | | tgtgacgtggcgcggggcgtgggaacggggcgggtgacgtagtagtgtggcggaagtgtgatgttgc |
| nucleotide | | | aagtgtggcggaacacatgtaagcgacggatgtggcaaaagtgacgtttttggtgtgcgccggtgta |
| sequence | | | cacaggaagtgacaattttcgcgcggttttaggcggatgttgtagtaaatttgggcgtaaccgagtaag |
| | | | atttggccattttcgcgggaaaactgaataagaggaagtgaaatctgaataattttgtgttactcatagc |
| | | | gcgtaatatttgtctagggccgcggggactttgaccgtttacgtggagactcgcccaggtgtttttctca |
| | | | ggtgttttccgcgttccgggtcaaagttggcgttttattattatagtcagtcgaagcttggatccggtacc |
| | | | tctagaattctcgagcggccgctagcgacatcgatcacaagtttgtacaaaaaagcaggctccaccat |
| | | | ggtaactataacggtcctaaggtagcgaagaaccaattcagtcgagcctttcactcattagatgcatgt |
| | | | cgttacataacttacggtaaatggcccgcctggctgaccgcccaacgacccccgcccattgacgtca |
| | | | ataatgacgtatgttcccatagtaacgccaatagggactttccattgacgtcaatgggtggagtatttac |
| | | | ggtaaactgcccacttggcagtacatcaagtgtatcatatgccaagtacgccccctattgacgtcaatg |
| | | | acggtaaatggcccgcctggcattatgcccagtacatgaccttatgggactttcctacttggcagtaca |
| | | | tctacgtattagtcatcgctattaccatggtgatgcggttttggcagtacatcaatgggcgtggatagcg |
| | | | gtttgactcacggggatttccaagtctccaccccattgacgtcaatgggagtttgttttggcaccaaaat |
| | | | caacgggactttccaaaatgtcgtaacaactccgccccattgacgcaaatgggcggtaggcgtgtac |
| | | | ggtgggaggtctatataagcagagctctccctatcagtgatagagatctccctatcagtgatagagatc |
| | | | gtcgacgagctcgtttagtgaaccgtcagatcgcctggagacgccatccacgctgttttgacctccat |
| | | | agaagacaccgggaccgatccagcctccggttaagcttggtacccccgccgccaccatgggagttt |
| | | | acgatggcagagagcacaccgtctgagcggccgctcgagcatgcatctagagggccctattctata |
| | | | gtgtcacctaaatgctagagctcgctgatcagcctcgactgtgccttctagttgccagccatctgttgttt |
| | | | gcccctcccccgtgccttccttgaccctggaaggtgccactcccactgtcctttcctaataaaatgagg |
| | | | aaattgcatcgcattgtctgagtaggtgtcattctattctggggggtggggggggcaggacagcaag |
| | | | ggggaggattgggaagacaatagcaggcatgctggggatgcggtgggctctatggcttctgaggc |
| | | | ggaaagaaccagctggggctcgaggggggatcgatccgtcgagatatctagggataacagggtaa |
| | | | ttagacccagctttcttgtacaaagtggtgatcgattcgacagatcactgaaatgtgtgggcgtggctta |
| | | | agggtgggaaagaatatataaggtgggggtcttatgtagttttgtatctgttttgcagcagccgccgcc |
| | | | gccatgagcaccaactcgtttgatggaagcattgtgagctcatatttgacaacgcgcatgcccccatg |
| | | | ggccggggtgcgtcagaatgtgatgggctccagcattgatggtcgccccgtcctgcccgcaaactct |
| | | | actaccttgacctacgagaccgtgtctggaacgccgttggagactgcagcctccgccgccgcttcag |
| | | | ccgctgcagccaccgcccgcgggattgtgactgactttgctttcctgagcccgcttgcaagcagtgc |
| | | | agcttcccgttcatccgcccgcgatgacaagttgacggctcttttggcacaattggattctttgacccgg |
| | | | gaacttaatgtcgtttctcagcagctgttggatctgcgccagcaggtttctgccctgaaggcttcctccc |
| | | | ctcccaatgcggtttaaaacataaataaaaaaccagactctgtttggatttggatcaagcaagtgtcttg |
| | | | ctgtctttatttaggggttttgcgcgcgcggtaggcccgggaccagcggtctcggtcgttgagggtcc |
| | | | tgtgtattttttccaggacgtggtaaaggtgactctggatgttcagatacatgggcataagcccgtctct |
| | | | ggggtggaggtagcaccactgcagagcttcatgctgcggggtggtgttgtagatgatccagtcgtag |
| | | | caggagcgctgggcgtggtgcctaaaaatgtctttcagtagcaagctgattgccaggggcaggccct |
| | | | tggtgtaagtgtttacaaagcggttaagctgggatgggtgcatacgtggggatatgagatgcatcttg |
| | | | gactgtatttttaggttggctatgttcccagccatatccctccggggattcatgttgtgcagaaccacca |
| | | | gcacagtgtatccggtgcacttgggaaatttgtcatgtagcttagaaggaaatgcgtggaagaacttg |
| | | | gagacgcccttgtgacctccaagattttccatgcattcgtccataatgatggcaatgggcccacgggc |
| | | | ggcggcctgggcgaagatatttctgggatcactaacgtcatagttgtgttccaggatgagatcgtcata |
| | | | ggccatttttacaaagcgcgggcggagggtgccagactgcggtataatggttccatccggcccagg |
| | | | ggcgtagttaccctcacagatttgcatttcccacgctttgagttcagatggggggatcatgtctacctgc |
| | | | ggggcgatgaagaaaacggtttccggggtaggggagatcagctgggaagaaagcaggttcctga |
| | | | gcagctgcgacttaccgcagccggtgggcccgtaaatcacacctattaccggctgcaactggtagtt |
| | | | aagagagctgcagctgccgtcatccctgagcaggggggccacttcgttaagcatgtccctgactcgc |
| | | | atgttttccctgaccaaatccgccagaaggcgctcgccgcccagcgatagcagttcttgcaaggaag |
| | | | caaagtttttcaacggtttgagaccgtccgccgtaggcatgcttttgagcgtttgaccaagcagttcca |
| | | | ggcggtcccacagctcggtcacctgctctacggcatctcgatccagcatatctcctcgtttcgcgggtt |
| | | | ggggcggctttcgctgtacggcagtagtcggtgctcgtccagacgggccagggtcatgtctttccac |
| | | | gggcgcagggtcctcgtcagcgtagtctgggtcacggtgaaggggtgcgctccgggctgcgcgct |
| | | | ggccagggtgcgcttgaggctggtcctgctggtgctgaagcgctgccggtcttcgccctgcgcgtc |
| | | | ggccaggtagcatttgaccatggtgtcatagtccagcccctccgcggcgtggcccttggcgcgcag |
| | | | cttgcccttggaggaggcgccgcacgaggggcagtgcagacttttgagggcgtagagcttgggcg |
| | | | cgagaaataccgattccggggagtaggcatccgcgccgcaggccccgcagacggtctcgcattcc |
| | | | acgagccaggtgagctctggccgttcggggtcaaaaaccaggtttcccccatgctttttgatgcgtttc |
| | | | ttacctctggtttccatgagccggtgtccacgctcggtgacgaaaaggctgtccgtgtccccgtataca |
| | | | gacttgagaggcctgtcctcgagcggtgttccgcggtcctcctcgtatagaaactcggaccactctga |
| | | | gacaaaggctcgcgtccaggccagcacgaaggaggctaagtgggaggggtagcggtcgttgtcc |
| | | | actagggggtccactcgctccagggtgtgaagacacatgtcgccctcttcggcatcaaggaaggtg |
| | | | attggtttgtaggtgtaggccacgtgaccgggtgttcctgaaggggggctataaaagggggtgggg |
| | | | gcgcgttcgtcctcactctcttccgcatcgctgtctgcgagggccagctgttggggtgagtactccctc |
| | | | tgaaaagcgggcatgacttctgcgctaagattgtcagtttccaaaaacgaggaggatttgatattcacc |
| | | | tggcccgcggtgatgcctttgagggtggccgcatccatctggtcagaaaagacaatctttttgttgtca |
| | | | agcttggtggcaaacgacccgtagagggcgttggacagcaacttggcgatggagcgcagggtttg |
| | | | gtttttgtcgcgatcggcgcgctccttggccgcgatgtttagctgcacgtattcgcgcgcaacgcacc |
| | | | gccattcgggaaagacggtggtgcgctcgtcgggcaccaggtgcacgcgccaaccgcggttgtgc |
| | | | agggtgacaaggtcaacgctggtggctacctctccgcgtaggcgctcgttggtccagcagaggcgg |
| | | | ccgcccttgcgcgagcagaatggcggtagggggtctagctgcgtctcgtccggggggtctgcgtcc |
| | | | acggtaaagaccccgggcagcaggcgcgcgtcgaagtagtctatcttgcatccttgcaagtctagcg |
| | | | cctgctgccatgcgcgggcggcaagcgcgcgctcgtatgggttgagtgggggaccccatggcatg |
| | | | gggtgggtgagcgcggaggcgtacatgccgcaaatgtcgtaaacgtagaggggctctctgagtatt |
| | | | ccaagatatgtagggtagcatcttccaccgcggatgctggcgcgcacgtaatcgtatagttcgtgcga |
| | | | gggagcgaggaggtcgggaccgaggttgctacggggggctgctctgctcggaagactatctgcc |
| | | | tgaagatggcatgtgagttggatgatatggttggacgctggaagacgttgaagctggcgtctgtgaga |
| | | | cctaccgcgtcacgcacgaaggaggcgtaggagtcgcgcagcttgttgaccagctcggcggtgac |
| | | | ctgcacgtctagggcgcagtagtccagggtttccttgatgatgtcatacttatcctgtccctttttttt |
| | | | ccacagctcgcggttgaggacaaactcttcgcggtctttccagtactcttggatcggaaacccgtcggcct |
| | | | ccgaacggtaagagcctagcatgtagaactggttgacggcctggtaggcgcagcatcccttttctac |
| | | | gggtagcgcgtatgcctgcgcggccttccggagcgaggtgtgggtgagcgcaaaggtgtccctga |
| | | | ccatgactttgaggtactggtatttgaagtcagtgtcgtcgcatccgccctgctcccagagcaaaaagt |
| | | | ccgtgcgctttttggaacgcggatttggcagggcgaaggtgacatcgttgaagagtatctttcccgcg |
| | | | cgaggcataaagttgcgtgtgatgcggaagggtcccggcacctcggaacggttgttaattacctggg |
| | | | cggcgagcacgatctcgtcaaagccgttgatgttgtggcccacaatgtaaagttccaagaagcgcgg |
| | | | gatgcccttgatggaaggcaattttttaagttcctcgtaggtgagctcttcaggggagctgagcccgtg |
| | | | ctctgaaagggcccagtctgcaagatgagggttggaagcgacgaatgagctccacaggtcacggg |
| | | | ccattagcatttgcaggtggtcgcgaaaggtcctaaactggcgacctatggccattttttctggggtgat |
| | | | gcagtagaaggtaagcgggtcttgttcccagcggtcccatccaaggttcgcggctaggtctcgcgcg |
| | | | gcagtcactagaggctcatctccgccgaacttcatgaccagcatgaagggcacgagctgcttcccaa |
| | | | aggcccccatccaagtataggtctctacatcgtaggtgacaaagagacgctcggtgcgaggatgcg |
| | | | agccgatcgggaagaactggatctcccgccaccaattggaggagtggctattgatgtggtgaaagta |
| | | | gaagtccctgcgacgggccgaacactcgtgctggcttttgtaaaaacgtgcgcagtactggcagcg |
| | | | gtgcacgggctgtacatcctgcacgaggttgacctgacgaccgcgcacaaggaagcagagtggga |
| | | | atttgagcccctcgcctggcgggtttggctggtggtcttctacttcggctgcttgtccttgaccgtctgg |
| | | | ctgctcgaggggagttacggtggatcggaccaccacgccgcgcgagcccaaagtccagatgtccg |
| | | | cgcgcggcggtcggagcttgatgacaacatcgcgcagatgggagctgtccatggtctggagctccc |
| | | | gcggcgtcaggtcaggcgggagctcctgcaggtttacctcgcatagacgggtcagggcgcgggct |
| | | | agatccaggtgatacctaatttccaggggctggttggtggcggcgtcgatggcttgcaagaggccgc |
| | | | atccccgcggcgcgactacggtaccgcgcggcggggggggccgcgggggtgtccttggatga |
| | | | tgcatctaaaagcggtgacgcgggcgagcccccggaggtagggggggctccggacccgccggg |
| | | | agagggggcaggggcacgtcggcgccgcgcgcgggcaggagctggtgctgcgcgcgtaggttg |
| | | | ctggcgaacgcgacgacgcggcggttgatctcctgaatctggcgcctctgcgtgaagacgacggg |
| | | | cccggtgagcttgaacctgaaagagagttcgacagaatcaatttcggtgtgttgacggcggcctgg |
| | | | cgcaaaatctcctgcacgtctcctgagttgtcttgataggcgatctcggccatgaactgctcgatctctt |
| | | | cctcctggagatctccgcgtccggctcgctccacggtggcggcgaggtcgttggaaatgcgggcca |
| | | | tgagctgcgagaaggcgttgaggcctccctcgttccagacgcggctgtagaccacgcccccttcgg |
| | | | catcgcgggcgcgcatgaccacctgcgcgagattgagctccacgtgccgggcgaagacggcgta |
| | | | gtttcgcaggcgctgaaagaggtagttgagggtggtggcggtgtgttctgccacgaagaagtacata |
| | | | acccagcgtcgcaacgtggattcgttgatatcccccaaggcctcaaggcgctccatggcctcgtaga |
| | | | agtccacggcgaagttgaaaaactgggagttgcgcgccgacacggttaactcctcctccagaagac |
| | | | ggatgagctcggcgacagtgtcgcgcacctcgcgctcaaaggctacaggggcctcttcttcttcttca |
| | | | atctcctcttccataagggcctccccttcttcttcttctggcggcggtgggggaggggggacacggcg |
| | | | gcgacgacggcgcaccgggaggcggtcgacaaagcgctcgatcatctccccgcggcgacggcg |
| | | | catggtctcggtgacggcgcggccgttctcgcgggggcgcagttggaagacgccgcccgtcatgt |
| | | | cccggttatgggttggggggggctgccatgcggcagggatacggcgctaacgatgcatctcaaca |
| | | | attgttgtgtaggtactccgccgccgagggacctgagcgagtccgcatcgaccggatcggaaaacc |
| | | | tctcgagaaaggcgtctaaccagtcacagtcgcaaggtaggctgagcaccgtgggggcggcagc |
| | | | gggcggcggtcggggttgtttctggcggaggtgctgctgatgatgtaattaaagtaggcggtcttgag |
| | | | acggcggatggtcgacagaagcaccatgtccttgggtccggcctgctgaatgcgcaggcggtcgg |
| | | | ccatgccccaggcttcgttttgacatcggcgcaggtctttgtagtagtcttgcatgagcctttctaccgg |
| | | | cacttcttcttctccttcctcttgtcctgcatctcttgcatctatcgctgcggcggcggcggagtttggcc |
| | | | gtaggtggcgccctcttcctcccatgcgtgtgaccccgaagcccctcatcggctgaagcagggctag |
| | | | gtcggcgacaacgcgctcggctaatatggcctgctgcacctgcgtgagggtagactggaagtcatc |
| | | | catgtccacaaagcggtggtatgcgcccgtgttgatggtgtaagtgcagttggccataacggaccag |
| | | | ttaacggtctggtgacccggctgcgagagctcggtgtacctgagacgcgagtaagccctcgagtca |
| | | | aatacgtagtcgttgcaagtccgcaccaggtactggtatcccaccaaaaagtgcggcggcggctgg |
| | | | cggtagaggggccagcgtagggtggccggggctccgggggcgagatcttccaacataaggcgat |
| | | | gatatccgtagatgtacctggacatccaggtgatgccggcggcggtggtggaggcgcgcggaaag |
| | | | tcgcggacgcggttccagatgttgcgcagcggcaaaaagtgctccatggtcgggacgctctggccg |
| | | | gtcaggcgcgcgcaatcgttgacgctctagaccgtgcaaaaggagagcctgtaagcgggcactctt |
| | | | ccgtggtctggtggataaattcgcaagggtatcatggcggacgaccggggttcgagccccgtatcc |
| | | | ggccgtccgccgtgatccatgcggttaccgcccgcgtgtcgaacccaggtgtgcgacgtcagacaa |
| | | | cgggggagtgctccttttggcttccttccaggcgcggcggctgctgcgctagcttttttggccactggc |
| | | | cgcgcgcagcgtaagcggttaggctggaaagcgaaagcattaagtggctcgctccctgtagccgg |
| | | | agggttattttccaagggttgagtcgcgggacccccggttcgagtctcggaccggccggactgcgg |
| | | | cgaacgggggtttgcctccccgtcatgcaagaccccgcttgcaaattcctccggaaacagggacga |
| | | | gccccttttttgcttttcccagatgcatccggtgctgcggcagatgcgcccccctcctcagcagcggc |
| | | | aagagcaagagcagcggcagacatgcagggcaccctcccctcctcctaccgcgtcaggaggggc |
| | | | gacatccgcggttgacgcggcagcagatggtgattacgaacccccgcggcgccgggcccggcac |
| | | | tacctggacttggaggagggcgagggcctggcgcggctaggagcgccctctcctgagcggcacc |
| | | | caagggtgcagctgaagcgtgatacgcgtgaggcgtacgtgccgcggcagaacctgtttcgcgac |
| | | | cgcgagggagaggagcccgaggagatgcgggatcgaaagttccacgcagggcgcgagctgcg |
| | | | gcatggcctgaatcgcgagcggttgctgcgcgaggaggactttgagcccgacgcgcgaaccggg |
| | | | attagtcccgcgcgcgcacacgtggcggccgccgacctggtaaccgcatacgagcagacggtgaa |
| | | | ccaggagattaactttcaaaaaagctttaacaaccacgtgcgtacgcttgtggcgcgcgaggaggtg |
| | | | gctataggactgatgcatctgtgggactttgtaagcgcgctggagcaaaacccaaatagcaagccgc |
| | | | tcatggcgcagctgttccttatagtgcagcacagcagggacaacgaggcattcagggatgcgctgct |
| | | | aaacatagtagagcccgagggccgctggctgctcgatttgataaacatcctgcagagcatagtggtg |
| | | | caggagcgcagcttgagcctggctgacaaggtggccgccatcaactattccatgcttagcctgggca |
| | | | agttttacgcccgcaagatataccataccccttacgttcccatagacaaggaggtaaagatcgaggg |
| | | | gttctacatgcgcatggcgctgaaggtgcttaccttgagcgacgacctgggcgtttatcgcaacgagc |
| | | | gcatccacaaggccgtgagcgtgagccggcggcgcgagctcagcgaccgcgagctgatgcaca |
| | | | gcctgcaaagggccctggctggcacgggcagcggcgatagagaggccgagtcctactttgacgc |
| | | | gggcgctgacctgcgctgggccccaagccgacgcgccctggaggcagctggggccggacctgg |
| | | | gctggcggtggcacccgcgcgcgctggcaacgtcggcggcgtggaggaatatgacgaggacgat |
| | | | gagtacgagccagaggacggcgagtactaagcggtgatgtttctgatcagatgatgcaagacgcaa |
| | | | cggacccggcggtgcgggcggcgctgcagagccagccgtccggccttaactccacggacgactg |
| | | | gcgccaggtcatggaccgcatcatgtcgctgactgcgcgcaatcctgacgcgttccggcagcagcc |
| | | | gcaggccaaccggctctccgcaattctggaagcggtggtcccggcgcgcgcaaaccccacgcac |
| | | | gagaaggtgctggcgatcgtaaacgcgctggccgaaaacagggccatccggcccgacgaggcc |
| | | | ggcctggtctacgacgcgctgcttcagcgcgtggctcgttacaacagcggcaacgtgcagaccaac |
| | | | ctggaccggctggtgggggatgtgcgcgaggccgtggcgcagcgtgagcgcgcgcagcagcag |
| | | | ggcaacctgggctccatggttgcactaaacgccttcctgagtacacagcccgccaacgtgccgcgg |
| | | | ggacaggaggactacaccaactttgtgagcgcactgcggctaatggtgactgagacaccgcaaagt |
| | | | gaggtgtaccagtctgggccagactattttttccagaccagtagacaaggcctgcagaccgtaaacct |
| | | | gagccaggctttcaaaaacttgcaggggctgtggggggtgcgggctcccacaggcgaccgcgcg |
| | | | accgtgtctagcttgctgacgcccaactcgcgcctgttgctgctgctaatagcgcccttcacggacag |
| | | | tggcagcgtgtcccgggacacatacctaggtcacttgctgacactgtaccgcgaggccataggtcag |
| | | | gcgcatgtggacgagcatactttccaggagattacaagtgtcagccgcgcgctggggcaggagga |
| | | | cacgggcagcctggaggcaaccctaaactacctgctgaccaaccggcggcagaagatcccctcgt |
| | | | tgcacagtttaaacagcgaggaggagcgcattttgcgctacgtgcagcagagcgtgagccttaacct |
| | | | gatgcgcgacggggtaacgcccagcgtggcgctggacatgaccgcgcgcaacatggaaccggg |
| | | | catgtatgcctcaaaccggccgtttatcaaccgcctaatggactacttgcatcgcgcggccgccgtga |
| | | | accccgagtatttcaccaatgccatcttgaacccgcactggctaccgccccctggtttctacaccggg |
| | | | ggattcgaggtgcccgagggtaacgatggattcctctgggacgacatagacgacagcgtgttttccc |
| | | | cgcaaccgcagaccctgctagagttgcaacagcgcgagcaggcagaggcggcgctgcgaaagg |
| | | | aaagcttccgcaggccaagcagcttgtccgatctaggcgctgcggccccgcggtcagatgctagta |
| | | | gcccatttccaagcttgatagggtctcttaccagcactcgcaccacccgcccgcgcctgctgggcga |
| | | | ggaggagtacctaaacaactcgctgctgcagccgcagcgcgaaaaaaacctgcctccggcatttcc |
| | | | caacaacgggatagagagcctagtggacaagatgagtagatggaagacgtacgcgcaggagcac |
| | | | agggacgtgccaggcccgcgcccgcccacccgtcgtcaaaggcacgaccgtcagcggggtctgg |
| | | | tgtgggaggacgatgactcggcagacgacagcagcgtcctggatttgggagggagtggcaacccg |
| | | | tttgcgcaccttcgccccaggctggggagaatgttttaaaaaaaaaaaaagcatgatgcaaaataaaa |
| | | | aactcaccaaggccatggcaccgagcgttggttttcttgtattccccttagtatgcggcgcgcggcgat |
| | | | gtatgaggaaggtcctcctccctcctacgagagtgtggtgagcgcggcgccagtggcggcggcgc |
| | | | tgggttctcccttcgatgctcccctggacccgccgtttgtgcctccgcggtacctgcggcctaccggg |
| | | | gggagaaacagcatccgttactctgagttggcacccctattcgacaccacccgtgtgtacctggtgga |
| | | | caacaagtcaacggatgtggcatccctgaactaccagaacgaccacagcaactttctgaccacggtc |
| | | | attcaaaacaatgactacagcccgggggaggcaagcacacagaccatcaatcttgacgaccggtcg |
| | | | cactggggcggcgacctgaaaaccatcctgcataccaacatgccaaatgtgaacgagttcatgtttac |
| | | | caataagtttaaggcgcgggtgatggtgtcgcgcttgcctactaaggacaatcaggtggagctgaaa |
| | | | tacgagtgggtggagttcacgctgcccgagggcaactactccgagaccatgaccatagaccttatga |
| | | | acaacgcgatcgtggagcactacttgaaagtgggcagacagaacggggttctggaaagcgacatc |
| | | | ggggtaaagtttgacacccgcaacttcagactggggtttgaccccgtcactggtcttgtcatgcctgg |
| | | | ggtatatacaaacgaagccttccatccagacatcattttgctgccaggatgcggggtggacttcaccc |
| | | | acagccgcctgagcaacttgttgggcatccgcaagcggcaacccttccaggagggctttaggatca |
| | | | cctacgatgatctggagggtggtaacattcccgcactgttggatgtggacgcctaccaggcgagctt |
| | | | gaaagatgacaccgaacaggggggggtggcgcaggcggcagcaacagcagtggcagcggcg |
| | | | cggaagagaactccaacgcggcagccgcggcaatgcagccggtggaggacatgaacgatcatgc |
| | | | cattcgcggcgacacctttgccacacgggctgaggagaagcgcgctgaggccgaagcagcggcc |
| | | | gaagctgccgcccccgctgcgcaacccgaggtcgagaagcctcagaagaaaccggtgatcaaac |
| | | | ccctgacagaggacagcaagaaacgcagttacaacctaataagcaatgacagcaccttcacccagt |
| | | | accgcagctggtaccttgcatacaactacggcgaccctcagaccggaatccgctcatggaccctgct |
| | | | ttgcactcctgacgtaacctgcggctcggagcaggtctactggtcgttgccagacatgatgcaagac |
| | | | cccgtgaccttccgctccacgcgccagatcagcaactttccggtggtgggcgccgagctgttgcccg |
| | | | tgcactccaagagcttctacaacgaccaggccgtctactcccaactcatccgccagtttacctctctga |
| | | | cccacgtgttcaatcgctttcccgagaaccagattttggcgcgcccgccagcccccaccatcaccac |
| | | | cgtcagtgaaaacgttcctgctctcacagatcacgggacgctaccgctgcgcaacagcatcggagg |
| | | | agtccagcgagtgaccattactgacgccagacgccgcacctgcccctacgtttacaaggccctggg |
| | | | catagtctcgccgcgcgtcctatcgagccgcactttttgagcaagcatgtccatccttatatcgcccag |
| | | | caataacacaggctggggcctgcgcttcccaagcaagatgtttggggggccaagaagcgctccg |
| | | | accaacacccagtgcgcgtgcgcgggcactaccgcgcgccctggggcgcgcacaaacgggcc |
| | | | gcactgggcgcaccaccgtcgatgacgccatcgacgcggtggtggaggaggcgcgcaactacac |
| | | | gcccacgccgccaccagtgtccacagtggacgcggccattcagaccgtggtgcgcggagcccgg |
| | | | cgctatgctaaaatgaagagacggcggaggcgcgtagcacgtcgccaccgccgccgacccggca |
| | | | ctgccgcccaacgcgcggcggcggccctgcttaaccgcgcacgtcgcaccggccgacggggg |
| | | | ccatgcgggccgctcgaaggctggccgcgggtattgtcactgtgccccccaggtccaggcgacga |
| | | | gcggccgccgcagcagccgcggccattagtgctatgactcagggtcgcaggggcaacgtgtattg |
| | | | ggtgcgcgactcggttagcggcctgcgcgtgcccgtgcgcacccgccccccgcgcaactagattg |
| | | | caagaaaaaactacttagactcgtactgttgtatgtatccagcggcggcggcgcgcaacgaagctat |
| | | | gtccaagcgcaaaatcaaagaagagatgctccaggtcatcgcgccggagatctatggccccccga |
| | | | agaaggaagagcaggattacaagccccgaaagctaaagcgggtcaaaaagaaaaagaaagatga |
| | | | tgatgatgaacttgacgacgaggtggaactgctgcacgctaccgcgcccaggcgacgggtacagt |
| | | | ggaaaggtcgacgcgtaaaacgtgttttgcgacccggcaccaccgtagtctttacgcccggtgagc |
| | | | gctccacccgcacctacaagcgcgtgtatgatgaggtgtacggcgacgaggacctgcttgagcagg |
| | | | ccaacgagcgcctcggggagtttgcctacggaaagcggcataaggacatgctggcgttgccgctg |
| | | | gacgagggcaacccaacacctagcctaaagcccgtaacactgcagcaggtgctgcccgcgcttgc |
| | | | accgtccgaagaaaagcgcggcctaaagcgcgagtctggtgacttggcacccaccgtgcagctga |
| | | | tggtacccaagcgccagcgactggaagatgtcttggaaaaaatgaccgtggaacctgggctggagc |
| | | | ccgaggtccgcgtgcggccaatcaagcaggtggcgccgggactgggcgtgcagaccgtggacgt |
| | | | tcagatacccactaccagtagcaccagtattgccaccgccacagagggcatggagacacaaacgtc |
| | | | cccggttgcctcagcggtggcggatgccgcggtgcaggcggtcgctgcggccgcgtccaagacct |
| | | | ctacggaggtgcaaacggacccgtggatgtttcgcgtttcagccccccggcgcccgcgccgttcga |
| | | | ggaagtacggcgccgccagcgcgctactgcccgaatatgccctacatccttccattgcgcctacccc |
| | | | cggctatcgtggctacacctaccgccccagaagacgagcaactacccgacgccgaaccaccactg |
| | | | gaacccgccgccgccgtcgccgtcgccagcccgtgctggccccgatttccgtgcgcagggtggct |
| | | | cgcgaaggaggcaggaccctggtgctgccaacagcgcgctaccaccccagcatcgtttaaaagcc |
| | | | ggtctttgtggttcttgcagatatggccctcacctgccgcctccgtttcccggtgccgggattccgagg |
| | | | aagaatgcaccgtaggaggggcatggccggccacggcctgacgggcggcatgcgtcgtgcgcac |
| | | | caccggcggcggcgcgcgtcgcaccgtcgcatgcgcggcggtatcctgcccctccttattccactg |
| | | | atcgccgcggcgattggcgccgtgcccggaattgcatccgtggccttgcaggcgcagagacactg |
| | | | attaaaaacaagttgcatgtggaaaaatcaaaataaaaagtctggactctcacgctcgcttggtcctgt |
| | | | aactattttgtagaatggaagacatcaactttgcgtctctggccccgcgacacggctcgcgcccgttca |
| | | | tgggaaactggcaagatatcggcaccagcaatatgagcggtggcgccttcagctggggctcgctgt |
| | | | ggagcggcattaaaaatttcggttccaccgttaagaactatggcagcaaggcctggaacagcagca |
| | | | caggccagatgctgagggataagttgaaagagcaaaatttccaacaaaaggtggtagatggcctgg |
| | | | cctctggcattagcggggtggtggacctggccaaccaggcagtgcaaaataagattaacagtaagct |
| | | | tgatccccgccctcccgtagaggagcctccaccggccgtggagacagtgtctccagaggggcgtg |
| | | | gcgaaaagcgtccgcgccccgacagggaagaaactctggtgacgcaaatagacgagcctccctc |
| | | | gtacgaggaggcactaaagcaaggcctgcccaccacccgtcccatcgcgcccatggctaccggag |
| | | | tgctgggccagcacacacccgtaacgctggacctgcctccccccgccgacacccagcagaaacct |
| | | | gtgctgccaggcccgaccgccgttgttgtaacccgtcctagccgcgcgtccctgcgccgcgccgcc |
| | | | agcggtccgcgatcgttgcggcccgtagccagtggcaactggcaaagcacactgaacagcatcgt |
| | | | gggtctgggggtgcaatccctgaagcgccgacgatgcttctgatagctaacgtgtcgtatgtgtgtca |
| | | | tgtatgcgtccatgtcgccgccagaggagctgctgagccgccgcgcgcccgctttccaagatggct |
| | | | accccttcgatgatgccgcagtggtcttacatgcacatctcgggccaggacgcctcggagtacctga |
| | | | gccccgggctggtgcagtttgcccgcgccaccgagacgtacttcagcctgaataacaagtttagaaa |
| | | | ccccacggtggcgcctacgcacgacgtgaccacagaccggtcccagcgtttgacgctgcggttcat |
| | | | ccctgtggaccgtgaggatactgcgtactcgtacaaggcgcggttcaccctagctgtgggtgataac |
| | | | cgtgtgctggacatggcttccacgtactttgacatccgcggcgtgctggacaggggccctacttttaa |
| | | | gccctactctggcactgcctacaacgccctggctcccaagggtgccccaaatccttgcgaatgggat |
| | | | gaagctgctactgctcttgaaataaacctagaagaagaggacgatgacaacgaagacgaagtagac |
| | | | gagcaagctgagcagcaaaaaactcacgtatttgggcaggcgccttattctggtataaatattacaaa |
| | | | ggagggtattcaaataggtgtcgaaggtcaaacacctaaatatgccgataaaacatttcaacctgaac |
| | | | ctcaaataggagaatctcagtggtacgaaacagaaattaatcatgcagctgggagagtcctaaaaaa |
| | | | gactaccccaatgaaaccatgttacggttcatatgcaaaacccacaaatgaaaatggagggcaaggc |
| | | | attcttgtaaagcaacaaaatggaaagctagaaagtcaagtggaaatgcaatttttctcaactactgag |
| | | | gcagccgcaggcaatggtgataacttgactcctaaagtggtattgtacagtgaagatgtagatataga |
| | | | aaccccagacactcatatttcttacatgcccactattaaggaaggtaactcacgagaactaatgggcc |
| | | | aacaatctatgcccaacaggcctaattacattgcttttagggacaattttattggtctaatgtattacaaca |
| | | | gcacgggtaatatgggtgttctggcgggccaagcatcgcagttgaatgctgttgtagatttgcaagac |
| | | | agaaacacagagctttcataccagcttttgcttgattccattggtgatagaaccaggtacttttctatgtg |
| | | | gaatcaggctgttgacagctatgatccagatgttagaattattgaaaatcatggaactgaagatgaactt |
| | | | ccaaattactgctttccactgggaggtgtgattaatacagagactcttaccaaggtaaaacctaaaaca |
| | | | ggtcaggaaaatggatgggaaaaagatgctacagaattttcagataaaaatgaaataagagttggaa |
| | | | ataattttgccatggaaatcaatctaaatgccaacctgtggagaaatttcctgtactccaacatagcgct |
| | | | gtatttgcccgacaagctaaagtacagtccttccaacgtaaaaatttctgataacccaaacacctacga |
| | | | ctacatgaacaagcgagtggtggctcccgggctagtggactgctacattaaccttggagcacgctgg |
| | | | tcccttgactatatggacaacgtcaacccatttaaccaccaccgcaatgctggcctgcgctaccgctc |
| | | | aatgttgctgggcaatggtcgctatgtgcccttccacatccaggtgcctcagaagttctttgccattaaa |
| | | | aacctccttctcctgccgggctcatacacctacgagtggaacttcaggaaggatgttaacatggttctg |
| | | | cagagctccctaggaaatgacctaagggttgacggagccagcattaagtttgatagcatttgcctttac |
| | | | gccaccttcttccccatggcccacaacaccgcctccacgcttgaggccatgcttagaaacgacacca |
| | | | acgaccagtcctttaacgactatctctccgccgccaacatgctctaccctatacccgccaacgctacc |
| | | | aacgtgcccatatccatcccctcccgcaactgggggctttccgcggctgggccttcacgcgcctta |
| | | | agactaaggaaaccccatcactgggctcgggctacgacccttattacacctactctggctctataccct |
| | | | acctagatggaaccttttacctcaaccacacctttaagaaggtggccattacctttgactcttctgtcagc |
| | | | tggcctggcaatgaccgcctgcttacccccaacgagtttgaaattaagcgctcagttgacggggagg |
| | | | gttacaacgttgcccagtgtaacatgaccaaagactggttcctggtacaaatgctagctaactataaca |
| | | | ttggctaccagggcttctatatcccagagagctacaaggaccgcatgtactccttctttagaaacttcca |
| | | | gcccatgagccgtcaggtggtggatgatactaaatacaaggactaccaacaggtgggcatcctaca |
| | | | ccaacacaacaactctggatttgttggctaccttgcccccaccatgcgcgaaggacaggcctaccct |
| | | | gctaacttcccctatccgcttataggcaagaccgcagttgacagcattacccagaaaaagtttctttgc |
| | | | gatcgcaccctttggcgcatcccattctccagtaactttatgtccatggggcactcacagacctgggc |
| | | | caaaaccttctctacgccaactccgcccacgcgctagacatgacttttgaggtggatcccatggacga |
| | | | gcccacccttctttatgttttgtttgaagtctttgacgtggtccgtgtgcaccagccgcaccgcggcgtc |
| | | | atcgaaaccgtgtacctgcgcacgcccttctcggccggcaacgccacaacataaagaagcaagca |
| | | | acatcaacaacagctgccgccatgggctccagtgagcaggaactgaaagccattgtcaaagatcttg |
| | | | gttgtgggccatattttttgggcacctatgacaagcgctttccaggctttgtttctccacacaagctcgcc |
| | | | tgcgccatagtcaatacggccggtcgcgagactgggggcgtacactggatggcctttgcctggaac |
| | | | ccgcactcaaaaacatgctacctctttgagccctttggcttttctgaccagcgactcaagcaggtttacc |
| | | | agtttgagtacgagtcactcctgcgccgtagcgccattgcttcttcccccgaccgctgtataacgctgg |
| | | | aaaagtccacccaaagcgtacaggggcccaactcggccgcctgtggactattctgctgcatgtttctc |
| | | | cacgcctttgccaactggccccaaactcccatggatcacaaccccaccatgaaccttattaccggggt |
| | | | acccaactccatgctcaacagtccccaggtacagcccaccctgcgtcgcaaccaggaacagctcta |
| | | | cagcttcctggagcgccactcgccctacttccgcagccacagtgcgcagattaggagcgccacttcttt |
| | | | ttgtcacttgaaaaacatgtaaaaataatgtactagagacactttcaataaaggcaaatgcttttatttgt |
| | | | acactctcgggtgattatttacccccacccttgccgtctgcgccgtttaaaaatcaaaggggttctgcc |
| | | | gcgcatcgctatgcgccactggcagggacacgttgcgatactggtgtttagtgctccacttaaactca |
| | | | ggcacaaccatccgcggcagctcggtgaagttttcactccacaggctgcgcaccatcaccaacgcg |
| | | | tttagcaggtcgggcgccgatatcttgaagtcgcagttggggcctccgccctgcgcgcgcgagttgc |
| | | | gatacacagggttgcagcactggaacactatcagcgccgggtggtgcacgctggccagcacgctct |
| | | | tgtcggagatcagatccgcgtccaggtcctccgcgttgctcagggcgaacggagtcaactttggtag |
| | | | ctgccttcccaaaaagggcgcgtgcccaggctttgagttgcactcgcaccgtagtggcatcaaaagg |
| | | | tgaccgtgcccggtctgggcgttaggatacagcgcctgcataaaagccttgatctgcttaaaagccac |
| | | | ctgagcctttgcgccttcagagaagaacatgccgcaagacttgccggaaaactgattggccggaca |
| | | | ggccgcgtcgtgcacgcagcaccttgcgtcggtgttggagatctgcaccacatttcggccccaccg |
| | | | gttcttcacgatcttggccttgctagactgctccttcagcgcgcgctgcccgttttcgctcgtcacatcca |
| | | | tttcaatcacgtgctccttatttatcataatgcttccgtgtagacacttaagctcgccttcgatctcagcgc |
| | | | agcggtgcagccacaacgcgcagcccgtgggctcgtgatgcttgtaggtcacctctgcaaacgact |
| | | | gcaggtacgcctgcaggaatcgccccatcatcgtcacaaaggtcttgttgctggtgaaggtcagctg |
| | | | caacccgcggtgctcctcgttcagccaggtcttgcatacggccgccagagcttccacttggtcaggc |
| | | | agtagtttgaagttcgcctttagatcgttatccacgtggtacttgtccatcagcgcgcgcgcagcctcca |
| | | | tgcccttctcccacgcagacacgatcggcacactcagcgggttcatcaccgtaatttcactttccgctt |
| | | | cgctgggctcttcctcttcctcttgcgtccgcataccacgcgccactgggtcgtcttcattcagccgcc |
| | | | gcactgtgcgcttacctcctttgccatgcttgattagcaccggtgggttgctgaaacccaccatttgtag |
| | | | cgccacatcttctctttcttcctcgctgtccacgattacctctggtgatgggggcgctcgggcttggga |
| | | | gaagggcgcttctttttcttcttgggcgcaatggccaaatccgccgccgaggtcgatggccgcgggc |
| | | | tgggtgtgcgcggcaccagcgcgtcttgtgatgagtcttcctcgtcctcggactcgatacgccgcctc |
| | | | atccgcttttttgggggcgcccggggaggcggcggcgacggggacggggacgacacgtcctcca |
| | | | tggttgggggacgtcgcgccgcaccgcgtccgcgctcgggggtggtttcgcgctgctcctcttcccg |
| | | | actggccatttccttctcctataggcagaaaaagatcatggagtcagtcgagaagaaggacagccta |
| | | | accgccccctctgagttcgccaccaccgcctccaccgatgccgccaacgcgcctaccaccttcccc |
| | | | gtcgaggcacccccgcttgaggaggaggaagtgattatcgagcaggacccaggttttgtaagcgaa |
| | | | gacgacgaggaccgctcagtaccaacagaggataaaaagcaagaccaggacaacgcagaggca |
| | | | aacgaggaacaagtcgggggggggacgaaaggcatggcgactacctagatgtgggagacgac |
| | | | gtgctgttgaagcatctgcagcgccagtgcgccattatctgcgacgcgttgcaagagcgcagcgatg |
| | | | tgcccctcgccatagcggatgtcagccttgcctacgaacgccacctattctcaccgcgcgtaccccc |
| | | | caaacgccaagaaaacggcacatgcgagcccaacccgcgcctcaacttctaccccgtatttgccgt |
| | | | gccagaggtgcttgccacctatcacatctttttccaaaactgcaagatacccctatcctgccgtgccaa |
| | | | ccgcagccgagcggacaagcagctggccttgcggcagggcgctgtcatacctgatatcgcctcgct |
| | | | caacgaagtgccaaaaatctttgagggtcttggacgcgacgagaagcgcgcggcaaacgctctgc |
| | | | aacaggaaaacagcgaaaatgaaagtcactctggagtgttggtggaactcgagggtgacaacgcg |
| | | | cgcctagccgtactaaaacgcagcatcgaggtcacccactttgcctacccggcacttaacctacccc |
| | | | ccaaggtcatgagcacagtcatgagtgagctgatcgtgcgccgtgcgcagcccctggagagggat |
| | | | gcaaatttgcaagaacaaacagaggagggcctacccgcagttggcgacgagcagctagcgcgctg |
| | | | gcttcaaacgcgcgagcctgccgacttggaggagcgacgcaaactaatgatggccgcagtgctcgt |
| | | | taccgtggagcttgagtgcatgcagcggttctttgctgacccggagatgcagcgcaagctagaggaa |
| | | | acattgcactacacctttcgacagggctacgtacgccaggcctgcaagatctccaacgtggagctct |
| | | | gcaacctggtctcctaccttggaattttgcacgaaaaccgccttgggcaaaacgtgcttcattccacgc |
| | | | tcaagggcgaggcgcgccgcgactacgtccgcgactgcgtttacttatttctatgctacacctggcag |
| | | | acggccatgggcgtttggcagcagtgcttggaggagtgcaacctcaaggagctgcagaaactgcta |
| | | | aagcaaaacttgaaggacctatggacggccttcaacgagcgctccgtggccgcgcacctggcgga |
| | | | catcattttccccgaacgcctgcttaaaaccctgcaacagggtctgccagacttcaccagtcaaagca |
| | | | tgttgcagaactttaggaactttatcctagagcgctcaggaatcttgcccgccacctgctgtgcacttcc |
| | | | tagcgactttgtgcccattaagtaccgcgaatgccctccgccgctttggggccactgctaccttctgca |
| | | | gctagccaactaccttgcctaccactctgacataatggaagacgtgagcggtgacggtctactggagt |
| | | | gtcactgtcgctgcaacctatgcaccccgcaccgctccctggtttgcaattcgcagctgcttaacgaa |
| | | | agtcaaattatcggtacctttgagctgcagggtccctcgcctgacgaaaagtccgcggctccggggtt |
| | | | gaaactcactccggggctgtggacgtcggcttaccttcgcaaatttgtacctgaggactaccacgccc |
| | | | acgagattaggttctacgaagaccaatcccgcccgcctaatgcggagcttaccgcctgcgtcattacc |
| | | | cagggccacattcttggccaattgcaagccatcaacaaagcccgccaagagtttctgctacgaaagg |
| | | | gacggggggtttacttggacccccagtccggcgaggagctcaacccaatccccccgccgccgcag |
| | | | ccctatcagcagcagccgcgggcccttgcttcccaggatggcacccaaaaagaagctgcagctgc |
| | | | cgccgccacccacggacgaggaggaatactgggacagtcaggcagaggaggttttggacgagga |
| | | | ggaggaggacatgatggaagactgggagagcctagacgaggaagcttccgaggtcgaagaggtg |
| | | | tcagacgaaacaccgtcaccctcggtcgcattcccctcgccggcgccccagaaatcggcaaccggt |
| | | | tccagcatggctacaacctccgctcctcaggcgccgccggcactgcccgttcgccgacccaaccgt |
| | | | agatgggacaccactggaaccagggccggtaagtccaagcagccgccgccgttagcccaagagc |
| | | | aacaacagcgccaaggctaccgctcatggcgcgggcacaagaacgccatagttgcttgcttgcaag |
| | | | actgtgggggcaacatctccttcgcccgccgctttcttctctaccatcacggcgtggccttcccccgta |
| | | | acatcctgcattactaccgtcatctctacagcccatactgcaccggcggcagcggcagcaacagcag |
| | | | cggccacacagaagcaaaggcgaccggatagcaagactctgacaaagcccaagaaatccacagc |
| | | | ggcggcagcagcaggaggaggagcgctgcgtctggcgcccaacgaacccgtatcgacccgcga |
| | | | gcttagaaacaggatttttcccactctgtatgctatatttcaacagagcaggggccaagaacaagagct |
| | | | gaaaataaaaaacaggtctctgcgatccctcacccgcagctgcctgtatcacaaaagcgaagatcag |
| | | | cttcggcgcacgctggaagacgcggaggctctcttcagtaaatactgcgcgctgactcttaaggact |
| | | | agtttcgcgccctttctcaaatttaagcgcgaaaactacgtcatctccagcggccacacccggcgcca |
| | | | gcacctgttgtcagcgccattatgagcaaggaaattcccacgccctacatgtggagttaccagccaca |
| | | | aatgggacttgcggctggagctgcccaagactactcaacccgaataaactacatgagcgcgggacc |
| | | | ccacatgatatcccgggtcaacggaatacgcgcccaccgaaaccgaattctcctggaacaggcggc |
| | | | tattaccaccacacctcgtaataaccttaatccccgtagttggcccgctgccctggtgtaccaggaaag |
| | | | tcccgctcccaccactgtggtacttcccagagacgcccaggccgaagttcagatgactaactcaggg |
| | | | gcgcagcttgcgggggctttcgtcacagggtgcggtcgcccgggcagggtataactcacctgaca |
| | | | atcagagggcgaggtattcagctcaacgacgagtcggtgagctcctcgcttggtctccgtccggacg |
| | | | ggacatttcagatcggcggcgccggccgctcttcattcacgcctcgtcaggcaatcctaactctgcag |
| | | | acctcgtcctctgagccgcgctctggaggcattggaactctgcaatttattgaggagtttgtgccatcg |
| | | | gtctactttaaccccttctcgggacctcccggccactatccggatcaatttattcctaactttgacgcggt |
| | | | aaaggactcggcggacggctacgactgaatgttaagtggagaggcagagcaactgcgcctgaaac |
| | | | acctggtccactgtcgccgccacaagtgctttgcccgcgactccggtgagttttgctactttgaattgcc |
| | | | cgaggatcatatcgagggcccggcgcacggcgtccggcttaccgcccagggagagcttgcccgta |
| | | | gcctgattcgggagtttacccagcgccccctgctagttgagcgggacaggggaccctgtgttctcact |
| | | | gtgatttgcaactgtcctaaccctggattacatcaagatctttgttgccatctctgtgctgagtataataaa |
| | | | tacagaaattaaaatatactggggctcctatcgccatcctgtaaacgccaccgtcttcacccgcccaa |
| | | | gcaaaccaaggcgaaccttacctggtacttttaacatctctccctctgtgatttacaacagtttcaaccca |
| | | | gacggagtgagtctacgagagaacctctccgagctcagctactccatcagaaaaaacaccaccctc |
| | | | cttacctgccgggaacgtacgagtgcgtcaccggccgctgcaccacacctaccgcctgaccgtaaa |
| | | | ccagactttttccggacagacctcaataactctgtttaccagaacaggaggtgagcttagaaaaccctt |
| | | | agggtattaggccaaaggcgcagctactgtggggtttatgaacaattcaagcaactctacgggctatt |
| | | | ctaattcaggtttctctagaaatggacggaattattacagagcagcgcctgctagaaagacgcagggc |
| | | | agcggccgagcaacagcgcatgaatcaagagctccaagacatggttaacttgcaccagtgcaaaa |
| | | | ggggtatcttttgtctggtaaagcaggccaaagtcacctacgacagtaataccaccggacaccgcctt |
| | | | agctacaagttgccaaccaagcgtcagaaattggtggtcatggtgggagaaaagcccattaccataa |
| | | | ctcagcactcggtagaaaccgaaggctgcattcactcaccttgtcaaggacctgaggatctctgcacc |
| | | | cttattaagaccctgtgcggtctcaaagatcttattccctttaactaataaaaaaaaataataaagcatca |
| | | | cttacttaaaatcagttagcaaatttctgtccagtttattcagcagcacctccttgccctcctcccagctct |
| | | | ggtattgcagcttcctcctggctgcaaactttctccacaatctaaatggaatgtcagtttcctcctgttcct |
| | | | gtccatccgcacccactatcttcatgttgttgcagatgaagcgcgcaagaccgtctgaagataccttca |
| | | | accccgtgtatccatatgacacggaaaccggtcctccaactgtgccttttcttactcctccctttgtatcc |
| | | | cccaatgggtttcaagagagtccccctggggtactctctttgcgcctatccgaacctctagttacctcca |
| | | | atggcatgcttgcgctcaaaatgggcaacggcctctctctggacgaggccggcaaccttacctccca |
| | | | aaatgtaaccactgtgagcccacctctcaaaaaaaccaagtcaaacataaacctggaaatatctgcac |
| | | | ccctcacagttacctcagaagccctaactgtggctgccgccgcacctctaatggtcgcgggcaacac |
| | | | actcaccatgcaatcacaggccccgctaaccgtgcacgactccaaacttagcattgccacccaagga |
| | | | cccctcacagtgtcagaaggaaagctagccctgcaaacatcaggccccctcaccaccaccgatagc |
| | | | agtacccttactatcactgcctcaccccctctaactactgccactggtagcttgggcattgacttgaaag |
| | | | agcccatttatacacaaaatggaaaactaggactaaagtacggggctcctttgcatgtaacagacgac |
| | | | ctaaacactttgaccgtagcaactggtccaggtgtgactattaataatacttccttgcaaactaaagttac |
| | | | tggagccttgggttttgattcacaaggcaatatgcaacttaatgtagcaggaggactaaggattgattct |
| | | | caaaacagacgccttatacttgatgttagttatccgtttgatgctcaaaaccaactaaatctaagactag |
| | | | gacagggccctctttttataaactcagcccacaacttggatattaactacaacaaaggcctttacttgttt |
| | | | acagcttcaaacaattccaaaaagcttgaggttaacctaagcactgccaaggggttgatgtttgacgct |
| | | | acagccatagccattaatgcaggagatgggcttgaatttggttcacctaatgcaccaaacacaaatcc |
| | | | cctcaaaacaaaaattggccatggcctagaatttgattcaaacaaggctatggttcctaaactaggaac |
| | | | tggccttagttttgacagcacaggtgccattacagtaggaaacaaaaataatgataagctaactttgtg |
| | | | gaccacaccagctccatctcctaactgtagactaaatgcagagaaagatgctaaactcactttggtctt |
| | | | aacaaaatgtggcagtcaaatacttgctacagtttcagttttggctgttaaaggcagtttggctccaatat |
| | | | ctggaacagttcaaagtgctcatcttattataagatttgacgaaaatggagtgctactaaacaattccttc |
| | | | ctggacccagaatattggaactttagaaatggagatcttactgaaggcacagcctatacaaacgctgtt |
| | | | ggatttatgcctaacctatcagcttatccaaaatctcacggtaaaactgccaaaagtaacattgtcagtc |
| | | | aagtttacttaaacggagacaaaactaaacctgtaacactaaccattacactaaacggtacacaggaa |
| | | | acaggagacacaactccaagtgcatactctatgtcattttcatgggactggtctggccacaactacattaa |
| | | | tgaaatatttgccacatcctcttacactttttcatacattgcccaagaataaagaatcgtttgtgttatgtt |
| | | | tcaacgtgtttatttttcaattgcagaaaatttcgaatcatttttcattcagtagtatagccccaccaccaca |
| | | | tagcttatacagatcaccgtaccttaatcaaactcacagaaccctagtattcaacctgccacctccctcc |
| | | | caacacacagagtacacagtcctttctccccggctggccttaaaaagcatcatatcatgggtaacaga |
| | | | catattcttaggtgttatattccacacggtttcctgtcgagccaaacgctcatcagtgatattaataaactc |
| | | | cccgggcagctcacttaagttcatgtcgctgtccagctgctgagccacaggctgctgtccaacttgcg |
| | | | gttgcttaacgggcggcgaaggagaagtccacgcctacatgggggtagagtcataatcgtgcatca |
| | | | ggatagggcggtggtgctgcagcagcgcgcgaataaactgctgccgccgccgctccgtcctgcag |
| | | | gaatacaacatggcagtggtctcctcagcgatgattcgcaccgcccgcagcataaggcgccttgtcc |
| | | | tccgggcacagcagcgcaccctgatctcacttaaatcagcacagtaactgcagcacagcaccacaa |
| | | | tattgttcaaaatcccacagtgcaaggcgctgtatccaaagctcatggggggaccacagaacccac |
| | | | gtggccatcataccacaagcgcaggtagattaagtggcgacccctcataaacacgctggacataaa |
| | | | cattacctcttttggcatgttgtaattcaccacctcccggtaccatataaacctctgattaaacatggcgc |
| | | | catccaccaccatcctaaaccagctggccaaaacctgcccgccggctatacactgcagggaaccgg |
| | | | gactggaacaatgacagtggagagcccaggactcgtaaccatggatcatcatgctcgtcatgatatc |
| | | | aatgttggcacaacacaggcacacgtgcatacacttcctcaggattacaagctcctcccgcgttagaa |
| | | | ccatatcccagggaacaacccattcctgaatcagcgtaaatcccacactgcagggaagacctcgca |
| | | | cgtaactcacgttgtgcattgtcaaagtgttacattcgggcagcagcggatgatcctccagtatggtag |
| | | | cgcgggtttctgtctcaaaaggaggtagacgatccctactgtacggagtgcgccgagacaaccgag |
| | | | atcgtgttggtcgtagtgtcatgccaaatggaacgccggacgtagtcatatttcctgaagcaaaacca |
| | | | ggtgcgggcgtgacaaacagatctgcgtctccggtctcgccgcttagatcgctctgtgtagtagttgta |
| | | | gtatatccactctctcaaagcatccaggcgccccctggcttcgggttctatgtaaactccttcatgcgcc |
| | | | gctgccctgataacatccaccaccgcagaataagccacacccagccaacctacacattcgttctgcg |
| | | | agtcacacacgggaggagcgggaagagctggaagaaccatgtttttttttttattccaaaagattatcc |
| | | | aaaacctcaaaatgaagatctattaagtgaacgcgctcccctccggtggcgtggtcaaactctacagc |
| | | | caaagaacagataatggcatttgtaagatgttgcacaatggcttccaaaaggcaaacggccctcacgt |
| | | | ccaagtggacgtaaaggctaaacccttcagggtgaatctcctctataaacattccagcaccttcaacc |
| | | | atgcccaaataattctcatctcgccaccttctcaatatatctctaagcaaatcccgaatattaagtccggc |
| | | | cattgtaaaaatctgctccagagcgccctccaccttcagcctcaagcagcgaatcatgattgcaaaaa |
| | | | ttcaggttcctcacagacctgtataagattcaaaagcggaacattaacaaaaataccgcgatcccgta |
| | | | ggtcccttcgcagggccagctgaacataatcgtgcaggtctgcacggaccagcgcggccacttccc |
| | | | cgccaggaaccatgacaaaagaacccacactgattatgacacgcatactcggagctatgctaacca |
| | | | gcgtagccccgatgtaagcttgttgcatgggcggcgatataaaatgcaaggtgctgctcaaaaaatc |
| | | | aggcaaagcctcgcgcaaaaaagaaagcacatcgtagtcatgctcatgcagataaaggcaggtaa |
| | | | gctccggaaccaccacagaaaaagacaccatttttctctcaaacatgtctgcgggtttctgcataaaca |
| | | | caaaataaaataacaaaaaaacatttaaacattagaagcctgtcttacaacaggaaaaacaacccttat |
| | | | aagcataagacggactacggccatgccggcgtgaccgtaaaaaaactggtcaccgtgattaaaaag |
| | | | caccaccgacagctcctcggtcatgtccggagtcataatgtaagactcggtaaacacatcaggttgat |
| | | | tcacatcggtcagtgctaaaaagcgaccgaaatagcccgggggaatacatacccgcaggcgtaga |
| | | | gacaacattacagcccccataggaggtataacaaaattaataggagagaaaaacacataaacacctg |
| | | | aaaaaccctcctgcctaggcaaaatagcaccctcccgctccagaacaacatacagcgcttccacag |
| | | | cggcagccataacagtcagccttaccagtaaaaaagaaaacctattaaaaaaacaccactcgacac |
| | | | ggcaccagctcaatcagtcacagtgtaaaaaagggccaagtgcagagcgagtatatataggactaa |
| | | | aaaatgacgtaacggttaaagtccacaaaaaacacccagaaaaccgcacgcgaacctacgcccag |
| | | | aaacgaaagccaaaaaacccacaacttcctcaaatcgtcacttccgttttcccacgttacgtcacttcc |
| | | | cattttaagaaaactacaattcccaacacatacaagttactccgccctaaaacctacgtcacccgcccc |
| | | | gttcccacgccccgcgccacgtcacaaactccaccccctcattatcatattggcttcaatccaaaataa |
| | | | ggtatattattgatgatgttaattaa |
| 5′ AAV | A47 | 47 | ctgcgcgctcgctcgctcactgaggccgcccgggcaaagcccgggcgtcgggcgacctttggtcg |
| nucleotide | | | cccggcctcagtgagcgagcgagcgcgcagagagggagtggccaactccatcactaggggttcct |
| sequence | | | tgtagttaatgattaacccgccatgctacttatctacgtagccatgctctaggaagatcggaattcgccc |
| excluding | | | ttaagctagctagttattaatagtaatcaattacggggtcattagttcatagcccatatatggagttccg |
| the | | | |
| minigene | | | |
| cassette | | | |
| 3′ AAV | A48 | 48 | ctcgagttaagggcgaattcccgataaggatcttcctagagcatggctacgtagataagtagcatggc |
| nucleotide | | | gggttaatcattaactacaaggaacccctagtgatggagttggccactccctctctgcgcgctcgctc |
| sequence | | | gctcactgaggccgggcgaccaaaggtcgcccgacgcccgggctttgcccgggcggcctcagtg |
| excluding | | | agcgagcgagcgcgcag |
| the | | | |
| minigene | | | |
| cassette | | | |
| AAV-A1 | A49 | 49 | ctgcgcgctcgctcgctcactgaggccgcccgggcaaagcccgggcgtgggcgacctttggtcg |
| minigene | | | cccggcctcagtgagcgagcgagcgcgcagagagggagtggccaactccatcactaggggttcct |
| nucleotide | | | tgtagttaatgattaacccgccatgctacttatctacgtagccatgctctaggaagatcggaattcgccc |
| sequence | | | ttaagctagctagttattaatagtaatcaattacggggtcattagttcatagcccatatatggagttccga |
| | | | caagtttgtacaaaaaagcaggctccaccatggtaactataacggtcctaaggtagcgaagaaccaa |
| | | | ttcagtcgagcctttcactcattagatgcatgtcgttacataacttacggtaaatggcccgcctggctga |
| | | | ccgcccaacgacccccgcccattgacgtcaataatgacgtatgttcccatagtaacgccaataggga |
| | | | ctttccattgacgtcaatgggtggagtatttacggtaaactgcccacttggcagtacatcaagtgtatca |
| | | | tatgccaagtacgccccctattgacgtcaatgacggtaaatggcccgcctggcattatgcccagtaca |
| | | | tgaccttatgggactttcctacttggcagtacatctacgtattagtcatcgctattaccatggtgatgcggt |
| | | | tttggcagtacatcaatgggcgtggatagcggtttgactcacggggatttccaagtctccaccccattg |
| | | | acgtcaatgggagtttgttttggcaccaaaatcaacgggactttccaaaatgtcgtaacaactccgccc |
| | | | cattgacgcaaatgggcggtaggcgtgtacggtgggaggtctatataagcagagctctccctatcag |
| | | | tgatagagatctccctatcagtgatagagatcgtcgacgagctcgtttagtgaaccgtcagatcgcctg |
| | | | gagacgccatccacgctgttttgacctccatagaagacaccgggaccgatccagcctccggttaagc |
| | | | ttggtacccccgccgccaccatgggagtttacgatggcagagagcacaccgtctgagcggccgctc |
| | | | gagcatgcatctagagggccctattctatagtgtcacctaaatgctagagctcgctgatcagcctcgac |
| | | | tgtgccttctagttgccagccatctgttgtttgcccctcccccgtgccttccttgaccctggaaggtgcc |
| | | | actcccactgtcctttcctaataaaatgaggaaattgcatcgcattgtctgagtaggtgtcattctattctg |
| | | | gggggtggggggggcaggacagcaagggggaggattgggaagacaatagcaggcatgctggg |
| | | | gatgcggtgggctctatggcttctgaggcggaaagaaccagctggggctcgaggggggatcgatc |
| | | | cgtcgagatatctagggataacagggtaattagacccagctttcttgtacaaagtggtctcgagttaag |
| | | | ggcgaattcccgataaggatcttcctagagcatggctacgtagataagtagcatggcgggttaatcatt |
| | | | aactacaaggaacccctagtgatggagttggccactccctctctgcgcgctcgctcgctcactgagg |
| | | | ccgggcgaccaaaggtcgcccgacgcccgggctttgcccgggcggcctcagtgagcgagcgag |
| | | | cgcgcag |
| 5′ ITR | A50 | 50 | ctgcgcgctcgctcgctcactgaggccgcccgggcaaagcccgggcgtcgggcgacctttggtcg |
| nucleotide | | | cccggcctcagtgagcgagcgagcgcgcagagagggagtggccaactccatcactaggggttcct |
| sequence | | | |
| 3′ ITR | A51 | 51 | aggaacccctagtgatggagttggccactccctctctgcgcgctcgctcgctcactgaggccgggc |
| nucleotide | | | gaccaaaggtcgcccgacgcccgggctttgcccgggcggcctcagtgagcgagcgagcgcgca |
| sequence | | | g |
| EF1 a | A122 | 122 | ggctccggtgcccgtcagtgggcagagcgcacatcgcccacagtccccgagaagttggggggag |
| promoter | | | gggtcggcaattgaaccggtgcctagagaaggtggcgcggggtaaactgggaaagtgatgtcgtgt |
| | | | actggctccgcctttttcccgagggtgggggagaaccgtatataagtgcagtagtcgccgtgaacgtt |
| | | | ctttttcgcaacgggtttgccgccagaacacaggtaagtgccgtgtgtggttcccgcgggcctggcct |
| | | | ctttacgggttatggcccttgcgtgccttgaattacttccacgcccctggctgcagtacgtgattcttgat |
| | | | cccgagcttcgggttggaagtgggtgggagagttcgaggccttgcgcttaaggagccccttcgcctc |
| | | | gtgcttgagttgaggcctggcctgggcgctggggccgccgcgtgcgaatctggtggcaccttcgcgcct |
| | | | gtctcgctgctttcgataagtctctagccatttaaaatttttgatgacctgctgcgacgctttttttctgg |
| | | | caagatagtcttgtaaatgcgggccaagatctgcacactggtatttcggtttttggggccgcggggg |
| | | | cgacggggcccgtgcgtcccagcgcacatgttcggcgaggggggcctgcgagcgcggccacc |
| | | | gagaatcggacgggggtagtctcaagctggccggcctgctctggtgcctggcctcgcgccgccgt |
| | | | gtatcgccccgccctgggcggcaaggctggcccggtcggcaccagttgcgtgagcggaaagatg |
| | | | gccgcttcccggccctgctgcagggagctcaaaatggaggacgcggcgctcgggagagcgggc |
| | | | gggtgagtcacccacacaaaggaaaagggcctttccgtcctcagccgtcgcttcatgtgactccacg |
| | | | gagtaccgggcgccgtccaggcacctcgattagttctcgagcttttggagtacgtcgtctttaggttgg |
| | | | ggggaggggttttatgcgatggagtttccccacactgagtgggtggagactgaagttaggccagcttgg |
| | | | cacttgatgtaattctccttggaatttgccctttttgagtttggatcttggttcattctcaagcctcagac |
| | | | agtggttcaaagtttttttcttccatttcaggtgtcgtga |

### EXAMPLES

These examples are provided for illustrative purposes only and not to limit the scope of the claims provided herein. These examples are provided for purposes of illustration only, and are not intended to be limiting unless otherwise specified. Thus, the present disclosure should in no way be construed as being limited to the following examples, but rather, should be construed to encompass any and all variations which become evident as a result of the teaching provided herein. Without further description, it is believed that one of ordinary skill in the art can, using the preceding description and the following illustrative examples, make and utilize the compounds of the present disclosure and practice the claimed methods.

### Example 1: Identification and Selection of Cancer-Specific CD8+ T Cell Epitopes

Initial candidates are chosen from antigen-presenting cells that express the HLA molecule(s) of interest and immunoaffinity purification of HLA class I molecules were conducted using standard molecular biology techniques. LC-MS/MS analysis of purified MHC eluate was conducted, and bioinformatics analysis was performed to identify initial candidates (Table 4).

TABLE 4
Selected peptide candidates based on
bioinformatics analysis

| SEQ | SEQ ID | | |
|---|---|---|---|
| Identifier | NO. | Sequence | HLA Restriction |
| A1 | 1 | GVYDGREHTV | HLA-A*02:01 |
| A2 | 2 | KLVELEHTL | HLA-A*02:01 |
| A3 | 3 | SPSSASLAL | HLA-B*07:02 |
| | | | HLA-B*35:03 |
| A4 | 4 | KPRPDVTNEL | HLA-B*07:02 |
| | | | HLA-B*35:03 |
| | | | HLA-C*07:02 |
| A5 | 5 | KEFAFLEHSL | HLA-B*40:01 |
| | | | HLA-B*40:02 |
| | | | HLA-B*44:02 |
| A6 | 6 | HELGFKVVL | HLA-B*40:01 |
| | | | HLA-B*40:02 |
| | | | HLA-B*44:02 |
| | | | HLA-B*18:01 |
| A7 | 7 | LKPDHIQR | HLA-A*03:01 |
| | | | HLA-A*11:01 |
| A8 | 8 | SLFGARPGR | HLA-A*03:01 |
| | | | HLA-A*11:01 |
| | | | HLA-A*31:01 |
| A9 | 9 | KTEVHGRLK | HLA-A*03:01 |
| | | | HLA-A*11:01 |
| A10 | 10 | NLRPPTQEL | HLA-B*07:02 |
| | | | HLA-B*35:03 |
| | | | HLA-C*07:02 |
| | | | HLA-C*03:03 |
| A11 | 11 | RVSLPKLGYK | HLA-A*03:01 |
| A12 | 12 | LRPPTQEL | HLA-C*07:02 |
| | | | HLA-B*35:03 |
| A13 | 13 | RPASPRPAP | HLA-B*07:02 |
| | | | HLA-B*35:03 |
| A14 | 14 | IATKIALQM | HLA-C*03:04 |
| A15 | 15 | SLYQTIRLK | HLA-A*03:01 |
| | | | HLA-A*11:01 |
| A16 | 16 | KTPLHTLLK | HLA-A*03:01 |
| | | | HLA-A*11:01 |
| A17 | 17 | RLHSFTLRQK | HLA-A*03:01 |
| | | | HLA-A*11:01 |
| A18 | 18 | RVFTSSLKTK | HLA-A*03:01 |
| | | | HLA-A*11:01 |
| A19 | 19 | RVLAKGLAK | HLA-A*03:01 |
| | | | HLA-A*11:01 |
| A20 | 20 | GPRPSPTRSV | HLA-B*07:02 |
| | | | HLA-B*35:03 |
| A21 | 21 | GQHLHLETF | HLA-B*15:01 |
| A22 | 22 | SPRSPSPSL | HLA-B*07:02 |
| | | | HLA-B*35:03 |
| A23 | 23 | VPQEAVRAPL | HLA-B*07:02 |
| | | | HLA-B*35:03 |
| A24 | 24 | IYPFINSH | HLA-C*07:02 |
| | | | HLA-B*35:03 |
| A25 | 25 | SPSSASLTL | HLA-B*07:02 |
| | | | HLA-B*35:03 |
| A26 | 26 | LNKVKTSL | HLA-B*08:01 |
| A27 | 27 | NLKTHLRL | HLA-B*08:01 |
| A28 | 28 | FRGVFVHRY | HLA-C*07:01 |
| A29 | 29 | TSGPVTEKY | HLA-B*35:01 |
| A30 | 30 | VVAAHLAGA | HLA-A*02:05 |
| A31 | 31 | KSYSKVLVR | HLA-A*03:01 |
| | | | HLA-A*11:01 |
| A32 | 32 | MTYKIHFKK | HLA-A*03:01 |
| | | | HLA-A*11:01 |
| A33 | 33 | VPASPALSR | HLA-B*07:02 |
| | | | HLA-B*35:03 |
| A34 | 34 | SPSARPPSL | HLA-B*07:02 |
| | | | HLA-B*35:03 |
| A35 | 35 | SPTLNVSAL | HLA-B*07:02 |
| | | | HLA-B*35:03 |

Immunogenicity of the potential peptide candidates were tested in vitro by testing for stimulation on HLA-matched peripheral blood lymphocytes of suspected or diagnosed oesophageal cancer patients. The results from the immunogenicity analysis are shown in Table 5.

TABLE 5
Results of immunogenicity analysis

| SEQ | SEQ | | Immunogenic Response |
|---|---|---|---|
| Identifier | ID | | (Positive Response/ |
| | NO. | Sequence | Total Participants) |
| A1 | 1 | GVYDGREHTV | 8/10 |
| A2 | 2 | KLVELEHTL | 8/10 |
| A3 | 3 | SPSSASLAL | 3/5 |
| A4 | 4 | KPRPDVTNEL | 4/5 |
| A5 | 5 | KEFAFLEHSL | 7/10 |
| A6 | 6 | HELGFKVVL | 7/10 |
| A7 | 7 | LKPDHIQR | 4/13 |
| A8 | 8 | SLFGARPGR | 6/9 |
| A9 | 9 | KTEVHGRLK | 4/9 |
| A10 | 10 | NLRPPTQEL | 4/5 |
| A11 | 11 | RVSLPKLGYK | 9/13 |
| A12 | 12 | LRPPTQEL | 4/6 |
| A13 | 13 | RPASPRPAP | 4/5 |
| A14 | 14 | IATKIALQM | 3/4 |
| A16 | 16 | KTPLHTLLK | 2/3 |
| A20 | 20 | GPRPSPTRSV | 2/4 |
| A22 | 22 | SPRSPSPSL | 4/5 |
| A23 | 23 | VPQEAVRAPL | 3/5 |
| A24 | 24 | IYPFINSH | 3/7 |
| A25 | 25 | SPSSASLTL | 3/5 |
| A28 | 28 | FRGVFVHRY | 3/5 |
| A29 | 29 | TSGPVTEKY | 3/4 |
| A30 | 30 | VVAAHLAGA | 10/11 |
| A31 | 31 | KSYSKVLVR | 5/10 |
| A32 | 32 | MTYKIHFKK | 5/11 |
| A33 | 33 | VPASPALSR | 6/13 |
| A34 | 34 | SPSARPPSL | 5/13 |
| A35 | 35 | SPTLNVSAL | 2/3 |

The peptides were further analyzed and selected and refined based on no or minimal the expression of the peptide sequence across healthy tissue samples in the GTEex database. Only peptides with minimal expression across all healthy tissues were included. of sequence profiles of normal tissues. To further validate the peptide sequences, evidence of broad prevalence and high expression levels in multiple samples of oesophageal tumour from The Cancer Genome Atlas (TCGA) and Allele Frequency Net Database (AFND) databases were evidenced. Results from the prevalence analysis are shown in Table 6.

TABLE 6
Results from prevalence analysis

| SEQ | | | | | | | | | Prevalence |
|---|---|---|---|---|---|---|---|---|---|
| ID | | HLA | Number | Chromo- | Immuno- | G-Tex | Gtex | TCGA | in TCGA |
| NO: | Sequence | Restriction | Locations | some | genicity | Rank | Comment | Rank | samples |
| 1 | GVYDGR | HLA- | 1 | ChrX | +++ | 154/2 | G-Tex | 17 | 20%-40% |
| | EHTV | A*02:01 | | | | | Good | | |
| 2 | KLVELEH | HLA- | 1 | ChrX | + | 317/6 | G-Tex | 7 | 90% |
| | TL | A*02:01 | | | | | Good | | |
| 3 | SPSSASL | HLA- | 2 | Chr7, | + | 141/1 | G-Tex | 16 | 30%-50% |
| | AL | B*07:02 | | Chr22 | | | Good | | |
| 4 | KPRPDVT | HLA- | 1 | Chr2 | ++ | 531/14 | G-Tex | 9 | 90%-100% |
| | NEL | B*07:02 | | | | | Good | | |
| 5 | KEFAFLE | HLA- | 1 | Chr8 | N/A | 400/8 | G-Tex | 11 | 80%-90% |
| | HSL | B*40:01 | | | | | Good | | |
| | | HLA- | | | | | | | |
| | | B*44:02 | | | | | | | |
| 6 | HELGFKV | HLA- | 1 | Chr12 | N/A | 387/7 | Gtex good - | 6 | 70%-90% |
| | VL | B*40:01 | | | | | high in | | |
| | | HLA- | | | | | intestine | | |
| | | B*44:02 | | | | | | | |
| | | HLA- | | | | | | | |
| | | B*18:01 | | | | | | | |
| 7 | LKPDHIQ | HLA- | 1 | Chr12 | N/A | 441/11 | Gtex good | 12 | 50-75% |
| | R | A*03:01 | | | | | | | |
| 8 | SLFGARP | HLA- | 1 | Chr15 | N/A | 553/15 | Gtex good - | 3 | 70%-90% |
| | GR | A*03:01 | | | | | consistent | | |
| | | HLA- | | | | | background | | |
| | | A*31:01 | | | | | expression | | |
| | | | | | | | levels | | |
| 9 | KTEVHGR | HLA- | 1 | Chr12 | N/A | 517/13 | Gtex good - | 6 | 70%-90% |
| | LK | A*03:01 | | | | | high in | | |
| | | | | | | | intestine | | |
| 10 | NLRPPTQ | HLA- | 1 | Clu-1 | + | 19 | | 4 | |
| | EL | B*07:02 | | | | | | | |
| 11 | RVSLPKL | HLA- | | | N/A | N/A | Not done | | N/A |
| | GYK | A*03:01 | | | | | | | |

### Example 2: Design and Production of Vectors

Based on the immunogenicity and prevalence analysis from Example 1, six peptides were selected for preparing vector constructs comprising the peptides (Table 7).

TABLE 7
Peptide candidates selected for preparing
vector constructs

| SEQ | SEQ | | |
|---|---|---|---|
| Identi- | ID | | HLA |
| fier | NO. | Sequence | Restriction |
| A1 | 1 | GVYDGREHTV | HLA-A*02:01 |
| A2 | 2 | KL VELEHTL | HLA-A*02:01 |
| A3 | 3 | SPSSASLAL | HLA-B*07:02 |
| A4 | 4 | KPRPDVTNEL | HLA-B*07:02 |
| A5 | 5 | KEFAFLEHSL | HLA-B*40:01 |
| | | | HLA-B*44:02 |
| A6 | 6 | HELGFKVVL | HLA-B*40:01 |
| | | | HLA-B*44:02 |
| | | | HLA-B*18:01 |

The epitope coding sequence for the minigenes were designed, human-codon sequence optimised and synthesised as a synthetic nucleotide sequence. Each minigene epitope coding sequence was then cloned separately into shuttle plasmids (pGeneMinigENTR4-monoene-vector) to create plasmids encoding the minigene epitope. These were inserted into a plasmid encoding the sequence of the well-characterised El-deleted replication-deficient Ad5 adenoviral vector. This plasmid encoding the partial Ad5 sequence and the minigene epitope was transfected into a HEK293-derived GMP-compliant cell line expressing El to allow required packaging of the Ad5-minigene epitope vector into the Ad5 viral particles.

### Example 3: Design and Production of Vectors

Based on the immunogenicity and prevalence analysis from Example 1, six peptides are selected for preparing vector constructs comprising the peptides (Table 8).

TABLE 8
Additional peptide candidates for
preparing vector constructs

| | SEQ ID NO. | Sequence | HLA Restriction |
|---|---|---|---|
| A7 | 7 | LKPDHIQR | HLA-A*03:01 |
| A8 | 8 | SLFGARPGR | HLA-A*03:01 |
| | | | HLA-A*31:01 |
| A9 | 9 | KTEVHGRLK | HLA-A*03:01 |
| A10 | 10 | NLRPPTQEL | HLA-B*07:02 |
| All | 11 | RVSLPKLGYK | HLA-A*03:01 |
| A12 | 12 | LRPPTQEL | HLA-C*07:02 |

The epitope coding sequence for the minigenes are designed, human-codon sequence optimised and synthesised as a synthetic nucleotide sequence. Each minigene epitope coding sequence are then cloned separately into shuttle plasmids (pGene-vector) to create plasmids encoding the minigene epitope. These are inserted into a plasmid encoding the sequence of the well-characterised El-deleted replication-deficient Ad5 adenoviral vector. This plasmid encoding the partial Ad5 sequence and the minigene epitope are transfected into a HEK293-derived GMP-compliant cell line expressing El to allow required packaging of the Ad5-minigene epitope vector into the Ad5 viral particles.

### Example 4: Design and Production of Vectors

Based on the immunogenicity and prevalence analysis from Example 1, six peptides are selected for preparing vector constructs comprising the peptides (Table 9).

TABLE 9
Additional peptide candidates for
preparing vector constructs

| SEQ | SEQ | | |
|---|---|---|---|
| Identi- | ID | | ULA |
| fier | NO. | Sequence | Restriction |
| A13 | 13 | RPASPRPAP | HLA-B*07:02 |
| A14 | 14 | IATKIALQM | HLA-C*03:04 |
| A15 | 15 | SLYQTIRLK | HLA-A*03:01 |
| A16 | 16 | KTPLHTLLK | HLA-A*03:01 |
| A17 | 17 | RLHSFTLRQK | HLA-A*03:01 |
| A18 | 18 | RVFTSSLKTK | HLA-A*03:01 |

MinigENTR4-monoeneRecombinant AAV encoding a peptide from Table 9 are generated by transfecting HEK 293 cells with three plasmids: (1) AAV-ITR plasmid containing the peptide of interest [AAV-ITR-peptide], (2) an adenovirus helper plasmid that encodes the E2A, E4 and VA adenoviral proteins that are required for AAV replication and (3) a helper plasmid encoding the rep and cap genes of AAV, required for packaging the AAV-ITR-minigene within the AAV viral particles.

### Example 5: Proof-of-Concept Phase I/IIa Clinical Trial Protocol for Administration of the Composition for Treating a Subject with Resectable Esophageal Cancer

This is a Phase I/II clinical trial for studying a vaccine comprising as a treatment for a surgically resectable esophageal cancer.

Target Population: First line treatment for patients newly identified with surgically resectable oesophageal adenocarcinoma scheduled for FLOT chemotherapy.

Expected Clinical Outcome: The administration of the drug products after removal of the primary tumor by surgical intervention, prevents formation of secondary or metastatic tumors by eliciting an immune response in the subject.

Phase 1—Safety Lead In—Enrol 3-5 patients to confirm the expected safety of The drug product DP1, DP2, and DP3 delivered in a prime/boost regimen following adjuvant and neoadjuvant chemotherapy. Safety evaluation after first 3 patients have one month post boost dose blood samples are analyzed.

Phase II: 60 subjects are enrolled for the trial (3:1 treated vs. placebo design).

Target population: Subjects with surgically resectable oesophageal adenocarcinoma scheduled for FLOT chemotherapy.

The primary objective of the trial is to evaluate safety and tolerability by determining the endpoints for incidence of serious adverse events (SAEs), incidence of Grade 3 or higher SAEs, incidence of immune-related adverse events (irAE), progression to surgical resection, and progression to neoadjuvant chemotherapy.

The secondary objective of the trial is to evaluate immunogenicity by longitudinal blood sampling to understand the kinetics of the vaccine-induced specific T cell response, including the impact of the booster dose. The tests include immunological tests for evidence of specific priming and expansion of naive and memory CD8+ and CD4+ T cell responses induced by administration of the vaccines.

The tertiary endpoints of the trial to evaluate tumoral immune response to the compositions by immunohistochemical techniques, and ex vivo expansion of composition induced T cells from tumor tissue single cell suspensions to enable detailed functional analyses, and RNA expression profiling.

The drug products 1, 2, and 3 each comprise (comprises two distinct therapeutic compositions each matched to a patient's HLA type. Patients receive between one and three drug products (DP1, DP2, DP3, DP1+DP2, DP1+DP3, DP2+DP3, or DP1+DP2+DP3) dependent on their HLA type. Each therapeutic composition comprises 1×10¹⁰ v.p. (viral particles), giving a total maximum dose of between 2 and 6×10¹⁰ v.p. per patient (dependent on HLA matching). Each drug product is filled in 2 ml vials, labeled, stored, and supplied to sites at less than −65° C. to be thawed prior to use. The placebo group patients receive sterile saline in equal volume.

Other interventions: In addition to DP1 and/or DP2, and/or DP3 (or placebo), subjects (patients identified with resectable oesophageal cancer) also receive standard FLOT chemotherapy and surgical intervention.

Treatment Duration: Prime and Boost dosing strategy—first dose of drug product(s) (prime dose) is administered 4 weeks after completion neoadjuvant FLOT chemotherapy course (4 cycles). The second dose of the drug product(s) (boost dose) is administered 2 weeks prior to adjuvant FLOT chemotherapy course (4 cycles) or at 1 week following post surgical recovery if further FLOT cycles are not undertaken.

Regimen: After initial diagnosis of esophageal carcinoma, a subject undergoes a diagnostic biopsy to obtain a tissue sample. HLA screening is performed at the diagnostic biopsy. Only subjects that have a match to at least one of the HLA types in the epitopes selected are included in the trial. Based on HLA screening, the prime dose composition for each subject is determined (DP1, DP2, DP3, DP1+DP2, DP1+DP3, DP2+DP3, or DP1+DP2+DP3). Subjects are treated with FLOT neoadjuvant chemotherapy 2 weeks after biopsy screening. FLOT neoadjuvant chemotherapy is four cycles, completed at one cycle per week. After chemotherapy, blood from both treated and placebo groups is collected for analysis for baseline establishment. Approximately 4 weeks after FLOT neoadjuvant chemotherapy (i.e., approximately 10 weeks after diagnostic biopsy), the first dose (prime dose comprising the drug product(s) or the first placebo dose) is administered to the subject via intravenous injection. Approximately 2 weeks after administration of the first dose, a blood sample is collected for analysis. Peripheral blood mononuclear cells (PBMCs) from blood is tested by ICS and/or tetramer/dextramer staining to detect vaccine specific T cells. Approximately 16 weeks after the first dose administration, the subject undergoes presurgical examination. Samples from the subject are collected for immunostaining and TCR sequencing. Following pre-surgical examination, the subject undergoes surgical resection to remove the primary tumor. Following surgical resection, the second dose (boost dose comprising the drug product(s) or the second placebo dose) is administered to the subject via intravenous injection. The second dose is administered either: (1) approximately 2 weeks prior to a second round of FLOT chemotherapy, or (2) approximately 1 week following post-surgical recovery if further FLOT cycles are not undertaken. The second round of FLOT chemotherapy is four cycles, completed at one cycle per week. Following the boost dose and second round of FLOT chemotherapy, the subject is monitored and the endpoints of the trial are assessed. Blood from subject is collected for further analysis via tetramer/Dextramer staining, T cell ICS, whole blood IFN-Y analysis, ELISA, ctDNA, epitope and HLA screening and immune response measurement using Ad5nAbs. One month after the boost dose, blood from subject is collected for further analysis via tetramer/Dextramer staining, T cell ICS, whole blood IFN-Y analysis, ELISA, ctDNA, epitope and HLA screening and immune response measurement using Ad5nAbs. Subject is monitored and tested for presence of formation of secondary or metastatic tumors on periodic interval.

Immune monitoring can be performed before, during, and/or after drug product administration. Such monitoring can inform safety and efficacy among other parameters.

### Example 6. Clinical Trial Design and Timepoints

The Example describes details of a clinical trial design to treat oesophageal adenocarcinoma.

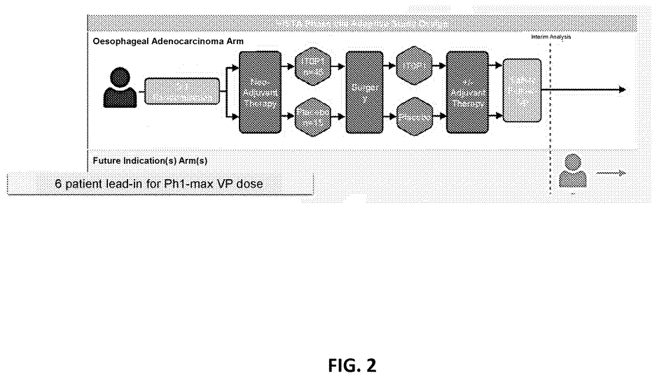

FIG. 2 provides an overview of the trial design. Patients are enrolled in an oesophageal adenocarcinoma arm of the study with a 3:1 randomisation. The patients receive neoadjuvant therapy and are split into two groups. One group of patients (n=45) receive iTOP-mediated delivery of the prime dose and a second group (n=15) receive placebo. The patients then receive surgical resection. Following surgery, patients in the ITOP group will receive a second iTOP-mediated delivery vaccine dose (e.g., boost dose) and patients in the placebo group will receive a second placebo dose. If necessary, patients will receive a second round of neoadjuvant therapy. Patients will receive safety check-in and follow-up accordingly.

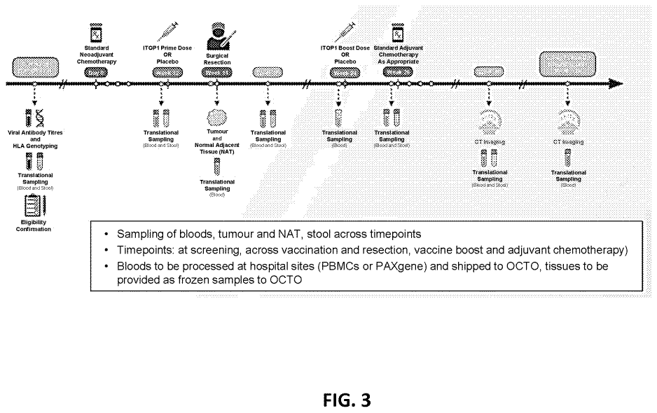

FIG. 3 provides a comprehensive overview of timepoints throughout the clinical trial. Timepoints include screening, across prime vaccination, resection, and boost vaccination, and adjuvant chemotherapy. Sampling of blood, tumor, normal adjacent tissue (NAT), and stool will be conducted across timepoints. Blood will be processed at hospital sites and tissues will be provided as frozen samples for analysis.

Patient screening will be conduced from 28 days to 1 day prior to study initiation (Day 0). Screening will include determination of viral antibody titres and HLA genotyping, translational sampling of blood and stool, and confirmation of eligibility. At Day 0, patients will undergo standard neoadjuvant chemotherapy. Patients will be split into two groups following chemotherapy. At Week 12, patients will receive either ITOP1 prime dose or placeb, and sampling of blood and stool will be conducted. At Week 14, patients will undergo surgical resection of the tumor. Tumor tissue and normal adjacent tissue will be collected. Blood sampling will also be performed. At Week 16, sampling of blood and stool will be conducted. At Week 24, a second ITOP vaccine dose (boost dose) or placebo will be administered to patients, and blood sampling will be conducted. At Week 26, patients may receive another round of standard adjuvant chemotherapy if appropriate. Blood and stool sampling will also be conducted at week 26. At Week 40, patients will undergo CT imaging, and blood and stool samples will be collected. Following Week 40, patients will receive safety follow-ups every 6 months until Week 116. Safety follow-up visits will include CT imaging and collection of blood samples.

The primary objective of the clinical trial will be to characterize the safety and tolerability of ITOP1 vaccine. This will include measures of adverse events and progression to surgery.

Secondary objectives of the clinical trial will include assessing the preliminary clinical efficacy and immunogenitcity of ITOP1 vaccine. This will include (i) recurrence free survival and overall survival, (ii) tumor and peripheral immune response to ITOP1 vaccine, and (iii) kinetics of ITOP1 induced specific T cell response, including the impact of the boost (e.g., second) dose.

Other exploratory endpoints will include: (i) presence of ITOP1 epitopes in tumors via immunopeptidomics (PRMS), (ii) tracking levels and kinetics of Ad5 neutralizing antibodies via hexon ELISA, (iii) examining ctDNA levels as an exploratory early endpoint, (iv) evaluating pre-vaccination immune responses, and (v) characterizing the tumor microenvironment. Whole blood monitoring may also be conducted.

### Example 7. Heterologoeous Prime-Boost Vaccinations

This Example provides proposed experiments evaluating the effects of different prime-boost vaccination schedules.

Prime vaccine doses are either viral (Ch adenoviral, ChAd) or mRNA (BNT). Boost vaccine doses are either viral (ChAd), mRNA (BNT or m1273), or peptide-based (NVX). Experiments are conducted to assess the effects of homologous vaccine administration, in which the same type of vaccine is administered as the prime and boost doses, and heterologous vaccine administration, in which different types of vaccines are administered as the prime and boost doses. Results are measured as spot-forming cells (SFC) per million of PBMCs, as identified via ELISpot assay. This assay allows for analysis of the aggregated T cell response per vaccine schedule condition and shows levels of cytokine secreting cells from a cell population. SFCs are quanitified after prime dose administration and after boost dose administration to examine the effects of different prime dose modalities, boost dose modalities, and prime/boost dose modality combinations.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein can be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1.-121. (canceled)

122. A method of treating a cancer in a human subject in need thereof, comprising administering to the human subject a pharmaceutical composition comprising:
(i) a polypeptide that is from 9 to 11 amino acids in length, wherein the polypeptide comprises a single cancer-specific CD8+ T cell epitope;
(ii) a recombinant polynucleotide comprising a sequence encoding a polypeptide that is from 9 to 11 amino acids in length, wherein the polypeptide comprises a single cancer-specific CD8+ T cell epitope;
(iii) a vector comprising (ii);
(iv) antigen presenting cells (APCs) comprising (i), (ii), or (iii); or
(v) T cells stimulated with APCs comprising (i), (ii), or (iii);
wherein:
(a) the human subject expresses an MHC encoded by an HLA-B*35:03 allele or an HLA-B*07:02 allele, and the single cancer-specific CD8+ T cell epitope has the amino acid sequence SPSSASLAL or KPRPDVTNEL;
(b) the human subject expresses an MHC encoded by an HLA-A*02:01 allele and the single cancer-specific CD8+ T cell epitope has the amino acid sequence GVYDGREHTV or KL VELEHTL;
(c) the human subject expresses an MHC encoded by an HLA-B*40:01 allele, an HLA-B*40:02 or an HLA-B*44:02 allele, and the single cancer-specific CD8+ T cell epitope has the amino acid sequence KEFAFLEHSL or HELGFKVVL;
(d) the human subject expresses an MHC encoded by an HLA-B*18:01 allele and the single cancer-specific CD8+ T cell epitope has the amino acid sequence HELGFKVVL; and/or
(e) the human subject expresses an MHC encoded by an HLA-A*03:01 allele and the single cancer-specific CD8+ T cell epitope has the amino acid sequence RVSLPKLGYK.

123. The method of claim 122, wherein the pharmaceutical composition comprises a vector comprising (ii), and wherein the pharmaceutical composition does not comprise one or more nucleic acid sequences encoding more than two single cancer-specific CD8+ T cell epitopes.

124. The method of claim 123, wherein the vector is a viral vector, and wherein the viral vector is an adenoviral vector.

125. The method of claim 124, wherein the adenoviral vector is a human serotype 5 (AdHu5) vector.

126. The method of claim 125, wherein the cancer is an esophageal cancer.

127. The method of claim 125, wherein the AdHu5 vector comprises (i) a deletion or a functional deletion in the El gene at an El gene locus, (ii) a deletion or a functional deletion in the E3 gene at an E3 gene locus, or (iii) a deletion or a functional deletion in the El gene at an El gene locus and a deletion or a functional deletion in the E3 gene at an E3 gene locus.

128. The method of claim 122, wherein the cancer is an esophageal cancer.

129. The method of claim 122, wherein the polypeptide circumvents normal antigen processing requirements for presentation of the single cancer-specific CD8+ T cell epitope by the MHC expressed by the human subject.

130. The method of claim 122, wherein administration of the pharmaceutical composition to the human subject induces a T cell response specific to the single cancer-specific CD8+ T cell epitope, and wherein the T cell response specific to the single cancer-specific CD8+ T cell epitope is an inflating memory CD8+ T cell response.

131. The method of claim 130, wherein the inflating memory CD8+ T cell response is characterized by production of CD8+/CD44+ T cells that are:
(i) CX3CR1+ and/or KLRG-1+, and
(ii) CD62L−, CD27-, and/or CD127.

132. The method of claim 130, wherein, after administration of the pharmaceutical composition to the human subject, the inflating memory CD8+ T cell response controls tumor growth in the human subject for at least 10 days.

133. The method of claim 122, wherein the method comprises administering a single dose of the pharmaceutical composition.

134. The method of claim 122, wherein the method comprises administering a first dose of the pharmaceutical composition and administering a second dose of the pharmaceutical composition.

135. The method of claim 134, wherein the second dose is administered at least 4 weeks after the first dose.

136. The method of claim 134, wherein the first dose has the same cancer-specific CD8+ T cell epitope as the second dose.

137. The method of claim 122, wherein the pharmaceutical composition comprises
(i) a first adenoviral vector comprising a sequence encoding the single cancer-specific CD8+ T cell epitope having the sequence SPSSASLAL, and
(ii) a second adenoviral vector comprising a sequence encoding the single cancer-specific CD8+ T cell epitope having the sequence KPRPDVTNEL, and
wherein the human subject in need thereof expresses an MHC encoded by an HLA-B*07:02 allele or an HLA-B*35:03 allele.

138. The method of claim 122, wherein the pharmaceutical composition comprises
(i) a first adenoviral vector comprising a sequence encoding the single cancer-specific CD8+ T cell epitope having the sequence GVYDGREHTV, and
(ii) a second adenoviral vector comprising a sequence encoding the single cancer-specific CD8+ T cell epitope having the sequence KLVELEHTL, and
wherein the human subject in need thereof expresses an MHC encoded by an HLA-A*02:01 allele.

139. The method of claim 122, wherein the pharmaceutical composition comprises
(i) a first adenoviral vector comprising a sequence encoding the single cancer-specific CD8+ T cell epitope having the sequence KEFAFLEHSL, and
(ii) a second adenoviral vector comprising a sequence encoding the single cancer-specific CD8+ T cell epitope having the sequence HELGFKVVL, and
wherein the human subject in need thereof expresses an MHC encoded by an HLA-B*40:01 allele, an HLA-B*40:02 allele, an HLA-B*44:02 allele, or an HLA-B* 18:01 allele.

140. The method of claim 122, wherein the pharmaceutical composition comprises
(i) a first adenoviral vector comprising a sequence encoding the single cancer-specific CD8+ T cell epitope having the sequence RVSLPKLGYK, and
(ii) a second adenoviral vector comprising a sequence encoding the single cancer-specific CD8+ T cell epitope having the sequence RVSLPKLGYK, and
wherein the human subject in need thereof expresses an MHC encoded by an HLA-A*03:01 allele.

141. A pharmaceutical composition comprising:
(i) a polypeptide that is from 9 to 11 amino acids in length, wherein the polypeptide comprises a single cancer-specific CD8+ T cell epitope;
(ii) a recombinant polynucleotide comprising a sequence encoding a polypeptide that is from 9 to 11 amino acids in length, wherein the polypeptide comprises a single cancer-specific CD8+ T cell epitope;
(iii) a vector comprising (ii);
(iv) antigen presenting cells (APCs) comprising (i), (ii), or (iii); or
(v) T cells stimulated with APCs comprising (i), (ii), or (iii);
wherein:
(a) the cancer-specific CD8+ T cell epitope has the amino acid sequence SPSSASLAL or KPRPDVTNEL;
(b) the cancer-specific CD8+ T cell epitope has the amino acid sequence GVYDGREHTV or KLVELEHTL;
(c) the cancer-specific CD8+ T cell epitope has the amino acid sequence KEFAFLEHSL or HELGFKVVL;
(d) the cancer-specific CD8+ T cell epitope has the amino acid sequence HELGFKVVL; or
(e) the cancer-specific CD8+ T cell epitope has the amino acid sequence RVSLPKLGYK.
